SEQUENCE LISTING (6 sequences)

SEQ ID NO: 1 - protein, 24 aa, Artificial
  Modified Insulin A chain
  MISC_FEATURE (1)..(1): Gly is absent or Gly
  MISC_FEATURE (2)..(2): Pro is absent or Pro
  MISC_FEATURE (3)..(3): Pro is absent or Pro
  MISC_FEATURE (11)..(11): Thr is Thr or His
  MISC_FEATURE (15)..(15): Ser is Ser, Asp or Glu
  MISC_FEATURE (16)..(16): Leu is Leu, Thr, Asn, Asp, Gln, His, Lys, Gly, Arg, Pro, Ser or Glu
  MISC_FEATURE (17)..(17): Tyr is Tyr, Thr, Asn, Asp, Gln, His, Lys, Gly, Arg, Pro, Ser or Glu
  MISC_FEATURE (18)..(18): Gln is Gln, Asp or Glu
  MISC_FEATURE (21)..(21): Asn is Asn, Lys or Gln
  MISC_FEATURE (24)..(24): Asn is Asn or Gln
GPPGIVEQCCTSICSLYQLENYCN

SEQ ID NO: 2 - protein, 35 aa, Artificial
  Modified Insulin B chain
  MISC_FEATURE (1)..(1): Gly is absent or Gly
  MISC_FEATURE (2)..(2): Pro is absent or Pro
  MISC_FEATURE (3)..(3): Pro is absent or Pro
  MISC_FEATURE (4)..(4): Glu is absent Phe or Glu
  MISC_FEATURE (5)..(5): Val is absent or Val
  MISC_FEATURE (6)..(6): Asn is absent, Asn or Gln
  MISC_FEATURE (7)..(7): Gln is Gln or Glu
  MISC_FEATURE (13)..(13): His is His, Asp, Pro or Glu
  MISC_FEATURE (19)..(19): Tyr is Tyr, Asp, Gln, His, Arg or Glu
  MISC_FEATURE (27)..(27): Phe is Phe or His
  MISC_FEATURE (28)..(28): Phe is Phe, Asn or His
  MISC_FEATURE (29)..(29): Tyr is absent, Tyr, His, Thr, Gly or Asp
  MISC_FEATURE (30)..(30): Thr is absent, Thr, Asn, Asp, Gln, His, Lys, Gly, Arg, Pro, Ser or Glu
  MISC_FEATURE (31)..(31): Pro is absent, Pro, His, Gly or Asp
  MISC_FEATURE (32)..(32): Lys is absent, Lys, Arg or Gln
  MISC_FEATURE (33)..(33): Thr is absent or Thr
  MISC_FEATURE (34)..(34): Leu is absent or Leu
  MISC_FEATURE (35)..(35): Glu is absent or Glu
GPPFVNEHLCGSHLVEALYLVCGERGFFYTPKTLE

SEQ ID NO: 3 - protein, 21 aa, Artificial
  Modified insulin A chain
  MISC_FEATURE (8)..(8): Thr is Thr or His
  MISC_FEATURE (12)..(12): Ser is Ser, Asp or Glu
  MISC_FEATURE (13)..(13): Leu is Leu, Thr, Asn, Asp, Gln, His, Lys, Gly, Arg, Pro, Ser or Glu
  MISC_FEATURE (14)..(14): Thr is Thr, Asn, Asp, Gln, His, Lys, Gly, Arg, Pro, Ser or Glu
  MISC_FEATURE (15)..(15): Gln is Gln, Asp or Glu
  MISC_FEATURE (18)..(18): Asn is Asn, Lys or Gln
  MISC_FEATURE (21)..(21): Asn is Asn or Gln
GIVEQCCTSICSLTQLENYCN

SEQ ID NO: 4 - protein, 30 aa, Artificial
  Modified insulin B chain
  MISC_FEATURE (1)..(1): Xaa is Phe or Glu
  MISC_FEATURE (3)..(3): Asn is Asn or Gln
  MISC_FEATURE (4)..(4): Gln is Gln or Glu
  MISC_FEATURE (10)..(10): His is His, Asp, Pro or Glu
  MISC_FEATURE (16)..(16): Tyr is Tyr, Asp, Gln, His, Arg or Glu
  MISC_FEATURE (24)..(24): Phe is Phe or His
  MISC_FEATURE (26)..(26): Tyr is absent, Tyr, His, Thr, Gly or Asp
  MISC_FEATURE (27)..(27): Thr is absent, Thr, Asn, Asp, Gln, His, Lys, Gly, Arg, Pro, Ser or Glu
  MISC_FEATURE (28)..(28): Pro is absent, Pro, His, Gly or Asp
  MISC_FEATURE (29)..(29): Lys is absent, Lys, Arg or Gln
  MISC_FEATURE (30)..(30): Thr is absent or Thr
FVNEHLCGSHLVEALYLVCGERGFHYTPKT

SEQ ID NO: 5 - protein, 21 aa, Artificial
  Modified A chain
  DISULFID (6)..(6)
  DISULFID (7)..(7)
  DISULFID (10)..(10)
  DISULFID (11)..(11)
  DISULFID (20)..(20)
GIVEQCCTSCCSLEQLENYCN

SEQ ID NO: 6 - protein, 29 aa, Artificial
  Modified B chain
  DISULFID (4)..(4)
  DISULFID (7)..(7)
  DISULFID (19)..(19)
  Lys (29)..(29): Lys is Lys-(N(epsilon)-Octadecanedioyl-gGlu- OEG-OEG)
FVNCHLCGSHLVEALYLVCGERGFHYTPK

(19) United States
(12) Patent Application Publication
(10) Pub. No.: US 2018/0000742 A1
(43) Pub. Date: Jan. 4, 2018

(54) Pharmaceutical Composition for Oral Insulin Administration Comprising a Tablet Core and a Polyvinyl Alcohol Coating

(71) Applicant: Novo Nordisk A/S, Bagsvaerd (DK)

(72) Inventors: Lars Hovgaard, Farum (DK); Hanne Refsgaard, Holte (DK); Thomas Boerglum Kjeldsen, Virum (DK); Peter Madsen, Bagsvaerd (DK); Giustino Di Pretoro, Antwerp (BE)

(21) Appl. No.: 15/546,299

(22) PCT Filed: Jan. 29, 2015
(86) PCT No.: PCT/EP2015/051813

§ 371 Date: Jul. 26, 2017

(51) Int. Cl. A61K 9/50; A61K 9/00; A61K 9/20; A61K 38/28

(52) U.S. Cl. CPC A61K 9/5026; A61K 38/28; A61K 9/5089; A61K 9/0053; A61K 9/2013; A61K 9/2018

(57) ABSTRACT

The present invention relates to a solid oral insulin composition comprising a salt of capric acid which enhances the bioavailability and/or the absorption of said acylated insulin in combination with a polyvinyl alcohol coating, which is soluble in aqueous media independent of pH.

### TECHNICAL FIELD

The present invention relates to a solid oral insulin composition consisting of a tablet core and a polyvinyl alcohol coating, wherein a tablet core comprises a salt of capric acid.

### BACKGROUND

Many pathological states due to deficiencies in or complete failure of the production of certain macromolecules (e.g. proteins and peptides) are treated with an invasive and inconvenient parenteral administration of therapeutic macromolecules. One example hereof is the administration of insulin in the treatment of insulin dependent patients, who are in need of one or more daily doses of insulin. The oral route is desirable for administration due to its non-invasive nature and has a great potential to decrease the patient's discomfort related to drug administration and to increased drug compliance. However, several barriers exist; such as the enzymatic degradation in the gastrointestinal (GI) tract, drug efflux pumps, insufficient and variable absorption from the intestinal mucosa, as well as first pass metabolism in the liver. Thus until now no products for oral delivery of insulins are found to be marketed.

One example of such macromolecules is human insulin which is degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

The pH of the gastrointestinal tract varies from quite acidic pH 1-3 in the stomach through pH 5.5 in the duodenum to pH 7.5 in the ileum. Then entering the colon pH drops to pH 5 before again increasing to pH 7 in the rectum (Dan Med Bull. 1999 June; 46(3):183-96. Intraluminal pH of the human gastrointestinal tract. Fallingborg J.)

Provision of a solid oral dosage form which would facilitate the administration of insulin is desirable. The advantages of solid oral dosage forms over other dosage forms include ease of manufacture and administration. There may also be advantages relating to convenience of administration increasing patient compliance.

US2008260820 discloses an oral dosage formulation comprising protease-resistant polypeptides which may contain an intestinal absorption enhancing agent including surfactants (e.g., sodium dodecyl sulfate, bile salts, palmitoylcamitine, and sodium salts of fatty acids); and toxins (e.g., zonula occludens toxin).

US2006/018874 and US2006/019874 disclose tablets containing sodium caprate and IN105 insulin. WO2010/032140 and WO2011/084618 disclose an insulin formulation comprising sodium caprate. WO2011/103920 discloses pharmaceutical compositions comprising a tablet core consisting of active pharmaceutical ingredient such as insulin, a penetration promoter, a bioavailability promoting agent, such as an enzyme inhibitor and a polymeric coating. WO0104195 A1 discloses polyvinyl alcohol coating.

The oral route of administration is rather complex and a need for establishment of an acceptable pharmaceutical composition suitable for the treatment of patients, with an effective bioavailability of insulins, is existent.

### SUMMARY

The present invention provides a pharmaceutical composition which is effective in providing therapeutically effective blood levels of acylated insulins in a subject, when administered to said subject's gastrointestinal tract (e.g. per os (oral administration) of a pharmaceutical composition according to the present invention).

One embodiment of the present invention concerns a pharmaceutical composition consisting of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms.

One embodiment of the present invention concerns a pharmaceutical composition consisting of one or more tablet core and a polyvinyl alcohol coating, wherein said one or more tablet core comprises a salt of a medium-chain fatty acid and acylated insulin, wherein said acylated insulin comprises one or more additional disulfide bonds.

One embodiment of the present invention concerns a pharmaceutical composition consisting of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising one or more tablets, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising up to three tablets, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising two tablet, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising 150 to 250 tablets, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting one or more tablets, wherein each tablet consists of one or more uncoated tablet core, wherein said one or more un coated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting up to three tablets, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting two tablets, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting between about 150 and about 250 tablets, wherein each tablet consists of one or more uncoated tablet core, wherein said tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting between about 140 and about 250 tablets weighing between about 3.0 and about 5.0 mg, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting between about 14 and about 470 tablets weighing between about 1.5 and about 50 mg, wherein each tablet consists of one or more uncoated tablet core, wherein said one or more uncoated tablet core comprises a salt of a medium-chain fatty acid and an acylated insulin, wherein said acylated insulin is a protease stabilised insulin comprising a linker and a fatty acid or fatty diacid chain having 14-22 carbon atoms and optionally comprising one or more additional disulfide bonds.

### BRIEF DESCRIPTION OF THE DRAWINGS

FIG. 1 shows the dissolution rate of compositions according to the present invention (tablet core+OPADRY®II—Yellow coating from Colorcon® (as sold in 2013)) and a pharmaceutical composition wherein no coating is applied on the tablet core.

FIG. 2A illustrates bioavailability of a pharmaceutical composition according to the present invention (tablet core+OPADRY®II—Yellow coating from Colorcon® (as sold in 2013)) compared to a tablet core with a sub coat of OPADRY®II—Yellow below an Acryl-EZE® 93O coating from Colorcon® (as sold in 2013).

FIG. 2B illustrates Tmax of a pharmaceutical composition according to the present invention (tablet core+OPADRY®II—Yellow coating from Colorcon® (as sold in 2013)) compared to a tablet core with a sub coat of OPADRY®II—Yellow below an Acryl-EZE® 93O coating from Colorcon® (as sold in 2013).

FIG. 3 shows the PK profiles for this acylated insulin in tablet cores with OPADRY®II—Yellow from Colorcon® (as sold in 2013) sub coat and a functional coat of Eudragit® FS30D from Evonik Industries (as sold in 2013), squares show the PK profile for tablets tested at time 0 and circles show the PK profile for tablets tested after 12 or more weeks storage at 5° C.

FIG. 4 shows in-vitro dissolution rate of A14E, B25H, B29K(N^{ε}Octade-canedioyl-γGlu-OEG-OEG), desB30 human insulin, (triangles) and sodium caprate (circles) from size 000 porcine gelatin capsules filled with mini-tablets (black lines) and monoliths (grey lines). Data reported as mean (n=3)±SD.

FIG. 5 shows in-vitro dissolution rate of A14E, B25H, B29K(N^{ε}Octade-canedioyl-γGlu-OEG-OEG), desB30 human insulin (triangles) and sodium caprate (circles) from size 000 porcine gelatin capsules containing Opadry-II coated mini-tablets (black lines) and monoliths (dark grey lines). Data reported as mean (n=3)±SD.

FIG. 6 shows in-vitro dissolution rate of A14E, B25H, B29K(ArOctadecane-dioyl-γGlu-OEG-OEG), desB30 human insulin (triangles) and sodium caprate (circles) from Opadry-II coated mini-tablets compressed in a monolith. Data reported as mean (n=3)±SD.

FIG. 7 shows in-vitro dissolution rate of A14E, B25H, B29K(ArOctadecane-dioyl-γGlu-OEG-OEG), desB30 human insulin from un-coated mini-tablets without capsule (black dotted line, triangles) or filled into size 00 capsules: porcine gelatin (black line, circles), HPMC (grey dotted line, triangles), Pullulan (grey line, squares) and fish gelatin (black line, squares). Data are reported as mean (n=3)±SD.

FIG. 8 shows in-vitro dissolution rate from un-coated mini-tablets filled in size 000 porcine gelatin capsules of: 1) acylated insulin A (A14E, B25H, B29K(N^{ε}Octade-canedioyl-γGlu-OEG-OEG), desB30 human insulin) (black line, triangles); 2) acylated insulin B (A14E, B25H, desB27, B29K(N-(eps)-(octadecandioyl-gGlu-2×OEG), desB30 human insulin) (black line, squares) and 3) sodium caprate (grey line, circles). Data are reported as mean (n=3)±SD.

FIG. 9 shows in-vitro dissolution rate of A14E, B16H, B25H, B29K(N-(eps)-(eicosanedioyl-gGlu-2×OEG), desB30 human insulin (triangles) and sodium caprate (circles) from size 000 porcine gelatin capsules containing un-coated mini-tablets. Data are reported as mean (n=3)±SD.

FIG. 10 shows in-vitro dissolution rate of A14E, B25H, desB27, B29K(N-(eps)-(octadecandioyl-gGlu), desB30 human insulin (triangles) and sodium caprate (circles) from size 000 porcine gelatin capsules containing un-coated mini-tablets. Data are reported as mean (n=3)±SD.

FIG. 11 shows in-vitro dissolution rate of A14E, B25H, desB27, B29K(N-(eps)-(octadecandioyl-gGlu-2×OEG), desB30 human insulin (triangles, black line) and sodium caprate (squares, grey line) from size 000 porcine gelatin capsules containing un-4.0 mm coated mini-tablets. Data are reported as mean (n=3)±SD

### DESCRIPTION

The present invention provides a pharmaceutical composition which is effective in providing therapeutically effective blood levels of acylated insulins in a subject, when administered to said subject's gastrointestinal tract (e.g. per os (oral administration) of a pharmaceutical composition according to the present invention).

One embodiment of the present invention concerns a pharmaceutical composition consisting of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms.

One embodiment of the present invention concerns a pharmaceutical composition consisting of one or more tablet core and a polyvinyl alcohol coating, wherein said one or more tablet core comprises a salt of a medium-chain fatty acid and acylated insulin, wherein said acylated insulin comprises one or more additional disulfide bonds.

One embodiment of the present invention concerns a pharmaceutical composition consisting of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising one or more tablets, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising up to three tablets, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising two tablet, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising 150 to 250 tablets, wherein each tablet consists of one or more tablet core and a polyvinyl alcohol coating, wherein said one or more tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting one or more tablets, wherein each tablet consists of one or more uncoated tablet core, wherein said one or more un coated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting up to three tablets, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting two tablets, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting between about 150 and about 250 tablets, wherein each tablet consists of one or more uncoated tablet core, wherein said tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting between about 150 and about 250 tablets weighing between about 3.0 and about 5.0 mg, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

One embodiment of the present invention concerns a pharmaceutical composition comprising multiparticulate system consisting up to about 300 tablets weighing between about 1.5 and about 50 mg, wherein each tablet consists of one or more uncoated tablet core, wherein said one or more uncoated tablet core comprises a salt of a medium-chain fatty acid and an acylated insulin, wherein said acylated insulin is a protease stablised insulin comprising a linker and a fatty acid or fatty diacid chain having 14-22 carbon atoms and optionally comprising one or more additional disulfide bonds.

It was surprisingly found that a pharmaceutical composition according to the embodiments of the present invention are suitable for administration of said acylated insulins to the GI tract (e.g. per os (oral administration)).

It was surprisingly found that the combination of the tablet core and polyvinyl alcohol coating according to the present invention and the oral bioavailability and PK/PD profile for said acylated insulins comprised in the tablet core of the pharmaceutical compositions according to the embodiments result in attractive overall PK/PD profiles for insulins for administering said acylated insulins to the GI tract (e.g. per os (oral administration)).

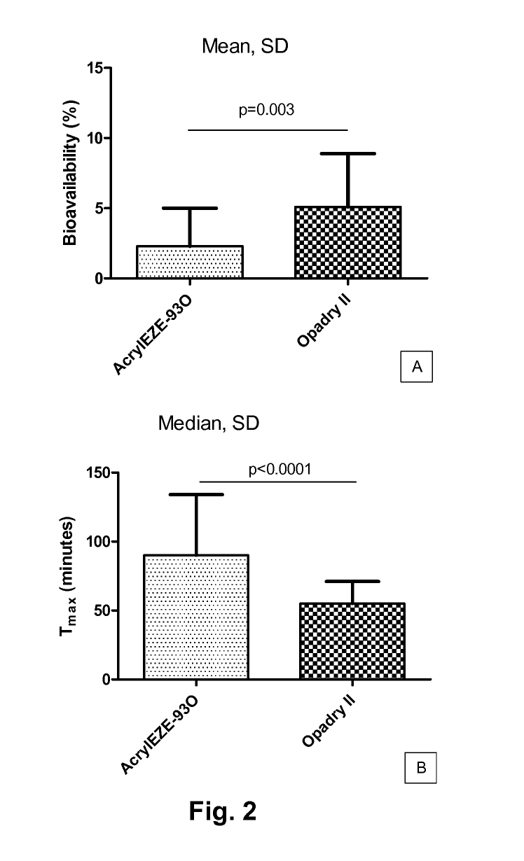

It was surprisingly found that a pharmaceutical composition according to the present invention comprising tablet core and a polyvinyl alcohol (polymer) coating (such as Opadry® II from Colorcon® (as sold in 2013)) presented a stable PK (see table 1) and bioavailability profile for said acylated insulin in Beagle dogs (see FIGS. 2A and 2B).

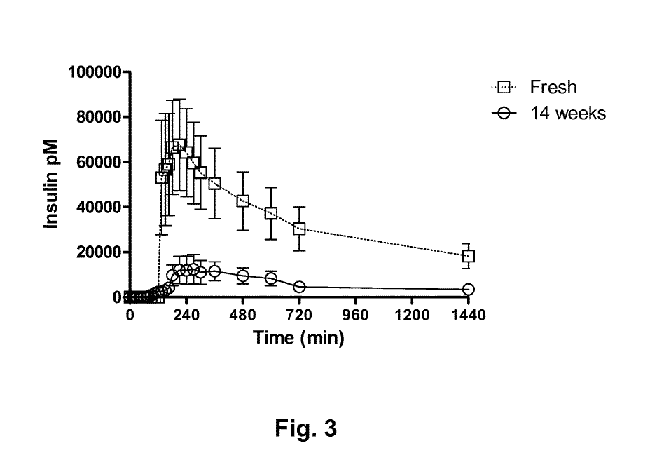

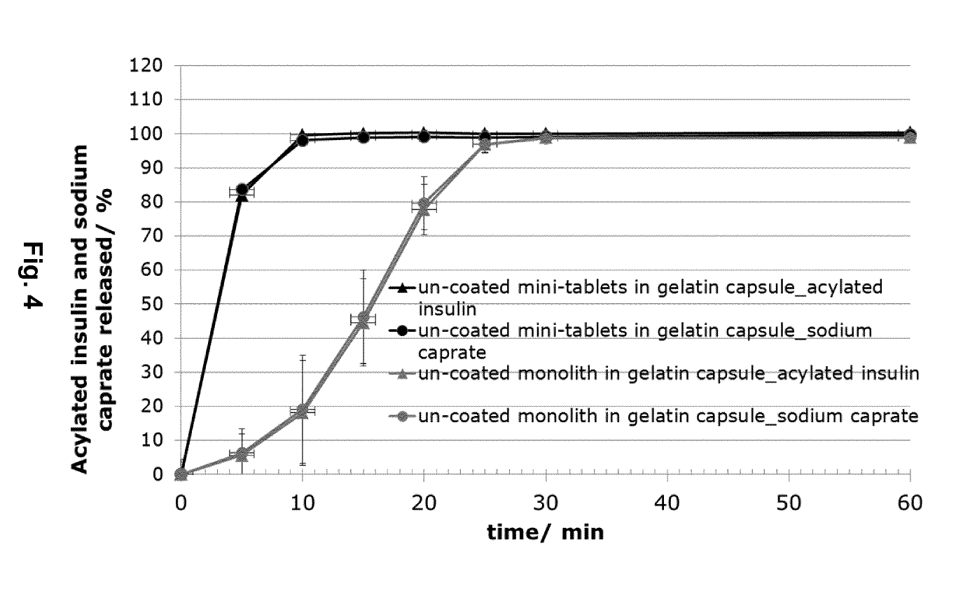

It was surprisingly found that the composition according to the present invention is more effective for increasing bioavailability and decreasing Tmax for said acylated insulin compared to composition wherein the tablet core according to the present invention was coated by an enteric coating such as Acryl-EZE®930 from Colorcon® (as sold in 2013) (see FIGS. 2A, 2B, 3A and 3B) or Eudragit® FS30D from Evonik Industries (as sold in 2013), see FIG. 4).

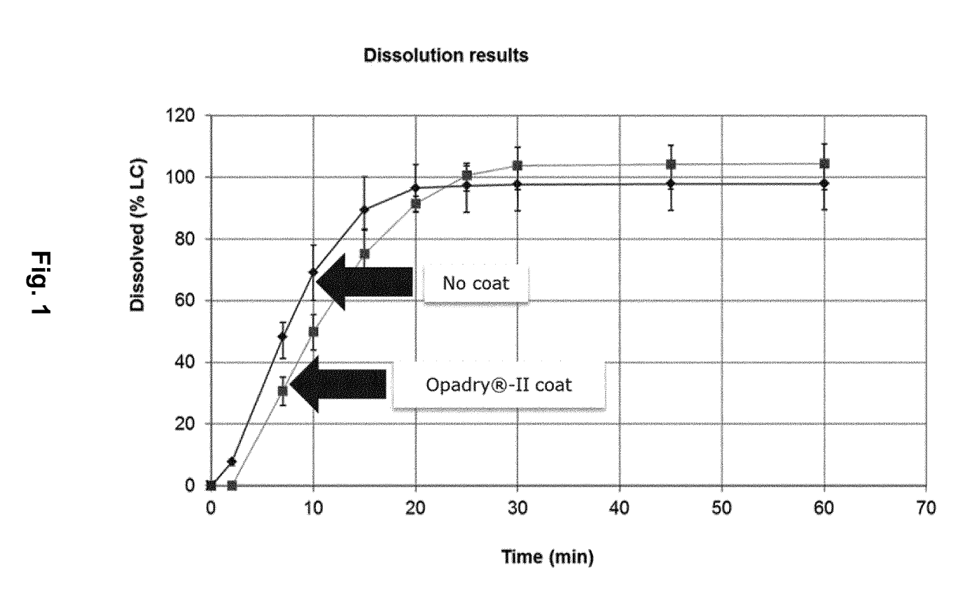

It was found that the addition of a polyvinyl alcohol coating, such as OPADRY®II—YELLOW from Colorcon® (as sold in 2013) did not statistically significant decrease the dissolution rate of the tablet core, relative to the dissolution rate of an un-coated tablet core (see FIG. 1).

It was surprisingly found that some of the embodiments of the present invention provide oral formulations which allow a meal after about 30 minutes of oral administration of said composition, whithout affecting the bioavailability/variation of the active substance i.e. the acylated insulin.

### Tablet Core

One embodiment of the present invention is a pharmaceutical composition comprising one or more tablets, wherein each tablet consists of a tablet core and a polyvinyl alcohol coating, wherein said tablet core comprises one or more acylated insulin and a salt of capric acid.

One embodiment of the present invention is a pharmaceutical composition comprising up to three tablets, wherein each tablet consists of a tablet core and a polyvinyl alcohol coating, wherein said tablet core comprises one or more acylated insulin and a salt of capric acid.

One embodiment of the present invention is a pharmaceutical composition comprising two tablets, wherein each tablet consists of a tablet core and a polyvinyl alcohol coating, wherein said tablet core comprises one or more acylated insulin and a salt of capric acid.

One embodiment of the present invention is a pharmaceutical composition comprising between 150 and 250 tablets, wherein each tablet consists of a tablet core and a polyvinyl alcohol coating, wherein said tablet core comprises one or more acylated insulin and a salt of capric acid.

One embodiment of the present invention is a pharmaceutical composition comprising between about 140 and about 250 tablets weighing between 3.0-5.0 mg, wherein each tablet consists of a tablet core and a polyvinyl alcohol coating, wherein said tablet core comprises one or more acylated insulin and a salt of capric acid.

One embodiment of the present invention is a pharmaceutical composition comprising between 150 and 250 tablets weighing about 3.6 mg, wherein each tablet consists of a tablet core and a polyvinyl alcohol coating, wherein said tablet core comprises one or more acylated insulin and a salt of capric acid.

One embodiment of the present invention is a pharmaceutical composition comprising one or more tablets, wherein each tablet consists of an uncoated tablet core, wherein said tablet core comprises one or more acylated insulin and a salt of capric acid.

One embodiment of the present invention is a pharmaceutical composition comprising up to three tablets, wherein each tablet consists of an uncoated tablet core, wherein said tablet core comprises one or more acylated insulin and a salt of capric acid.

One embodiment of the present invention is a pharmaceutical composition comprising two tablets, wherein each tablet consists of an uncoated tablet core, wherein said tablet core comprises one or more acylated insulin and a salt of capric acid.

One embodiment of the present invention is a pharmaceutical composition comprising between 150 and 250 tablets, wherein each tablet consists of an uncoated tablet core, wherein said tablet core comprises one or more acylated insulin and a salt of capric acid.

One embodiment of the present invention is a pharmaceutical composition comprising between about 140 and about 250 tablets weighing between about 3.0-5.0 mg, wherein each tablet consists of an uncoated tablet core, wherein said tablet core comprises one or more acylated insulin and a salt of capric acid.

One embodiment of the present invention is a pharmaceutical composition comprising between 150 and 250 tablets weighing about 3.6 mg, wherein each tablet consists of an uncoated tablet core, wherein said tablet core comprises one or more acylated insulin and a salt of capric acid.

One embodiment of the present invention is a pharmaceutical composition comprising between about 14 and about 470 tablets weighing between about 1.5-50 mg, wherein each tablet consists of an uncoated tablet core, wherein said tablet core comprises one or more acylated insulin and a salt of capric acid.

One embodiment of the present invention is a pharmaceutical composition comprising between about 14 and about 470 tablets weighing between about 1.5-50 mg, wherein each tablet consists of an uncoated tablet core, wherein said tablet core comprises one or more acylated insulin and a salt of capric acid.

One embodiment of the present invention is a pharmaceutical composition comprising between about 14 and about 70 tablets weighing between 10-50 mg, wherein each tablet consists of an uncoated tablet core, wherein said tablet core comprises one or more acylated insulin and a salt of capric acid.

In one embodiment the salt of capric acid comprised in the present invention is in the form of a salt. In one embodiment the salt of capric acid comprised in the present invention is in the form of a sodium salt.

In one embodiment a tablet core according to this invention comprises one or more acylated insulin and a sodium salt of capric acid.

In one embodiment a tablet core according to this invention contains a salt of capric acid. In one embodiment a tablet core according to this invention contains a sodium salt of capric acid.

In one embodiment a tablet core according to the present invention comprises 50-85% (w/w) salt of capric acid. In one embodiment the tablet core according to the present invention comprises 70%-85 (w/w) salt of capric acid. In one embodiment the tablet core according to the present invention comprises 75%-85 (w/w) salt of capric acid. In one embodiment the tablet core according to the present invention comprises about 70% (w/w) salt of capric acid. In one embodiment the tablet core according to the present invention comprises less than 75% (w/w) salt of capric acid. In one embodiment the tablet core according to the present invention comprises less than 80% (w/w) salt of capric acid. In one embodiment the tablet core according to the present invention comprises less than 85% (w/w) salt of capric acid.

In one embodiment a tablet core comprises one or more acylated insulin and a salt of capric acid wherein said acylated insulin is a protease stabilised insulin comprising a linker and a fatty acid or fatty diacid chain having 14-22 carbon atoms.

In one embodiment a tablet core comprises one or more acylated insulin and a sodium salt of capric acid wherein said acylated insulin is a protease stabilised insulin comprising a linker and a fatty acid or fatty diacid chain having 14-22 carbon atoms In one embodiment a tablet core comprises a salt of a medium-chain fatty acid and an acylated insulin, wherein said acylated insulin is a protease stabilised insulin comprising one or more additional disulfide bonds.

In one embodiment a tablet core comprises a salt of a medium-chain fatty acid and an acylated insulin, wherein said acylated insulin is a protease stabilised insulin comprising a linker and a fatty acid or fatty diacid chain having 14-22 carbon atoms and optionally comprising one or more additional disulfide bonds. In one embodiment a tablet core comprises a salt of a medium-chain fatty acid and an acylated insulin, wherein said acylated insulin is a protease stabilised insulin comprising a linker and a fatty acid or fatty diacid chain having 14-22 carbon atoms and optionally comprises one or more additional disulfide bonds. In one embodiment a tablet core comprises one or more acylated insulin and a salt of capric acid wherein said acylated insulin is a protease stabilised insulin comprising a linker and a fatty acid or fatty diacid chain having 14-22 carbon atoms.

In one embodiment a tablet core one or more acylated insulin and a sodium salt of capric acid wherein said acylated insulin is a protease stabilised insulin comprising a linker and a fatty acid or fatty diacid chain having 14-22 carbon atoms.

In one embodiment a tablet core comprises one or more acylated insulin and a salt of capric acid wherein said acylated insulin comprises one or more additional disulfide bonds.

In one embodiment a tablet core comprises one or more acylated insulin and a sodium salt of capric acid wherein said acylated insulin comprises one or more additional disulfide bonds.

In one embodiment a tablet core comprises one or more acylated insulin and a salt of capric acid wherein said acylated insulin is a protease stabilised insulin comprising a linker and a fatty acid or fatty diacid chain having 14-22 carbon atoms and optionally comprising one or more additional disulfide bonds.

In one embodiment a tablet core comprises one or more acylated insulin and a sodium salt of capric acid wherein said acylated insulin is a protease stabilised insulin comprising a linker and a fatty acid or fatty diacid chain having 14-22 carbon atoms and optionally comprising one or more additional disulfide bonds. In one embodiment a tablet core according to the present invention weights about 600-900 mg and about 600-1300 mg.

In one embodiment a tablet core of this invention weights about 250-475 mg. In one embodiment a pharmaceutical composition according to the present invention consisting of a tablet core and a polyvinyl alcohol coating weights about 600-900 mg.

In one embodiment a pharmaceutical composition according to the present invention consisting of a tablet core and a polyvinyl alcohol coating weights about 280-500 mg In one embodiment a tablet core of this invention weights about 710 mg. In one embodiment a tablet core of this invention weights about 355 mg. In one embodiment a tablet core of this invention weights about 237 mg. In one embodiment a tablet core of this invention weighs about 600-800 mg. In one embodiment a tablet core of this invention weights about 200-380 mg.

In one embodiment a tablet core of this invention weights about 1.5-50 mg. In one embodiment a tablet core of this invention weights about 3.0-5.0 mg. In one embodiment a tablet core of this invention weights about 3.6 mg. In one embodiment a pharmaceutical composition according to the present invention consisting of a tablet core and a polyvinyl alcohol coating weighs about 745 mg. In one embodiment a pharmaceutical composition according to the present invention consisting of a tablet core and a polyvinyl alcohol coating weighs about 742 mg.

In one embodiment a pharmaceutical composition according to the present invention consisting of a tablet core and a polyvinyl alcohol coating weighs about 373 mg. In one embodiment a pharmaceutical composition according to the present invention consisting of a tablet core and a polyvinyl alcohol coating weighs about 258 mg.

In one embodiment a pharmaceutical composition according to the present invention consisting of a tablet core and a polyvinyl alcohol coating weighs about 240-400 mg.

In one embodiment a tablet core comprises about 77% (w/w) salt of capric acid. In one embodiment a tablet core comprises about 0.5% (w/w) stearic acid.

In one embodiment a tablet core comprises about 22.5% (w/w) sorbitol. In one embodiment a tablet core comprises about 20.5% (w/w) sorbitol. In one embodiment the sorbitol amount is adjusted relative to the amount of active ingredient. In one embodiment the sorbitol amount is adjusted relative to the amount of acylated insulin. In one embodiment the sorbitol amount is adjusted relative to the amount of acylated insulin after the principle of quantum satis (QS) meaning the amount which is needed to obtain a tablet with the desired weight. In one embodiment a tablet core comprises about 22.5% (w/w) sorbitol, when the amount of active ingredient is about 0% (w/w). In one embodiment a tablet core comprises about 20.5% (w/w) sorbitol, when the amount of active ingredient is about 0% (w/w). In one embodiment a tablet core comprises about 22.5% (w/w) sorbitol, when the amount of acylated insulin is about 0% (w/w). In one embodiment a tablet core comprises about 20.5% (w/w) sorbitol, when the amount of acylated insulin is about 0% (w/w). In one embodiment the sorbitol amount is adjusted relative to the amount of active ingredient, wherein the amount of active ingredient is at least about 0.5% (w/w). In one embodiment the sorbitol amount is adjusted relative to the amount of active ingredient, wherein the amount of active ingredient is at least 0.5% (w/w). In one embodiment the sorbitol amount is adjusted relative to the amount of active ingredient, wherein the amount of active ingredient is about 0-22.5% (w/w). In one embodiment the sorbitol amount is adjusted relative to the amount of active ingredient, wherein the amount of active ingredient is about 0-20.5% (w/w).

In one embodiment a tablet core comprises about 21.0% (w/w) sorbitol, when the amount of acylated insulin is about 0.5% (w/w). In one embodiment a tablet core comprises about 20.5% (w/w) sorbitol, when the amount of acylated insulin is about 2% (w/w). In one embodiment a tablet core comprises about 19.5% (w/w) sorbitol, when the amount of acylated insulin is about 3% (w/w). In one embodiment a tablet core comprises about 22.5-X % (w/w) sorbitol, wherein X is the amount of acylated insulin. In one embodiment a tablet core comprises about 20.5-X % (w/w) sorbitol, wherein X is the amount of acylated insulin. In one embodiment a tablet core comprises about 22.5-X % (w/w) sorbitol, wherein X is the amount of acylated insulin and X is from about 0-22.5. In one embodiment a tablet core comprises about 20.5-X % (w/w) sorbitol, wherein X is the amount of acylated insulin and X is from about 0-20.5. In one embodiment a tablet core comprises about 22.5-X % (w/w) sorbitol, wherein X is the amount of acylated insulin and X is about 0, 0.5, 1, 1.5, 2, 2.5, 3.0, 3.5, 4.0, 4.5 or 5.0. In one embodiment a tablet core comprises about 20.5-X % (w/w) sorbitol, wherein X is the amount of acylated insulin and X is about 0, 0.5, 1, 1.5, 2, 2.5, 3.0, 3.5, 4.0, 4.5 or 5.0. In one embodiment a tablet core comprises about 22.5-X % (w/w) sorbitol, wherein X is the amount of acylated insulin and X is about 5.5, 6, 6.5, 7, 7.5, 8, 8.5, 9.0, 9.5 or 10.0. In one embodiment a tablet core comprises about 22.5-X % (w/w) sorbitol, wherein X is the amount of acylated insulin and X is about 5.5, 6, 6.5, 7, 7.5, 8, 8.5, 9.0, 9.5 or 10.0. In one embodiment a tablet core comprises about 20.5-X % (w/w) sorbitol, wherein X is the amount of acylated insulin and X is about 10.5, 11, 11.5, 12, 12.5, 13, 13.5, 14.0, 14.5 or 15.0. In one embodiment a tablet core comprises about 22.5-X % (w/w) sorbitol, wherein X is the amount of acylated insulin and X is about 15.5, 16, 16.5, 17, 17.5, 18, 18.5, 19.0, 20.5, 21.0, 21.5, 22.0 or 22.5. In one embodiment a tablet core comprises about 20.5-X % (w/w) sorbitol, wherein X is the amount of acylated insulin and X is about 15.5, 16, 16.5, 17, 17.5, 18, 18.5, 19.0 or 20.5. In one embodiment a tablet core of the present invention comprises a salt of capric acid and one or more excipients.

In one embodiment some of the ingredients in a pharmaceutical composition according to the present invention are mucoadhesive. In one embodiment on or more of the ingredients in a pharmaceutical composition according to the present invention are mucoadhesive. In one embodiment none of the ingredients in a pharmaceutical composition according to the present invention are mucoadhesive. In one embodiment none of the excipients in a pharmaceutical composition according to the present invention are mucoadhesive. In one embodiment none of the ingredients in a tablet core according to the present invention are mucoadhesive. In one embodiment none of the excipients in a tablet according to the present invention are mucoadhesive. In one embodiment none of the ingredients in a polyvinyl alcohol coating according to the present invention are mucoadhesive. In one embodiment none of the excipients in a polyvinyl alcohol coating according to the present invention are mucoadhesive. In one embodiment excipients comprised in a tablet core according to the present invention have a molecular weight below 1000 g/mol. In one embodiment excipients comprised in a tablet core according to the present invention have a molecular weight below 900 g/mol. In one embodiment excipients comprised in a tablet core according to the present invention have a molecular weight below 800 g/mol. In one embodiment excipients comprised in a tablet core according to the present invention have a molecular weight below 700 g/mol. In one embodiment excipients comprised in a tablet core according to the present invention have a molecular weight below 600 g/mol. In one embodiment excipients comprised in a tablet core according to the present invention have a molecular weight below 500 g/mol. In one embodiment excipients comprised in a tablet core according to the present invention have a molecular weight below 400 g/mol. In one embodiment excipients comprised in a tablet core according to the present invention have a molecular weight below 300 g/mol.

In one embodiment one or more excipients comprised in a tablet core according to the present invention have a molecular weight below 1000 g/mol. In one embodiment one or more excipients comprised in a tablet core according to the present invention have a molecular weight below 900 g/mol. In one embodiment one or more excipients comprised in a tablet core according to the present invention have a molecular weight below 800 g/mol. In one embodiment one or more excipients comprised in a tablet core according to the present invention have a molecular weight below 700 g/mol. In one embodiment one or more excipients comprised in a tablet core according to the present invention have a molecular weight below 600 g/mol. In one embodiment one or more excipients comprised in a tablet core according to the present invention have a molecular weight below 500 g/mol. In one embodiment one or more excipients comprised in a tablet core according to the present invention have a molecular weight below 400 g/mol. In one embodiment one or more excipients comprised in a tablet core according to the present invention have a molecular weight below 300 g/mol. In one embodiment one or more excipients comprised in a tablet core according to the present invention have a molecular weight above 1000 g/mol. In one embodiment one or more excipients comprised in a tablet core according to the present invention have a molecular weight above 900 g/mol. In one embodiment one or more excipients comprised in a tablet core according to the present invention have a molecular weight above 800 g/mol. In one embodiment one or more excipients comprised in a tablet core according to the present invention have a molecular weight above 700 g/mol. In one embodiment one or more excipients comprised in a tablet core according to the present invention have a molecular weight above 600 g/mol. In one embodiment one or more excipients comprised in a tablet core according to the present invention have a molecular weight above 500 g/mol. In one embodiment one or more excipients comprised in a tablet core according to the present invention have a molecular weight above 400 g/mol. In one embodiment one or more excipients comprised in a tablet core according to the present invention have a molecular weight above 300 g/mol. In one embodiment one or more dry ingredients comprised in a tablet core according to the present invention have a molecular weight below 1000 g/mol. In one embodiment all dry ingredients comprised in a tablet core according to the present invention have a molecular weight below 900 g/mol. In one embodiment all dry ingredients comprised in a tablet core according to the present invention have a molecular weight below 800 g/mol. In one embodiment all dry ingredients comprised in a tablet core according to the present invention have a molecular weight below 700 g/mol. In one embodiment all dry ingredients comprised in a tablet core according to the present invention have a molecular weight below 600 g/mol. In one embodiment all dry ingredients comprised in a tablet core according to the present invention have a molecular weight below 500 g/mol. In one embodiment all dry ingredients comprised in a tablet core according to the present invention have a molecular weight below 400 g/mol. In one embodiment all dry ingredients comprised in a tablet core according to the present invention have a molecular weight below 300 g/mol.

In one embodiment none of the active ingredients or the excipients in the tablet core according to the present invention exert any water uptake. In one embodiment the active ingredients and the excipients in the tablet core exert zero water uptake. In one embodiment the active ingredients and the excipients in the tablet core exert 0-9% water uptake. In one embodiment the active ingredients and the excipients in the tablet core exert below 10% water uptake. In one embodiment the active ingredients and the excipients in the tablet core exert below 9% water uptake. In one embodiment the active ingredients and the excipients in the tablet core exert below 7% water uptake.

### Coating

One embodiment of the present invention regards a pharmaceutical composition comprising a tablet core and a polyvinyl alcohol coating, wherein said polyvinyl alcohol coating according to the present invention dissolves in aqueous medium independent of pH, i.e. is an immediate release coating.

One embodiment of the present invention is a pharmaceutical composition consisting of a tablet core and a polyvinyl alcohol coating, wherein said polyvinyl alcohol coating dissolves at all pH values. One embodiment of the present invention is a pharmaceutical composition consisting of a tablet core and a polyvinyl alcohol coating, wherein said polyvinyl alcohol coating dissolves independently of the pH in the solution aqueous medium. One embodiment of the present invention is a pharmaceutical composition consisting of a tablet core and a polyvinyl alcohol coating, wherein said polyvinyl alcohol coating dissolves throughout the entire pH range. One embodiment of the present invention is a pharmaceutical composition consisting of a tablet core and a polyvinyl alcohol coating, wherein said polyvinyl alcohol coating independently of the pH in the solution aqueous medium.

One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and wherein said pharmaceutical composition comprises a polyvinyl alcohol coating which dissolves at all pH values. One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and wherein said pharmaceutical composition comprises a polyvinyl alcohol coating which dissolves independently of the pH in the solution aqueous medium. One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and wherein said pharmaceutical composition comprises a polyvinyl alcohol coating which dissolves throughout the entire pH range.

One embodiment of the present invention concerns a pharmaceutical composition consisting of a tablet core and a polyvinyl alcohol coating, wherein said tablet core comprises a salt of a medium-chain fatty acid and an acylated insulin, wherein said acylated insulin comprises one or more additional disulfide bonds and wherein said pharmaceutical composition comprises a polyvinyl alcohol coating which dissolves independently of the pH in the solution aqueous medium.

One embodiment of the present invention concerns a pharmaceutical composition consisting of a tablet core and a polyvinyl alcohol coating, wherein said tablet core comprises a salt of a medium-chain fatty acid and an acylated insulin, wherein said acylated insulin comprises one or more additional disulfide bonds and wherein said pharmaceutical composition comprises a polyvinyl alcohol coating which dissolves at all pH values.

One embodiment of the present invention concerns a pharmaceutical composition consisting of a tablet core and a polyvinyl alcohol coating, wherein said tablet core comprises a salt of a medium-chain fatty acid and an acylated insulin, wherein said acylated insulin comprises one or more additional disulfide bonds and wherein said pharmaceutical composition comprises a polyvinyl alcohol coating which dissolves independently of the pH in the solution aqueous medium.

One embodiment of the present invention concerns a pharmaceutical composition consisting of a tablet core and a polyvinyl alcohol coating, wherein said tablet core comprises a salt of a medium-chain fatty acid and an acylated insulin, wherein said acylated insulin is a protease stabilised insulin comprising a linker and a fatty acid or fatty diacid chain having 14-22 carbon atoms and optionally comprises one or more additional disulfide bonds and wherein said pharmaceutical composition comprises a polyvinyl alcohol coating which dissolves at all pH values.

One embodiment of the present invention concerns a pharmaceutical composition consisting of a tablet core and a polyvinyl alcohol coating, wherein said tablet core comprises a salt of a medium-chain fatty acid and an acylated insulin, wherein said acylated insulin is a protease stabilised insulin comprising a linker and a fatty acid or fatty diacid chain having 14-22 carbon atoms and optionally comprises one or more additional disulfide bonds and wherein said pharmaceutical composition comprises a polyvinyl alcohol coating which dissolves independently of the pH in the solution aqueous medium.

One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and wherein said pharmaceutical composition comprises a polyvinyl alcohol coating which dissolves at all pH values. One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and wherein said pharmaceutical composition comprises a polyvinyl alcohol coating which dissolves independently of the pH in the solution aqueous medium. One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and wherein said pharmaceutical composition comprises a polyvinyl alcohol coating which dissolves throughout the entire pH range.

One embodiment of the present invention is a pharmaceutical composition consisting of a tablet core and a polyvinyl alcohol coating, wherein said tablet core comprises one or more acylated insulin and a salt of capric acid wherein said acylated insulin comprises one or more additional disulfide bonds and wherein said pharmaceutical composition comprises a polyvinyl alcohol coating which dissolves at all pH values. One embodiment of the present invention is a pharmaceutical composition consisting of a tablet core and a polyvinyl alcohol coating, wherein said tablet core comprises one or more acylated insulin and a salt of capric acid wherein said acylated insulin comprises one or more additional disulfide bonds and wherein said pharmaceutical composition comprises a polyvinyl alcohol coating which dissolves independently of the pH in the solution aqueous medium. One embodiment of the present invention is a pharmaceutical composition consisting of a tablet core and a polyvinyl alcohol coating, wherein said tablet core comprises one or more acylated insulin and a salt of capric acid wherein said acylated insulin comprises one or more additional disulfide bonds and wherein said pharmaceutical composition comprises a polyvinyl alcohol coating which is dissolving throughout the entire pH range.

One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and optionally comprising one or more additional disulfide bonds and wherein said pharmaceutical composition comprises a polyvinyl alcohol coating which dissolves at all pH values. One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and optionally comprising one or more additional disulfide bonds and wherein said pharmaceutical composition comprises a polyvinyl alcohol coating which dissolves independently of the pH in the solution aqueous medium. One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and optionally comprising one or more additional disulfide bonds and wherein said pharmaceutical composition comprises a polyvinyl alcohol coating which is dissolving throughout the entire pH range.

One embodiment of the present invention is a pharmaceutical composition consisting of a tablet core and a polyvinyl alcohol coating, wherein said tablet core comprises one or more acylated insulin and a salt of capric acid wherein said acylated insulin is a protease stabilised insulin comprising a linker and a fatty acid or fatty diacid chain having 14-22 carbon atoms and optionally comprising one or more additional disulfide bonds and wherein said pharmaceutical composition comprises a polyvinyl alcohol coating which is an immediate release coating.

One embodiment of the present invention regards a pharmaceutical composition comprising a tablet core and a polyvinyl alcohol coating, wherein said coating comprises polyvinyl alcohol polymer. One embodiment of the present invention regards a pharmaceutical composition comprising a tablet core and a polyvinyl alcohol coating, wherein said coating comprises polyvinyl alcohol polymer. In one embodiment a polyvinyl alcohol coating is an aqueous coating. In one embodiment a polyvinyl alcohol coating is an aqueous coating according to polyvinyl alcohol coating as disclosed in WO0104195 A1. In one embodiment a polyvinyl alcohol coating is an aqueous coating according to polyvinyl alcohol coating as exemplified in WO0104195 A1. In one embodiment a polyvinyl alcohol coating according to the present invention is an immediate release coating. In one embodiment a polyvinyl alcohol coating dissolves in aqueous medium. In one embodiment a polyvinyl alcohol coating dissolves in water. In one embodiment a polyvinyl alcohol polymer in a polyvinyl alcohol coating according to the present invention is soluble in aqueous medium.

In one embodiment a polyvinyl alcohol coating according to the present invention dissolves at all pH values. In one embodiment a polyvinyl alcohol coating according to the present invention dissolves in aqueous medium independent of the pH in the medium. In one embodiment a polyvinyl alcohol coating according to the present invention dissolves in aqueous medium throughout the entire pH range. In one embodiment a polyvinyl alcohol coating according to the present invention dissolves at any pH in aqueous medium.

One embodiment of the present invention regards a pharmaceutical composition wherein a polyvinyl alcohol coating comprises at least 25-55% polyvinyl alcohol polymer.

One embodiment of the present invention regards a pharmaceutical composition wherein a polyvinyl alcohol coating comprises at least 38-46% polyvinyl alcohol polymer.

In one embodiment a polyvinyl alcohol coating is an OPADRY®II—coating comprising polyvinyl alcohol polymer (such as e.g. from Colorcon® (as sold in 2013). In one embodiment a polyvinyl alcohol coating is an OPADRY®II—comprising polyvinyl alcohol polymer and is pigmented (such as e.g. from Colorcon® (as sold in 2013)). In one embodiment a polyvinyl alcohol coating is an OPADRY®II—comprising polyvinyl alcohol polymer and is clear (such as e.g. from Colorcon® (as sold in 2013)). In one embodiment a polyvinyl alcohol coating is an OPADRY®II—Yellow coating (such as e.g. from Colorcon® (as sold in 2013). In one embodiment a polyvinyl alcohol coating is an OPADRY®II—yellow coating comprising polyvinyl alcohol polymer (such as e.g. from Colorcon® (as sold in 2013)).

In one embodiment a polyvinyl alcohol coating is an OPADRY®II—clear coating comprising polyvinyl alcohol (such as e.g. from Colorcon® (as sold in 2013)). In one embodiment a pharmaceutical composition and/or a polyvinyl alcohol coating according to the present invention comprises excipients known to the person skilled in the art.

In one embodiment a pharmaceutical composition according to the present invention comprises polymers that may be used in aqueous coating processes, wherein said polymers may be in the form of dispersions or solutions. In one embodiment polymers according to the present invention are polyvinyl alcohol polymers. In one embodiment polymers according to the present invention are polyvinyl alcohol polymers forming a film. In one embodiment polymers according to the present invention are polymers as present in a polyvinyl alcohol coating such as e.g. OPADRY®II—Yellow as e.g. sold by Colorcon in 2013. In one embodiment a polyvinyl alcohol coating according to the present invention comprises polymers that may be used in aqueous coating processes, wherein said polymers may be in the form of dispersions or solutions.

In one embodiment a polyvinyl alcohol coating according to the present invention comprises excipients as known to the person skilled in the art. Non-limiting examples of such known excipients are disclosed in “Direct compression and the role of filler-binders” (p 173-217): by B. A. C. Carlin, in “Disintegrants in tabletting” (p 217-251): by R. C. Moreton, and in “Lubricants, glidants and adherents” (p 251-269), by N. A. Armstrong, in Pharmaceutical dosage forms: Tablets“, Informa Healthcare, N.Y., vol 2, 2008, L. L. Augsburger and S. W. Hoag”, and incorporated herein by reference.

In one embodiment a polyvinyl alcohol coating of a pharmaceutical composition according to the present invention is coated on to the surface of a tablet core according to the present invention in an amount of about 0-10% (w/w) relative to the tablet core. In one embodiment a polyvinyl alcohol coating of a pharmaceutical composition according to the present invention is coated on to an outer surface of a tablet core according to the present invention in an amount of about 0% (w/w) relative to the tablet core. In one embodiment a polyvinyl alcohol coating of a pharmaceutical composition according to the present invention is coated on to an outer surface of a tablet core according to the present invention in an amount of about 2% (w/w) relative to the tablet core. In one embodiment a polyvinyl alcohol coating of a pharmaceutical composition according to the present invention is coated on to an outer surface of a tablet core according to the present invention in an amount of about 4% (w/w) relative to the tablet core. In one embodiment a polyvinyl alcohol coating of a pharmaceutical composition according to the present invention is coated on to an outer surface of a tablet core according to the present invention in an amount of about 4.5% (w/w) relative to the tablet core. In one embodiment a polyvinyl alcohol coating of a pharmaceutical composition according to the present invention is coated on to an outer surface of a tablet core according to the present invention in an amount of about 5% (w/w) relative to the tablet core. In one embodiment a polyvinyl alcohol coating of a pharmaceutical composition according to the present invention is coated on to an outer surface of a tablet core according to the present invention in an amount of about 6% (w/w) relative to the tablet core. In one embodiment a polyvinyl alcohol coating of a pharmaceutical composition according to the present invention is coated on to an outer surface of a tablet core according to the present invention in an amount of about 8% (w/w) relative to the tablet core. In one embodiment a polyvinyl alcohol coating of a pharmaceutical composition according to the present invention is coated on to an outer surface of a tablet core according to the present invention in an amount of about 10% (w/w) relative to the tablet core. In one embodiment excipients are added to a polyvinyl alcohol dispersion.

In one embodiment excipients are added to a polyvinyl alcohol dispersion in the amount of about 10% (w/w) of the total dry coating material in said polyvinyl alcohol dispersion. In one embodiment excipients are added to a polyvinyl alcohol dispersion in the amount of about 10% (w/w) of the total dry coating material in said polyvinyl alcohol dispersion, wherein said total dry coating material in said polyvinyl alcohol dispersion comprises a polyvinyl alcohol polymer as defined in the present invention.

In one embodiment excipients are added to a polyvinyl alcohol dispersion in the amount of about 10% (w/w) of the total dry coating material in said polyvinyl alcohol dispersion, wherein said total dry coating material in said polyvinyl alcohol dispersion comprises a polyvinyl alcohol polymer as disclosed in WO0104195 A1.

In one embodiment excipients are added to a polyvinyl alcohol dispersion in the amount of about 10% (w/w) of the total dry coating material in said polyvinyl alcohol dispersion, wherein said total dry coating material in said polyvinyl alcohol dispersion comprises a polyvinyl alcohol polymer as exemplified in WO0104195 A1.

In one embodiment excipients are added to said polyvinyl alcohol dispersion in the amount of about 10% (w/w) of the total dry coating material in said polyvinyl alcohol dispersion, wherein said total dry coating material in said polyvinyl alcohol dispersion comprises polyvinyl alcohol polymer(s) such as comprised in OPADRY®II—coatings such as e.g. from Colorcon® (as sold in 2013).

In one embodiment excipients are added to said polyvinyl alcohol dispersion in the amount of about 10% (w/w) of the total dry coating material in said polyvinyl alcohol polyvinyl polymer, wherein said total dry coating material in said polyvinyl alcohol dispersion comprises polyvinyl alcohol polymer(s) different from the one comprised in OPADRY®II—coatings such as e.g. from Colorcon® (as sold in 2013).

In one embodiment excipients are added to said polyvinyl alcohol dispersion in the amount of about 10% (w/w) of the total dry coating material in said polyvinyl alcohol dispersion, wherein said total dry coating material in said polyvinyl alcohol dispersion comprises polyvinyl alcohol polymer(s) such as comprised in OPADRY®II—Yellow coatings such as e.g. from Colorcon® (as sold in 2013). In one embodiment excipients are added to said polyvinyl alcohol dispersion in the amount of about 10% (w/w) of the total dry coating material in said polyvinyl alcohol polyvinyl polymer, wherein said total dry coating material in said polyvinyl alcohol dispersion comprises polyvinyl alcohol polymer(s) different from the one comprised in OPADRY®II—Yellow coatings such as e.g. from Colorcon® (as sold in 2013).

In one embodiment excipients are added to said polyvinyl alcohol dispersion in the amount of about 10% (w/w) of the total dry coating material in said polyvinyl alcohol dispersion, wherein said total dry coating material in said polyvinyl alcohol dispersion comprises a polyvinyl alcohol different from the one comprised in OPADRY®II—coatings such as e.g. OPADRY®II—Yellow from Colorcon® (as sold in 2013) and wherein said polyvinyl alcohol coating dissolves at any pH. In one embodiment excipients are added to said polyvinyl alcohol dispersion in the amount of about 10% (w/w) of the total dry coating material in said polyvinyl alcohol, wherein said total dry coating material in said polyvinyl alcohol dispersion comprises a polyvinyl alcohol different from the one comprised in OPADRY®II—coatings such as e.g. OPADRY®II—Yellow from Colorcon® (as sold in 2013) resulting in an immediate release coating.

### Contact Between Tablet Core and Coating

When referring to the contact between a polyvinyl alcohol coating and the tablet core, if not indicated otherwise the contact is in the interface between the two interfaces and thus an inner surface of a polyvinyl alcohol coating and an outer surface of a tablet core.

Thus one embodiment of the present invention regards a pharmaceutical composition wherein an inner surface of a polyvinyl alcohol coating is at least partly in direct contact with an outer surface of a tablet core. Alternatively this could be described as; one embodiment of the present invention regards a pharmaceutical composition wherein a polyvinyl alcohol coating is at least partly in direct contact with a tablet core. Another alternative way to describe the same contact could be; one embodiment of the present invention regards a pharmaceutical composition wherein a polyvinyl alcohol coating is at least partly in direct contact with an outer surface of a tablet core.

One embodiment of the present invention regards a pharmaceutical composition wherein a polyvinyl alcohol coating is in direct contact with 0% or more of an outer surface of a tablet core. One embodiment of the present invention regards a pharmaceutical composition wherein a polyvinyl alcohol coating is in direct contact with 1% or more of an outer surface of a tablet core. One embodiment of the present invention regards a pharmaceutical composition wherein a polyvinyl alcohol coating is in direct contact with 10% or more of an outer surface of a tablet core. One embodiment of the present invention regards a pharmaceutical composition wherein a polyvinyl alcohol coating is in direct contact with 20% or more of an outer surface of a tablet core. One embodiment of the present invention regards a pharmaceutical composition wherein a polyvinyl alcohol coating is in direct contact with 30% or more of an outer surface of a tablet core.

One embodiment of the present invention regards a pharmaceutical composition wherein a polyvinyl alcohol coating is in direct contact with 40% or more of an outer surface of a tablet core. One embodiment of the present invention regards a pharmaceutical composition wherein a polyvinyl alcohol coating is in direct contact with 50% or more of an outer surface of a tablet core. One embodiment of the present invention regards a pharmaceutical composition wherein a polyvinyl alcohol coating is in direct contact with 60% or more of an outer surface of a tablet core. One embodiment of the present invention regards a pharmaceutical composition wherein a polyvinyl alcohol coating is in direct contact with 70% or more of an outer surface of a tablet core. One embodiment of the present invention regards a pharmaceutical composition wherein a polyvinyl alcohol coating is in direct contact with 80% or more of an outer surface of a tablet core. One embodiment of the present invention regards a pharmaceutical composition wherein a polyvinyl alcohol coating is in direct contact with 85% or more of an outer surface of a tablet core. One embodiment of the present invention regards a pharmaceutical composition wherein a polyvinyl alcohol coating is in direct contact with 90% or more of an outer surface of a tablet core. One embodiment of the present invention regards a pharmaceutical composition wherein a polyvinyl alcohol coating is in direct contact with 95% or more of an outer surface of a tablet core. One embodiment of the present invention regards a pharmaceutical composition wherein a polyvinyl alcohol coating is in direct contact with 99% or more of an outer surface of a tablet core. One embodiment of the present invention regards a pharmaceutical composition wherein a polyvinyl alcohol coating is in direct contact with 100% of an outer surface of a tablet core.

One embodiment of the present invention regards a pharmaceutical composition wherein a polyvinyl alcohol coating is in direct contact with the majority of the surface of a tablet core. One embodiment of the present invention regards a pharmaceutical composition wherein a polyvinyl alcohol coating is in direct contact with most of the surface of a tablet core. One embodiment of the present invention regards a pharmaceutical composition wherein a polyvinyl alcohol coating is in direct contact with some of the surface of a tablet core. In one embodiment one or more additional non-functional coatings may be added on top of a polyvinyl alcohol coating according to the present invention. In one embodiment one or more additional non-functional coatings may be added on below a polyvinyl alcohol coating according to the present invention.

One embodiment of the present invention regards a pharmaceutical composition wherein no additional non-functional coating is applied between a polyvinyl alcohol coating and a tablet core. One embodiment of the present invention regards a pharmaceutical composition wherein no continuous additional non-functional coating is applied between a polyvinyl alcohol coating and a tablet core. One embodiment of the present invention regards a pharmaceutical composition wherein no uninterrupted additional non-functional coating is applied between a polyvinyl alcohol coating and a tablet core. One embodiment of the present invention regards a pharmaceutical composition wherein no discontinuous additional non-functional coating is applied between a polyvinyl alcohol coating and a tablet core. One embodiment of the present invention regards a pharmaceutical composition wherein no interrupted additional non-functional coating is applied between a polyvinyl alcohol coating and a tablet core.

One embodiment of the present invention regards a pharmaceutical composition wherein an additional non-functional coating is applied between a polyvinyl alcohol coating and a tablet core. One embodiment of the present invention regards a pharmaceutical composition wherein a continuous additional non-functional coating is applied between a polyvinyl alcohol coating and a tablet core. One embodiment of the present invention regards a pharmaceutical composition wherein an uninterrupted additional non-functional coating is applied between a polyvinyl alcohol coating and a tablet core. One embodiment of the present invention regards a pharmaceutical composition wherein a discontinuous additional non-functional coating is applied between a polyvinyl alcohol coating and a tablet core. One embodiment of the present invention regards a pharmaceutical composition wherein an interrupted additional non-functional coating is applied between a polyvinyl alcohol coating and a tablet core.

One embodiment of the present invention regards a pharmaceutical composition wherein a polyvinyl alcohol coating is in direct contact with the majority of the caprate exposed at an outer surface of a tablet core.

One embodiment of the present invention regards a pharmaceutical composition wherein a polyvinyl alcohol coating is in direct contact with the majority of the caprate and acylated insulin exposed at an outer surface of a tablet core.

One embodiment of the present invention regards a pharmaceutical composition comprising a tablet core and a polyvinyl alcohol coating, wherein said polyvinyl alcohol coating is in direct contact with the majority of the caprate and acylated insulin exposed at an outer surface of said tablet core, wherein said acylated insulin is a protease stabilised insulin comprising a linker and a fatty acid or fatty diacid chain having 14-22 carbon atoms.

One embodiment of the present invention regards a pharmaceutical composition comprising a tablet core and a polyvinyl alcohol coating, wherein said polyvinyl alcohol coating is in direct contact with the majority of the caprate and acylated insulin exposed at an outer surface of said tablet core, wherein said acylated insulin comprises one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin exposed at an outer surface of said tablet core, wherein said acylated insulin is a protease stabilised insulin comprising a linker and a fatty acid or fatty diacid chain having 14-22 carbon atoms and optionally comprising one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of said tablet core, wherein said acylated insulin is a protease stabilised insulin comprising a linker and a fatty acid or fatty diacid chain having 14-22 carbon atoms.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of said tablet core, wherein said acylated insulin comprises one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of said tablet core, wherein said acylated insulin is a protease stabilised insulin comprising a linker and a fatty acid or fatty diacid chain having 14-22 carbon atoms and optionally comprising one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin exposed at an outer surface of said tablet core, wherein said acylated insulin is a protease stabilised insulin comprising a linker and a fatty acid or fatty diacid chain having 14-22 carbon atoms.

One embodiment of the present invention regards a pharmaceutical composition comprising a tablet core and a polyvinyl alcohol coating, wherein said polyvinyl alcohol coating is in direct contact with the majority of the caprate and acylated insulin exposed at an outer surface of said tablet core, wherein said acylated insulin comprises one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin exposed at an outer surface of said tablet core, wherein said acylated insulin is a protease stabilised insulin comprising a linker and a fatty acid or fatty diacid chain having 14-22 carbon atoms and optionally comprising one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of said tablet core, wherein said acylated insulin is a protease stabilised insulin comprising a linker and a fatty acid or fatty diacid chain having 14-22 carbon atoms.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of said tablet core, wherein said acylated insulin comprises one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of said tablet core, wherein said acylated insulin is a protease stabilised insulin comprising a linker and a fatty acid or fatty diacid chain having 14-22 carbon atoms optionally comprising one or more additional disulfide bonds.

One embodiment of the present invention regards a pharmaceutical composition comprising a tablet core and a polyvinyl alcohol coating, wherein said polyvinyl alcohol coating is in direct contact with the majority of the caprate and acylated insulin and any additional excipients comprised in said tablet core which are exposed at an outer surface of a tablet core.

One embodiment of the present invention regards a pharmaceutical composition comprising a tablet core and a polyvinyl alcohol coating, wherein said polyvinyl alcohol coating is in direct contact with the majority of the caprate, acylated insulin and any additional excipients comprised in said tablet core which are exposed at an outer surface of a tablet core.

One embodiment of the present invention regards a pharmaceutical composition comprising a tablet core and a polyvinyl alcohol coating, wherein said polyvinyl alcohol coating is in direct contact with the majority of the caprate, acylated insulin, sorbitol and stearic acid comprised in said tablet core which are exposed at an outer surface of said tablet core.

One embodiment of the present invention regards a pharmaceutical composition comprising a tablet core and a polyvinyl alcohol coating, wherein said polyvinyl alcohol coating is in direct contact with the majority of all ingredients comprised in said tablet core exposed at an outer surface of said tablet core.

### Pharmaceutical Composition

In one embodiment a pharmaceutical composition according to the present invention is a solid oral composition. In one embodiment a pharmaceutical composition according to the present invention is a tablet. In one embodiment a pharmaceutical composition according to the present invention comprises multiple tablets. In one embodiment a tablet core of a pharmaceutical composition according to the present invention is a tablet weighing about 1.5 and about 900 mg. In one embodiment a tablet core of a pharmaceutical composition according to the present invention is a tablet weighing between up to about 50 mg. In one embodiment a tablet core of a pharmaceutical composition according to the present invention is a tablet weighing between up to about 1.5-50 mg. In one embodiment a tablet core of a pharmaceutical composition according to the present invention is a tablet weighing between about 50 mg and about 600 mg. In one embodiment the present invention relates to a coated or uncoated tablet core weighing between about 100 mg and about 600 mg and may herein be denominated “monolith midi tablet” or “midi tablet”. In one embodiment the present invention relates to a coated or uncoated tablet core weighing between about 600 mg and about 900 mg. In one embodiment the present invention relates to a coated or uncoated tablet core of weighing between about 600 mg and 1300 mg, preferably ranging from 600 mg to 900 mg and may herein be denominated “monolith” or “monolith tablet”. In one embodiment the present invention relates to a tablet core weighing between about 3.0 and about 5.0 mg. In one embodiment the present invention relates to a tablet core weighing about 3.6 mg. In one embodiment the present invention relates to a tablet core weighing up to about 50 mg and may herein be denominated “mini-tablet(s)”. In one embodiment the present invention relates to a tablet core weighing between about 3.0 and about 5.0 mg and may herein be denominated “mini-tablet(s)”. In one embodiment the present invention relates to a tablet core weighing about 3.6 mg and may herein also be denominated “mini-tablet(s)”. In one embodiment the present invention relates to mini-tablets which are uncoated tablet cores. In one embodiment the present invention relates to mini-tablets which are coated tablet cores, coated with a polyvinyl alcohol coating as defined in this application.

In one embodiment the present invention relates to mini-tablets which are compressed into a fast disintegrating tablet in the size of a midi-tablet or monolith tablet.

In one embodiment of this invention up to about 300 mini-tablets are compressed into a fast disintegrating tablet in the size of a midi-tablet or monolith tablet.

In one embodiment of this invention up to about 300 mini-tablets are provided in a capsule. In one embodiment up to about six midi-tablets are provided in a capsule.

In one embodiment of this invention up to about three midi-tablets are provided in a capsule.

In one embodiment of this invention two midi-tablets are provided in a capsule. In one embodiment of this invention a tablet core of a pharmaceutical composition according to the present invention is a tablet weighing up to about 900 mg.

In one embodiment of this invention about 20-300 tablet cores of this invention each weighing between about 1.5-50 mg are provided in one or more capsules.

In one embodiment of this invention about 20-100 tablet cores of this invention each weighing between about 1.5-50 mg are provided in one or more capsules.

In one embodiment of this invention about 150-250 tablet cores of this invention each weighing between about 1.5-50 mg are provided in one or more capsules.

In one embodiment of this invention about 100-250 tablet cores of this invention each weighing between about 3.0-10 mg are provided in one or more capsules.

In one embodiment of this invention about 20-300 tablet cores of this invention each weighing between about 3.0-10 mg are provided in one or more capsules.

In one embodiment of this invention about 150-250 tablet cores of this invention each weighing between about 3.0-10 mg are provided in one or more capsules.

In one embodiment of this invention about 20-100 tablet cores of this invention each weighing between about 3.0-10 mg are provided in one or more capsules.

In one embodiment of this invention about 100-250 tablet cores of this invention each weighing between about 3.0-10 mg are provided in one or more capsules.

In one embodiment of this invention about 150-250 tablet cores of this invention each weighing about 3.6 mg are provided in one or more capsules.

In one embodiment of this invention about 150-250 tablet cores of this invention each weighing between about 3.0-5.0 mg are provided in one or more capsules.

In one embodiment of this invention about 150-250 tablet cores of this invention each weighing about 3.6 mg are provided in one or more capsules.

In one embodiment of this invention about 600-900 mg tablet cores of this invention, wherein each tablet core weighs between about 3.0-5.0 mg are provided in one or more capsules. In one embodiment of this invention about 600-900 mg tablet cores of this invention, wherein each tablet core weighs about 3.6 mg are provided in one or more capsules.

In one embodiment of this invention about 710 mg tablet cores of this invention, wherein each tablet core weighs between about 3.0-5.0 mg are provided in one or more capsules. In one embodiment of this invention about 710 mg tablet cores of this invention, wherein each tablet core weighs about 3.6 mg are provided in one or more capsules.

In one embodiment of this invention about 588 mg tablet cores of this invention, wherein each tablet core weighs between about 3.0-5.0 mg are provided in one or more capsules. In one embodiment of this invention about 710 mg tablet cores of this invention, wherein each tablet core weighs about 3.6 mg are provided in one or more capsules.

In one embodiment of this invention about 600 mg tablet cores of this invention, wherein each tablet core weighs between about 3.0-5.0 mg are provided in one or more capsules. In one embodiment of this invention about 710 mg tablet cores of this invention, wherein each tablet core weighs about 3.6 mg are provided in one or more capsules.

In one embodiment a tablet core of a pharmaceutical composition according to the present invention is a multiparticulate system. In one embodiment a tablet core of a pharmaceutical composition according to the present invention is provided in a capsule. In one embodiment a tablet core of a pharmaceutical composition according to the present invention is a multiparticulate system, wherein said multiparticulate system may be compressed into the form of a tablet or contained in a capsule. In one embodiment, said compressed tablet is fast disintegrating.

In one embodiment a tablet core according to the present invention comprises one or more layers. In one embodiment a tablet core according to the present invention comprises one or more tablets. In one embodiment a tablet core according to the present invention comprises up to three tablets. In one embodiment a tablet core according to the present invention comprises two tablets. The tablet may be a single or multilayer tablet having a compressed multiparticulate system in one, all or none of the layers.

In one embodiment a tablet core according to the present invention is a multiparticulate system comprising tablets or particles of the same dimensions. In one embodiment a tablet core according to the present invention is a multiparticulate system comprising tablets or particles of various dimensions. In one embodiment tablets or particles of multiparticulate systems according to the present invention are uncoated and provided in a capsule. In one embodiment tablets or particles of multiparticulate systems according to the present invention are coated with a polyvinyl alcohol coating and provided in a capsule.

In one embodiment tablets or particles of multiparticulate systems according to the present invention are coated with a polyvinyl alcohol coating. In one embodiment tablets or particles of multiparticulate systems according to the present invention are coated with a polyvinyl alcohol coating, wherein a polyvinyl alcohol coating is an Opdary®II Yellow coating such as e.g. from Colorcon® (as sold in 2013).

In one embodiment tablets or particles of multiparticulate systems according to the present invention are individually coated with a polyvinyl alcohol coating. In one embodiment tablets or particles of multiparticulate systems according to the present invention are individually coated with a polyvinyl alcohol coating, before compressed into a tablet, which may be fast disintegrating and have the size of a midi tablet or monolith tablet, i.e. weigh between about 50 to about 600 mg or about 600 to about 900 mg

In one embodiment individually coated tablets or particles of a multiparticulate system according to the present invention are compressed into a tablet core. In one embodiment individually tablets or coated particles of a multiparticulate system according to the present invention are compressed into a tablet core and the resulting tablet core is not coated with another layer of polyvinyl alcohol coating. In one embodiment individually coated tablets or particles of a multiparticulate system according to the present invention are compressed into a tablet core and said resulting tablet core is also coated with a polyvinyl alcohol coating. In one embodiment tablets or particles of multiparticulate systems according to the present invention are individually coated with polyvinyl alcohol coating and compressed into a tablet and said resulting tablet is coated with an additional non-functional coating.

In one embodiment tablets or particles of multiparticulate systems according to the present invention are collectively coated with a polyvinyl alcohol coating. In one embodiment tablets or particles of multiparticulate systems according to the present invention are collectively coated with a polyvinyl alcohol coating, after being compressed into a tablet.

In one embodiment a pharmaceutical composition according to the present invention comprises a tablet core, wherein said tablet core comprises a salt of capric acid and one or more acylated insulins, wherein at least one acylated insulin is one or more acylated insulin as described herein. In one embodiment a pharmaceutical composition according to the present invention comprises a tablet core, wherein said tablet core comprises a salt of capric acid and insulin and one or more excipients. In one embodiment a pharmaceutical composition according to the present invention comprises a tablet core, wherein said tablet core comprises a salt of capric acid, acylated insulin and one or more excipients, such as but not limited to sorbitol, magnesium stearate, stearate and stearic acid.

In one embodiment of this invention more than one tablet cores weighing below 50 mg are compressed into a tablet, which may be fast disintegrating and have the size of a midi tablet or monolith tablet, i.e. weigh between about 50 to 600 mg, about 100 mg to about 600 mg or about 600 to about 900 mg or about 600 to about 1300 mg.

In one embodiment a pharmaceutical composition according to the present invention comprises a tablet core, wherein said tablet core comprises one or more excipients, such as polyols and/or lubricants. In one embodiment a pharmaceutical composition according to the present invention comprises polyols. In one embodiment a pharmaceutical composition according to the present invention comprises a tablet core, wherein said tablet core comprises polyols, such as, but not limited to sorbitol and mannitol. In one embodiment a pharmaceutical composition according to the present invention comprises polyols, wherein said polyols are selected from the group consisting of sorbitol, mannitol or mixtures thereof.

In one embodiment a pharmaceutical composition according to the present invention comprises a tablet core, wherein said tablet core comprises lubricants, such as, but not limited to stearic acid, magnesium stearate, stearate and colloidal silica. In one embodiment a pharmaceutical composition according to the present invention comprises lubricants, wherein said lubricants are selected from the group consisting of stearic acid, magnesium stearate, stearate or mixtures thereof.

In certain embodiments of the present invention, the pharmaceutical composition comprises a tablet core, wherein said tablet core may comprise additional excipients commonly found in a pharmaceutical composition, examples of such excipients include, but are not limited to enzyme inhibitors, stabilisers, preservatives, flavours, sweeteners and other components as described in ‘Handbook of Pharmaceutical Excipients’ Ainley Wade, Paul J. Weller, Arthur H. Kibbe, 3^{rd} edition, American Pharmacists Association (2000), which is hereby incorporated by reference or—‘Handbook of Pharmaceutical Excipients’ Rowe et al., Eds., 4th Edition, Pharmaceutical Press (2003), which is hereby incorporated by reference.

In one embodiment a pharmaceutical composition according to the present invention comprises excipients known to the person skilled in the art.

In one embodiment a pharmaceutical composition according to the present invention comprises excipients as known to the person skilled in the art. Non-limiting examples of such known excipients are disclosed in “Direct compression and the role of filler-binders” (p 173-217): by B. A. C. Carlin, in “Disintegrants in tabletting” (p 217-251): by R. C. Moreton, and in “Lubricants, glidants and adherents” (p 251-269), by N. A. Armstrong, in Pharmaceutical dosage forms: Tablets“, Informa Healthcare, N.Y., vol 2, 2008, L. L. Augsburger and S. W. Hoag”, and incorporated herein by reference.

In one embodiment a pharmaceutical composition according to the present invention is in the form of a solid oral formulation. In one embodiment a pharmaceutical composition according to the present invention is manufactured into a tablet. In one embodiment a pharmaceutical composition according to the present invention is manufactured into a tablet for oral administration.

In one embodiment the capsule in which a pharmaceutical composition according to the present invention is provided in is selected from the group of capsules known to the person skilled in the art.

In one embodiment the capsule in which a pharmaceutical composition according to the present invention is provided in is selected from the group of capsules commercially available in 2015.

In one embodiment the capsule in which a pharmaceutical composition according to the present invention is provided in is selected from the group of capsules comprising gelatin or gelatin-like material.

In one embodiment the capsule in which a pharmaceutical composition according to the present invention is provided in is selected from the group of capsules; fish-gelatin, HMPC, pullan, procine gelatin.

In one embodiment the capsule in which a pharmaceutical composition according to the present invention is provided in releases its content within 10 minutes after oral administration.

In one embodiment the capsule in which a pharmaceutical composition according to the present invention is provided in releases its content within 5 minutes after oral administration.

### Use of the Composition of the Invention

In one embodiment, a pharmaceutical composition according to the invention is used for the preparation of a medicament for the treatment or prevention of hyperglycaemia, type 2 diabetes mellitus, impaired glucose tolerance and type 1 diabetes mellitus.

In one embodiment a pharmaceutical composition according to the present invention shows a Tmax between about 45-75 minutes after oral administration to a Beagle dog. In one embodiment a pharmaceutical composition according to the present invention shows a Tmax at about 45 minutes after oral administration to a Beagle dog. In one embodiment a pharmaceutical composition according to the present invention shows a Tmax at about 50. In one embodiment a pharmaceutical composition according to the present invention shows a Tmax after about 55 minutes after oral administration to a Beagle dog. In one embodiment a pharmaceutical composition according to the present invention shows a Tmax at about 60. In one embodiment a pharmaceutical composition according to the present invention shows a Tmax after about 65 minutes after oral administration to a Beagle dog. In one embodiment a pharmaceutical composition according to the present invention shows a Tmax after about 70 minutes after oral administration to a Beagle dog. In one embodiment a pharmaceutical composition according to the present invention shows a Tmax after about 75 minutes after oral administration to a Beagle dog.

In one embodiment the present invention provides an oral formulation which allows a meal after 30 minutes of oral administration of said composition, whithout affecting the bioavailability/variation of the active substance i.e. the acylated insulin.

### Method of Production

One embodiment of the present invention regards a method for manufacture of compositions according to the present invention. In one embodiment a polyvinyl alcohol coating of the present inventions is performed by any methods known to the person skilled in the art.

In one embodiment the coating of the present invention is performed by any method disclosed in “Coating processes and equipment, by D. M. Jones in “Pharmaceutical dosage forms: Tablets”, Informa Healthcare, N.Y., vol 1, 2008 p 373-399, L. L. Augsburger and S. W. Hoag”, incorporated herein by reference. In one embodiment the tablet core is a tablet core manufactured by suitable methods for formulation solid oral compositions.

In one embodiment an insulin powder is sieved before formulation. In one embodiment a sorbitol (or any other equivalent excipient) powder is sieved before formulation. In one embodiment sorbitol and insulin powder are mixed together. In one embodiment equal amounts of sorbitol and insulin powder are mixed together. In one embodiment equal amounts of sorbitol and insulin powder are mixed by hand.

In one embodiment sorbitol and insulin powders are mixed by hand. In one embodiment sorbitol and insulin powders are initially mixed by hand. In one embodiment sorbitol and insulin powders are mixed by hand and by an automatized mixing process. In one embodiment sorbitol and insulin powders are mixed by hand and by an automatized mixing process, wherein said automatized mixing process is performed in a Tubular-mixer.

In one embodiment sorbitol and insulin powders are mixed by an automatized mixing process. In one embodiment sorbitol and insulin powders are mixed by an automatized mixing process, wherein said automatized mixing process is performed in a Tubular-mixer.

In one embodiment sorbitol and insulin powders are initially mixed by hand, followed by an automatized mixing process. In one embodiment sorbitol and insulin powders are initially mixed by hand until blended together well. In one embodiment sorbitol and insulin powders are initially mixed by hand until blended together well, followed by an automatized mixing process. In one embodiment sorbitol and insulin powders are initially mixed by hand, followed by an automatized mixing process, wherein said automatized mixing process is performed in a Tubular-mixer.

In one embodiment sorbitol and insulin powders are initially mixed by hand until blended together well, wherein the degree of blending of said sorbitol and insulin powder is evaluated by eyeballing. In one embodiment sorbitol and insulin powders are initially mixed by hand until blended well, wherein the degree of blending of said sorbitol and insulin powder is evaluated by eyeballing, followed by an automatized mixing process.

In one embodiment equal amounts of sorbitol and insulin powder are mixed by hand and another portion of sorbitol is added in an amount twice as high as the first addition of sorbitol, which then is also stirred well by hand. When said last addition of sorbitol is admixed well, the powder is then subjected to mechanical mixing in a Turbula-mixer or any equivalent mixer to finalise the mixing process, resulting in a homogenous powder.

In one embodiment a salt of capric acid is added to said homogenous powder of sorbitol and insulin in amounts of 1:1. The addition may be performed in two steps and the mixing may initially performed by hand and finalised by mechanical mixing in a Turbula-mixer or any other automatized mixing device. The addition may be performed in two steps and the mixing is initially performed by hand and finalised by mechanical mixing in a Turbula-mixer or any equivalent mixer.

The powder may then be compressed in a tablet press as known to the person skilled in the art, resulting in a tablet core according to the present invention. The powder may then be compressed in a rotary tablet press as known to the person skilled in the art, resulting in a tablet core according to the present invention. The powder may then be compressed in a single punch tablet press as known to the person skilled in the art, resulting in a tablet core according to the present invention. The powder may then be compressed in an excenter tablet press as known to the person skilled in the art, resulting in a tablet core according to the present invention.

In one embodiment a polyvinyl alcohol coating may be coated on top of a tablet core according to the present invention. In one embodiment polyvinyl alcohol coating may be coated on top of a tablet according to the present invention. In one embodiment a polyvinyl alcohol coating may be coated on top of an outer surface of a tablet core according to the present invention.

In one embodiment a polyvinyl alcohol dispersion or a dry polymer is coated on top of a tablet core according to this invention. In one embodiment a polyvinyl alcohol dispersion or a dry polymer is coated on top of a tablet according to this invention.

In one embodiment a polyvinyl alcohol dispersion is filtrated through a mesh filter prior to the actual coating prior to the actual coating procedure.

In one embodiment a polyvinyl alcohol dispersion is stirred prior to a filtration through a mesh filter, prior to the actual coating procedure. In one embodiment a polyvinyl alcohol dispersion is stirred prior to a filtration through an about 0.24 mm mesh filter, prior to the actual coating procedure.

In one embodiment a polyvinyl alcohol dispersion comprising further excipients is filtrated through a mesh filter prior to the actual coating prior to the actual coating procedure.

In one embodiment a polyvinyl alcohol dispersion further comprising further excipients is stirred prior to a filtration through a mesh filter, prior to the actual coating procedure. In one embodiment a polyvinyl alcohol dispersion further comprising further excipients is stirred prior to a filtration through an about 0.24 mm mesh filter, prior to the actual coating procedure.

In one embodiment the actual coating procedure of tablet cores or tablets according to the present invention is performed in a pan coater or fluid bed coater. In one embodiment the actual coating procedure of tablet cores or tablets according to the present invention is performed in a pan coater or fluid bed coater by spraying a polyvinyl alcohol dispersion through a spray nozzle. In one embodiment the actual coating procedure of tablet cores or tablets according to the present invention is performed in a pan coater or fluid bed coater by spraying a polyvinyl alcohol dispersion further comprising further excipients through a spray nozzle.

In one embodiment said coating processes and equipment may be used as disclosed by D. M. Jones in “Pharmaceutical dosage forms: Tablets”, Informa Healthcare, N.Y., vol. 1, 2008 p 373-399, L. L. Augsburger and S. W. Hoag”, which hereby in incorporated by reference.

For the production of smaller tablets, please refer to the methods provided herein.

### Acylated Insulin

In one embodiment a tablet core according to the present invention comprises an acylated insulin as defined in the following pages.

In one embodiment the acylated insulins for use in the pharmaceutical composition of the invention are stabilised against proteolytic degradation, i.e. against rapid degradation in the gastro intestinal (GI) tract or elsewhere in the body. Acylated insulins stabilised against proteolytic degradation are herein denominated “protease stabilised insulin” or “proteolytically stable insulin”. An acylated insulin which is stabilised against proteolytic degradation is herein to be understood as an acylated insulin, which is subjected to slower degradation by one or more proteases relative to human insulin. In one embodiment an acylated insulin in a pharmaceutical composition according to the invention is subjected to slower degradation by one or more proteases relative to human insulin. In a further embodiment of the invention an acylated insulin in a pharmaceutical composition according to the invention is stabilised against degradation by one or more enzymes selected from the group consisting of: pepsin (such as e.g. the isoforms pepsin A, pepsin B, pepsin C and/or pepsin F), chymotrypsin (such as e.g. the isoforms chymotrypsin A, chymotrypsin B and/or chymotrypsin C), trypsin, Insulin-Degrading Enzyme (IDE), elastase (such as e.g. the isoforms pancreatic elastase I and/or II), carboxypeptidase (e.g. the isoforms carboxypeptidase A, carboxypeptidase A2 and/or carboxypeptidase B), aminopeptidase, cathepsin D and other enzymes present in intestinal extracts derived from rat, pig or human.

In one embodiment an acylated insulin in a pharmaceutical composition according to the invention is stabilised against degradation by one or more enzymes selected from the group consisting of: chymotrypsin, trypsin, Insulin-Degrading Enzyme (IDE), elastase, carboxypeptidases, aminopeptidases and cathepsin D. In a further embodiment an acylated insulin in a pharmaceutical composition according to the invention is stabilised against degradation by one or more enzymes selected from the group consisting of: chymotrypsin, carboxypeptidases and IDE. In a yet further embodiment an acylated insulin in a pharmaceutical composition according to the invention is stabilised against degradation by one or more enzymes selected from: chymotrypsin and IDE. In a yet further embodiment an acylated insulin in a pharmaceutical composition according to the invention is stabilised against degradation by one or more enzymes selected from: chymotrypsin and carboxypeptidases.

T½ may be determined as described in example 102 of WO2011/161125 as a measure of the proteolytical stability of an acylated insulin in a pharmaceutical composition according to the invention towards protease enzymes such as chymotrypsin, pepsin and/or carboxypeptidase A or towards a mixture of enzymes such as tissue extracts (from liver, kidney, duodenum, jejunum, ileum, colon, stomach, etc.). In one embodiment of the invention T½ is increased relative to human insulin. In a further embodiment T½ is increased relative to the acylated insulin without one or more additional disulfide bonds. In a yet further embodiment T½ is increased at least 2-fold relative to human insulin. In a yet further embodiment T½ is increased at least 2-fold relative to the acylated insulin without one or more additional disulfide bonds. In a yet further embodiment T½ is increased at least 3-fold relative to human insulin. In a yet further embodiment T½ is increased at least 3-fold relative to the acylated insulin without one or more additional disulfide bonds. In a yet further embodiment T½ is increased at least 4-fold relative to human insulin. In a yet further embodiment T½ is increased at least 4-fold relative to the acylated insulin without one or more additional disulfide bonds. In a yet further embodiment T½ is increased at least 5-fold relative to human insulin. In a yet further embodiment T½ is increased at least 5-fold relative to the acylated insulin without one or more additional disulfide bonds. In a yet further embodiment T½ is increased at least 10-fold relative to human insulin. In a yet further embodiment T½ is increased at least 10-fold relative to the acylated insulin without one or more additional disulfide bonds. T½ may also be expressed as the relative T½, relative to a proteolytically stabilised insulin analogue, A14E, B25H, desB30 human insulin as described in example 102 of WO2011/161125.

In one embodiment, an acylated insulin may have increased solubility relative to human insulin. In a further embodiment, an acylated insulin has increased solubility relative to human insulin at pH 3-9. In a yet further embodiment, an acylated insulin has increased solubility relative to human insulin at pH 4-8.5. In a still further embodiment, an acylated insulin has increased solubility relative to human insulin at pH 4-8. In a yet further embodiment, an acylated insulin has increased solubility relative to human insulin at pH 4.5-8. In a further embodiment, an acylated insulin has increased solubility relative to human insulin at pH 5-8. In a yet further embodiment, an acylated insulin has increased solubility relative to human insulin at pH 5.5-8. In a further embodiment, an acylated insulin has increased solubility relative to human insulin at pH 6-8. In one embodiment, an acylated insulin has increased solubility relative to human insulin at pH 2-4.

In one embodiment, an acylated insulin may have increased solubility relative to the parent insulin. In a further embodiment, an acylated insulin has increased solubility relative to the parent insulin at pH 3-9. In a yet further embodiment an acylated insulin has increased solubility relative to parent insulin at pH 4-8.5. In a still further embodiment, an acylated insulin has increased solubility relative to parent insulin at pH 4-8. In a yet further embodiment, an acylated insulin has increased solubility relative to parent insulin at pH 4.5-8. In a still further embodiment, an acylated insulin has increased solubility relative to parent insulin at pH 5-8. In a yet further embodiment, an acylated insulin has increased solubility relative to parent insulin at pH 5.5-8. In a further embodiment, an acylated insulin has increased solubility relative to parent insulin at pH 6-8. In one embodiment, an acylated insulin has increased solubility relative to parent insulin at pH 2-4.

In one embodiment, the solution may be subjected to centrifugation for 20 minutes at 30,000 g and then the insulin concentration in the supernatant may be determined by RP-HPLC. If this concentration is equal within experimental error to the insulin concentration originally used to make the composition, then the insulin is fully soluble in the composition of the invention. In one embodiment, the solubility of the insulin in a pharmaceutical composition of the invention may simply be determined by examining by eye the container in which the composition is contained. The insulin is soluble if the solution is clear to the eye and no particulate matter is either suspended or precipitated on the sides/bottom of the container.

In one embodiment an acylated insulin of the present invention has a side chain. In one embodiment the side chain is attached to the epsilon amino group of a lysine residue. In one embodiment a side chain according to the present invention is an acyl moiety. In one embodiment the side chain is attached to the epsilon amino group of a lysine residue in an insulin analogue. In one embodiment the side chain is attached to the epsilon amino group of a lysine residue in the B-chain of an insulin analogue.

In a further embodiment of the invention, a fatty diacid of a side chain in an acylated insulin in a pharmaceutical composition according to the present invention has from 6 to 40 carbon atoms. In a further embodiment of the invention, a fatty diacid of a side chain in an acylated insulin in a pharmaceutical composition according to the present invention has from 8 to 26 carbon atoms. In a further embodiment of the invention a fatty diacid of a side chain in an acylated insulin in a pharmaceutical composition according to the present invention has from 8 to 22 carbon atoms. In a further embodiment of the invention, a fatty diacid of a side chain in an acylated insulin in a pharmaceutical composition according to the present invention has from 14 to 22 carbon atoms. In a further embodiment of the invention, a fatty diacid of a side chain in an acylated insulin in a pharmaceutical composition according to the present invention has from 16 to 22 carbon atoms. In a further embodiment of the invention, a fatty diacid of a side chain in an acylated insulin in a pharmaceutical composition according to the present invention has from 16 to 20 carbon atoms. In a further embodiment of the invention, a fatty diacid of a side chain in an acylated insulin in a pharmaceutical composition according to the present invention has from 16 to 18 carbon atoms. In a further embodiment of the invention, a fatty diacid of a side chain in an acylated insulin in a pharmaceutical composition according to the present invention has 16 carbon atoms. In a further embodiment of the invention, a fatty diacid of a side chain in an acylated insulin in a pharmaceutical composition according to the present invention has 18 carbon atoms. In a further embodiment of the invention, a fatty diacid of a side chain in an acylated insulin in a pharmaceutical composition according to the present invention has 20 carbon atoms. In a further embodiment of the invention, a fatty diacid of a side chain in an acylated insulin in a pharmaceutical composition according to the present invention has 22 carbon atoms.

In one embodiment a tablet core according to the present invention comprises an acylated insulin as disclosed and claimed in patent applications WO2009/115469 or WO2011/161125. Methods for preparation of such insulins as well as assays for characterizing such insulins, such as physical and chemical stability as well as potency and T½ are provided in patent applications WO2009/115469 or WO2011/161125. In one embodiment a tablet core according to the present invention comprises an acylated insulin selected from the examples of patent applications WO2009/115469 or WO2011/161125.

In one embodiment an acylated insulin is an acylated insulin analogue, wherein said acylated insulin analogue comprises an A-chain amino acid sequence of formula 1:

Xaa_{A(-2)}-Xaa_{A(-1)}-Xaa_{A0}-Gly-Ile-Val-Glu-Gln-Cys-Cys-Xaa_{A8}-Ser-Ile-Cys-Xaa_{A12}-Xaa_{A13}-Xaa_{A14}-Xaa_{A15}-Leu-Glu-Xaa_{A18}-Tyr-Cys-Xaa_{A21} Formula (1) (SEQ ID No:1)

and a B-chain amino acid sequence of formula 2:

Xaa_{B(-2)}-Xaa_{B(-1)}-Xaa_{B0}-Xaa_{B1}-Xaa_{B2}-Xaa_{B3}-Xaa_{B4}-His-Leu-Cys-Gly-Ser-Xaa_{B10}-Leu-Val-Glu-Ala-Leu-Xaa_{B16}-Leu-Val-Cys-Gly-Glu-Arg-Gly-Xaa_{B24}-Xaa_{B25}-Xaa_{B26}-Xaa_{B27}-Xaa_{B28}-Xaa_{B29}-Xaa_{B30}-Xaa_{B31}-Xaa_{B32} Formula (2) (SEQ ID No:2)

wherein
Xaa_{A(-2)} is absent or Gly;
Xaa_{A(-1)} is absent or Pro;
Xaa_{A8} is absent or Pro;
Xaa_{A8} is independently selected from Thr and His;
Xaa_{A12} is independently selected from Ser, Asp and Glu;
Xaa_{A13} is independently selected from Leu, Thr, Asn, Asp, Gln, His, Lys, Gly, Arg, Pro, Ser and Glu;
Xaa_{A14} is independently selected from Tyr, Thr, Asn, Asp, Gln, His, Lys, Gly, Arg, Pro, Ser and Glu;
Xaa_{A15} is independently selected from Gln, Asp and Glu;
Xaa_{A18} is independently selected from Asn, Lys and Gln;
Xaa_{A21} is independently selected from Asn and Gln;
Xaa_{B(-2)} is absent or Gly;
Xaa_{B(-1)} is absent or Pro;
Xaa_{B0} is absent or Pro;
Xaa_{B1} is absent or independently selected from Phe and Glu;
Xaa_{B2} is absent or Val;
Xaa_{B3} is absent or independently selected from Asn and Gln;
Xaa_{B4} is independently selected from Gln and Glu;
Xaa_{B10} is independently selected from His, Asp, Pro and Glu;
Xaa_{B16} is independently selected from Tyr, Asp, Gln, His, Arg, and Glu;
Xaa_{B24} is independently selected from Phe and His;
Xaa_{B28} is independently selected from Asn, Phe and His;
Xaa_{B26} is absent or independently selected from Tyr, His, Thr, Gly and Asp;
Xaa_{B27} is absent or independently selected from Thr, Asn, Asp, Gln, His, Lys, Gly, Arg, Pro, Ser and Glu;
Xaa_{B28} is absent or independently selected from Pro, His, Gly and Asp;
Xaa_{B29} is absent or independently selected from Lys, Arg and Gln; and, preferably, Xaa_{B29} is absent or independently selected from Lys and Gln;
Xaa_{B38} is absent or Thr;
Xaa_{B31} is absent or Leu;
Xaa_{B32} is absent or Glu;
wherein the A-chain amino acid sequence and the B-chain amino acid sequence are connected by disulfide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulfide bridge.

In one embodiment, an derivative is an acylated insulin analogue, wherein said acylated insulin analogue comprises an A-chain amino acid sequence of formula 3:

Gly-Ile-Val-Glu-Gln-Cys-Cys-Xaa_{A8}-Ser-Ile-Cys-Xaa_{A12}-Xaa_{A13}-Xaa_{A14}-Xaa_{A18}-Leu-Glu-Xaa_{A18}-Tyr-Cys-Xaa_{A21} Formula (3) (SEQ ID No:3)

and a B-chain amino acid sequence of formula 4:

Xaa_{B1}-Val-Xaa_{B3}-Xaa_{B4}-His-Leu-Cys-Gly-Ser-Xaa_{A10}-Leu-Val-Glu-Ala-Leu-Xaa_{B16}-Leu-Val-Cys-Gly-Glu-Arg-Gly-Xaa_{B24}-His-Xaa_{B26}-Xaa_{B27}-Xaa_{B28}-Xaa_{B29}-Xaa_{B30} Formula (4) (SEQ ID No:4)

wherein
Xaa_{A8} is independently selected from Thr and His;
Xaa_{A12} is independently selected from Ser, Asp and Glu;
Xaa_{A13} is independently selected from Leu, Thr, Asn, Asp, Gln, His, Lys, Gly, Arg, Pro, Ser and Glu;
Xaa_{A14} is independently selected from Thr, Asn, Asp, Gln, His, Lys, Gly, Arg, Pro, Ser and Glu;
Xaa_{A18} is independently selected from Gln, Asp and Glu;
Xaa_{A18} is independently selected from Asn, Lys and Gln;
Xaa_{A21} is independently selected from Asn, and Gln;
Xaa_{B1} is independently selected from Phe and Glu;
Xaa_{B3} is independently selected from Asn and Gln;
Xaa_{B4} is independently selected from Gln and Glu;
Xaa_{B18} is independently selected from His, Asp, Pro and Glu;
Xaa_{B18} is independently selected from Tyr, Asp, Gln, His, Arg, and Glu;
Xaa_{B24} is independently selected from Phe and His;
Xaa_{B26} is absent or independently selected from Tyr, His, Thr, Gly and Asp;
Xaa_{B27} is absent or independently selected from Thr, Asn, Asp, Gln, His, Lys, Gly, Arg, Pro, Ser and Glu;
Xaa_{B28} is absent or independently selected from Pro, His, Gly and Asp;
Xaa_{B29} is absent or independently selected from Lys, Arg and Gln; and, preferably, Xaa_{B29} is absent or independently selected from Lys and Gln;
Xaa_{B30} is absent or Thr;
wherein the A-chain amino acid sequence and the B-chain amino acid sequence are connected by disulfide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulfide bridge.

In one embodiment, an acylated insulin is an acylated insulin analogue wherein

Xaa_{A8} is independently selected from Thr and His;
Xaa_{A12} is independently selected from Ser and Glu;
Xaa_{A13} is independently selected from Leu, Thr, Asn, Asp, Gln, His, Lys, Gly, Arg, Pro, Ser and Glu;
Xaa_{A14} is independently selected from Asp, His, and Glu;
Xaa_{A15} is independently selected from Gln and Glu;
Xaa_{A18} is independently selected from Asn, Lys and Gln;
Xaa_{A21} is independently selected from Asn, and Gln;
Xaa_{B1} is independently selected from Phe and Glu;
Xaa_{B3} is independently selected from Asn and Gln;
Xaa_{B4} is independently selected from Gln and Glu;
Xaa_{B10} is independently selected from His, Asp, Pro and Glu;
Xaa_{B16} is independently selected from Tyr, Asp, Gln, His, Arg, and Glu;
Xaa_{B24} is independently selected from Phe and His;
Xaa_{B26} is independently selected from Phe, Asn and His;
Xaa_{B26} is independently selected from Tyr, Thr, Gly and Asp;
Xaa_{B27} is independently selected from Thr, Asn, Asp, Gln, His, Lys, Gly, Arg, and Glu;
Xaa_{B28} is independently selected from Pro, Gly and Asp;
Xaa_{B29} is independently selected from Lys and Gln;
Xaa_{B38} is absent or Thr;
wherein the A-chain amino acid sequence and the B-chain amino acid sequence are connected by disulfide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulfide bridge.

An acylated insulin may have increased apparent potency and/or bioavalability relative to the parent insulin when compared upon measurement.

For the sake of convenience, here follows the names of codable, natural amino acids with the usual three letter codes & one letter codes in parenthesis: Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (His & H), lysine (Lys & K), arginine (Arg & R), glutamine (Gln & Q), asparagine (Asn & N), glutamic acid (Glu & E), aspartic acid (Asp & D), serine (Ser & S) and threonine (Thr & T). If, due to typing errors, there are deviations from the commonly used codes, the commonly used codes apply. The amino acids present in the acylated insulins for use in this invention are, preferably, amino acids which may be coded for by a nucleic acid. In one embodiment insulin or an insulin analogue or derivative is substituted by Gly, Glu, Asp, His, Gln, Asn, Ser, Thr, Lys, Arg and/or Pro and/or Gly, Glu, Asp, His, Gln, Asn, Ser, Thr, Lys, Arg and/or Pro is added to insulin or an insulin analogue or derivative. In one embodiment insulin or an insulin analogue or derivative is substituted by Glu, Asp, His, Gln, Asn, Lys and/or Arg, and/or Glu, Asp, His, Gln, Asn, Lys and/or Arg is added to an acylated insulin.

In one embodiment, an acylated insulin for a pharmaceutical composition according to this invention is an acylated insulin analogue comprising an insulin analogue before acylation and a side chain, wherein said insulin analogue before acylation is selected from the group consisting of: A14E, B25H, desB30 human insulin; A14H, B25H, desB30 human insulin; A14E, B1E, B25H, desB30 human insulin; A14E, B16E, B25H, desB30 human insulin; A14E, B25H, B28D, desB30 human insulin; A14E, B25H, B27E, desB30 human insulin; A14E, B1E, B25H, B27E, desB30 human insulin; A14E, B1E, B16E, B25H, B27E, desB30 human insulin; A8H, A14E, B25H, desB30 human insulin; A8H, A14E, B25H, B27E, desB30 human insulin; A8H, A14E, B1E, B25H, desB30 human insulin; A8H,
A14E, B1E, B25H, B27E, desB30 human insulin; A8H, A14E, B1E, B16E, B25H, B27E, desB30 human insulin; A8H, A14E, B16E, B25H, desB30 human insulin; A14E, B25H, B26D, desB30 human insulin; A14E, B1E, B27E, desB30 human insulin; A14E, B27E, desB30 human insulin; A14E, B28D, desB30 human insulin; A14E, B28E, desB30 human insulin; A14E, B1E, B28E, desB30 human insulin; A14E, B1E, B27E, B28E, desB30 human insulin; A14E, B1E, B25H, B28E, desB30 human insulin; A14E, B1E, B25H, B27E, B28E, desB30 human insulin; A14D, B25H, desB30 human insulin; B25N, B27E, desB30 human insulin; A8H, B25N, B27E, desB30 human insulin; A14E, B27E, B28E, desB30 human insulin; A14E, B25H, B28E, desB30 human insulin; B25H, B27E, desB30 human insulin; B1E, B25H, B27E, desb30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; A(−1)P, A(0)P, A14E, B25H, desB30 human insulin; A14E, B(−1)P, B(0)P, B25H, desB30 human insulin; A(−1)P, A(0)P, A14E, B(−1)P, B(0)P, B25H, desB30 human insulin; A14E, B25H, B30T, B31L, B32E human insulin; A14E, B25H human insulin; A14E, B16H, B25H, desB30 human insulin; A14E, B10P, B25H, desB30 human insulin; A14E, B10E, B25H, desB30 human insulin; A14E, B4E, B25H, desB30 human insulin; A14H, B16H, B25H, desB30 human insulin; A14H, B10E, B25H, desB30 human insulin; A13H, A14E, B10E, B25H, desB30 human insulin; A13H, A14E, B25H, desB30 human insulin; A14E, A18Q, B3Q, B25H, desB30 human insulin; A14E, B24H, B25H, desB30 human insulin; A14E, B25H, B26G, B27G, B28G, desB30 human insulin; A14E, B25H, B26G, B27G, B28G, B29R, desB30 human insulin; A14E, A21G, B25H, B26G, B27G, B28G, desB30 human insulin; A14E, A21G, B25H, B26G, B27G, B28G, B29R, desB30 human insulin; A14E, A18Q, A21Q, B3Q, B25H, desB30 human insulin; A14E, A18Q, A21Q, B3Q, B25H, B27E, desB30 human insulin; A14E, A18Q, B3Q, B25H, desB30 human insulin; A13H,
A14E, B1E, B25H, desB30 human insulin; A13N, A14E, B25H, desB30 human insulin; A13N, A14E, B1E, B25H, desB30 human insulin; A(−2)G, A(−1)P, A(0)P,
A14E, B25H, desB30 human insulin; A14E, B(−2)G, B(−1)P, B(0)P, B25H, desB30 human insulin; A(−2)G, A(−1)P, A(0)P, A14E, B(−2)G, B(−1)P, B(0)P, B25H, desB30 human insulin; A14E, B27R, B28D, B29K, desB30 human insulin; A14E, B25H, B27R, B28D, B29K, desB30 human insulin; A14E, B25H, B26T, B27R, B28D, B29K, desB30 human insulin; A14E, B25H, B27R, desB30 human insulin; A14E, B25H, B27H, desB30 human insulin; A14E, A18Q, B3Q, B25H, desB30 human insulin; A13E, A14E, B25H, desB30 human insulin; A12E, A14E, B25H, desB30 human insulin; A15E, A14E, B25H, desB30 human insulin; A13E, B25H, desB30 human insulin; A12E, B25H, desB30 human insulin; A15E, B25H, desB30 human insulin; A14E, B25H, desB27, desB30 human insulin; A14E, B25H, B26D, B27E, desB30 human insulin; A14E, B25H, B27R, desB30 human insulin; A14E, B25H, B27N, desB30 human insulin; A14E, B25H, B27D, desB30 human insulin; A14E, B25H, B27Q, desB30 human insulin; A14E, B25H, B27E, desB30 human insulin; A14E, B25H, B27G, desB30 human insulin; A14E, B25H, B27H, desB30 human insulin; A14E, B25H, B27K, desB30 human insulin; A14E, B25H, B27P, desB30 human insulin; A14E, B25H, B27S, desB30 human insulin; A14E, B25H, B27T, desB30 human insulin; A13R, A14E, B25H, desB30 human insulin; A13N,
A14E, B25H, desB30 human insulin; A13D, A14E, B25H, desB30 human insulin; A13Q, A14E, B25H, desB30 human insulin; A13E, A14E, B25H, desB30 human insulin; A13G, A14E, B25H, desB30 human insulin; A13H, A14E, B25H, desB30 human insulin; A13K, A14E, B25H, desB30 human insulin; A13P, A14E, B25H, desB30 human insulin; A13S, A14E, B25H, desB30 human insulin; A13T, A14E, B25H, desB30 human insulin; A14E, B16R, B25H, desB30 human insulin; A14E, B16D, B25H, desB30 human insulin; A14E, B16Q, B25H, desB30 human insulin; A14E, B16E, B25H, desB30 human insulin; A14E, B16H, B25H, desB30 human insulin; A14R, B25H, desB30 human insulin; A14N, B25H, desB30 human insulin; A14D, B25H, desB30 human insulin; A14Q, B25H, desB30 human insulin; A14E, B25H, desB30 human insulin; A14G, B25H, desB30 human insulin; A14H, B25H, desB30 human insulin; A8H, B10D, B25H human insulin; and A8H, A14E, B10E, B25H, desB30 human insulin and this embodiment may, optionally, comprise A14E, B25H, B29R, desB30 human insulin; B25H, desB30 human insulin; and B25N, desB30 human insulin.

In one embodiment, an acylated insulin for use in a pharmaceutical composition according to this invention is an acylated insulin analogue comprising an insulin analogue before acylation and a side chain, wherein said insulin analogue before acylation is selected from the group consisting of: A14E, B25H, desB30 human insulin, A14E, B16H, B25H, desB30 human insulin, A14E, B25H, desB27, desB30 human insulin and A14E, desB27, desB30 human insulin.

In one embodiment an acylated insulin in a pharmaceutical composition according to the invention has two or more cysteine substitutions, the three disulfide bonds of human insulin retained and a side chain which is attached to the epsilon amino group of a lysine residue such as in the B-chain.

Disulfide bonds are derived by the coupling of two thiol groups and are herein to be understood as the linkage between two sulfur atoms, i.e. a structure having the overall connectivity R-S-S-R. Disulfide bonds may also be called connecting disulfide bonds, SS-bonds or disulfide bridges. A disulfide bond is created by the introduction of two cysteine amino acid residues to a peptide with subsequent oxidation of the two thiol groups to a disulfide bond. Such oxidation may be performed chemically (as known by persons skilled in the art) or may happen during insulin expression in e.g. yeast.

In one embodiment an acylated insulin in a pharmaceutical composition according to the invention is a acylated insulin wherein two amino acid residues have been substituted by cysteine residues, a side chain has been introduced and optionally the amino acid in position B30 has been deleted relative to the amino acid sequence of human insulin.

In one embodiment an acylated insulin in a pharmaceutical composition according to the invention comprises a side chain and between 2 and 9 mutations relative to human insulin wherein at least two substitutions are to cysteine residues, alternatively an acylated insulin in a pharmaceutical composition according to the invention comprises a side chain and between 2 and 8 mutations relative to human insulin wherein at least two substitutions are to cysteine residues, alternatively a side chain and between 2 and 7 mutations relative to human insulin wherein at least two substitutions are to cysteine residues, alternatively a side chain and between 2 and 6 mutations relative to human insulin wherein at least two substitutions are to cysteine residues, alternatively a side chain and between 2 and 5 mutations relative to human insulin wherein at least two substitutions are to cysteine residues, alternatively a side chain and between 2 and 4 mutations relative to human insulin wherein at least two substitutions are to cysteine residues, alternatively a side chain and between 2 and 3 mutations relative to human insulin wherein at least two substitutions are to cysteine residues, or alternatively a side chain and 2 cysteine substitutions relative to human insulin.

In one embodiment an acylated insulin in a pharmaceutical composition according to the invention is an insulin analogue (as defined above) containing one or more additional disulfide bond(s) relative to human insulin and containing a side chain attached to the epsilon amino group of a lysine residue present in the B-chain of the molecule

When introducing cysteine residues into the acylated insulin without one or more additional disulfide bonds, the cysteine residues are placed in the three dimensional structure of the folded insulin analogue to allow for the formation of one or more additional disulfide bonds. For example, if placing two new cysteine residues, the proximity of the new cysteine residues in the three dimensional structure is such that a disulfide bond may be formed between the two new cysteine residues.

The number of disulfide bonds in a protein (such as insulin) may be readily determined by accurate intact mass measurements as described, for example in the Examples. The disulfide bonds connectivity may be verified (determined) by standard techniques known in the art, such as peptide mapping. The general strategy for disulfide bond mapping in an insulin peptide includes the following steps: 1) Fragmentation of the non-reduced insulin into disulfide bonded peptides containing, if possible, only a single disulfide bond per peptide. The chosen conditions is also such that rearrangement of disulfide bonds is avoided, 2) Separation of disulfide bonded peptides from each other. 3) Identification of the cysteine residues involved in the individual disulfide bonds.

In one embodiment of the invention an acylated insulin which has a side chain and at least two cysteine substitutions is provided, where the three disulfide bonds of human insulin are retained.

In one embodiment of the invention an acylated insulin which has two or more cysteine substitutions is provided, where the three disulfide bonds of human insulin are retained, and wherein at least one amino acid residue in a position selected from the group consisting of A9, A10 and A12 of the A-chain is substituted with a cysteine, at least one amino acid residue in a position selected from the group consisting of B1, B2, B3, B4, B5 and B6 of the B-chain is substituted with a cysteine, a side chain is attached to the epsilon amino group of a lysine residue in the B-chain and optionally the amino acid in position B30 is deleted.

In one embodiment of the invention the amino acid residue in position A10 of the A-chain is substituted with a cysteine, at least one amino acid residue in a position selected from the group consisting of B1, B2, B3, and B4 of the B-chain is substituted with a cysteine, a side chain is attached to the epsilon amino group of a lysine residue in the B-chain and optionally the amino acid in position B30 is deleted.

In one embodiment of the invention at least one amino acid residue in a position selected from the group consisting of A9, A10 and A12 of the A-chain is substituted with a cysteine, at least one amino acid residue in a position selected from the group consisting of B1, B2, B3, B4, B5 and B6 of the B-chain is substituted with a cysteine, at least one amino acid residue in a position selected from the group consisting of A14, A21, B1, B3, B10, B16, B22, B25, B26, B27, B28, B29, B30, B31, B32 is substituted with an amino acid which is not a cysteine, a side chain is attached to the epsilon amino group of a lysine residue in the B-chain and optionally the amino acid in position B30 is deleted.

It is understood that when B1 or B3 is cysteine, the same amino acid cannot be an amino acid which is not cysteine, whereas if e.g. B1 is cysteine B3 may according to the embodiment of the invention be substituted with an amino acid which is not a cysteine and vice versa. In one embodiment of the invention, the amino acid residue in position A10 of the A-chain is substituted with a cysteine, at least one amino acid residue in a position selected from the group consisting of B1, B2, B3, and B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one embodiment of the invention, the amino acid residue in position A10 of the A-chain is substituted with a cysteine, at least one amino acid residue in a position selected from the group consisting of B3 and B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one embodiment of the invention, the amino acid residue in position A10 of the A-chain is substituted with a cysteine, the amino acid residue in position B3 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one embodiment of the invention, the amino acid residue in position A10 of the A-chain is substituted with a cysteine, the amino acid residue in B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted.

An additional disulfide bond obtained by the invention may be connecting two cysteines of the same chain, i.e. two cysteines in the A-chain or two cysteines in the B-chain of the insulin, or connecting a cysteine in the A-chain with a cysteine in the B-chain of the insulin. In one embodiment, an acylated insulin in a pharmaceutical composition according to the invention is obtained, wherein at least one additional disulfide bond is connecting two cysteines in the A-chain or connecting two cysteines in the B-chain. In one embodiment, an acylated insulin in a pharmaceutical composition according to the invention is obtained, wherein at least one additional disulfide bond is connecting a cysteine in the A-chain with a cysteine in the B-chain.

In one embodiment of the invention, cysteines are substituted into two positions of the acylated insulin, where the positions are selected from the group consisting of:
A10C, B1C;
A10C, B2C;
A10C, B3C;
A10C, B4C;
A10C, B5C; and
B1C, B4C.

In one embodiment of the invention, cysteines are substituted into two positions of the insulin analogue, where the positions are selected from the group consisting of:
A10C, B1C;
A10C, B2C;
A10C, B3C;
A10C, B4C; and
B1C, B4C.

In one embodiment of the invention, cysteines are substituted into two positions of the acylated insulin, where the positions are selected from the group consisting of:
A10C, B1C;
A10C, B2C;
A10C, B3C; and
A10C, B4C.

In one embodiment of the invention, cysteines are substituted into two positions of the insulin analogue, where the positions are selected from the group consisting of:
A10C, B3C; and
A10C, B4C.

In one embodiment of the invention, cysteines are substituted into two positions of the insulin analogue, where the positions are A10C and B3C.

In one embodiment of the invention, cysteines are substituted into two positions of the insulin analogue, where the positions are A10C and B4C.

In one embodiment of the invention, acylated insulins of the invention comprise in addition to the cysteine substitutions one or more amino acids selected from the group consisting of: A8H, A14E, A14H, A18L, A21G, BIG, B3Q, B3E, B3T, B3V, B3K, B3L, B16H, B16E, B22E, B24G, B25A, B25H, B25N, B27E, B27D, B27P, B28D, B28E, B28K, desB1, desB24, desB25, desB27 and desB30. In one embodiment of the invention, acylated insulins of the invention comprise in addition to the cysteine substitutions one or more amino acids selected from the group consisting of: A8H, A14E, A21G, desB1, BIG, B3Q, B3E, B10E, B16H, B16E, B24G, B25H, B25A, B25N, B25G, desB27, B27E, B28E, B28D, and desB30.

In one embodiment of the invention, acylated insulins of the invention comprise in addition to the cysteine substitutions one or more amino acids selected from the group consisting of: A21G, desB1, BIG, B3Q, B3S, B3T and B3E.

In one embodiment of the invention, acylated insulins of the invention comprise in addition to the cysteine substitutions one or more amino acids selected from the group consisting of: A8H, A14E, A14H, B16H, B10E, B16E, B25H, B25A, B25N, B27E, B27P, desB27, B28E and desB30.

In one embodiment of the invention, acylated insulins of the invention comprise in addition to the cysteine substitutions one or more amino acids selected from the group consisting of: B28E, B28D, desB27, desB30 and A14E.

In one embodiment of the invention, acylated insulins of the invention comprise in addition to the cysteine substitutions one or more amino acids selected from the group consisting of: B3K, B29E, B27E, B27D, desB27, B28E, B28D, B28K and B29P

In one embodiment of the invention, acylated insulins of the invention comprise in addition to the cysteine substitutions a C-peptide connecting the C-terminus of the B-chain with the N-terminus of the A-chain (to form a so called single-chain acylated insulin). In one embodiment of the invention, the parent insulin is selected from the group consisting of single chain insulin analogues. In one embodiment of the invention, the parent insulin is selected from the group consisting of single chain insulin analogues listed in WO2007096332, WO2005054291 or WO2008043033, which patents are herein specifically incorporated by reference.

In one embodiment of the invention, an acylated insulin is obtained which comprises two cysteine substitutions resulting in one additional disulfide bond relative to human insulin.

In one embodiment an acylated insulin in a pharmaceutical composition according to the invention has two or more cysteine substitutions in addition to the three disulfide bonds of human insulin which are retained.

In one embodiment of the invention, the sites of cysteine substitutions are chosen in such a way that the introduced cysteine residues are placed in the three dimensional structure of the folded acylated insulin to allow for the formation of one or more additional disulfide bonds.

Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, a term like A10C designates that the amino acid in the A10 position is cysteine. Using the three letter codes for amino acids, the corresponding expression is A10Cys.

By “desB30”, “B(1-29)” or “desThrB30” is meant a natural insulin B chain or an analogue thereof lacking the B30 (threonine, Thr) amino acid and “A(1-21)” means the natural insulin A chain. Thus, e.g., A10C, B1C, desB30 human insulin or alternatively A10Cys,B1Cys,desB30 human insulin (or alternatively CysA10,CysB1,desThrB30 human insulin) is an analogue of human insulin where the amino acid in position 10 in the A chain is substituted with cysteine, the amino acid in position 1 in the B chain is substituted with cysteine, and the amino acid in position 30 (threonine, Thr) in the B chain is deleted.

Herein, the naming of the peptides or proteins is done according to the following principles: The names are given as mutations and modifications (such as acylations) relative to the parent peptide or protein such as human insulin. For the naming of the acyl moiety, the naming is done according to IUPAC nomenclature and in other cases as peptide nomenclature. For example, naming the acyl moiety:

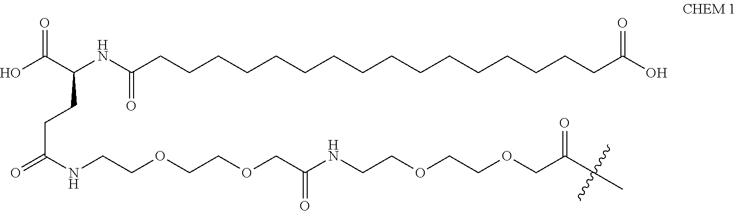 [SMILES: *C(=O)COCCOCCNC(=O)COCCOCCNC(=O)CC[C@H](NC(=O)CCCCCCCCCCCCCCCCC(=O)O)C(=O)O]

may e.g. be “octadecanedioyl-γGlu-OEG-OEG”, “octadecanedioyl-gGlu-OEG-OEG”, “octadecanedioyl-gGlu-2×OEG”, or “17-carboxyheptadecanoyl-γGlu-OEG-OEG”, wherein OEG is short hand notation for the amino acid residue, 8-amino-3,6-dioxaoctanoic acid, —NH(CH₂)₂O(CH₂)₂OCH₂CO—, and γGlu (or gGlu) is short hand notation for the amino acid gamma L-glutamic acid moiety.

One example is the insulin of example 1 in patent application WO2011/161125 (with the sequence/structure given below) is named “A10C, A14E, B4C, B25H, B29K(N^{ε}Octadecanedioyl-γGlu-OEG-OEG), desB30 human insulin” to indicate that the amino acid in position A10 in human insulin, has been mutated to C; A14, Y in human insulin, has been mutated to E; the amino acid in position B4, Q in human insulin, has been mutated to C; the amino acid in position B25, F in human insulin, has been mutated to H, the amino acid in position B29, K as in human insulin, has been modified by acylation on the epsilon nitrogen in the lysine residue of B29, denoted N^{ε}, by the residue octadecanedioyl-γGlu-OEG-OEG, and the amino acid in position B30, T in human insulin, has been deleted. Asterisks in the formula below indicate that the residue in question is different (i.e. mutated) as compared to human insulin. The disulfide bonds as found in human insulin are shown with sulphur atoms, and the additional disulfide bond of the invention is shown with a line.

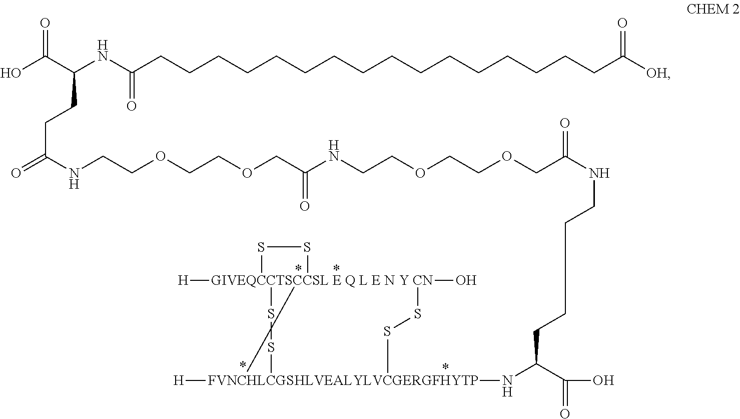

(SEQ ID NO: 5 and 6, 5 is the modified A chain and 6, the modified B chain of Chem 2)

In addition, the insulins of the invention may also be named according to IUPAC nomenclature (OpenEye, IUPAC style). According to this nomenclature, the above acylated insulin with an additional disulfide bridge is assigned the following name:

N{Epsilon-B29}-[2-[2-[2-[[2-[2-[2-[[(45)-4-carboxy-4-(17-carboxyheptadecanoylamino)butanoyl]amino]ethoxy]ethoxy]-acetyl]amino]ethoxy]ethoxy]acetyl]-[CysA10,GluA14,CysB4,HisB25],desThrB30-Insulin (human).

Herein, the term “amino acid residue” is an amino acid from which a hydroxy group has been removed from a carboxy group and/or from which a hydrogen atom has been removed from an amino group.

In one embodiment of the invention, the acylated insulin in a pharmaceutical composition according to the invention comprises a side chain in the form of an acyl group on e.g. the ε-amino group of a Lys residue of the insulin amino acid sequence. In one embodiment the acylated insulin comprises an albumin binding residue, i.e. a residue which under in vivo conditions binds to albumin when attached to a peptide or protein.

In a still further particular embodiment the albumin binding moiety comprises a portion in between the protracting moiety and the point of attachment to the peptide, which portion may be referred to as a “linker”, “linker moiety”, “spacer”, or the like. The linker may be optional, and hence in that case the albumin binding moiety may be identical to the protracting moiety.

In one embodiment, the albumin binding residue is a lipophilic residue. In a further embodiment, the lipophilic residue is attached to the insulin amino acid sequence via a linker.

In a further embodiment of the invention, the albumin binding residue is negatively charged at physiological pH. In another embodiment of the invention, the albumin binding residue comprises a group which may be negatively charged. One preferred group which may be negatively charged is a carboxylic acid group.

In one embodiment, the albumin binding residue is an α,ω-fatty diacid residue. In a further embodiment of the invention, the α,ω-fatty diacid residue of the lipophilic residue in the acylated insulin has from 6 to 40 carbon atoms, from 8 to 26 carbon atoms or from 8 to 22 carbon atoms, or from 14 to 22 carbon atoms, or from 16 to 22 carbon atoms, or from 16 to 20 carbon atoms, or from 16 to 18 carbon atoms, or 16 carbon atoms, or 18 carbon atoms, or 20 carbon atoms, or 22 carbon atoms.

In one embodiment, the α,ω-fatty diacid residue of the lipophilic residue in the acylated insulin has 18 carbon atoms. In one embodiment the tablet core of the present invention comprises an acylated insulin, wherein the α,ω-fatty diacid residue of the lipophilic residue has 18 carbon atoms and provides higher values of acylated insulin bioavailability relative to those comprising 20 carbon atoms. In one embodiment, the α,ω-fatty diacid residue in the acylated insulin of the lipophilic residue has 20 carbon atoms. In one embodiment the tablet core of the present invention comprises an acylated insulin, wherein the α,ω-fatty diacid residue of the lipophilic residue has 20 carbon atoms and provides lower values of acylated insulin bioavailability relative to those comprising 18 carbon atoms. In one embodiment the tablet core of the present invention comprises an acylated insulin, wherein the α,ω-fatty diacid residue of the lipophilic residue has 20 carbon atoms and provides lower values of acylated insulin bioavailability, having a longer PK/PD profile relative to those comprising 18 carbon atoms.

In another embodiment of the invention, the albumin binding residue is an acyl group of a straight-chain or branched alkane α,ω-dicarboxylic acid. In a further embodiment the albumin binding residue is an acyl group of a straight-chain or branched alkane α,ω-dicarboxylic acid which includes an amino acid portion such as e.g. a gamma-Glu (γGlu) portion. In yet a further embodiment the albumin binding residue is an acyl group of a straight-chain or branched alkane α,ω-dicarboxylic acid which includes two amino acid portions such as e.g. a gamma-Glu (γGlu) portion and a 8-amino-3,6-dioxaoctanoic acid (OEG) portion. In yet a further embodiment the albumin binding residue is an acyl group of a straight-chain or branched alkane α,ω-dicarboxylic acid which includes more amino acid portions such as e.g. one gamma-Glu (γGlu) portion and consecutive 8-amino-3,6-dioxaoctanoic acid (OEG) portions.

In one embodiment, the acyl moiety attached to the parent (e.g.protease stabilised) insulin analogue has the general formula:

Acy-AA1ₙ-AA2ₘ-AA3ₚ- CHEM 3

wherein n is 0 or an integer in the range from 1 to 3; m is 0 or an integer in the range from 1 to 10; p is 0 or an integer in the range from 1 to 10; Acy is a fatty acid or a fatty diacid comprising from about 8 to about 24 carbon atoms such as from about 14 to about 22 carbon atoms; AA1 is a neutral linear or cyclic amino acid residue; AA2 is an acidic amino acid residue; AA3 is a neutral, alkyleneglycol-containing amino acid residue; the order by which AA1, AA2 and AA3 appears in the formula may be interchanged independently; AA2 may occur several times along the formula (e.g., Acy-AA2-AA3₂-AA2-); AA2 may occur independently (=being different) several times along the formula (e.g., Acy-AA2-AA3₂-AA2-); the connections between Acy, AA1, AA2 and/or AA3 are amide (peptide) bonds which, formally, may be obtained by removal of a hydrogen atom or a hydroxyl group (water) from each of Acy, AA1, AA2 and AA3; and attachment to the insulin analogue may be from the C-terminal end of a AA1, AA2, or AA3 residue in the acyl moiety of CHEM 3 or from one of the side chain(s) of an AA2 residue present in the moiety of CHEM 3.

In another embodiment, the acyl moiety attached to the parent insulin analogue has the general formula Acy-AA1ₙ-AA2ₘ-AA3ₚ- CHEM 3, wherein AA1 is selected from Gly, D- or L-Ala, βAla, 4-aminobutyric acid, 5-aminovaleric acid, 6-aminohexanoic acid, D- or L-Glu-α-amide, D- or L-Glu-γ-amide, D- or L-Asp-α-amide, D- or L-Asp-β-amide, or a group of one of the formula:

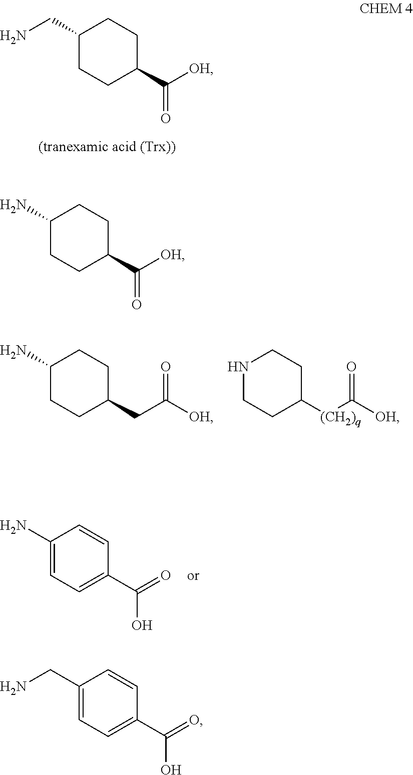

from which a hydrogen atom and/or a hydroxyl group has been removed and wherein q is 0, 1, 2, 3 or 4 and, in this embodiment, AA1 may, alternatively, be 7-aminoheptanoic acid or 8-aminooctanoic acid.

In another embodiment, the acyl moiety attached to the parent insulin analogue has the general formula Acy-AA1ₙ-AA2ₘ-AA3ₚ- (CHEM 3), wherein AA1 is as defined above and AA2 is selected from L- or D-Glu, L- or D-Asp, L- or D-homoGlu or any of the following:

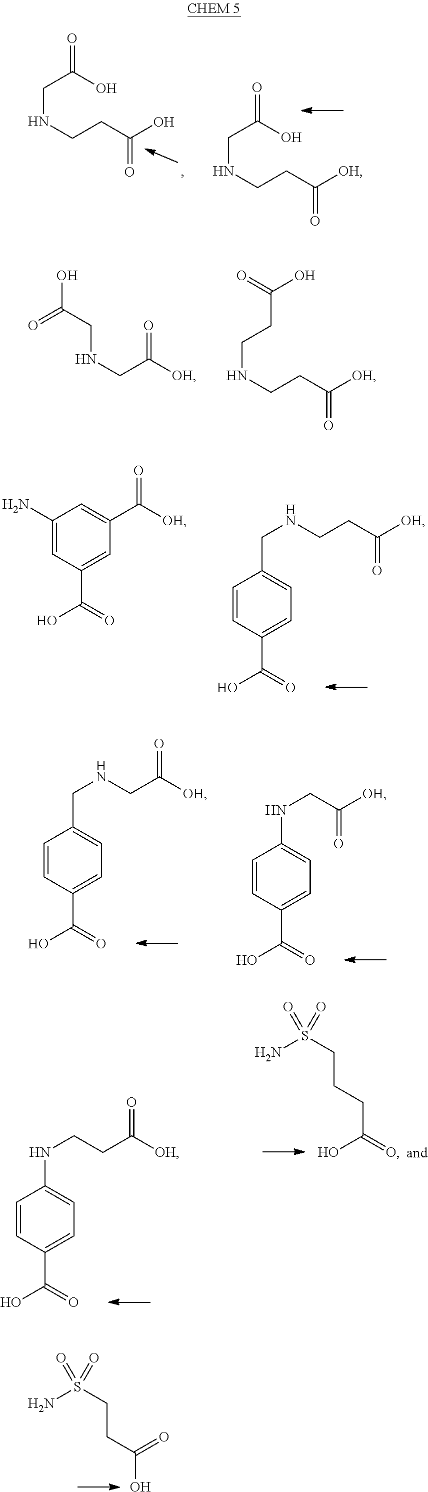

from which a hydrogen atom and/or a hydroxyl group has been removed and wherein the arrows indicate the attachment point to the amino group of AA1, AA2, AA3, or to the amino group of the insulin analogue.

In one embodiment, the neutral cyclic amino acid residue designated AA1 is an amino acid containing a saturated 6-membered carbocyclic ring, optionally containing a nitrogen hetero atom, and preferably the ring is a cyclohexane ring or a piperidine ring. Preferably, the molecular weight of this neutral cyclic amino acid is in the range from about 100 to about 200 Da.

The acidic amino acid residue designated AA2 is an amino acid with a molecular weight of up to about 200 Da comprising two carboxylic acid groups and one primary or secondary amino group. Alternatively, acidic amino acid residue designated AA2 is an amino acid with a molecular weight of up to about 250 Da comprising one carboxylic acid group and one primary or secondary sulphonamide group.

The neutral, alkyleneglycol-containing amino acid residue designated AA3 is an alkyleneglycol moiety, optionally an oligo- or polyalkyleneglycol moiety containing a carboxylic acid functionality at one end and an amino group functionality at the other end.

Herein, the term alkyleneglycol moiety covers mono-alkyleneglycol moieties as well as oligo-alkyleneglycol moieties. Mono- and oligoalkyleneglycols comprises mono- and oligoethyleneglycol based, mono- and oligopropyleneglycol based and mono- and oligobutyleneglycol based chains, i.e., chains that are based on the repeating unit —CH₂CH₂O—, —CH₂CH₂CH₂O— or —CH₂CH₂CH₂CH₂O—. The alkyleneglycol moiety is monodisperse (with well defined length/molecular weight). Monoalkyleneglycol moieties comprise —OCH₂CH₂O—, —OCH₂CH₂CH₂O— or —OCH₂CH₂CH₂CH₂O-containing different groups at each end.

As mentioned herein, the order by which AA1, AA2 and AA3 appears in the acyl moiety with CHEM 3 (Acy-AA1ₙ-AA2ₘ-AA3ₚ-) may be interchanged independently. Consequently, the formula Acy-AA1ₙ-AA2ₘ-AA3ₚ- also covers moieties like, e.g., the formula Acy-AA2ₘ-AA1ₙ-AA3ₚ-, the formula Acy-AA2-AA3ₙ-AA2-, and the formula Acy-AA3ₚ-AA2ₘ-AA1ₙ-, wherein Acy, AA1, AA2, AA3, n, m and p are as defined herein.

As mentioned herein, the connections between the moieties Acy, AA1, AA2 and/or AA3 are formally obtained by amide bond (peptide bond) formation (—CONH—) by removal of water from the parent compounds from which they formally are build. This means that in order to get the complete formula for the acyl moiety with the formula CHEM 3 (Acy-AA1ₙ-AA2ₘ-AA3ₚ-, wherein Acy, AA1, AA2, AA3, n, m and p are as defined herein), one has, formally, to take the compounds given for the terms Acy, AA1, AA2 and AA3 and remove a hydrogen and/or hydroxyl from them and, formally, to connect the building blocks so obtained at the free ends so obtained.

Non-limiting, specific examples of the acyl moieties of CHEM 3 Acy-AA1ₙ-AA2ₘ-AA3ₚ- which may be present in the acylated insulin analogues of this invention are listed in WO 2009/115469 A1, pp. 27-43:

Any of the above non-limiting specific examples of acyl moieties of the formula Acy-AA1ₙ-AA2ₘ-AA3ₚ- may be attached to an epsilon amino group of a lysine residue present in any of the above non-limiting specific examples of parent insulin analogues thereby giving further specific examples of acylated insulin analogues of this invention.

The parent insulin analogues may be converted into the acylated insulins containing additional disulfide bonds of this invention by introducing of the desired group of the formula Acy-AA1ₙ-AA2ₘ-AA3ₚ- in the lysine residue. The desired group of the formula Acy-AA1ₙ-AA2ₘ-AA3ₚ- may be introduced by any convenient method and many methods are disclosed in the prior art for such reactions. More details appear from the examples herein.

Non-limiting, specific examples of the acyl moieties of the formula Acy-AA1ₙ-AA2ₘ-AA3ₚ- which may be present in the acylated insulin analogues of this invention are the following:

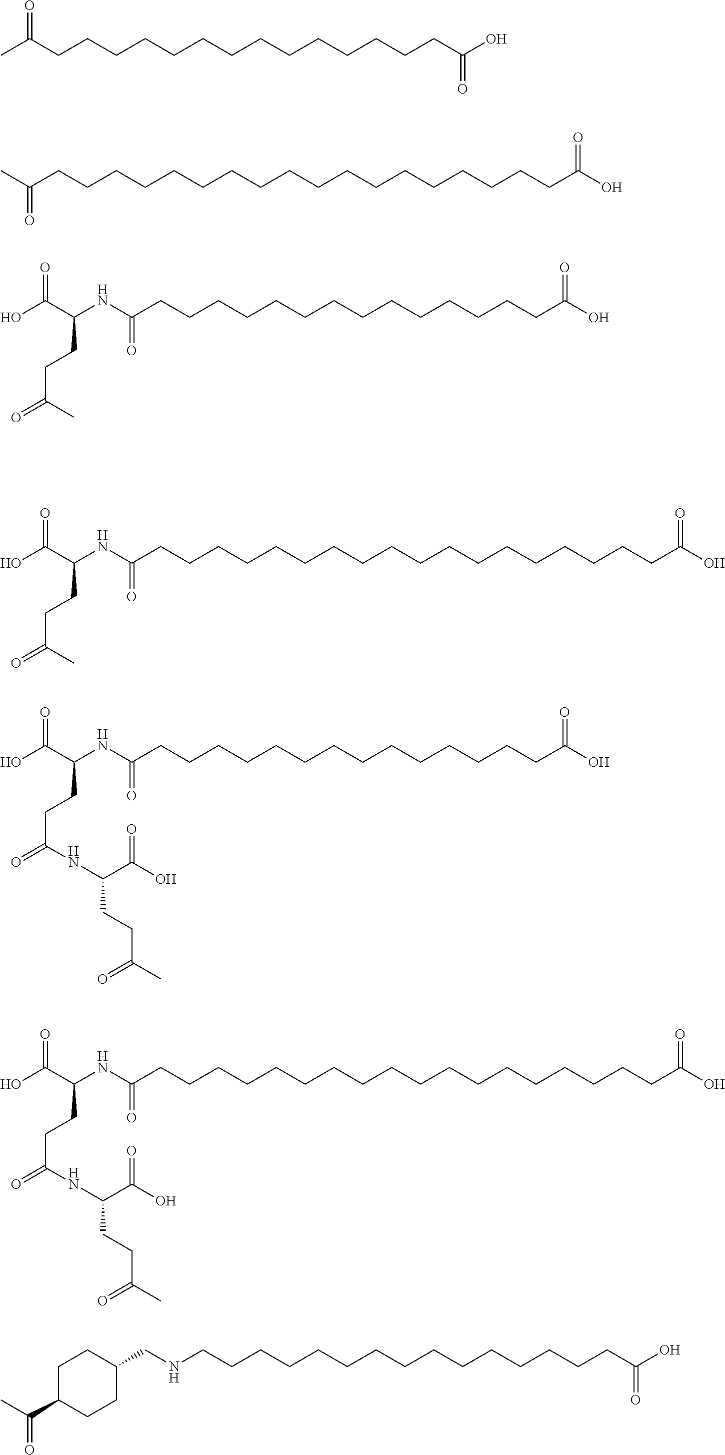

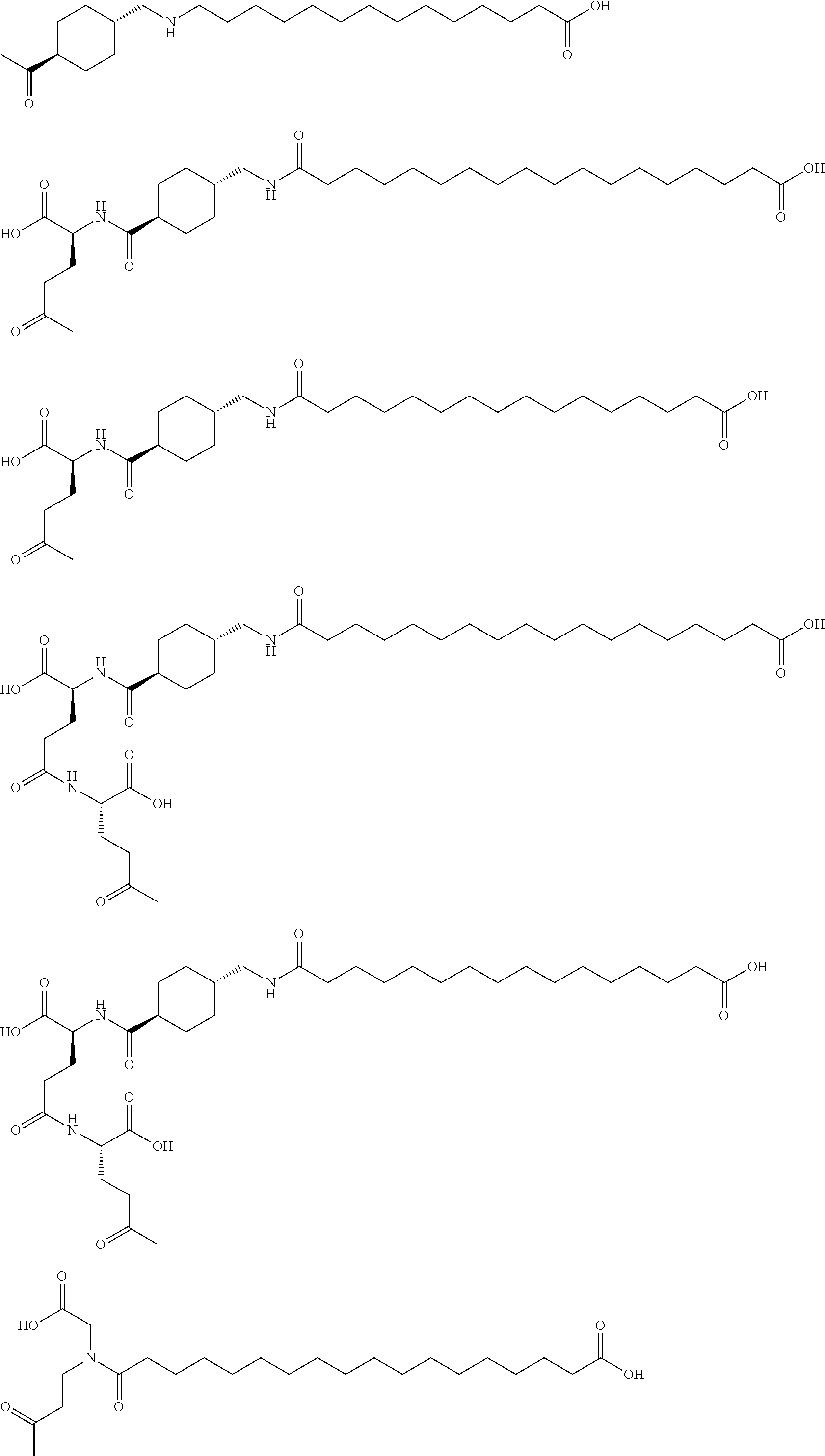 [SMILES: CC(=O)[C@H]1CC[C@H](CNCCCCCCCCCCCCCC(=O)O)CC1 ; CC(=O)CC[C@H](NC(=O)[C@H]1CC[C@H](CNC(=O)CCCCCCCCCCCCCCCCC(=O)O)CC1)C(=O)O ; CC(=O)CC[C@H](NC(=O)[C@H]1CC[C@H](CNC(=O)CCCCCCCCCCCCCCC(=O)O)CC1)C(=O)O ; CC(=O)CC[C@H](NC(=O)CC[C@H](NC(=O)[C@H]1CC[C@H](CNC(=O)CCCCCCCCCCCCCCCCC(=O)O)CC1)C(=O)O)C(=O)O ; CC(=O)CC[C@H](NC(=O)CC[C@H](NC(=O)[C@H]1CC[C@H](CNC(=O)CCCCCCCCCCCCCCC(=O)O)CC1)C(=O)O)C(=O)O ; CC(=O)CCN(CC(=O)O)C(=O)CCCCCCCCCCCCCCCCC(=O)O]

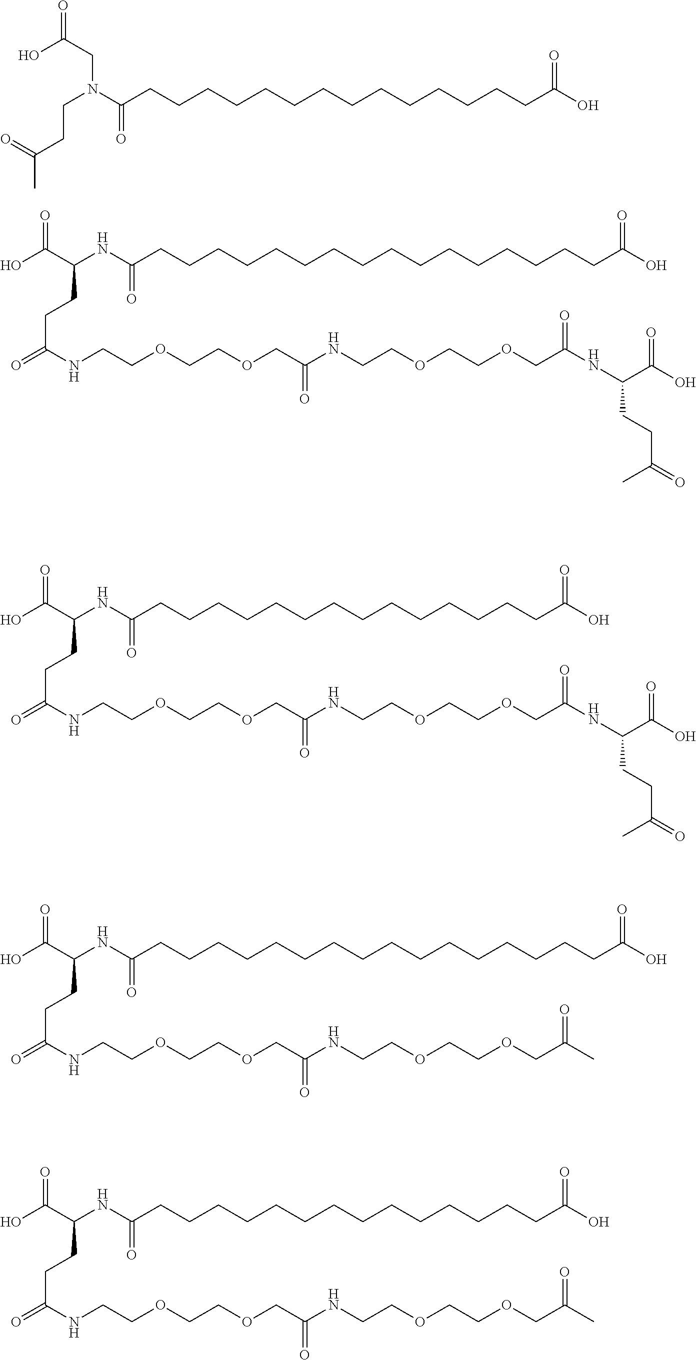

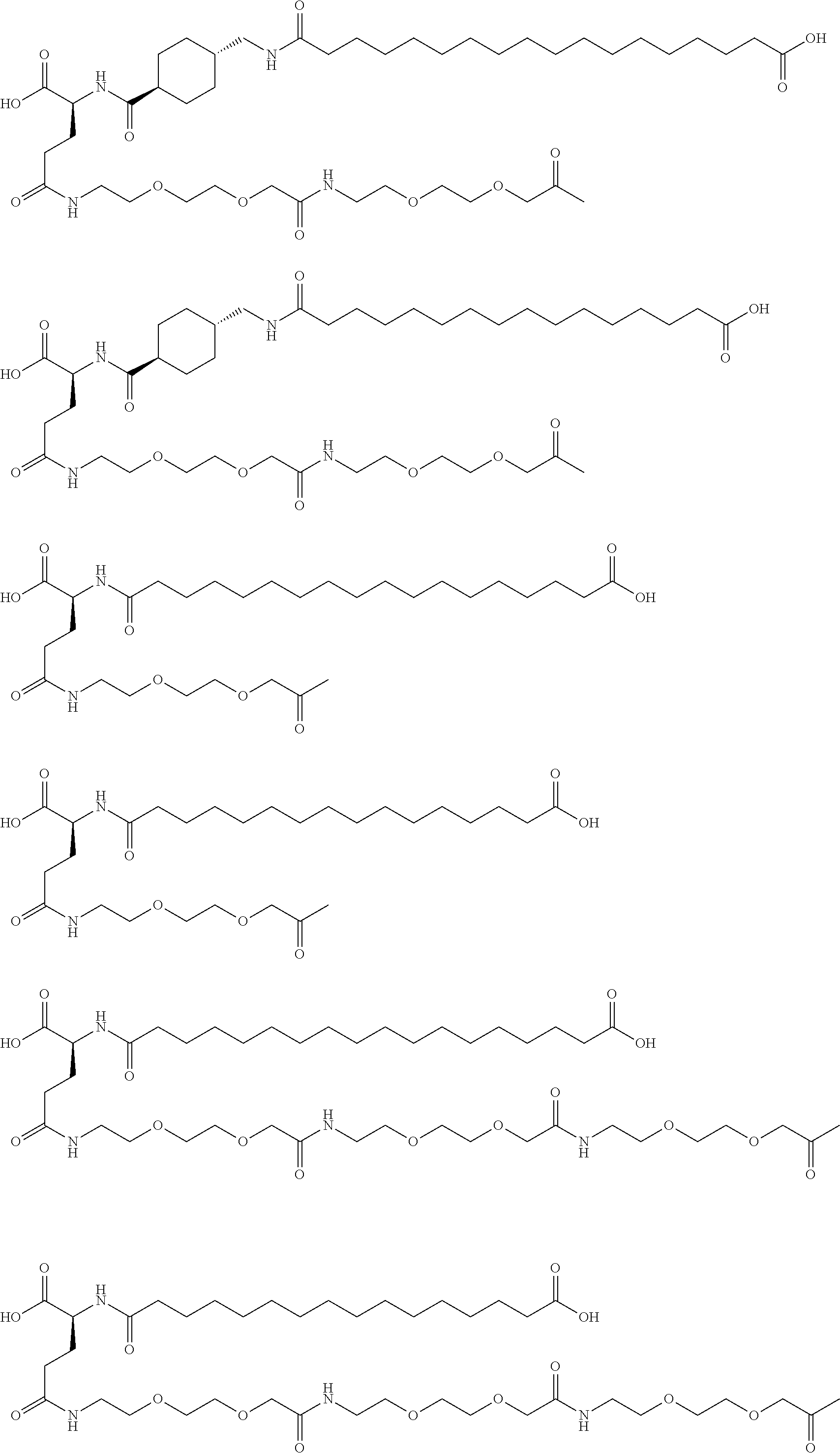

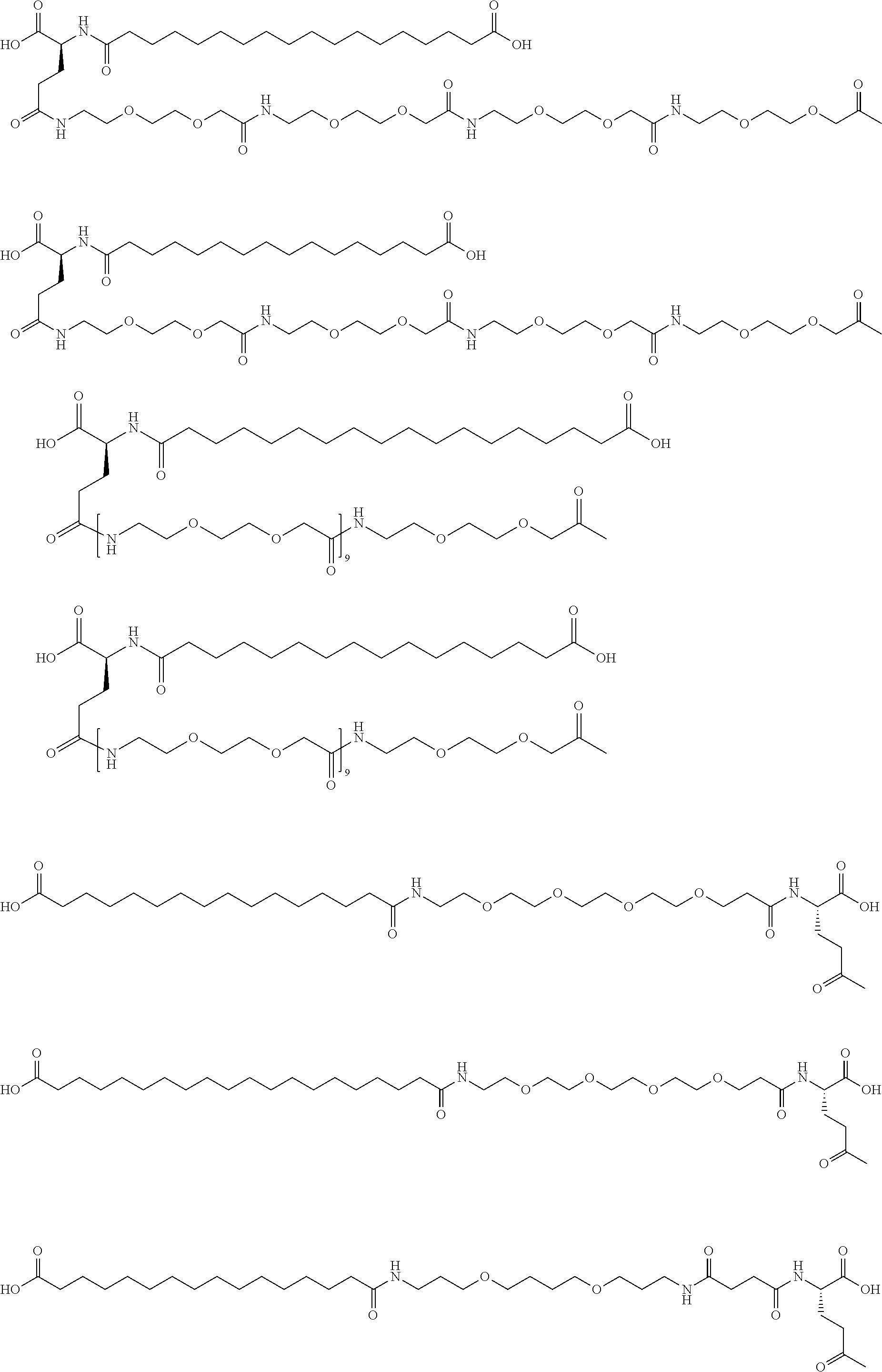

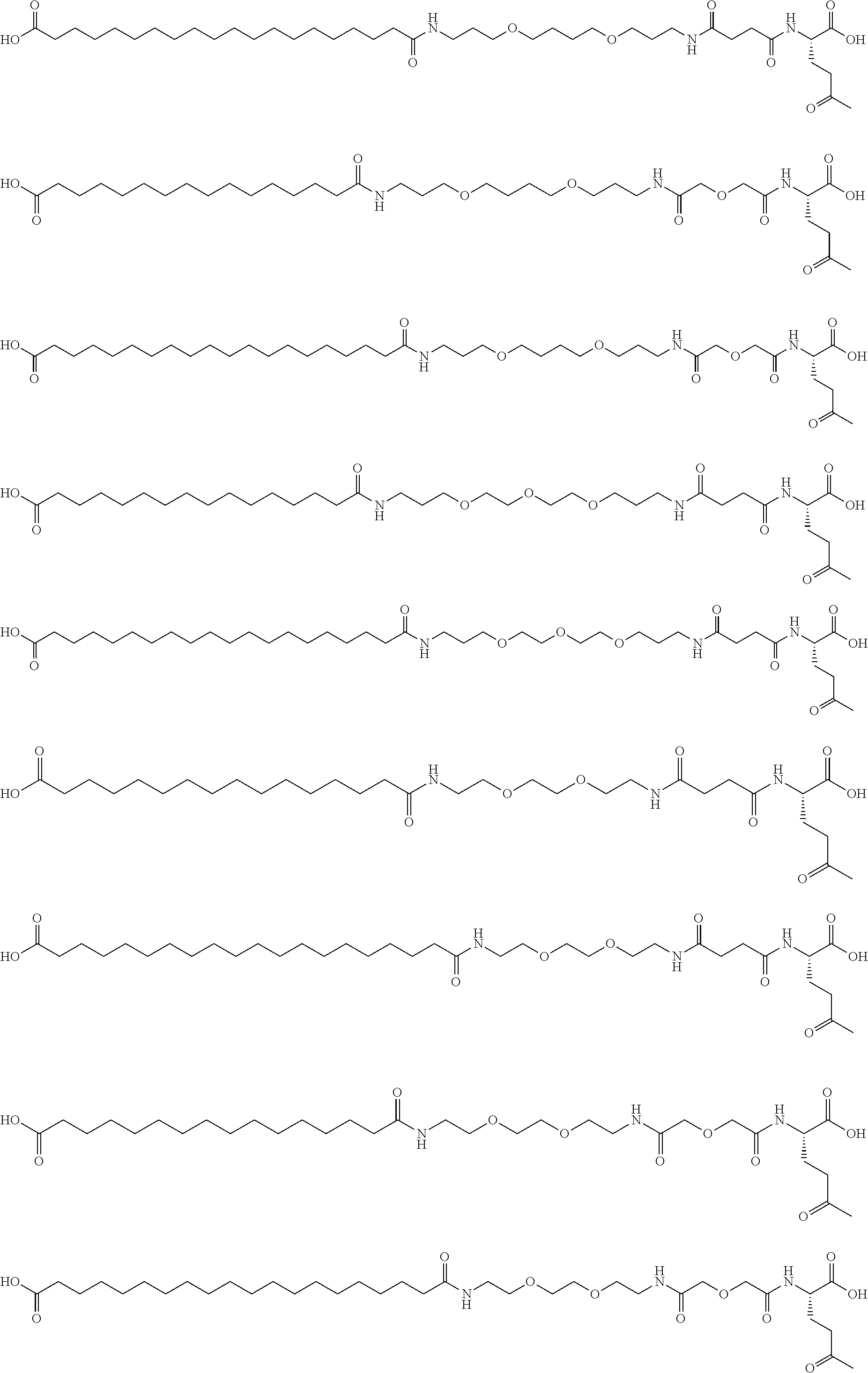

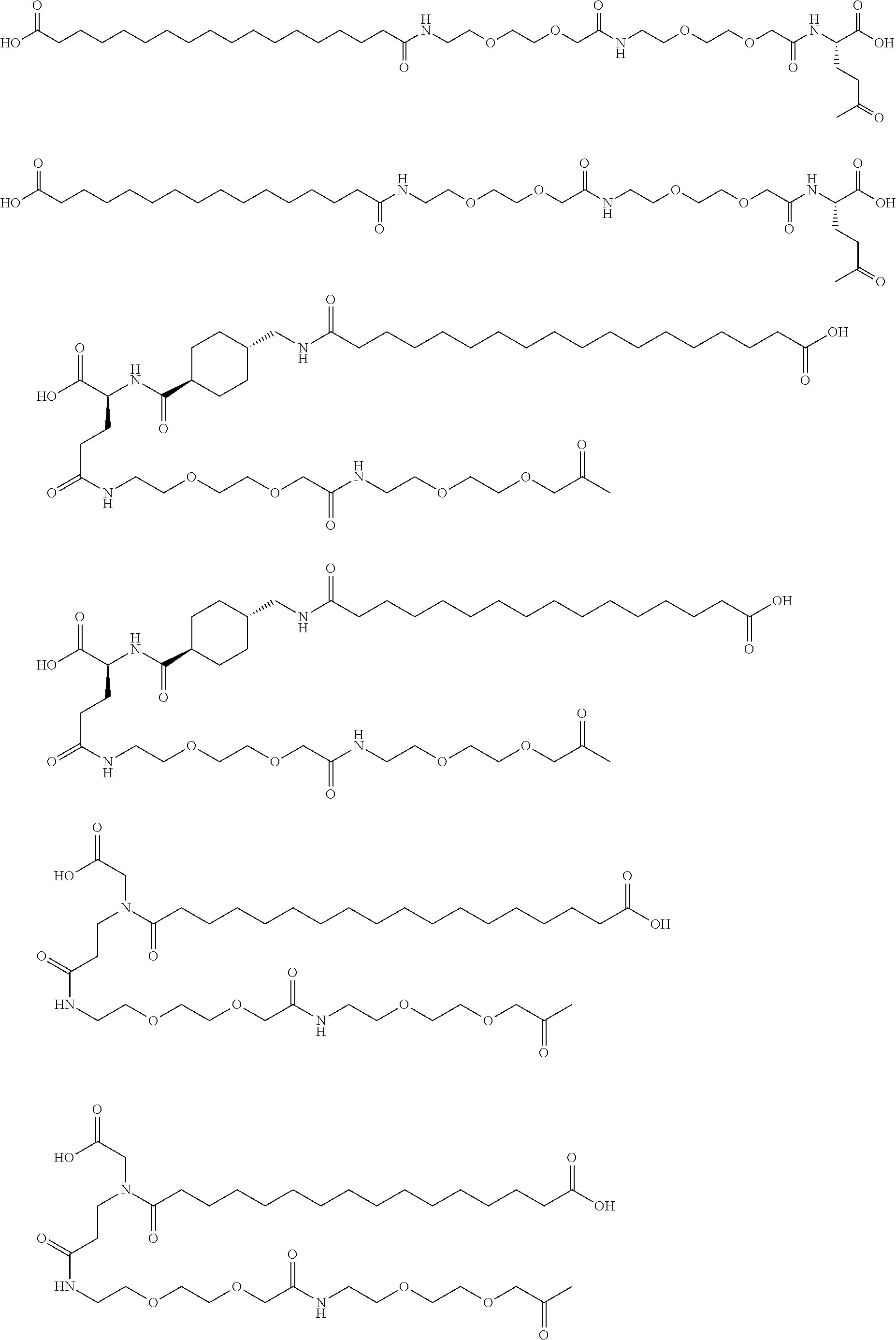

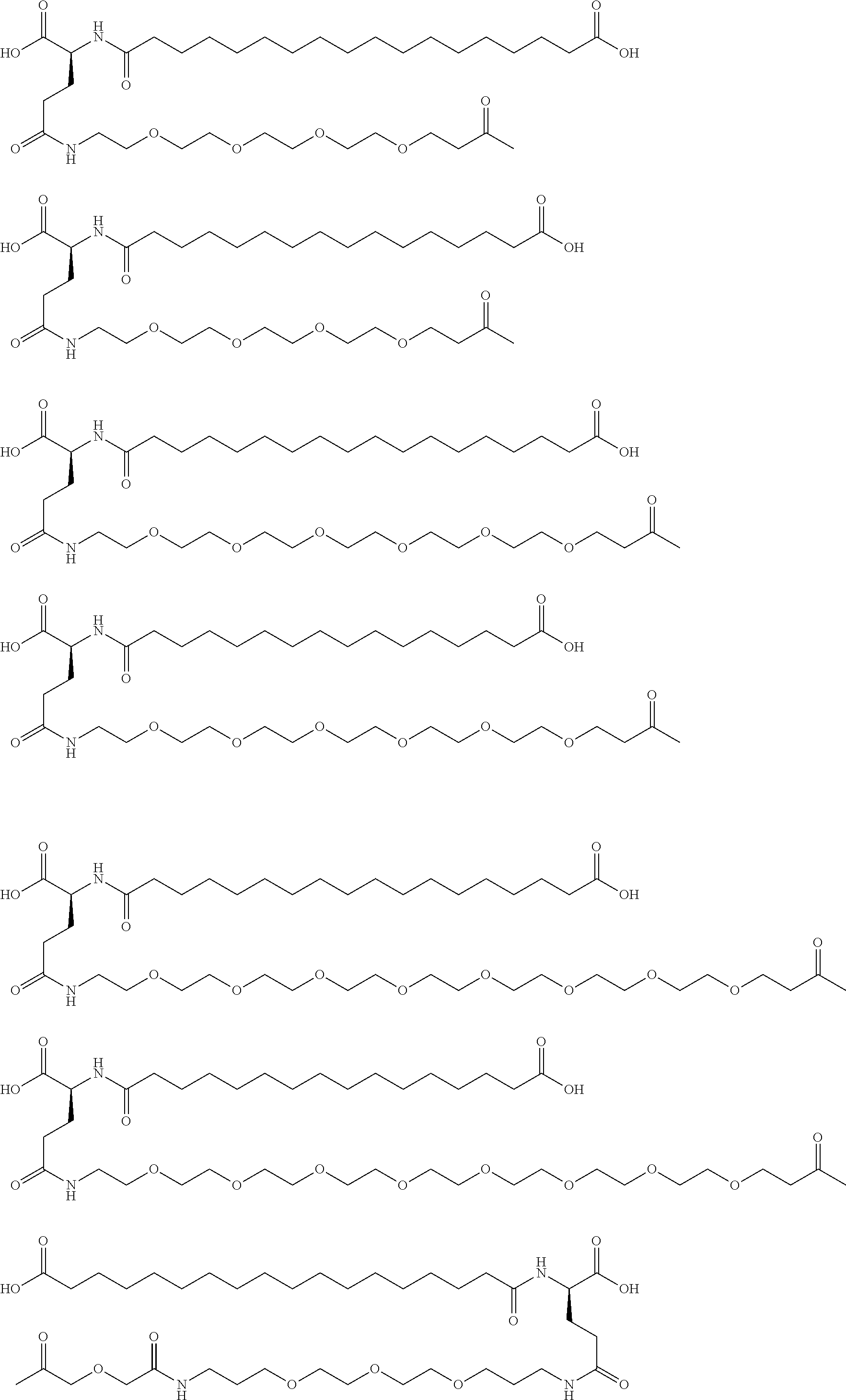

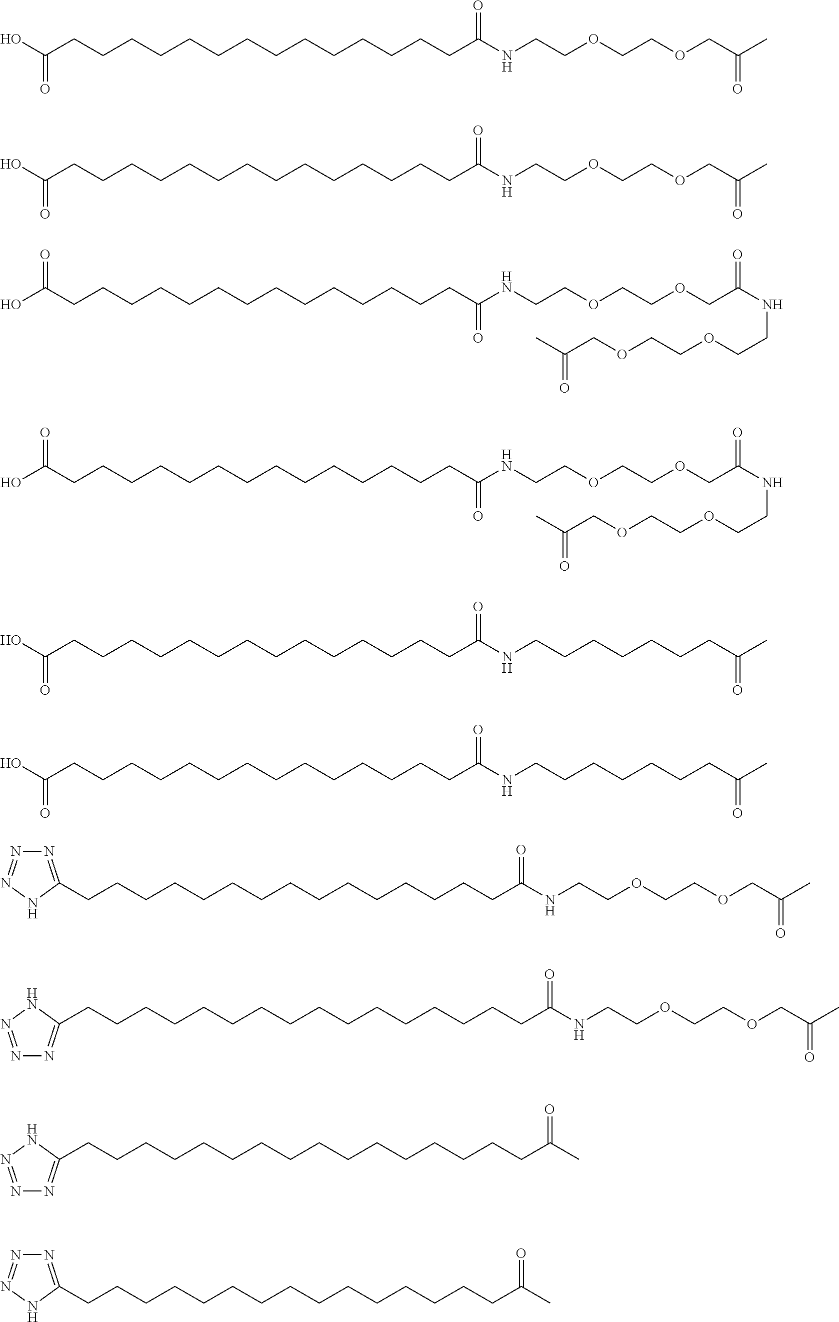

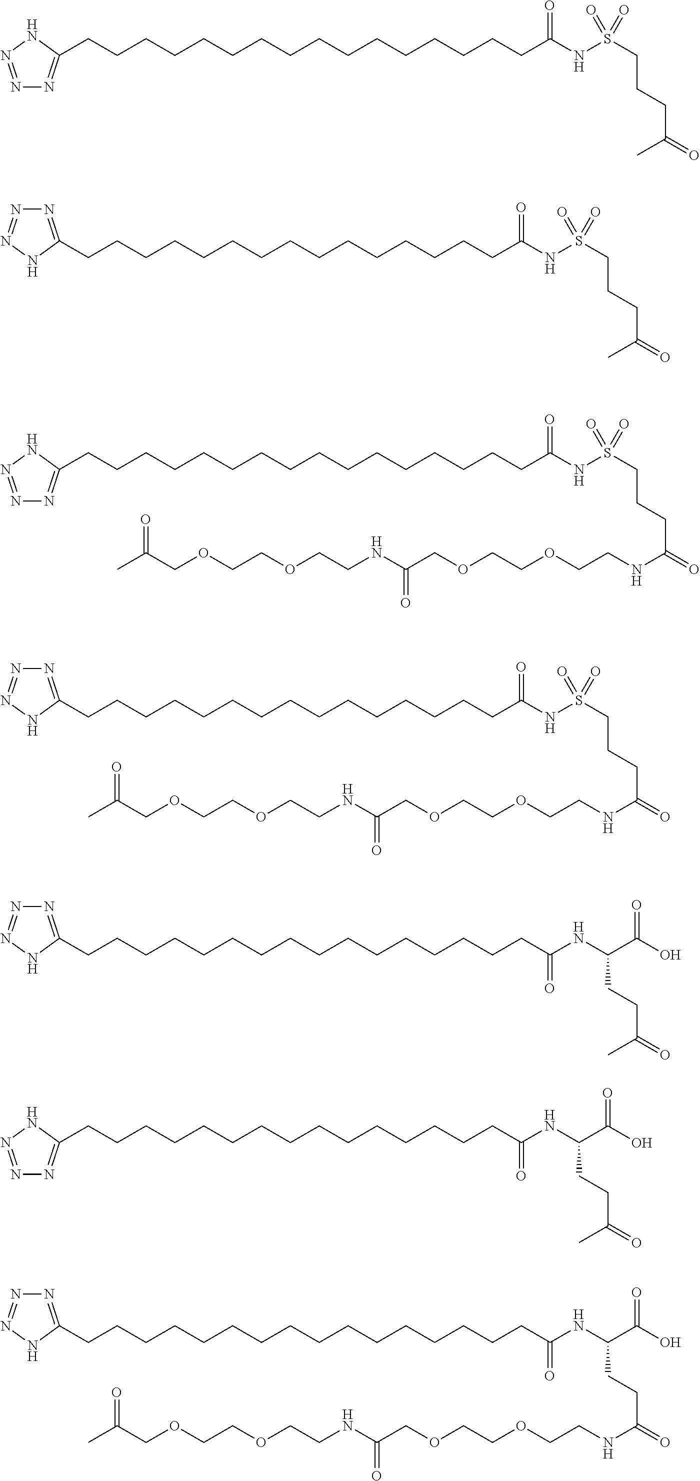

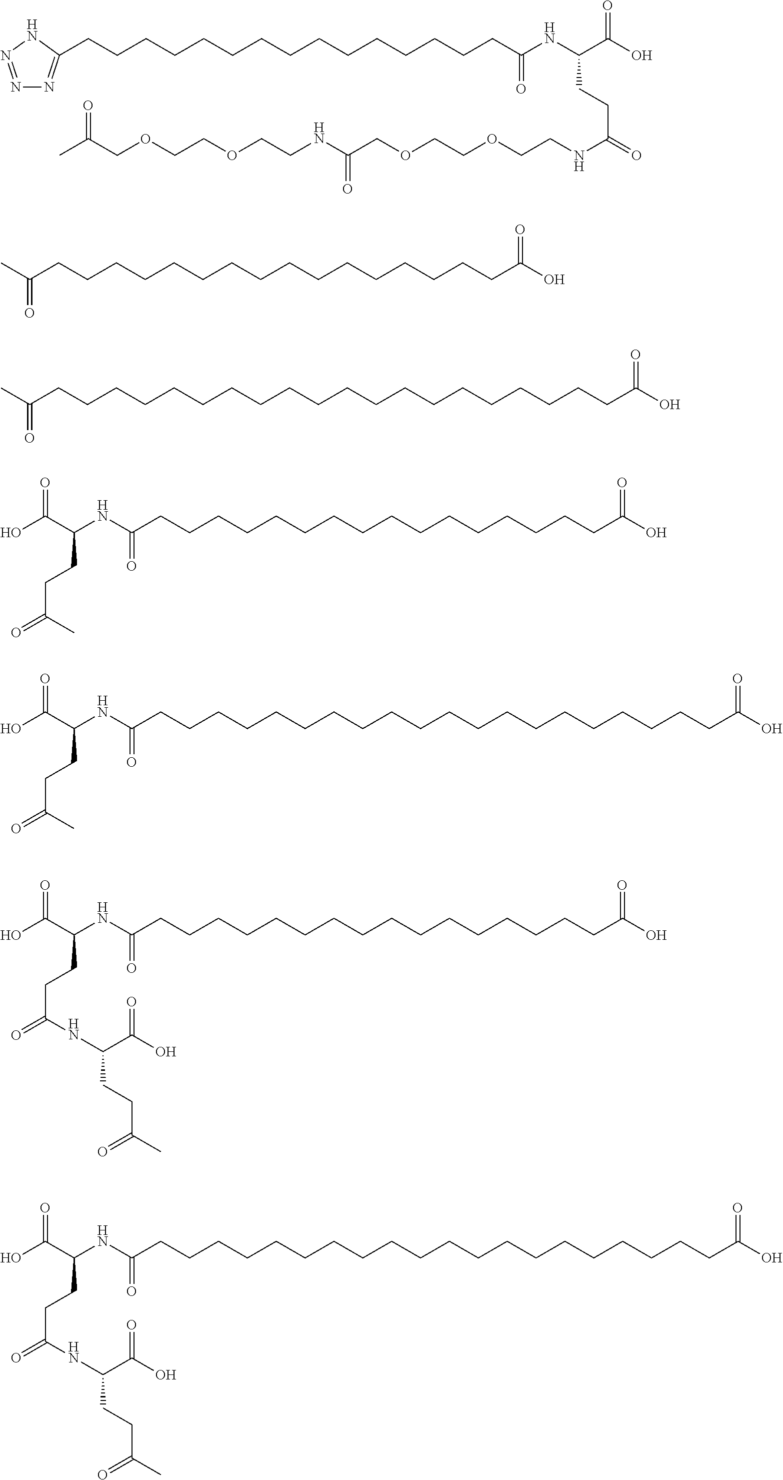

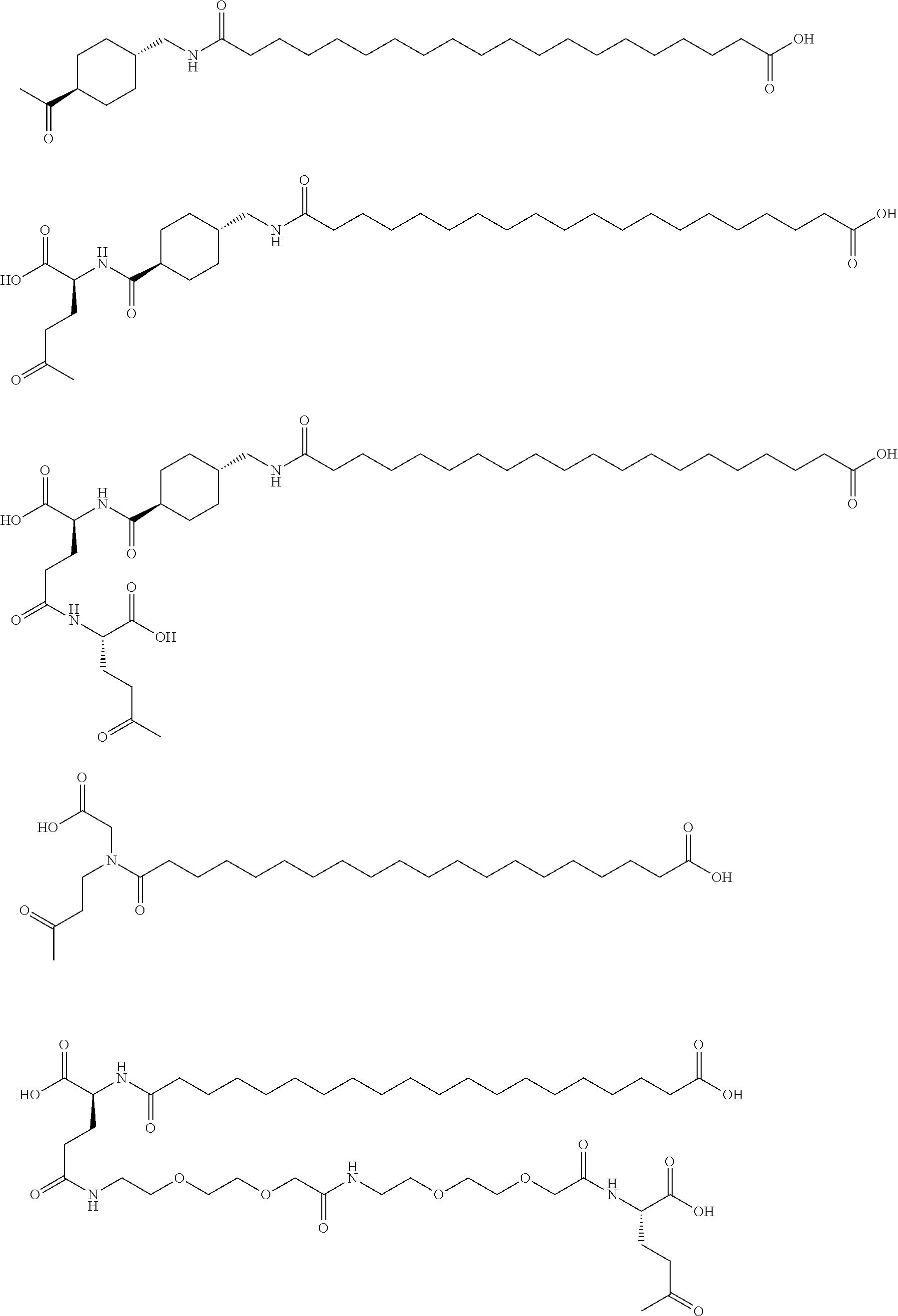

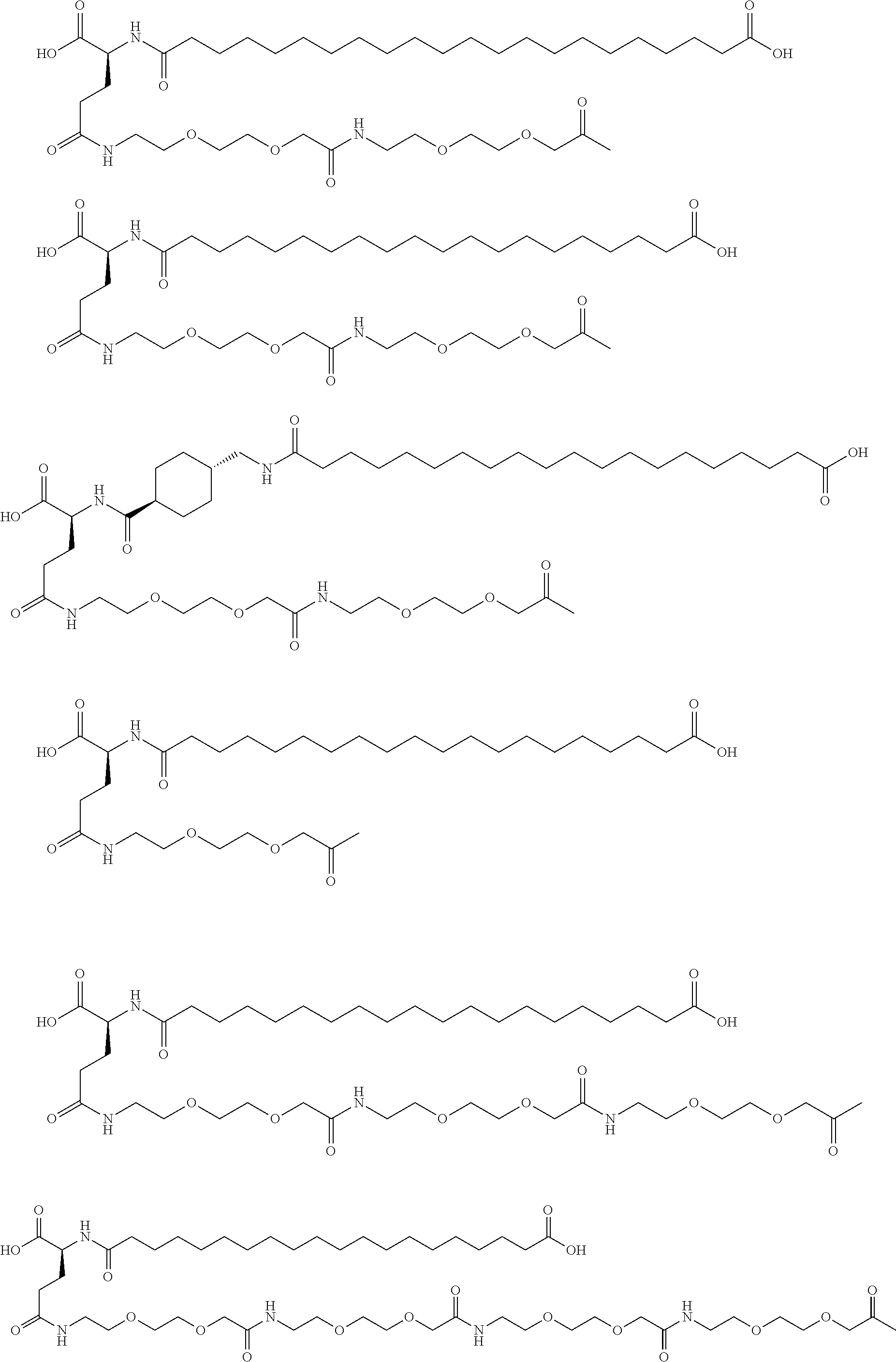

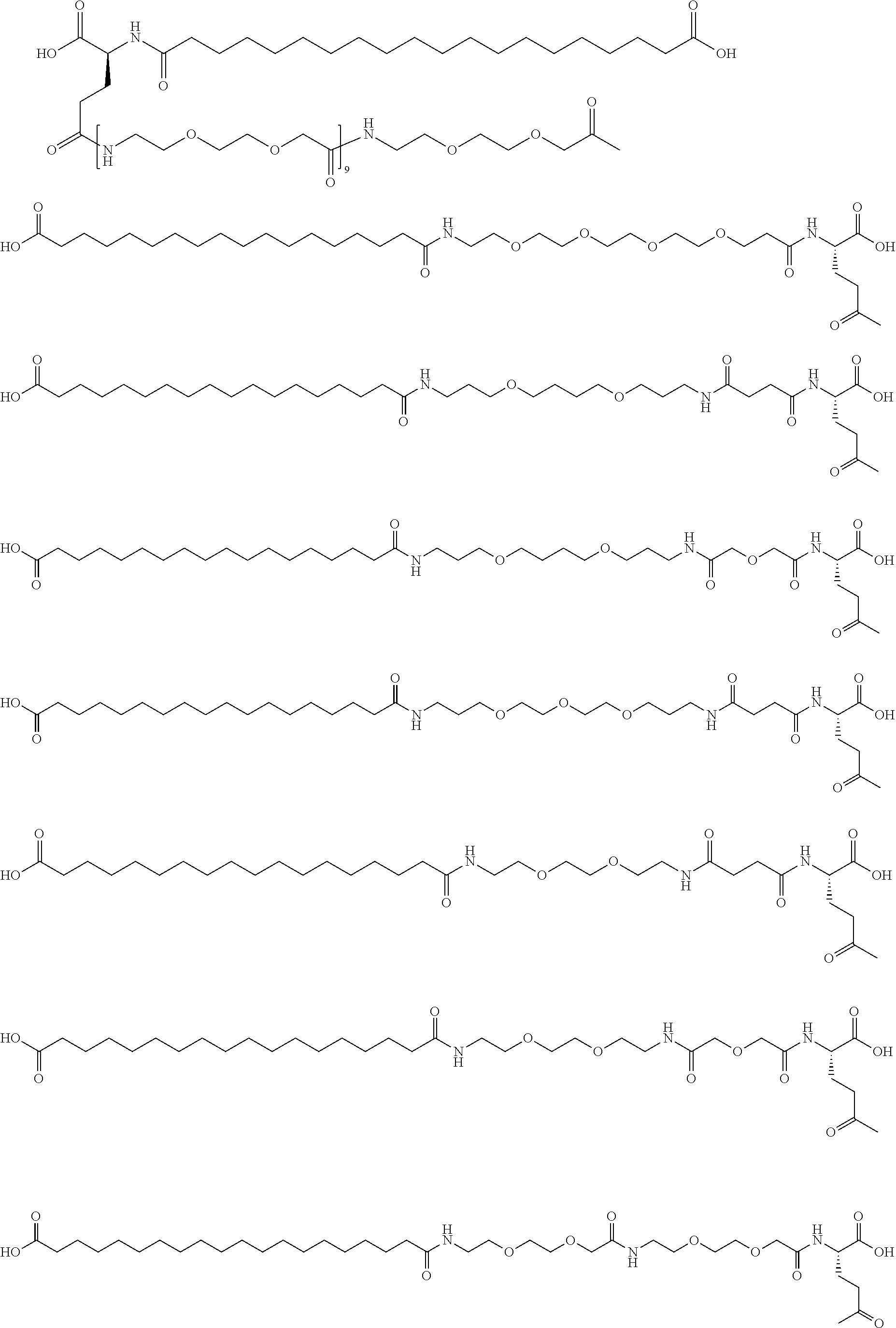

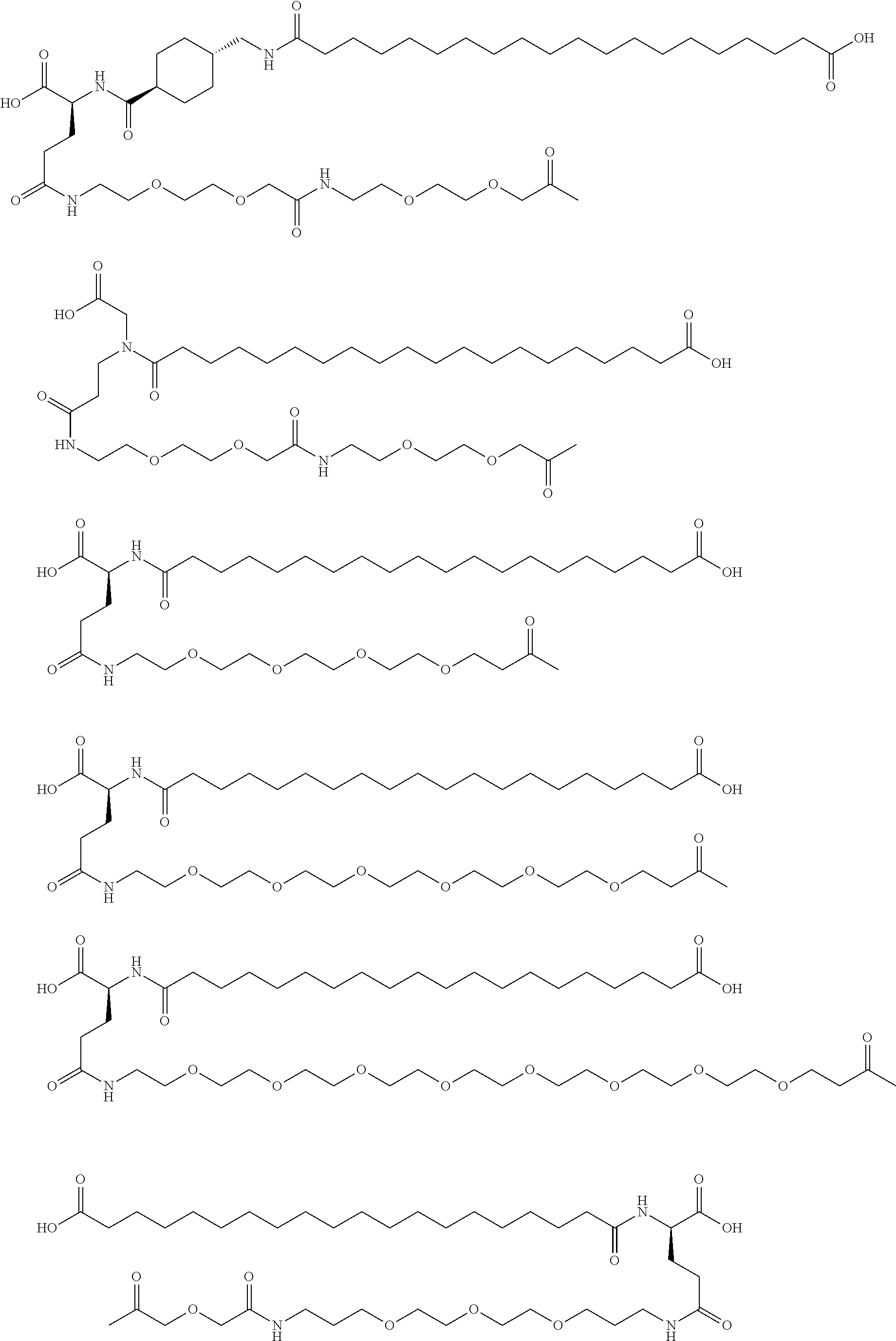

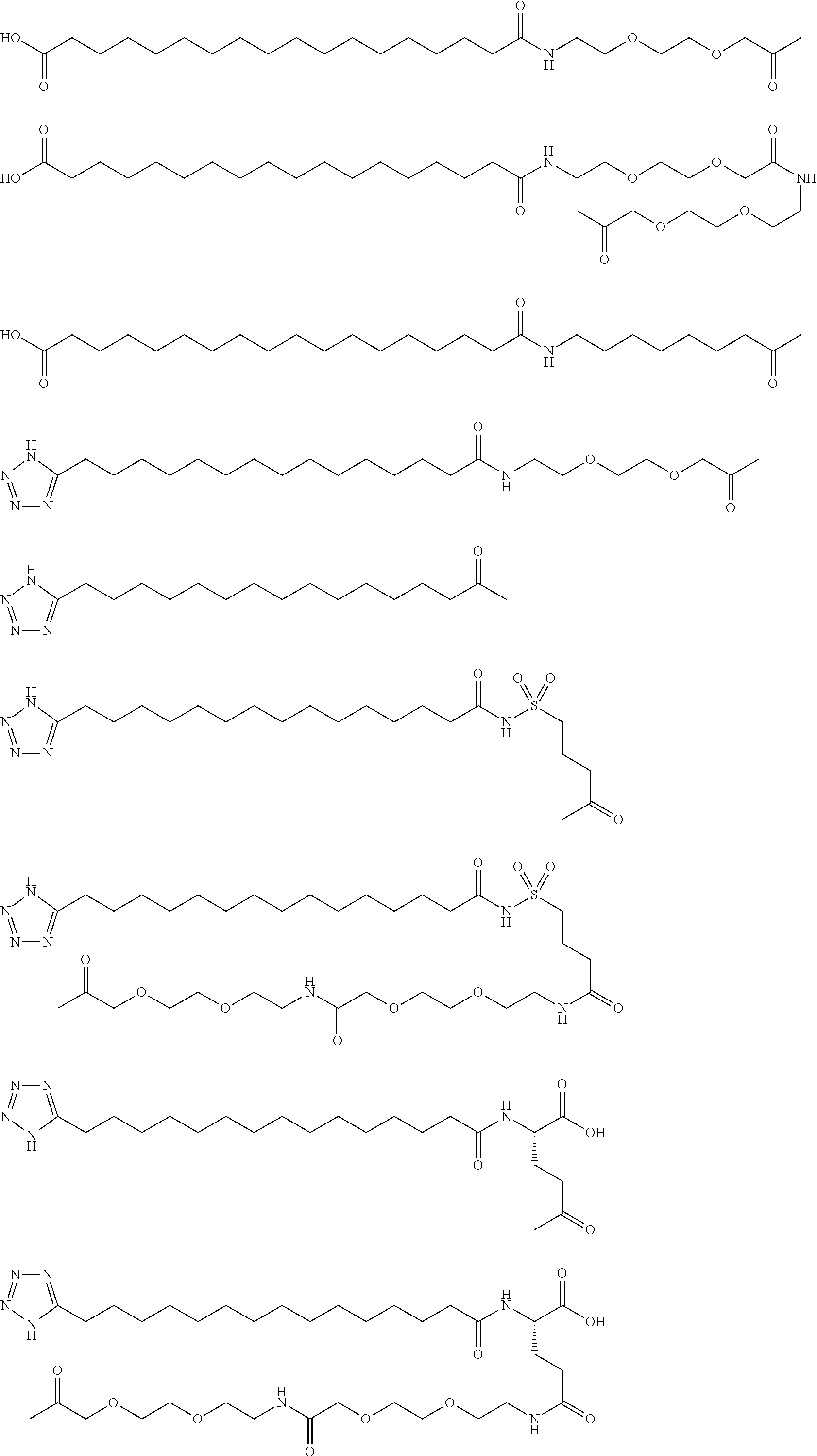

Any of the above non-limiting specific examples of side chains of the formula Acy-AA1ₙ-AA2ₘ-AA3ₚ- may be attached to an epsilon amino group of a lysine residue present in any of the above non-limiting specific examples of acylated insulin analogues thereby giving further specific examples of acylated insulin analogues of this invention.

Any of the above non-limiting specific examples of side chains of the formula Acy-AA1ₙ-AA2ₘ-AA3ₚ- may be attached to an alpha amino group of an A1 residue present in any of the above non-limiting specific examples of acylated insulin analogues thereby giving further specific examples of acylated insulin analogues of this invention.

In one embodiment, acylated insulins in a pharmaceutical composition according to the invention, i.e. protease stabilised and/or containing one or more additional disulfide bonds, are more protracted than similar acylated insulins which are not protease stabilised or which are without one or more additional disulfide bonds. With “more protracted” is herein meant that they have a longer elimination half-life or in other words an insulin effect for an extended period, i.e. a longer duration of action.

stabilisestabilise

A non-limiting example of lipophilic substituents which may be used according to the invention may also be found in the patent application WO 2009/115469, including as the lipophilic substituents of the acylated polypeptides as described in the passage beginning on page 25, line 3 of WO 2009/115469.

A non-limiting list of examples of acylated insulins in the form of acylated insulin analogues which may be modified by cysteine substitutions according to the invention may e.g. be found in WO 2009/115469 A1.

In one embodiment a tablet core according to the present invention comprises an acylated insulin, which is selected from the group consisting of:
1. A14E,B25H,B29K(N^{ε}-Hexadecandioyl),desB30 human insulin,
2. A14E,B25H,B29K(N^{ε}Octadecandioyl-γGlu),desB30 human insulin,
3. A14E,B25H,B29K(N^{ε}Eicosanedioyl-γGlu),desB30 human insulin,
4. A14E,B25H,B29K(N^{ε}3-Carboxy-5-octadecanedioylaminobenzoyl),desB30 human insulin,
5. A14E,B25H,B29K(N^{ε}—N-octadecandioyl-N-(2-carboxyethyl)glycyl),desB30 human insulin
6. A14E,B25H,B29K(N^{ε}(N-Octadecandioyl-N-carboxymethyl)-beta-alanyl),desB30 human insulin,
7. A14E,B25H,B29K(N^{ε}4-([4-({19-Carboxynonadecanoylamino}methyl)trans-cyclohexanecarbonyl]-γGlu),desB30 human insulin,
8. A14E,B25H,B29K(N^{ε}Heptadecanedioyl-γGlu),desB30 human insulin,
9. A14E,B25H,B29K(N^{ε}Octadecanedioyl-γGlu-OEG-OEG),desB30 human insulin,
10. A14E,B25H,B29K(N^{ε}Myristyl),desB30 human insulin,
11. A14E,B25H,B29K(N^{ε}Eicosanedioyl-γGlu-γGlu),desB30 human insulin,
12. A14E,B25H,B29K(N^{ε}4-([4-({19-Carboxynonadecanoylamino}methyl)trans-cyclohexanecarbonyl]-γGlu-γGlu),desB30 human insulin,
13. A14E,B25H,B29K(N^{ε}Octadecanedioyl-γGlu-γGlu),desB30 human insulin,
14. A14E,B28D,B29K(N^{ε}octadecandioyl-γGlu),desB30 human insulin,
15. A14E,B25H,B29K(N^{ε}octadecandioyl-γGlu-PEG7),desB30 human insulin,
16. A14E,B25H,B29K(N^{ε}eicosanedioyl-γGlu-OEG-OEG), desB30 human insulin,
17. A14E,B25H,B29K(N^{ε}eicosanedioyl-γGlu-(3-(2-{2-[2-(2-aminoethoxy)ethoxy]ethoxy}ethoxy)propionyl-γGlu),desB30 human insulin,
18. A14E,B25H,B29K(N^{ε}Hexadecanedioyl-γGlu-OEG-OEG),desB30 human insulin,
19. A14E,B25H,B29K(N^{ε}Hexadecanedioyl-γGlu),desB30 human insulin,
20. A14E,B25H,B29K(N^{ε}heptadecanedioyl-γGlu-OEG-OEG),desB30 human insulin,
21. A14E,B25H,B29K(N^{ε}octadecanedioyl-γGlu-γGlu-γGlu-γGlu),desB30 human insulin,
22. A14E,B25H,B29K(N^{ε}Eicosanedioyl-γGlu-γGlu-γGlu),desB30 human insulin,
23. A14E,B25H,B27E,B29K(N^{ε}Octadecanedioyl-γGlu-OEG-OEG),desB30 human insulin,
24. A14E,B25H,B26G,B27G,B28G,B29K(N^{ε}Octadecanedioyl-γGlu-OEG-OEG),desB30 human insulin,
25. A14E,B16H,B25H,B29K(N^{ε}Octadecanedioyl-γGlu-OEG-OEG),desB30 human insulin,
26. A14E,B16E,B25H,B29K(N^{ε}Octadecanedioyl-γGlu-OEG-OEG),desB30 human insulin,
27. A14E,B16H,B25H,B29K(N^{ε}Hexadecanedioyl-γGlu),desB30 human insulin,
28. A14E,B25H,B29K(N^{ε}Eicosanedioyl-γGlu-OEG-γGlu),desB30 human insulin,
29. A14E,B16E,B25H,B29K(N^{ε}Hexadecandioyl-γGlu),desB30 human insulin,
30. A14E,B16H,B25H,B29K(N^{ε}Octadecanedioyl-γGlu-γGlu-γGlu),desB30 human insulin,
31. A14E,B25H,B26G,B27G,B28G,B29K(N^{ε}Hexadecandioyl-γGlu),desB30 human insulin,
32. A14E,B16H,B25H,B29K(N^{ε}Octadecanedioyl-γGlu-γGlu),desB30 human insulin,
33. A14E,B16H,B25H,B29K(N(eps)Eicosanedioyl-γGlu-OEG-OEG),desB30 human insulin,
34. A14E,B25H,B29K(N^{ε}Octadecanedioyl-OEG-γGlu-γGlu),desB30 human insulin,
35. A14E,A18L,B25H,B29K(N^{ε}Eicosanedioyl-γGlu-OEG-OEG),desB30 human insulin,
36. A14E,A18L,B25H,B29K(N^{ε}Octadecanedioyl-γGlu-OEG-OEG),desB30 human insulin,
37. A14E,B25H,B27E,B29K(N^{ε}Eicosanedioyl-γGlu-OEG-OEG),desB30 human insulin,
38. A1G(N^{α}Octadecandioyl-γGlu-OEG-OEG),A14E,B25H,B29R,desB30 human insulin,
39. A14E,B1F(N^{α}Octadecandioyl-γGlu-OEG-OEG),B25H,B29R,desB30 human insulin,
40. A1G(N^{α}Hexadecandioyl-γGlu),A14E,B25H,B29R,desB30 human insulin,
41. A14E,B25H,B29K(N^{ε}Octadecanedioyl-γGlu-Abu-Abu-Abu-Abu),desB30 human insulin,
42. A14E,B25H,B29K(N^{α}Eicosanedioyl),desB30 human insulin,
43. A14E,B25H,B29K(N^{α}4-[16-(1H-Tetrazol-5-yl)hexadecanoylsulfamoyl]butanoyl), desB30 human insulin,
44. A1G(N^{α}Octadecandioyl-γGlu-OEG-OEG),A14E,A21G,B25H,desB30 human insulin,
45. A14E,B25H,B29K(N^{ε}Eicosanedioyl-OEG),desB30 human insulin,
46. A14E,B25H,B27K(N^{ε}Octadecanedioyl-γGlu-OEG-OEG),desB28,desB29,desB30 human insulin,
47. A14E,B25H,B29K(N^{ε}(5-Eicosanedioylaminoisophthalic acid)),desB30 human insulin,
48. A14E,B25H,B29K(N^{ε}Octadecanedioyl),desB30 human insulin,
49. A14E,B29K(N^{ε}Octadecanedioyl-γGlu-OEG-OEG),desB30 human insulin,
50. A14E,B25H,B26G,B27G,B28G,B29K(N^{ε}Eicosanedioyl-γGlu-OEG-OEG),desB30 human insulin,
51. A14E,B25H,B29K(N^{ε}Octadecanedioyl-γGlu-OEG),desB30 human insulin,
52. A14E,B25H,B29K(N^{ε}Eicosanedioyl-OEG-OEG),desB30 human insulin,
53. A14E,B25H,B29K(N^{ε}Eicosanedioyl-Aoc),desB30 human insulin,
54. A14E,B25H,B26G,B27G,B28G,B29K(N^{ε}Eicosanedioyl-γGlu-γGlu),desB30 human insulin,
55. A14E,B25H,B26G,B27G,B28G,B29K(N^{ε}Eicosanedioyl-γGlu-γGlu),desB30 human insulin,
56. A14E,B25H,B29K(N^{ε}Octadecanedioyl-OEG),desB30 human insulin,
57. A14E,B25H,desB27,B29K(N^{ε}Octadecanedioyl-γGlu-OEG-OEG),desB30 human insulin,
58. A14E,B25H,B16H,B29K(N^{ε}Octadecanedioyl-γGlu),desB30 human insulin,
59. A1G(N^{α}Octadecanedioyl),A14E,B25H,B29R,desB30 human insulin,
60. A14E,B16H,B25H,B29K(N^{ε}Eicosanedioyl-γGlu),desB30 human insulin,
61. A14E,B25H,B27K(N^{ε}Eicosanedioyl-γGlu),desB28,desB29,desB30 human insulin,
62. A14E,B25H,B29K(N^{ε}Octadecanedioyl-γGlu-γGlu-γGlu),desB30 human
63. A14E,B25H,B26G,B27G,B28G,B29K(N^{ε}Octadecandioyl-γGlu),desB30 human insulin,
64. A14E,B25H,B26G,B27G,B28G,B29K(N^{ε}Eicosanedioyl-γGlu),desB30 human insulin,
65. A14E,B25H,B26G,B27G,B28G,B29K(N^{ε}Octadecandioyl),desB30 human insulin,
66. A14E,B25H,B26G,B27G,B28G,B29K(N^{ε}Eicosanedioyl),desB30 human insulin,
67. A14E,B25H,B29K(N^{ε}Docosanedioyl-γGlu),desB30 human insulin,
68. A14E,B25H,B29K(N^{ε}Docosanedioyl-γGlu-γGlu),desB30 human insulin,
69. A14E,B25H,B29K(N^{ε}Icosanedioyl-γGlu-OEG-OEG-γGlu),desB30 human insulin,
70. A14E,B25H,B29K(N^{ε}Octadecanedioyl-γGlu-OEG-OEG-γGlu),desB30 human insulin,
71. A14E,B25H,B29K(N^{ε}(N-Icosanedioyl-N-carboxymethyl)-βAla),desB30 human insulin,
72. A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
73. A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
74. A14E,B25H,B29K(N^{ε}Octadecandioyl-γGlu-(3-(2-{2-[2-(2-aminoethoxy)ethoxy]ethoxy}ethoxy)propionyl),desB30 human insulin,
75. A14E,B25H,B29K(N^{ε}Octadecandioyl-γGlu-(3-(2-{2-[2-(2-aminoethoxy)ethoxy]ethoxy}ethoxy)propionyl-γGlu),desB30 human insulin,
76. A14E,B25H,B29K(N^{ε}Icosanedioyl-γGlu-(3-(2-{2-[2-(2-aminoethoxy)ethoxy]ethoxy}ethoxy)propionyl),desB30 human insulin,
77. A14E,B25H,B29K(N^{ε}4-([4-({17-Carboxynonadecanoylamino}methyl)trans-cyclohexanecarbonyl]-γGlu),desB30 human insulin,
78. A14E,B25H,B29K(N^{ε}4-([4-({17-Carboxyheptadecanoylamino}methyl)trans-cyclohexanecarbonyl]-γGlu-γGlu),desB30 human insulin,
79. A14E,B28D,B29K(N^{ε}hexadecandioyl-γGlu),desB30 human insulin,
80. A14E,B28D,B29K(N^{ε}Eicosanedioyl-γGlu),desB30 human insulin,
81. A14E,B28D,B29K(N^{ε}Octadecandioyl-γGlu-OEG-OEG),desB30 human insulin,
82. A14E,B28D,B29K(N^{ε}Eicosanedioyl-γGlu-OEG-OEG),desB30 human insulin,
83. A14E,B28E,B29K(N^{ε}Hexadecandioyl-γGlu),desB30 human insulin,
84. A14E,B28E,B29K(N^{ε}Octadecandioyl-γGlu),desB30 human insulin,
85. A14E,B28E,B29K(N^{ε}Eicosanedioyl-γGlu),desB30 human insulin,
86. A14E,B28E,B29K(N^{ε}Octadecandioyl-γGlu-OEG-OEG),desB30 human insulin,
87. A14E,B28E,B29K(N^{ε}Eicosanedioyl-γGlu-OEG-OEG),desB30 human insulin,
88. A14E,B1E,B28E,B29K(N^{ε}Hexadecandioyl-γGlu),desB30 human insulin,
89. A14E,B1E,B28E,B29K(N^{ε}Octadecandioyl-γGlu),desB30 human insulin,
90. A14E,B1E,B28E,B29K(N^{ε}Eicosanedioyl-γGlu),desB30 human insulin,
91. A14E,B1E,B28E,B29K(N^{ε}Hexadecandioyl-γGlu-OEG-OEG),desB30 human insulin,
92. A14E,B1E,B28E,B29K(N^{ε}Octadecandioyl-γGlu-OEG-OEG),desB30 human insulin,
93. A14E,B1E,B28E,B29K(N^{ε}Eicosanedioyl-γGlu-OEG-OEG),desB30 human insulin,
94. A14E,B1E,B27E,B28E,B29K(N^{ε}Hexadecandioyl-γGlu),desB30 human insulin,
95. A14E,B1E,B27E,B28E,B29K(N^{ε}Octadecandioyl-γGlu),desB30 human insulin,
96. A14E,B1E,B27E,B28E,B29K(N^{ε}Eicosanedioyl-γGlu),desB30 human insulin,
97. A14E,B1E,B27E,B28E,B29K(N^{ε}Hexadecandioyl-γGlu-OEG-OEG),desB30 human insulin,
98. A14E,B1E,B27E,B28E,B29K(N^{ε}Octadecandioyl-γGlu-OEG-OEG),desB30 human insulin,
99. A14E,B1E,B27E,B28E,B29K(N^{ε}Eicosanedioyl-γGlu-OEG-OEG),desB30 human insulin,
100. A14E,B1E,B25H,B28E,B29K(N^{ε}Hexadecandioyl-γGlu),desB30 human insulin,
101. A14E,B1E,B25H,B28E,B29K(N^{ε}Octadecandioyl-γGlu),desB30 human insulin,
102. A14E,B1E,B25H,B28E,B29K(N^{ε}Eicosanedioyl-γGlu),desB30 human insulin,
103. A14E,B1E,B25H,B28E,B29K(N^{ε}Hexadecandioyl-γGlu-OEG-OEG),desB30 human insulin,
104. A14E,B1E,B25H,B28E,B29K(N^{ε}Octadecandioyl-γGlu-OEG-OEG),desB30
105. A14E,B1E,B25H,B28E,B29K(N^{ε}Eicosanedioyl-γGlu-OEG-OEG),desB30 human insulin,
106. A14E,B1E,B25H,B27E,B28E,B29K(N^{ε}Hexadecandioyl-γGlu),desB30 human insulin,
107. A14E,B1E,B25H,B27E,B28E,B29K(N^{ε}Octadecandioyl-γGlu),desB30 human insulin,
108. A14E,B1E,B25H,B27E,B28E,B29K(N^{ε}Eicosanedioyl-γGlu),desB30 human insulin,
109. A14E,B1E,B25H,B27E,B28E,B29K(N^{ε}Hexadecandioyl-γGlu-OEG-OEG),desB30 human insulin,
110. A14E,B1E,B25H,B27E,B28E,B29K(N^{ε}Octadecandioyl-γGlu-OEG-OEG),desB30 human insulin,
111. A14E,B1E,B25H,B27E,B28E,B29K(N^{ε}Eicosanedioyl-γGlu-OEG-OEG),desB30 human insulin,
112. A14E,B28D,B29K(N^{ε}Hexadecanedioyl-γGlu-OEG-OEG),desB30 human insulin,
113. A14E,B28E,B29K(N^{ε}Hexadecanedioyl-γGlu-OEG-OEG),desB30 human insulin,
114. B25N,B27E,B29K(N^{ε}Eicosanedioyl-γGlu-OEG-OEG),desB30 human insulin,
115. B25N,B27E,B29K(N^{ε}Octadecanedioyl-γGlu-OEG-OEG),desB30 human insulin,
116. B25N,B27E,B29K(N^{ε}Hexadecanedioyl-γGlu-OEG-OEG),desB30 human insulin,
117. B25N,B27E,B29K(N^{ε}Eicosanedioyl-γGlu),desB30 human insulin,
118. B25N,B27E,B29K(N^{ε}Octadecanedioyl-γGlu),desB30 human insulin,
119. B25N,B27E,B29K(N^{ε}Hexadecanedioyl-γGlu),desB30 human insulin,
120. A8H,B25N,B27E,B29K(N^{ε}Eicosanedioyl-γGlu-OEG-OEG),desB30 human insulin,
121. A8H,B25N,B27E,B29K(N^{ε}Octadecanedioyl-γGlu-OEG-OEG),desB30 human insulin,
122. A8H,B25N,B27E,B29K(N^{ε}Hexadecanedioyl-γGlu-OEG-OEG),desB30 human insulin,
123. A8H,B25N,B27E,B29K(N^{ε}Eicosanedioyl-γGlu),desB30 human insulin,
124. A8H,B25N,B27E,B29K(N^{ε}Octadecanedioyl-γGlu),desB30 human insulin,
125. A8H,B25N,B27E,B29K(N^{ε}Hexadecanedioyl-γGlu),desB30 human insulin,
126. 14E,B25H,B29K(N^{ε}(N-Icosanedioyl-N-carboxymethyl)-βAla-OEG-OEG),desB30 human insulin,
127. A14E,B25H,B29K(N^{ε}(N-Octadecanedioyl-N-carboxymethyl)-βAla-OEG-OEG),desB30 human insulin,
128. A14E,B25H,B29K(N^{ε}(N-Hexadecanedioyl-N-carboxymethyl)-βAla-OEG-OEG),desB30 human insulin,
129. A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
130. A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
131. A14E,B16H,B25H,B29K(N^{ε}Octadecanedioyl-γGlu-2-[(3-{2-[2-(3-aminopropoxy)ethoxy]ethoxy}propylcarbamoyl)methoxy]acetyl),desB30 human insulin,
132. A14E, B16H, B25H, B29K(N^{ε}Eicosanedioyl-γGlu-2-[(3-{2-[2-(3-aminopropoxy)ethoxy]ethoxy}propylcarbamoyl)methoxy]acetyl),desB30 human insulin,
133. B25H,B29K(N^{ε}Octadecanedioyl-γGlu-OEG-OEG),desB30 human insulin,
134. B25H,B29K(N^{ε}Eicosanedioyl-γGlu-OEG-OEG),desB30 human insulin,
135. B25H,B29K(N^{ε}Octadecanedioyl-γGlu),desB30 human insulin,
136. B25H,B29K(N^{ε}Eicosanedioyl-γGlu),desB30 human insulin,
137. B25H,B29K(N^{ε}Octadecanedioyl),desB30 human insulin,
138. B25H,B29K(N^{ε}Eicosanedioyl),desB30 human insulin,
139. B25H,B29K(N^{ε}Octadecanedioyl-γGlu-OEG-OEG),desB30 human insulin,
140. B25H,B29K(N^{ε}Eicosanedioyl-γGlu-OEG-OEG),desB30 human insulin,
141. B25H,B29K(N^{ε}Octadecanedioyl-γGlu),desB30 human insulin,
142. B25H,B29K(N^{ε}Eicosanedioyl-γGlu),desB30 human insulin,
143. 21G,B25H,B29K(N^{ε}Octadecanedioyl),desB30 human insulin,
144. A21G,B25H,B29K(N^{ε}Eicosanedioyl),desB30 human insulin,
145. A21G,B25H,B29K(N^{ε}Octadecanedioyl-γGlu-OEG-OEG),desB30 human insulin,
146. A21G,B25H,B29K(N^{ε}Eicosanedioyl-γGlu-OEG-OEG),desB30 human insulin,
147. A21G,B25H,B29K(N^{ε}Octadecanedioyl-γGlu),desB30 human insulin,
148. A21G,B25H,B29K(N^{ε}Eicosanedioyl-γGlu),desB30 human insulin,
149. A14E,B25H,desB27,B29K(N^{ε}Octadecanedioyl),desB30 human insulin,
150. A14E,B25H,desB27,B29K(N^{ε}Eicosanedioyl),desB30 human insulin,
151. A14E,B25H,desB27,B29K(N^{ε}Octadecanedioyl-γGlu),desB30 human insulin,
152. A14E,B25H,desB27,B29K(N^{ε}Eicosanedioyl-γGlu),desB30 human insulin,
153. A14E,B25H,desB27,B29K(N^{ε}Eicosanedioyl-γGlu-OEG-OEG),desB30 human insulin,
154. A14E,A21G,B25H,desB27,B29K(N^{ε}Octadecanedioyl),desB30 human insulin,
155. A14E,A21G,B25H,desB27,B29K(N^{ε}Eicosanedioyl),desB30 human insulin,
156. A14E,A21G,B25H,desB27,B29K(N^{ε}Octadecanedioyl-γGlu),desB30 human insulin,
157. A14E,B25H,desB27,B29K(N^{ε}Eicosanedioyl-γGlu),desB30 human insulin,
158. A14E,A21G,B25H,desB27,B29K(N^{ε}Octadecanedioyl-γGlu-OEG-OEG),desB30 human insulin,
159. A14E,A21G,B25H,desB27,B29K(N^{ε}Eicosanedioyl-γGlu-OEG-OEG),desB30 human insulin,
160. A14E,A21G,B25H,B29K(N^{ε}Octadecanedioyl-γGlu-OEG-OEG),desB30 human insulin,
161. A14E,A21G,B25H,B29K(N^{ε}Eicosanedioyl-γGlu-OEG-OEG),desB30 human insulin,
162. A14E,A21G,B25H,B29K(N^{ε}Eicosanedioyl-γGlu),desB30 human insulin,
163. A14E,A21G,B25H,B29K(N^{ε}Eicosanedioyl),desB30 human insulin,
164. A14E,A21G,B25H,B29K(N^{ε}Octadecanedioyl-γGlu),desB30 human insulin,
165. A14E,A21G,B25H,B29K(N^{ε}Octadecanedioyl),desB30 human insulin,
166. A14E,B25H,B26G,B27G,B28G,B29K(N^{ε}Octadecanedioyl-γGlu),desB30 human insulin,
167. A14E,B25H,B26G,B27G,B28G,B29K(N^{ε}Octadecanedioyl),desB30 human insulin,
168. A14E,B25H,B26G,B27G,B28G,B29K(N^{ε}Eicosanedioyl-γGlu),desB30 human insulin,
169. A14E,B25H,B26G,B27G,B28G,B29K(N^{ε}Eicosanedioyl),desB30 human insulin,
170. A1G(N^{α}Octadecandioyl-γGlu),A14E,B25H,B26G,B27G,B28G,desB30 human insulin,
171. A1G(N^{α}Eicosanedioyl-γGlu),A14E,B25H,B26G,B27G,B28G,desB30 human insulin,
172. A1G(N^{α}Octadecandioyl-γGlu),A14E,B25H,B26G,B27G,B28G,B29R,desB30 human insulin,
173. A1G(N^{α}Eicosanedioyl-γGlu),A14E,B25H,B26G,B27G,B28G,B29R,desB30 human insulin,
174. A1G(N^{α}Octadecandioyl),A14E,B25H,B26G,B27G,B28G,desB30 human insulin,
175. A1G(N^{α}Eicosanedioyl),A14E,B25H,B26G,B27G,B28G,desB30 human insulin,
176. A1G(N^{α}Octadecandioyl),A14E,B25H,B26G,B27G,B28G,B29R,desB30 human insulin and
177. A1G(N^{α}Eicosanedioyl),A14E,B25H,B26G,B27G,B28G,B29R,desB30 human insulin.

In one embodiment a tablet core according to the present invention comprises a protease stabilised acylated insulin, which is selected from the group consisting of:
1. A14E,B25H,B29K(N^{ε}—Hexadecandioyl),desB30 human insulin,
2. A14E,B25H,B29K(N^{ε}Octadecandioyl-γGlu),desB30 human insulin,
3. A14E,B25H,B29K(N^{ε}Eicosanedioyl-γGlu),desB30 human insulin,
4. A14E,B25H,B29K(N^{ε}3-Carboxy-5-octadecanedioylaminobenzoyl),desB30 human insulin,
5. A14E,B25H,B29K(N^{ε}—N-octadecandioyl-N-(2-carboxyethyl)glycyl),desB30 human insulin
6. A14E,B25H,B29K(N^{ε}(N-Octadecandioyl-N-carboxymethyl)-beta-alanyl),desB30 human insulin,
7. A14E,B25H,B29K(N^{ε}4-([4-({19-Carboxynonadecanoylamino}methyl)trans-cyclohexanecarbonyl]-γGlu),desB30 human insulin,
8. A14E,B25H,B29K(N^{ε}Heptadecanedioyl-γGlu),desB30 human insulin,
9. A14E,B25H,B29K(N^{ε}Octadecanedioyl-γGlu-OEG-OEG),desB30 human insulin,
10. A14E,B25H,B29K(N^{ε}Myristyl),desB30 human insulin,
11. A14E,B25H,B29K(N^{ε}Eicosanedioyl-γGlu-γGlu),desB30 human insulin,
12. A14E,B25H,B29K(N^{ε}4-([4-({19-Carboxynonadecanoylamino}methyl)trans-cyclohexanecarbonyl]-γGlu-γGlu),desB30 human insulin,
13. A14E,B25H,B29K(N^{ε}Octadecanedioyl-γGlu-γGlu),desB30 human insulin,
14. A14E,B28D,B29K(N^{ε}octadecandioyl-γGlu),desB30 human insulin,
15. A14E,B25H,B29K(N^{ε}octadecandioyl-γGlu-PEG7),desB30 human insulin,
16. A14E,B25H,B29K(N^{ε}eicosanedioyl-γGlu-OEG-OEG), desB30 human insulin,
17. A14E,B25H,B29K(N^{ε}eicosanedioyl-γGlu-(3-(2-{2-[2-(2-aminoethoxy)ethoxy]ethoxy}ethoxy)propionyl-γGlu),desB30 human insulin,
18. A14E,B25H,B29K(N^{ε}Hexadecanedioyl-γGlu-OEG-OEG),desB30 human insulin,
19. A14E,B25H,B29K(N^{ε}Hexadecanedioyl-γGlu),desB30 human insulin,
20. A14E,B25H,B29K(N^{ε}heptadecanedioyl-γGlu-OEG-OEG),desB30 human insulin,
21. A14E,B25H,B29K(N^{ε}octadecanedioyl-γGlu-γGlu-γGlu-γGlu),desB30 human insulin,
22. A14E,B25H,B29K(N^{ε}Eicosanedioyl-γGlu-γGlu-γGlu),desB30 human insulin,
23. A14E,B25H,B27E,B29K(N^{ε}Octadecanedioyl-γGlu-OEG-OEG),desB30 human insulin,
24. A14E,B25H,B26G,B27G,B28G,B29K(N^{ε}Octadecanedioyl-γGlu-OEG-OEG),desB30 human insulin,
25. A14E,B16H,B25H,B29K(N^{ε}Octadecanedioyl-γGlu-OEG-OEG),desB30 human insulin,
26. A14E,B16E,B25H,B29K(N^{ε}Octadecanedioyl-γGlu-OEG-OEG),desB30 human insulin,
27. A14E,B16H,B25H,B29K(N^{ε}Hexadecanedioyl-γGlu),desB30 human insulin,
28. A14E,B25H,B29K(N^{ε}Eicosanedioyl-γGlu-OEG-γGlu),desB30 human insulin,
29. A14E,B16E,B25H,B29K(N^{ε}Hexadecandioyl-γGlu),desB30 human insulin,
30. A14E,B16H,B25H,B29K(N^{ε}Octadecanedioyl-γGlu-γGlu-γGlu),desB30 human insulin,
31. A14E,B25H,B26G,B27G,B28G,B29K(N^{ε}Hexadecandioyl-γGlu),desB30 human
32. A14E,B16H,B25H,B29K(N^{ε}Octadecanedioyl-γGlu-γGlu),desB30 human insulin,
33. A14E,B16H,B25H,B29K(N(eps)Eicosanedioyl-γGlu-OEG-OEG),desB30 human insulin,
34. A14E,B25H,B29K(N^{ε}Octadecanedioyl-OEG-γGlu-γGlu),desB30 human insulin,
35. A14E,A18L,B25H,B29K(N^{ε}Eicosanedioyl-γGlu-OEG-OEG),desB30 human insulin,
36. A14E,A18L,B25H,B29K(N^{ε}Octadecanedioyl-γGlu-OEG-OEG),desB30 human insulin,
37. A14E,B25H,B27E,B29K(N^{ε}Eicosanedioyl-γGlu-OEG-OEG),desB30 human insulin,
38. A1G(N^{α}Octadecandioyl-γGlu-OEG-OEG),A14E,B25H,B29R,desB30 human insulin,
39. A14E,B1F(N^{α}Octadecandioyl-γGlu-OEG-OEG),B25H,B29R,desB30 human insulin,
40. A1G(N^{α}Hexadecandioyl-γGlu),A14E,B25H,B29R,desB30 human insulin,
41. A14E,B25H,B29K(N^{ε}Octadecanedioyl-γGlu-Abu-Abu-Abu-Abu),desB30 human insulin,
42. A14E,B25H,B29K(N^{α}Eicosanedioyl),desB30 human insulin,
43. A14E,B25H,B29K(N^{α}4-[16-(1H-Tetrazol-5-yl)hexadecanoylsulfamoyl]butanoyl), desB30 human insulin,
44. A1G(N^{α}Octadecandioyl-γGlu-OEG-OEG),A14E,A21G,B25H,desB30 human insulin,
45. A14E,B25H,B29K(N^{ε}Eicosanedioyl-OEG),desB30 human insulin,
46. A14E,B25H,B27K(N^{ε}Octadecanedioyl-γGlu-OEG-OEG),desB28,desB29,desB30 human insulin,
47. A14E,B25H,B29K(N^{ε}(5-Eicosanedioylaminoisophthalic acid)),desB30 human insulin,
48. A14E,B25H,B29K(N^{ε}Octadecanedioyl),desB30 human insulin,
49. A14E,B29K(N^{ε}Octadecanedioyl-γGlu-OEG-OEG),desB30 human insulin,
50. A14E,B25H,B26G,B27G,B28G,B29K(N^{ε}Eicosanedioyl-γGlu-OEG-OEG),desB30 human insulin,
51. A14E,B25H,B29K(N^{ε}Octadecanedioyl-γGlu-OEG),desB30 human insulin,
52. A14E,B25H,B29K(N^{ε}Eicosanedioyl-OEG-OEG),desB30 human insulin,
53. A14E,B25H,B29K(N^{ε}Eicosanedioyl-Aoc),desB30 human insulin,
54. A14E,B25H,B26G,B27G,B28G,B29K(N^{ε}Eicosanedioyl-γGlu-γGlu),desB30 human insulin,
55. A14E,B25H,B26G,B27G,B28G,B29K(N^{ε}Eicosanedioyl-γGlu-γGlu),desB30 human insulin,
56. A14E,B25H,B29K(N^{ε}Octadecanedioyl-OEG),desB30 human insulin,
57. A14E,B25H,desB27,B29K(N^{ε}Octadecanedioyl-γGlu-OEG-OEG),desB30 human insulin,
58. A14E,B25H,B16H,B29K(N^{ε}Octadecanedioyl-γGlu),desB30 human insulin,
59. A1G(N^{α}Octadecanedioyl),A14E,B25H,B29R,desB30 human insulin,
60. A14E,B16H,B25H,B29K(N^{ε}Eicosanedioyl-γGlu),desB30 human insulin,
61. A14E,B25H,B27K(N^{ε}Eicosanedioyl-γGlu),desB28,desB29,desB30 human insulin,
62. A14E,B25H,B29K(N^{ε}Octadecanedioyl-γGlu-γGlu-γGlu),desB30 human insulin,
63. A14E,B25H,B26G,B27G,B28G,B29K(N^{ε}Octadecandioyl-γGlu),desB30 human insulin,
64. A14E,B25H,B26G,B27G,B28G,B29K(N^{ε}Eicosanedioyl-γGlu),desB30 human insulin,
65. A14E,B25H,B26G,B27G,B28G,B29K(N^{ε}Octadecandioyl),desB30 human insulin,
66. A14E,B25H,B26G,B27G,B28G,B29K(N^{ε}Eicosanedioyl),desB30 human insulin,
67. A14E,B25H,B29K(N^{ε}Docosanedioyl-γGlu),desB30 human insulin,
68. A14E,B25H,B29K(N^{ε}Docosanedioyl-γGlu-γGlu),desB30 human insulin,
69. A14E,B25H,B29K(N^{ε}Icosanedioyl-γGlu-OEG-OEG-γGlu),desB30 human insulin,
70. A14E,B25H,B29K(N^{ε}Octadecanedioyl-γGlu-OEG-OEG-γGlu),desB30 human insulin,
71. A14E,B25H,B29K(N^{ε}(N-Icosanedioyl-N-carboxymethyl)-βAla),desB30
72. A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
73. A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
74. A14E,B25H,B29K(N^{ε}Octadecandioyl-γGlu-(3-(2-{2-[2-(2-aminoethoxy)ethoxy]ethoxy}ethoxy)propionyl),desB30 human insulin,
75. A14E,B25H,B29K(N^{ε}Octadecandioyl-γGlu-(3-(2-{2-[2-(2-aminoethoxy)ethoxy]ethoxy}ethoxy)propionyl-γGlu),desB30 human insulin,
76. A14E,B25H,B29K(N^{ε}Icosanedioyl-γGlu-(3-(2-{2-[2-(2-aminoethoxy)ethoxy]ethoxy}ethoxy)propionyl),desB30 human insulin,
77. A14E,B25H,B29K(N^{ε}4-([4-({17-Carboxynonadecanoylamino}methyl)trans-cyclohexanecarbonyl]-γGlu),desB30 human insulin,
78. A14E,B25H,B29K(N^{ε}4-([4-({17-Carboxyheptadecanoylamino}methyl)trans-cyclohexanecarbonyl]-γGlu-γGlu),desB30 human insulin,
79. A14E,B28D,B29K(N^{ε}hexadecandioyl-γGlu),desB30 human insulin,
80. A14E,B28D,B29K(N^{ε}Eicosanedioyl-γGlu),desB30 human insulin,
81. A14E,B28D,B29K(N^{ε}Octadecandioyl-γGlu-OEG-OEG),desB30 human insulin,
82. A14E,B28D,B29K(N^{ε}Eicosanedioyl-γGlu-OEG-OEG),desB30 human insulin,
83. A14E,B28E,B29K(N^{ε}Hexadecandioyl-γGlu),desB30 human insulin,
84. A14E,B28E,B29K(N^{ε}Octadecandioyl-γGlu),desB30 human insulin,
85. A14E,B28E,B29K(N^{ε}Eicosanedioyl-γGlu),desB30 human insulin,
86. A14E,B28E,B29K(N^{ε}Octadecandioyl-γGlu-OEG-OEG),desB30 human insulin,
87. A14E,B28E,B29K(N^{ε}Eicosanedioyl-γGlu-OEG-OEG),desB30 human insulin,
88. A14E,B1E,B28E,B29K(N^{ε}Hexadecandioyl-γGlu),desB30 human insulin,
89. A14E,B1E,B28E,B29K(N^{ε}Octadecandioyl-γGlu),desB30 human insulin,
90. A14E,B1E,B28E,B29K(N^{ε}Eicosanedioyl-γGlu),desB30 human insulin,
91. A14E,B1E,B28E,B29K(N^{ε}Hexadecandioyl-γGlu-OEG-OEG),desB30 human insulin,
92. A14E,B1E,B28E,B29K(N^{ε}Octadecandioyl-γGlu-OEG-OEG),desB30 human
93. A14E,B1E,B28E,B29K(N^{ε}Eicosanedioyl-γGlu-OEG-OEG),desB30 human insulin,
94. A14E,B1E,B27E,B28E,B29K(N^{ε}Hexadecandioyl-γGlu),desB30 human insulin,
95. A14E,B1E,B27E,B28E,B29K(N^{ε}Octadecandioyl-γGlu),desB30 human insulin,
96. A14E,B1E,B27E,B28E,B29K(N^{ε}Eicosanedioyl-γGlu),desB30 human insulin,
97. A14E,B1E,B27E,B28E,B29K(N^{ε}Hexadecandioyl-γGlu-OEG-OEG),desB30 human insulin,
98. A14E,B1E,B27E,B28E,B29K(N^{ε}Octadecandioyl-γGlu-OEG-OEG),desB30 human insulin,
99. A14E,B1E,B27E,B28E,B29K(N^{ε}Eicosanedioyl-γGlu-OEG-OEG),desB30 human insulin,
100. A14E,B1E,B25H,B28E,B29K(N^{ε}Hexadecandioyl-γGlu),desB30 human insulin,
101. A14E,B1E,B25H,B28E,B29K(N^{ε}Octadecandioyl-γGlu),desB30 human insulin,
102. A14E,B1E,B25H,B28E,B29K(N^{ε}Eicosanedioyl-γGlu),desB30 human insulin,
103. A14E,B1E,B25H,B28E,B29K(N^{ε}Hexadecandioyl-γGlu-OEG-OEG),desB30 human insulin,
104. A14E,B1E,B25H,B28E,B29K(N^{ε}Octadecandioyl-γGlu-OEG-OEG),desB30 human insulin,
105. A14E,B1E,B25H,B28E,B29K(N^{ε}Eicosanedioyl-γGlu-OEG-OEG),desB30 human insulin,
106. A14E,B1E,B25H,B27E,B28E,B29K(N^{ε}Hexadecandioyl-γGlu),desB30 human insulin,
107. A14E,B1E,B25H,B27E,B28E,B29K(N^{ε}Octadecandioyl-γGlu),desB30 human insulin,
108. A14E,B1E,B25H,B27E,B28E,B29K(N^{ε}Eicosanedioyl-γGlu),desB30 human insulin,
109. A14E,B1E,B25H,B27E,B28E,B29K(N^{ε}Hexadecandioyl-γGlu-OEG-OEG),desB30 human insulin,
110. A14E,B1E,B25H,B27E,B28E,B29K(N^{ε}Octadecandioyl-γGlu-OEG-OEG),desB30 human insulin,
111. A14E,B1E,B25H,B27E,B28E,B29K(N^{ε}Eicosanedioyl-γGlu-OEG-OEG),desB30
112. A14E,B28D,B29K(N^{ε}Hexadecanedioyl-γGlu-OEG-OEG),desB30 human insulin,
113. A14E,B28E,B29K(N^{ε}Hexadecanedioyl-γGlu-OEG-OEG),desB30 human insulin,
114. B25N,B27E,B29K(N^{ε}Eicosanedioyl-γGlu-OEG-OEG),desB30 human insulin,
115. B25N,B27E,B29K(N^{ε}Octadecanedioyl-γGlu-OEG-OEG),desB30 human insulin,
116. B25N,B27E,B29K(N^{ε}Hexadecanedioyl-γGlu-OEG-OEG),desB30 human insulin,
117. B25N,B27E,B29K(N^{ε}Eicosanedioyl-γGlu),desB30 human insulin,
118. B25N,B27E,B29K(N^{ε}Octadecanedioyl-γGlu),desB30 human insulin,
119. B25N,B27E,B29K(N^{ε}Hexadecanedioyl-γGlu),desB30 human insulin,
120. A8H,B25N,B27E,B29K(N^{ε}Eicosanedioyl-γGlu-OEG-OEG),desB30 human insulin,
121. A8H,B25N,B27E,B29K(N^{ε}Octadecanedioyl-γGlu-OEG-OEG),desB30 human insulin,
122. A8H,B25N,B27E,B29K(N^{ε}Hexadecanedioyl-γGlu-OEG-OEG),desB30 human insulin,
123. A8H,B25N,B27E,B29K(N^{ε}Eicosanedioyl-γGlu),desB30 human insulin,
124. A8H,B25N,B27E,B29K(N^{ε}Octadecanedioyl-γGlu),desB30 human insulin,
125. A8H,B25N,B27E,B29K(N^{ε}Hexadecanedioyl-γGlu),desB30 human insulin,
126. 14E,B25H,B29K(N^{ε}(N-Icosanedioyl-N-carboxymethyl)-βAla-OEG-OEG),desB30 human insulin,
127. A14E,B25H,B29K(N^{ε}(N-Octadecanedioyl-N-carboxymethyl)-βAla-OEG-OEG),desB30 human insulin,
128. A14E,B25H,B29K(N^{ε}(N-Hexadecanedioyl-N-carboxymethyl)-βAla-OEG-OEG),desB30 human insulin,
129. A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
130. A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30
131. A14E,B16H,B25H,B29K(N^{ε}Octadecanedioyl-γGlu-2-[(3-{2-[2-(3-aminopropoxy)ethoxy]ethoxy}propylcarbamoyl)methoxy]acetyl),desB30 human insulin,
132. A14E, B16H, B25H, B29K(N^{ε}Eicosanedioyl-γGlu-2-[(3-{2-[2-(3-aminopropoxy)ethoxy]ethoxy}propylcarbamoyl)methoxy]acetyl),desB30 human insulin,
133. B25H,B29K(N^{ε}Octadecanedioyl-γGlu-OEG-OEG),desB30 human insulin,
134. B25H,B29K(N^{ε}Eicosanedioyl-γGlu-OEG-OEG),desB30 human insulin,
135. B25H,B29K(N^{ε}Octadecanedioyl-γGlu),desB30 human insulin,
136. B25H,B29K(N^{ε}Eicosanedioyl-γGlu),desB30 human insulin,
137. B25H,B29K(N^{ε}Octadecanedioyl),desB30 human insulin,
138. B25H,B29K(N^{ε}Eicosanedioyl),desB30 human insulin,
139. B25H,B29K(N^{ε}Octadecanedioyl-γGlu-OEG-OEG),desB30 human insulin,
140. B25H,B29K(N^{ε}Eicosanedioyl-γGlu-OEG-OEG),desB30 human insulin,
141. B25H,B29K(N^{ε}Octadecanedioyl-γGlu),desB30 human insulin,
142. B25H,B29K(N^{ε}Eicosanedioyl-γGlu),desB30 human insulin,
143. 21G,B25H,B29K(N^{ε}Octadecanedioyl),desB30 human insulin,
144. A21G,B25H,B29K(N^{ε}Eicosanedioyl),desB30 human insulin,
145. A21G,B25H,B29K(N^{ε}Octadecanedioyl-γGlu-OEG-OEG),desB30 human insulin,
146. A21G,B25H,B29K(N^{ε}Eicosanedioyl-γGlu-OEG-OEG),desB30 human insulin,
147. A21G,B25H,B29K(N^{ε}Octadecanedioyl-γGlu),desB30 human insulin,
148. A21G,B25H,B29K(N^{ε}Eicosanedioyl-γGlu),desB30 human insulin,
149. A14E,B25H,desB27,B29K(N^{ε}Octadecanedioyl),desB30 human insulin,
150. A14E,B25H,desB27,B29K(N^{ε}Eicosanedioyl),desB30 human insulin,
151. A14E,B25H,desB27,B29K(N^{ε}Octadecanedioyl-γGlu),desB30 human insulin,
152. A14E,B25H,desB27,B29K(N^{ε}Eicosanedioyl-γGlu),desB30 human insulin,
153. A14E,B25H,desB27,B29K(N^{ε}Eicosanedioyl-γGlu-OEG-OEG),desB30 human insulin,
154. A14E,A21G,B25H,desB27,B29K(N^{ε}Octadecanedioyl),desB30 human insulin,
155. A14E,A21G,B25H,desB27,B29K(N^{ε}Eicosanedioyl),desB30 human insulin,
156. A14E,A21G,B25H,desB27,B29K(N^{ε}Octadecanedioyl-γGlu),desB30 human
157. A14E,B25H,desB27,B29K(N^{ε}Eicosanedioyl-γGlu),desB30 human insulin,
158. A14E,A21G,B25H,desB27,B29K(N^{ε}Octadecanedioyl-γGlu-OEG-OEG),desB30 human insulin,
159. A14E,A21G,B25H,desB27,B29K(N^{ε}Eicosanedioyl-γGlu-OEG-OEG),desB30 human insulin,
160. A14E,A21G,B25H,B29K(N^{ε}Octadecanedioyl-γGlu-OEG-OEG),desB30 human insulin,
161. A14E,A21G,B25H,B29K(N^{ε}Eicosanedioyl-γGlu-OEG-OEG),desB30 human insulin,
162. A14E,A21G,B25H,B29K(N^{ε}Eicosanedioyl-γGlu),desB30 human insulin,
163. A14E,A21G,B25H,B29K(N^{ε}Eicosanedioyl),desB30 human insulin,
164. A14E,A21G,B25H,B29K(N^{ε}Octadecanedioyl-γGlu),desB30 human insulin,
165. A14E,A21G,B25H,B29K(N^{ε}Octadecanedioyl),desB30 human insulin,
166. A14E,B25H,B26G,B27G,B28G,B29K(N^{ε}Octadecanedioyl-γGlu),desB30 human insulin,
167. A14E,B25H,B26G,B27G,B28G,B29K(N^{ε}Octadecanedioyl),desB30 human insulin,
168. A14E,B25H,B26G,B27G,B28G,B29K(N^{ε}Eicosanedioyl-γGlu),desB30 human insulin,
169. A14E,B25H,B26G,B27G,B28G,B29K(N^{ε}Eicosanedioyl),desB30 human insulin,
170. A1G(N^{α}Octadecandioyl-γGlu),A14E,B25H,B26G,B27G,B28G,desB30 human insulin,
171. A1G(N^{α}Eicosanedioyl-γGlu),A14E,B25H,B26G,B27G,B28G,desB30 human insulin,
172. A1G(N^{α}Octadecandioyl-γGlu),A14E,B25H,B26G,B27G,B28G,B29R,desB30 human insulin,
173. A1G(N^{α}Eicosanedioyl-γGlu),A14E,B25H,B26G,B27G,B28G,B29R,desB30 human insulin,
174. A1G(N^{α}Octadecandioyl),A14E,B25H,B26G,B27G,B28G,desB30 human insulin,
175. A1G(N^{α}Eicosanedioyl),A14E,B25H,B26G,B27G,B28G,desB30 human insulin,
176. A1G(N^{α}Octadecandioyl),A14E,B25H,B26G,B27G,B28G,B29R,desB30 human insulin and
177. A1G(N^{α}Eicosanedioyl),A14E,B25H,B26G,B27G,B28G,B29R,desB30 human insulin.

In one embodiment a tablet core according to the present invention comprises an acylated insulin selected from the group consisting of:
1. A10C,A14E,B4C,B25H,B29K(N^{ε}Octadecanedioyl-γGluγGlu-OEG-OEG),desB30 human insulin,
2. A10C,A14E,B3C,B25H,B29K(N(eps)Octadecanedioyl-γGluγGlu),desB30 human insulin,
3. A10C,A14E,B3C,B25H,B29K(N^{ε}Octadecanedioyl),desB30 human insulin,
4. A10C,A14E,B3C,B25H,B29K(N^{ε}Octadecanedioyl-γGluγGlu),desB30 human insulin,
5. A10C,A14E,desB1,B4C,B25H,B29K(N^{ε}Octadecanedioyl-γGluγGlu-OEG-OEG),desB30 human insulin,
6. A10C,A14H,B4C,B25H,B29K(N^{ε}Octadecanedioyl-γGlu-OEG-OEG),desB30 human insulin,
7. A10C,A14E,B3C,B25H,B29K(N^{ε}Eicosanedioyl-γGlu-OEG-OEG),desB30 human insulin,
8. A10C,A14E,B1C, B25H,B29K(N^{ε}Eicosanedioyl-γGlu-OEG-OEGOEG-OEG),desB30 human insulin,
9. A10C,A14E,B4C B25H,B29K(N^{ε}Octadecanedioyl-γGlu),desB30 human insulin,
10. A10C,A14E, B3C,B25H,B29K(N^{ε}Octadecanedioyl-γGlu-OEG-OEG),desB30 human insulin,
11. A10C,A14E,B3C,B25H,B29K(N^{ε}Octadecanedioyl-γGlu-γGlu),desB30 human insulin,
12. A10C,A14E,B4C,B25H,desB27,B29K(N^{ε}Octadecanedioyl-γGlu),desB30 human insulin,
13. A10C,A14E,B4C,B25H,B29K(N^{ε}Octadecanedioyl),desB30 human insulin,
14. A10C,A14E,B4C,B25H,B29K(N^{ε}Octadecanedioyl-γGlu-γGlu),desB30 human insulin,
15. A10C,A14E,B2C,B25H,B29K(N^{ε}Octadecanedioyl-γGlu-OEG-OEG),desB30 human insulin,
16. A10C,A14E,B1C,B25H,B29K(N^{ε}Octadecanedioyl-γGlu-OEG-OEG),desB30 human insulin,
17. A10C,A14E,B3C,B16H,B25H,B29K(N^{ε}Eicosanedioyl-γGlu-OEG-OEG),desB30 human
18. A10C,A14E, B4C,B25H,B29K(N^{ε}Myristyl),desB30 human insulin,
19. A10C,B4C, B29K(N^{ε}Myristyl),desB30 human insulin,
20. A10C,A14E,B3C,B25H,desB27,B29K(N(eps)octadecanedioyl-γGlu),desB30 human insulin,
21. A10C,A14E,B3C,B25H,desB27,B29K(N(eps)octadecanedioyl-γGlu-OEG-OEG),desB30 human insulin,
22. A10C,A14E,B3C,B25H,B29K(N^{ε}Eicosanedioyl-γGlu),desB30 human insulin,
23. A10C,A14E,B4C,B25H,B29K(N(eps)eicosanedioyl-γGlu-OEG-OEG),desB30 human insulin,
24. A10C,A14E,B3C,B25H,desB27,B29K(N(eps)eicosanedioyl-γGlu),desB30 human insulin,
25. A10C,A14E,B3C,B25H,desB27, B29K(N(eps)eicosanedioyl-γGlu-OEG-OEG),desB30 human insulin,
26. A10C,A14E, 4C,B25H,B29K(N^{ε}Hexadecanedioyl-γGlu),desB30 human insulin,
27. A10C,A14E,B4C,B25H,B29K(N^{ε}Hexadecanedioyl-γGlu-OEG-OEG),desB30 human insulin,
28. A10C,A14E,B4C,B25H,B29K(N^{ε}Hexadecanedioyl),desB30 human insulin,
29. A10C,A14E,B4C,B25H,B29K(N^{ε}Hexadecanedioyl-γGlu-γGlu),desB30 human insulin,
30. A10C,A14E,B4C,B25H,desB27,B29K(N^{ε}Octadecanedioyl-γGlu-OEG-OEG),desB30 human insulin,
31. A10C,A14E,B4C,B25H,desB27,B29K(N^{ε}Octadecanedioyl-γGlu-γGlu),desB30 human insulin,
32. A10C,A14E,B4C,B25H,desB27,B29K(N^{ε}Hexadecanedioyl-γGlu-OEG-OEG),desB30 human insulin,
33. A10C,A14E,B4C,B25H,desB27,B29K(N^{ε}Hexadecanedioyl-γGlu),desB30 human insulin,
34. A10C,A14E,B3C,B25H,B29K(N^{ε}Hexadecanedioyl-γGlu),desB30 human insulin,
35. A10C,A14E,B3C,B25H,B29K(N^{ε}Hexadecanedioyl-γGlu-OEG-OEG),desB30 human insulin,
36. A10C,A14E,B2C,B25H,B29K(N^{ε}Hexadecanedioyl-γGlu-OEG-OEG),desB30 human insulin,
37. A10C,A14E,B2C,B25H,B29K(N^{ε}Hexadecanedioyl-γGlu),desB30 human insulin,
38. A10C,A14E,B2C,B25H,B29K(N^{ε}Octadecanedioyl-γGlu),desB30 human insulin,
39. A10C,A14E,B1C,B25H,B29K(N^{ε}Octadecanedioyl-γGlu),desB30 human insulin,
40. A10C,A14E,B1C,B25H,B29K(N^{ε}Hexadecanedioyl-γGlu-OEG-OEG),desB30 human insulin,
41. A10C,A14E,B1C,B25H,B29K(N^{ε}Hexadecanedioyl-γGlu),desB30 human insulin,
42. A10C,B1C,B29K(N^{ε}Hexadecanedioyl-γGlu-OEG-OEG),desB30 human insulin,
43. A10C,B1C,B29K(N^{ε}Hexadecanedioyl-γGlu),desB30 human insulin,
44. A10C,B1C,B29K(N^{ε}Octadecanedioyl-γGlu-OEG-OEG),desB30 human insulin,
45. A10C,B1C,B29K(N^{ε}Octadecanedioyl-γGlu),desB30 human insulin,
46. A10C,B2C,B29K(N^{ε}Octadecanedioyl-γGlu-OEG-OEG),desB30 human insulin,
47. A10C,B2C,B29K(N^{ε}Octadecanedioyl-γGlu),desB30 human insulin,
48. A10C,B2C,B29K(N^{ε}Hexadecanedioyl-γGlu-OEG-OEG),desB30 human insulin,
49. A10C,B2C,B29K(N^{ε}Hexadecanedioyl-γGlu),desB30 human insulin,
50. A10C,B3C,B29K(N^{ε}Hexadecanedioyl-γGlu),desB30 human insulin,
51. 10C,B3C B29K(N^{ε}Hexadecanedioyl-γGlu-OEG-OEG),desB30 human insulin,
52. A10C,B3C,B29K(N^{ε}Octadecanedioyl-γGlu-OEG-OEG),desB30 human insulin,
53. A10C,B3C,B29K(N^{ε}Octadecanedioyl-γGlu),desB30 human insulin,
54. A10C,B4C,B29K(N^{ε}Octadecanedioyl-γGlu),desB30 human insulin,
55. A10C,B4C,B29K(N^{ε}Octadecanedioyl-γGlu-OEG-OEG),desB30 human insulin,
56. A10C,B4C B29K(N^{ε}Hexadecanedioyl-γGlu-OEG-OEG),desB30 human insulin,
57. A10C,B4C,B29K(N^{ε}Hexadecanedioyl-γGlu),desB30 human insulin,
58. A10C,A14E,B1C,B16H,B25H,B29K(N^{ε}eicosanedioyl-γGlu-OEG-OEG),desB30 human insulin,
59. A10C,A14E,B1C,B16H,B25H,B29K(N^{ε}eicosanedioyl-γGlu),desB30 human insulin,
60. A10C,A14E,B1C,B16H,B25H,B29K(N^{ε}Octadecanedioyl-γGlu-OEG-OEG),desB30 human insulin,
61. A10C,A14E,B1C,B16H,B25H,B29K(N^{ε}Octadecanedioyl-γGlu),desB30 human insulin,
62. A10C A14E,B1C,B16H,B25H, B29K(N^{ε}Hexadecanedioyl-γGlu),desB30 human insulin,
63. A10C,A14E,B1C,B16H, B25H,B29K(N^{ε}Hexadecanedioyl-γGlu-OEG-OEG),desB30 human insulin,
64. A10C,A14E,B2C,B16H,B25H,B29K(N^{ε}Hexadecanedioyl-γGlu-OEG-OEG),desB30 human insulin,
65. A10C,A14E,B2C,B16H,B25H,B29K(N^{ε}Hexadecanedioyl-γGlu),desB30 human insulin,
66. A10C,A14E,B2C,B16H,B25H,B29K(N^{ε}Octadecanedioyl-γGlu),desB30 human insulin,
67. A10C,A14E,B2C,B16H,B25H,B29K(N^{ε}Octadecanedioyl-γGlu-OEG-OEG),desB30 human insulin,
68. A10C,A14E,B2C,B16H,B25H,B29K(N^{ε}Eicosanedioyl-γGlu-OEG-OEG),desB30 human insulin,
69. A10C,A14E,B2C,B16H,B25H,B29K(N^{ε}Eicosanedioyl-γGlu),desB30 human insulin,
70. A10C,A14E,B3C,B16H,B25H,B29K(N^{ε}Eicosanedioyl-γGlu),desB30 human insulin,
71. A10C,A14E,B3C,B16H,B25H,B29K(N^{ε}Octadecanedioyl-γGlu-OEG-OEG),desB30 human insulin,
72. A10C,A14E,B3C,B16H, B25H,B29K(N^{ε}Octadecanedioyl-γGlu),desB30 human insulinA10C,A14E,B3C,B16H,B25H,B29K(N^{ε}Hexadecanedioyl-γGlu),desB30 human insulin,
74. A10C,A14E,B3C,B16H,B25H,B29K(N^{ε}Hexadecanedioyl-γGlu-OEG-OEG),desB30 human insulin,
75. A10C,A14E,B4C,B16H,B25H,B29K(N^{ε}Hexadecanedioyl-γGlu-OEG-OEG),desB30 human insulin,
76. A10C,A14E,B4C,B16H,B25H,B29K(N^{ε}Hexadecanedioyl-γGlu),desB30 human insulin,
77. A10C,A14E,B4C,B16H,B25H,B29K(N^{ε}Octadecanedioyl-γGlu),desB30 human insulin,
78. A10C,A14E,B4C,B16H,B25H B29K(N^{ε}Octadecanedioyl-γGlu-OEG-OEG),desB30 human insulin,
79. A10C,A14E,B4C,B16H B25H,B29K(N^{ε}Eicosanedioyl-γGlu-OEG-OEG),desB30 human insulin,
80. A10C,A14E,B4C,B16H,B25H,B29K(N^{ε}Eicosanedioyl-γGlu),desB30 human insulin,
81. A10C,A14E,B1C,B25H,B29K(N(eps)eicosanedioyl-γGlu),desB30 human insulin,
82. A10C,A14E,B2C,B25H,B29K(N(eps)eicosanedioyl-γGlu),desB30 human insulin,
83. A10C,A14E,B2C,B25H,B29K(N(eps)eicosanedioyl-γGlu-OEG-OEG),desB30 human insulin,
84. A10C,A14E,B4C,B25H,desB27,B29K(N(eps)eicosanedioyl-γGlu),desB30 human insulin,
85. A10C,A14E,B4C,B25H,desB27,B29K(N(eps)eicosanedioyl-γGlu-OEG-OEG),desB30 human insulin,
86. A10C,A14E,B4C,B25H,B29K(N(eps)eicosanedioyl-γGlu),desB30 human insulin,
87. A10C,A14E,B3C,B25H,desB27,B29K(N(eps)hexadecanedioyl-γGlu),desB30 human
88. A10C,A14E,B3C,B25H,desB27,B29K(N(eps)hexadecanedioyl-γGlu-OEG-OEG),desB30 human insulin,
89. A10C,A14E,B3C,desB27,B29K(N(eps)hexadecanedioyl-γGlu),desB30 human insulin,
90. A10C,A14E,B3C,desB27,B29K(N(eps)hexadecanedioyl-γGlu-OEG-OEG),desB30 human insulin,
91. A10C,A14E,B3C,desB27,B29K(N(eps)octadecanedioyl-γGlu),desB30 human insulin,
92. A10C,A14E,B3C,desB27,B29K(N(eps)octadecanedioyl-γGlu-OEG-OEG),desB30 human insulin,
93. A10C,A14E,B3C,desB27,B29K(N(eps)eicosanedioyl-γGlu),desB30 human insulin,
94. A10C,A14E,B3C,desB27,B29K(N(eps)eicosanedioyl-γGlu-OEG-OEG),desB30 human insulin,
95. A10C,A14E,B3C,B16H,B25H,B29K(N(eps)eicosanedioyl-γGlu-γGlu),desB30 human insulin,
96. A10C,A14E,B3C,B16E,B25H,B29K(N(eps)eicosanedioyl-γGlu-OEG-OEG),desB30 human insulin,
97. A10C,A14E,B4C,B16E,B25H,B29K(N(eps)eicosanedioyl-γGlu-OEG-OEG),desB30 human insulin,
98. A10C,A14E,B3C,B16H,B25H,B29K(N(eps)eicosanedioyl-γGlu-γGlu),desB30 human insulin and
99. A10C,A14E,B4C,B16E,B25H,B29K(N(eps)eicosanedioyl-γGlu-γGlu),desB30 human insulin.

In one embodiment a tablet core according to the present invention comprises an acylated insulin selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B3C,B25H,B29K(N^{ε}Octadecanedioyl),desB30 human insulin,
A10C,A14E,B3C,B25H,B29K(N^{ε}Octadecanedioyl-γGlu),desB30 human insulin,
A10C,A14H,B4C,B25H,B29K(N^{ε}Octadecanedioyl-γGlu-OEG-OEG),desB30 human insulin,
A10C,A14E,B3C,B25H,B29K(N^{ε}Eicosanedioyl-γGlu-OEG-OEG),desB30 human
A10C,A14E,B4C B25H,B29K(N^{ε}Octadecanedioyl-γGlu),desB30 human insulin,
A10C,A14E, B3C,B25H,B29K(N^{ε}Octadecanedioyl-γGlu-OEG-OEG),desB30 human insulin,
A10C,A14E,B3C,B25H,B29K(N^{ε}Octadecanedioyl-γGlu-γGlu),desB30 human insulin,
A10C,A14E,B4C,B25H,desB27,B29K(N^{ε}Octadecanedioyl-γGlu),desB30 human insulin,
A10C,A14E,B4C,B25H,B29K(N^{ε}Octadecanedioyl),desB30 human insulin,
A10C,A14E,B4C,B25H,B29K(N^{ε}Octadecanedioyl-γGlu-γGlu),desB30 human insulin,
A10C,A14E,B3C,B16H,B25H,B29K(N^{ε}Eicosanedioyl-γGlu-OEG-OEG),desB30 human insulin,
A10C,A14E, B4C,B25H,B29K(N^{ε}Myristyl),desB30 human insulin,
A10C,B4C, B29K(N^{ε}Myristyl),desB30 human insulin,
A10C,A14E,B3C,B25H,desB27,B29K(N(eps)octadecanedioyl-γGlu),desB30 human insulin,
A10C,A14E,B3C,B25H,desB27,B29K(N(eps)octadecanedioyl-γGlu-OEG-OEG),desB30 human insulin,
A10C,A14E,B3C,B25H,B29K(N^{ε}Eicosanedioyl-γGlu),desB30 human insulin,
A10C,A14E,B4C,B25H,B29K(N(eps)eicosanedioyl-γGlu-OEG-OEG),desB30 human insulin,
A10C,A14E,B3C,B25H,desB27,B29K(N(eps)eicosanedioyl-γGlu),desB30 human insulin,
A10C,A14E,B3C,B25H,desB27, B29K(N(eps)eicosanedioyl-γGlu-OEG-OEG),desB30 human insulin,
A10C,A14E, 4C,B25H,B29K(N^{ε}Hexadecanedioyl-γGlu),desB30 human insulin,
A10C,A14E,B4C,B25H,B29K(N^{ε}Hexadecanedioyl-γGlu-OEG-OEG),desB30 human insulin,
A10C,A14E,B4C,B25H,B29K(N^{ε}Hexadecanedioyl),desB30 human insulin,
A10C,A14E,B4C,B25H,B29K(N^{ε}Hexadecanedioyl-γGlu-γGlu),desB30 human
A10C,A14E,B4C,B25H,desB27,B29K(N^{ε}Octadecanedioyl-γGlu-OEG-OEG),desB30 human insulin,A10C,A14E,B4C,B25H,desB27,B29K(N^{ε}Octadecanedioyl-γGlu-γGlu),desB30 human insulin,
A10C,A14E,B4C,B25H,desB27,B29K(N^{ε}Hexadecanedioyl-γGlu-OEG-OEG),desB30 human insulinA10C,A14E,B4C,B25H,desB27,B29K(N^{ε}Hexadecanedioyl-γGlu),desB30 human insulin,
A10C,A14E,B3C,B25H,B29K(N^{ε}Hexadecanedioyl-γGlu),desB30 human insulin,
A10C,A14E,B3C,B25H,B29K(N^{ε}Hexadecanedioyl-γGlu-OEG-OEG),desB30 human insulin,
A10C,B3C,B29K(N^{ε}Hexadecanedioyl-γGlu),desB30 human insulin,
10C,B3C B29K(N^{ε}Hexadecanedioyl-γGlu-OEG-OEG),desB30 human insulin,
A10C,B3C,B29K(N^{ε}Octadecanedioyl-γGlu-OEG-OEG),desB30 human insulin,
A10C,B3C,B29K(N^{ε}Octadecanedioyl-γGlu),desB30 human insulin,
A10C,B4C,B29K(N^{ε}Octadecanedioyl-γGlu),desB30 human insulin,
A10C,B4C,B29K(N^{ε}Octadecanedioyl-γGlu-OEG-OEG),desB30 human insulin,
A10C,B4C B29K(N^{ε}Hexadecanedioyl-γGlu-OEG-OEG),desB30 human insulin,
A10C,B4C,B29K(N^{ε}Hexadecanedioyl-γGlu),desB30 human insulin,
A10C,A14E,B3C,B16H,B25H,B29K(N^{ε}Eicosanedioyl-γGlu),desB30 human insulin,
A10C,A14E,B3C,B16H,B25H,B29K(N^{ε}Octadecanedioyl-γGlu-OEG-OEG),desB30 human insulin,
A10C,A14E,B3C,B16H, B25H,B29K(N^{ε}Octadecanedioyl-γGlu),desB30 human
insulinA10C,A14E,B3C,B16H,B25H,B29K(N^{ε}Hexadecanedioyl-γGlu),desB30 human insulin,
A10C,A14E,B3C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu),desB30 human insulin,
A10C,A14E,B4C,B16H,B25H,B29K(N^{ε}Octadecanedioyl-γGlu),desB30 human insulin,
A10C,A14E,B4C,B16H,B25H B29K(N^{ε}Octadecanedioyl-γGlu-OEG-OEG),desB30
A10C,A14E,B4C,B16H B25H,B29K(N^{ε}Eicosanedioyl-γGlu-OEG-OEG),desB30 human insulin,
A10C,A14E,B4C,B16H,B25H,B29K(N^{ε}Eicosanedioyl-γGlu),desB30 human insulin,
A10C,A14E,B4C,B25H,desB27,B29K(N(eps)eicosanedioyl-γGlu),desB30 human insulin,
A10C,A14E,B4C,B25H,desB27,B29K(N(eps)eicosanedioyl-γGlu-OEG-OEG),desB30 human insulin,
A10C,A14E,B4C,B25H,B29K(N(eps)eicosanedioyl-γGlu),desB30 human insulin,
A10C,A14E,B3C,B25H,desB27,B29K(N(eps)hexadecanedioyl-γGlu),desB30 human insulin,
A10C,A14E,B3C,B25H,desB27,B29K(N(eps)hexadecanedioyl-γGlu-OEG-OEG),desB30 human insulin,
A10C,A14E,B3C,desB27,B29K(N(eps)hexadecanedioyl-γGlu),desB30 human insulin,
A10C,A14E,B3C,desB27,B29K(N(eps)hexadecanedioyl-γGlu-OEG-OEG),desB30 human insulin,
A10C,A14E,B3C,desB27,B29K(N(eps)octadecanedioyl-γGlu),desB30 human insulin,
A10C,A14E,B3C,desB27,B29K(N(eps)octadecanedioyl-γGlu-OEG-OEG),desB30 human insulin,
A10C,A14E,B3C,desB27,B29K(N(eps)eicosanedioyl-γGlu),desB30 human insulin,
A10C,A14E,B3C,desB27,B29K(N(eps)eicosanedioyl-γGlu-OEG-OEG),desB30 human insulin,
A10C,A14E,B3C,B16H,B25H,B29K(N(eps)eicosanedioyl-γGlu-γGlu),desB30 human insulin,
A10C,A14E,B3C,B16E,B25H,B29K(N(eps)eicosanedioyl-γGlu-OEG-OEG),desB30 human insulin,
A10C,A14E,B4C,B16E,B25H,B29K(N(eps)eicosanedioyl-γGlu-OEG-OEG),desB30 human insulin,
A10C,A14E,B3C,B16H,B25H,B29K(N(eps)eicosanedioyl-γGlu-γGlu),desB30 human insulin,
A10C,A14E,B4C,B16E,B25H,B29K(N(eps)eicosanedioyl-γGlu-γGlu),desB30
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N(eps)Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin,
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N(eps)octadecanedioyl-γGlu-OEG-OEG),desB30 human insulin,
A10C,A14E,B3C,B25H,desB27, B29K(N(eps)eicosanedioyl-γGlu-OEG-OEG),desB30 human insulin,
A10C,A14E,B3C,B16H,B25H,B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N(eps)eicosanedioyl-γGlu-OEG-OEG),desB30 human insulin.

In one embodiment a tablet core according to the present invention comprises an acylated insulin selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N(eps)octadecanedioyl-γGlu-OEG-OEG),desB30 human insulin,
A10C,A14E,B3C,B25H,desB27, B29K(N(eps)eicosanedioyl-γGlu-OEG-OEG),desB30 human insulin,
A10C,A14E,B3C,B16H,B25H,B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N(eps)eicosanedioyl-γGlu-OEG-OEG),desB30 human insulin.

In one embodiment a tablet core according to the present invention comprises a protease stabilised acylated insulin, which is selected from the group consisting of:
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N(eps)Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin.

### Kit

In one embodiment the present invention relates to a kit comprising an oral pharmaceutical composition as described herein and instructions for use.

In one embodiment the present invention relates to a kit comprising an oral pharmaceutical composition in the form of one or more tablets as described herein and instructions for use. In one embodiment the present invention relates to a kit comprising an oral pharmaceutical composition in the form of one or more tablets each comprising one or more uncoated or coated tablet core as described herein and instructions for use. In one embodiment the present invention relates to a kit comprising an oral pharmaceutical composition in the form of one or more capsulels each comprising one or more uncoated or coated tablet core as described herein and instructions for use.

In one embodiment said oral pharmaceutical composition comprised in said kit is provided in a blisterpack.

In one embodiment said oral pharmaceutical composition comprised in said kit is provided in a container.

In one embodiment said oral pharmaceutical composition comprised in said kit is provided in a container of plastics or glas or a combination thereof.

In one embodiment said oral pharmaceutical composition comprised in said kit is provided in a container.

### Terms and Definitions

With “insulin”, “an insulin” or “the insulin” as used herein is meant human insulin, porcine insulin or bovine insulin with disulfide bridges between CysA7 and CysB7 and between CysA20 and CysB19 and an internal disulfide bridge between CysA6 and CysA11 or an insulin analogue or derivative thereof. The term “insulin”, “an insulin” or “the insulin” further includes “insulin analogues”. The term “human insulin” as used herein means the human insulin hormone in which the two dimensional and three dimensional structures and properties are well-known. The three dimensional structure of human insulin has been e.g. determined by NMR and X-ray crystallography under many different conditions and many of these structures are deposited in the Protein data bank (http://www.rcsb.org). Non-limiting examples of a human insulin structure is the T6 structure (http://www.rcsb.org/pdb/explore.do?structureId=1MSO) and the R6 structure (http://www.rcsb.org/pdb/explore.do?structureId=1EV3). Human insulin has two polypeptide chains, named the A-chain and the B-chain. The A-chain is a 21 amino acid peptide and the B-chain is a 30 amino acid peptide, the two chains being connected by disulfide bonds: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain. Thus “an acylated insulin where the three disulfide bonds of human insulin are retained” is herein understood as an acylated insulin comprising the three disulfide bonds of human insulin, i.e. a disulfide bond between the cysteine in position 7 of the A-chain and the cysteine in position 7 of the B-chain, a disulfide bond between the cysteine in position 20 of the A-chain and the cysteine in position 19 of the B-chain and a disulfide bond between the cysteines in position 6 and 11 of the A-chain. In the human body, the insulin hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains.

As used in this specification and appended embodiments, the singular forms “a”, “an” and “the” include plural referents unless the content clearly dictates otherwise. Thus, for example, reference to “an insulin” includes one or more insulins and a mixture of one or more insulins, and the like.

The term “insulin peptide” as used herein means a peptide which is either human insulin or an analogue or a derivative thereof with insulin activity.

The term “insulin analogue” as used herein means a modified insulin wherein one or more amino acid residues of the insulin have been substituted by other amino acid residues and/or wherein one or more amino acid residues have been deleted from the insulin and/or wherein one or more amino acid residues have been added and/or inserted to the insulin. An insulin analogue as used herein is a polypeptide which has a molecular structure which formally may be derived from the structure of a naturally occurring insulin, for example that of human insulin, by deleting and/or substituting at least one amino acid residue occurring in the natural insulin and/or by adding at least one amino acid residue.

In one embodiment an insulin analogue according to the invention comprises less than 8 modifications (substitutions, deletions, additions) relative to human insulin. In one embodiment an insulin analogue comprises less than 7 modifications (substitutions, deletions, additions) relative to human insulin. In one embodiment an insulin analogue comprises less than 6 modifications (substitutions, deletions, additions) relative to human insulin. In one embodiment an insulin analogue comprises less than 5 modifications (substitutions, deletions, additions) relative to human insulin. In one embodiment an insulin analogue comprises less than 4 modifications (substitutions, deletions, additions) relative to human insulin. In one embodiment an insulin analogue comprises less than 3 modifications (substitutions, deletions, additions) relative to human insulin. In one embodiment an insulin analogue comprises less than 2 modifications (substitutions, deletions, additions) relative to human insulin.

Modifications in the insulin molecule are denoted stating the chain (A or B), the position, and the one or three letter code for the amino acid residue substituting the native amino acid residue.

“Derivative of insulin”, “acylated insulin” or “insulin derivative” are used herein as synonyms and is according to the invention naturally occurring human insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin. Non-limiting examples of such side chains may be found in the form of attachment of amides, carbohydrates, alkyl groups, acyl groups, esters, PEGylations, and the like.

A derivative of insulin is thus human insulin or an insulin analogue which comprises at least one covalent modification such as a side chain attached to one or more amino acids of the insulin peptide.

In one embodiment an insulin derivative according to the invention is an insulin analogue comprising at least two cysteine substitutions, wherein the insulin analogue is acylated in one or more amino acids of the insulin peptide.

Thus, the term “acylated insulin” covers modification of human insulin or an insulin analogue by attachment of one or more side chains via a linker to the insulin and term “acylated insulin” as used herein is thus included in “insulin derivatives”.

The term “linker” is herein used for a portion in between the side chain and the point of attachment to the insulin peptide, which portion may also be referred to as “linker moiety”, “spacer”, or the like. The linker may be optional. In one embodiment, the linker comprises a neutral linear or cyclic amino acid residue, an acidic amino acid residue and/or a neutral, alkyleneglycol-containing amino acid residue, where the order by which these residues appear may be interchanged independently. The connections between the residues, the side chain and the insulin peptide are amide (peptide) bonds.

The term “parent insulin” as used herein is intended to mean an insulin optionally with one or more additional disulfide bonds relative to i.e. human insulin, desB30 human insulin or an insulin analogue with one or more additional disulfide bonds, before being acylated with a side chain.

The term “protease” or a “protease enzyme” as used herein refers to enzymes is a digestive enzyme which degrades proteins and peptides and which is found in various tissues of the human body such as e.g. the stomach (pepsin), the intestinal lumen (chymotrypsin, trypsin, elastase, carboxypeptidases, etc.) or mucosal surfaces of the GI tract (aminopeptidases, carboxypeptidases, enteropeptidases, dipeptidyl peptidases, endopeptidases, etc.), the liver (Insulin degrading enzyme, cathepsin D etc.), and in other tissues.

By “increased solubility at a given pH” is meant that a larger concentration of an acylated insulin dissolves in an aqueous or buffer solution at the pH of the solution relative to the parent insulin. Methods for determining whether the insulin contained in a solution is dissolved are known in the art.

When used herein the term “additional disulfide bonds” or “additional disulfide bridge” are used as synonyms and mean one or more disulfide bonds which are not present in human insulin or insulin analogues comprising the same disulfide bonds (also known as bridges) as human insulin.

The term “acylated insulin without one or more additional disulfide bonds” as used herein is intended to mean an acylated insulin having the three disulfide bonds naturally present in human insulin, i.e. a first bridge between the cysteine in position 7 of the A-chain and the cysteine in position 7 of the B-chain, a second bridge between the cysteine in position 20 of the A-chain and the cysteine in position 19 of the B-chain and a third bridge between the cysteines in position 6 and 11 of the A-chain, and a side chain attached to the insulin but no further disulfide bonds/bridges

The term “side chain” is herein intended to mean a fatty acid or diacid (optionally via one or more linkers) coupled to the parent insulin (i.e. insulin peptide before acylation) for use in the invention, such as to the epsilon amino group of a lysine present in the B-chain of the parent insulin. The fatty acid or diacid part of the side chain is conferring affinity to serum albumin, and the linkers act either to modify (e.g. increase) the affinity for albumin, modify solubility of the acylated insulin, and/or modulate (increase/decrease) the affinity of the acylated insulin for the insulin receptor.

With the term “cysteine substitution” is herein meant replacing an amino acid which is present in human insulin with a cysteine. For example, isoleucine in position 10 in the A chain (IleA10) and glutamine in position 4 of the B chain of human insulin (GlnB4) may each be replaced by a cysteine residue. With the term “other amino acid residue substitution” is herein meant replacing an amino acid which is present in human insulin with an amino acid which is not cysteine. A “lipophilic substituent” or “lipophilic residue” is herein understood as a side chain consisting of a fatty acid or a fatty diacid attached to the insulin, optionally via a linker, in an amino acid position such as LysB29, or equivalent. With the term “oral bioavailability” is herein meant the fraction of the administered dose of drug that reaches the systemic circulation after having been administered orally. By definition, when a medication is administered intravenously, its bioavailability is 100%.

Generally, the term bioavailability refers to the fraction of an administered dose of the active pharmaceutical ingredient (API, i.e. the protease stabilised insulin), such as a derivative of the invention that reaches the systemic circulation unchanged. By definition, when an API is administered intravenously, its bioavailability is 100%. However, when it is administered via other routes (such as orally), its bioavailability decreases (due to incomplete absorption and first-pass metabolism). Knowledge about bioavailability is essential when calculating dosages for non-intravenous routes of administration.

Absolute oral bioavailability compares the bioavailability (estimated as the area under the curve, or AUC) of the API in systemic circulation following oral administration, with the bioavailability of the same API following intravenous administration. It is the fraction of the API absorbed through non-intravenous administration compared with the corresponding intravenous administration of the same API. The comparison must be dose normalised if different doses are used; consequently, each AUC is corrected by dividing the corresponding dose administered.

A plasma API concentration vs. time plot is made after both oral and intravenous administration. The absolute bioavailability (F) is the dose-corrected AUC-oral divided by AUC-intravenous.

Standard assays for measuring insulin bioavailability are known to the person skilled in the art and include inter alia measurement of the relative areas under the curve (AUC) for the concentration of the insulin in question administered orally and intra venously (i.v.) in the same species. Quantitation of acylated insulin concentrations in blood (plasma) samples may be done using for example antibody assays (ELISA) or by mass spectrometry.

However, when a drug is administered orally the bioavailability of the active ingredient decreases due to incomplete absorption and first-pass metabolism. The biological activity of an insulin peptide may be measured in an assay as known by a person skilled in the art as e.g. described in WO 2005012347. The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol. The term “polypeptide” and “peptide” as used herein means a compound composed of at least two constituent amino acids connected by peptide bonds. The constituent amino acids may be from the group of the amino acids encoded by the genetic code and they may be natural amino acids which are not encoded by the genetic code, as well as synthetic amino acids. Commonly known natural amino acids which are not encoded by the genetic code are e.g., γ-carboxyglutamate, ornithine, phosphoserine, D-alanine and D-glutamine. Commonly known synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid, anthranilic acid.

The term “Protein” as used herein means a biochemical compound consisting of one or more polypeptides.

The term “drug”, “therapeutic”, “medicament” or “medicine” when used herein refer to an active ingredient such as e.g. an acylated insulin used in a pharmaceutical composition.

The term “enteric coating” as used herein means a polymer coating that controls disintegration and release of the solid oral dosage form. The site of disintegration and release of the solid dosage form may be customized depending on the enteric coating ability to resist disintegration in a specific pH range.

The term “PK/PD profile” as used herein means pharmacokinetic/pharmacodynamic profile and is known to the person skilled in the art. The pharmacokinetic (PK) profile of an acylated insulin of a pharmecutical composition of the present invention may suitably be determined by in vivo PK studies. These studies are performed in order to evaluate how the acylated insulin is absorbed, distributed and eliminated from the body and how these processes affected the plasma concentration-time profile of the acylated insulin. In discovery and preclinical phase of drug development numerous methods and animal models may be utilized to understand the PK properties for the acylated insulin. For example, the beagle dog may be used to evaluate the PK properties of an acylated insulin in a pharmaceutical composition of the invention following oral administration.

Standard assays for measuring insulin pharmacokinetics are known to the person skilled in the art and include inter alia measurement of the concentration of the insulin in question administered orally and intra venously (i.v.) in the same species. Quantitation of acylated insulin concentrations in blood (plasma) samples may be done using for example antibody assays (ELISA) or by mass spectrometry.

Similarly, the pharmacodynamic (PD) profile of an acylated insulin of a pharmaceutical composition of the present invention may suitably be determined by the study of the biochemical and physiological effects of said acylated insulin on the body and the mechanisms of drug action and the relationship between drug concentration and effect.

The term “Tmax” as used herein means the time after administration of a drug when the maximum plasma concentration is reached (i.e. Cmax).

The term “Cmax” as used herein means the peak plasma concentration of a drug, i.e. insulin.

The term “empty stomach” as used herein means that the Beagle dog has no food contents in its stomach that may interfere with the absorption or disintegration/dissolution of a pharmaceutical composition according to the present invention.

Herein, the term “fatty acid” covers a linear or branched, aliphatic carboxylic acids having at least two carbon atoms and being saturated or unsaturated. The term “fatty acid” as used herein does also include the term “fatty diacid” as defined below. Non limiting examples of fatty acids are myristic acid, palmitic acid, and stearic acid.

Herein, the term “fatty diacid” covers a linear or branched, aliphatic dicarboxylic acids having at least two carbon atoms and being saturated or unsaturated. Non limiting examples of fatty diacids are hexanedioic acid, octanedioic acid, decanedioic acid, dodecanedioic acid, tetradecanedioic acid, hexadecanedioic acid, heptadecanedioic acid, octadecanedioic acid, and eicosanedioic acid.

The term “medium-chain fatty acid” is herein used to mean a fatty acid having a medium length carbon chain such as e.g. carbon chains with between 6 to 12 carbon atoms. Non limiting examples of medium-chain fatty acids include hexanoic acid, octanoic acid, decanoic acid and dodecanoic acid.

Herein, the term “dispersion” means a dispersion, an emulsion or a system consisting of two non-miscible components.

Herein, the term “dissolution” means the process of dissolving a solid substance into a solvent to make a solution.

In one embodiment a polyvinyl alcohol coating material is dispersed or dissolved in aqueous medium, such as but not limited to water, resulting in “polyvinyl alcohol dispersion”. The term “polyvinyl alcohol dispersion” as used herein includes solutions and dispersion, i.e. situations where a polyvinyl alcohol coating is partly or completely dissolved in said aqueous medium. In one embodiment a dispersion of water and said polyvinyl alcohol coating material is placed in a beaker on a suitable stirring apparatus.

The term “disintegration”, “disintegrating”, “disintegrate” or “disintegrated” as used herein and when referring to a coating, is to be understood as said coating being disintegrated into components, wherein some or all of the components are completely dissolved into the medium triggering said disintegration.

Herein, the term “protease stabilised insulin” means the insulin having an improved stability against degradation from proteases relative to human insulin. A protease stabilised insulin may e.g. be stabilised by substitution(s), Addition(s) and/or deletion(s) relative to human insulin. Non-limiting examples of protease stabilised insulins may e.g. be found in WO 08/034881 and WO 09/115469.

Herein the term “immediate release coating” is used as the term is known to the person skilled in the art. Thus this term discloses coatings that are released immediately when contacted with any solution, being pH independent, including prime coating systems.

The term “about” as used herein means in reasonable vicinity of the stated numerical value, such as plus or minus 10%. The terms “mainly” and “majority” as used herein is a quantification to indicate; a part, area, size and frequency that is greater than 50% including about 60%, 70%, 80%, 90% or more relative to the context that it refers to.

The term “stability” is herein used for a pharmaceutical composition comprising modified insulin to describe the shelf life of the composition.

The term “stabilised” or “stable” when referring to an acylated insulin thus refers to a pharmaceutical composition with increased chemical stability or increased physical and chemical stability relative to a pharmaceutical composition comprising a non-stabilised insulin.

The term “chemical stability” of an insulin as used herein refers to chemical covalent changes in the protein structure leading to formation of chemical degradation products with potential less biological potency and/or potential increased immunogenic properties compared to the native protein structure. Various chemical degradation products may be formed depending on the type and nature of the native protein and the environment to which the protein is exposed. Elimination of chemical degradation may most probably not be completely avoided and increasing amounts of chemical degradation products is often seen during storage and use of the pharmaceutical composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation can be mentioned as another variant of chemical degradation. The chemical stability of the acylated insulin may be evaluated by measuring the amount of the chemical degradation products at various time-points after exposure to different environmental conditions (the formation of degradation products may often be accelerated by for instance increasing temperature). The amount of each individual degradation product is often determined by separation of the degradation products depending on molecule size, hydrophilicity, hydrophobicity, and/or charge using various chromatography techniques (e.g. SEC-HPLC and/or RP-HPLC).

Hence, as outlined above, “stabilised” or “stable” when referring to a pharmaceutical composition refers to a pharmaceutical composition comprising an insulin with increased chemical stability or increased physical and chemical stability relative to the corresponding non-modified parent protein. In general, a pharmaceutical composition must be stable during use and storage (in compliance with recommended use and storage conditions) until the expiration date is reached.

The term “direct contact” as used herein refers to the contact between a polyvinyl alcohol coating of the present invention and the tablet core of the present invention. As used herein “direct contact” means that there is no physical barrier between the interface of outer surface of the tablet core and an inner surface of a polyvinyl alcohol coating. Thus when the tablet core according to the present invention is “partly in direct contact” with a polyvinyl alcohol coating according to the present invention, then at least some areas in the interface between the tablet core and a polyvinyl alcohol have are free of physical barriers in contrast to other areas of varying size which may comprise any kind of physical barrier. When “majority” as used herein is used in the context of “a polyvinyl alcohol coating is at least partly in direct contact the majority of an outer surface of the tablet core” it is meant to indicate that the sum of area of direct contact between an outer surface of the tablet core and an inner surface of a polyvinyl alcohol coating is greater than the sum of area where a physical barrier exists in the interface between these two surfaces. The term “physical barrier” as used herein covers any kind of physical barrier which diminishes or influences the physical contact between an outer surface of the tablet core and an inner surface of a polyvinyl alcohol coating.

When used in formulations “mucoadhesive” properties may be introduced to a formulation by use of various polymeric compounds. Typically poly-anions e.g. poly-acrylic acids exert this property. The mucoadhesive property is inherently dependent on the interpenetration of the polymeric compounds both in the bio-mucosa and the formulation. In this way a physical bridge is made possible due to the large size of the polymer molecules. Low molecular weight compounds e.g. sodium caprate or sorbitol will therefore, not exert mucoadhesive properties. Molecules considered “non-mucoadhesive” are molecules with a molecular weight of below 1000 g/mol. We hereby include that molecules with a molecular weight below 900 g/mol, 800 g/mol, 700 g/mol, 600 g/mol, 500 g/mol, 400 g/mol and 300 g/mol are included in this definition of molecules considered non-mucoadhesive in this patent application.

The term “uncoated tablet core” as used herein refers to tablet core which has not been coated with any coating, (e.g. a polyvinyl alcohol coating). The term “coated tablet core” as used herein refers to tablet core which has been coated with a polyvinyl alcohol coating and thus includes tablet cores which consist of a tablet core and a polyvinyl alcohol coating. Thus the term “tablet core” comprise both “coated tablet core” and “uncoated tablet core” cover tablet cores which can be either coated or uncoated, unless otherwise specified. The term “tablet” when used without further specification, is the finished product that will be administered, thus is may be a “uncoated tablet core” or a “coated tablet core” if administered as such. Further one or more uncoated or coated “tablets” can be administered at the same time (i.e. simultaneously) by either being provided as one or more tablets swallowed at the same time, or by being provided in for example in a capsule as described in some of the examples herein or more than one uncoated or coated tablet cores can be compressed into one “tablet”, which can have any size or weight, such as midi tablet or monolith tablet size and will then be relatively faster disintegrating compared to the same size/weight of a regular tablet comprising of only one tablet core of the equal size/weight as the sum of said one or more tablet cores.

which The term “polyvinyl alcohol coating” as used herein refers to a coating or film coating which comprises one or more types of polyvinyl alcohol polymers. The term “polyvinyl alcohol coating” as used herein includes coating comprising about 25-55% (w/w), preferably 38-46% (w/w) polyvinyl alcohol polymer, the term also includes what the skilled person in the art appreciates as a “polyvinyl alcohol film”. Thus the terms “polyvinyl alcohol coating” and “polyvinyl alcohol film” are treated as synonyms in this application. The term “coating based on polyvinyl alcohol polymer” as used herein refers to a coating which comprises polyvinyl alcohol copolymers, i.e. comprises more than 20% (w/w) or more polyvinyl alcohol and thus is covered by the term “polyvinyl alcohol coating”.

The term “polyvinyl alcohol coating material” as used herein refers to the material which is purchased or produced, often a dry powder and comprises all components of a polyvinyl alcohol coating. Examples of polyvinyl alcohol coatings are given in WO0104195 A1.

One example of a commercially available polyvinyl alcohol coating is Opadry® II Yellow from Colorcon®, 85F32410 (as sold in 2013).

In one embodiment a polyvinyl alcohol coating material is dispersed in aqueous medium to form “polyvinyl alcohol dispersion” for coating to be coated on top of a tablet or tablet core, where the copolymer material may form a polyvinyl alcohol coating or film.

The term “anionic copolymer coating” as used herein refers to a coating or film coating which comprises about 80% (w/w) or more anionic copolymer. In one embodiment the term “anionic copolymer coating” includes coatings such as Eudragit®FS30D from Evonik Industries (as sold in 2013) and Acryl-EZE® 930 from Colorcon® (as sold in 2013) coatings. The term “anionic copolymer coating” as used herein includes coating comprising about 80%, 90% or 100% anionic copolymer. The term “coating based on anionic copolymer” as used herein refers to a coating which primarily comprises anionic copolymer, i.e. comprises about 80% (w/w) or more anionic copolymer and thus is covered by the term “anionic copolymer coating”.

The term “copolymer coating material” as used herein refers to the material which is purchased or produced, often a dry powder and comprises all components of the copolymer coating. This copolymer coating material is suspended for coating on top of a tablet or tablet core, where the copolymer material may form the copolymer coating.

The term “functional” when referring to a coating is intended to indicate that said coating dissolves in aqueous medium at specific pH intervals of said medium and/or time windows.

According to the above, the term “non-functional” when referring to a coating is intended to indicate that said coating dissolves in aqueous medium regardless of the pH values of said medium. Functionality does herein not relate to changing of physical properties for the composition such as e.g. moisture barrier.

The term “additional separating layer” as used herein refers to any non-functional coating, such as another type of PVA coating or any other coating which is known by the skilled person as a non-functional coating and may also qualify as a sub coat for enteric coatings. A specific example of such a standard separating layer is OPADRY®II—Yellow from Colorcon® (as sold in 2013), which the skilled person in the art appreciates to be a commonly (i.e. standard) used sub coat for enteric coatings in oral formulations.

The term “additional non-functional coating” as used herein refers to any non-functional coating, such as another type of PVA coating or any other coating which is known by the skilled person as a non-functional coating and may also qualify as a sub coat for enteric coatings. A specific example of such a non-functional coating is the polyvinyl alcohol coating OPADRY®II—Yellow from Colorcon® (as sold in 2013), which the skilled person in the art appreciates to be a commonly (i.e. standard) used sub coat for enteric coatings in oral formulations.

The term “insulin powder” as used herein refers to the active pharmaceutical ingredient (API), which has been dried and is stored in the form of a powder, in this case the API is acylated insulin, and therefore the powder is a “insulin powder”.

The term “sorbitol powder” as used herein refers to any sorbitol or equivalent excipient, such as mannitol, which is dried and stored in the form of a powder.

The Following is a Non-Limiting List of Aspects Further Comprised within the Scope of the Invention:
1. A pharmaceutical composition comprising one or more tablet core, wherein each tablet core comprises a salt of a medium-chain fatty acid and one or more acylated insulin and optionally comprises a polyvinyl alcohol coating and wherein said acylated insulin comprises one or more an additional disulfide bridges or, wherein said acylated insulin is a protease stabilised acylated insulin comprising a linker and a fatty acid or fatty diacid side chain having 14-22 carbon atoms and optionally further comprising one or more an additional disulfide bonds.
1A. A pharmaceutical composition comprising one or more tablet core wherein each tablet core comprises a salt of a medium-chain fatty acid and an insulin derivative and optionally comprises a polyvinyl alcohol coating, and wherein said insulin derivative comprises one or more an additional disulfide bridges or, wherein said insulin derivative is an acylated insulin comprising a linker and a fatty acid or fatty diacid side chain having 14-22 carbon atoms and optionally further comprising one or more an additional disulfide bonds.
2. The pharmaceutical composition according to aspect 1, wherein said one or more acylated insulin means two different acylated insulin compounds, i.e. acylated insulin A and acylated insulin B.
3. The pharmaceutical composition according to aspect 1A, wherein said one or more insulin derivative means two different insulin derivatives, i.e. acylated insulin A and acylated insulin B.
4. The pharmaceutical composition according to any one of the preceding aspects, wherein said optional polyvinyl alcohol coating dissolves in aqueous medium at any pH.
5. The pharmaceutical composition according any one of the preceding aspects, wherein optional said polyvinyl alcohol coating comprises about 25-55% polyvinyl alcohol.
6. The pharmaceutical composition according to any one of the preceding aspects, wherein said optional polyvinyl alcohol coating comprises about 38-46% polyvinyl alcohol.
7. The pharmaceutical composition according to any one of the preceding aspects wherein said optional polyvinyl alcohol coating is an OPADRY® II coating (from Colorcon® as sold in 2013).
8. The pharmaceutical composition according to any one of the preceding aspects, wherein said optional polyvinyl alcohol is selected from immediate release coatings comprising polyvinyl alcohol coatings, such as OPADRY®II-clear or OPADRY®II—pigmented, wherein said OPADRY®II—pigmented can be OPADRY®II—Yellow, (OPADRY®II—clear, OPADRY®II—pigmented and OPADRY®II—Yellow from Colorcon® as sold in 2013).
9. The pharmaceutical composition according to any one of the preceding aspects, wherein said medium-chain fatty acid is capric acid.
10. The pharmaceutical composition according to any one of the preceding aspects, wherein said salt of a medium-chain fatty acid is a sodium caprate, i.e. sodium salt of capric acid.
11. The pharmaceutical composition according to any of the preceding aspects, wherein said one or more tablet core further comprises, sorbitol, stearic acid and insulin.
12. The pharmaceutical composition according to any of the preceding aspects, wherein said one or more tablet core further comprises other pharmaceutically acceptable excipients.
13. The pharmaceutical composition according to any of the preceding aspects, wherein said one or more tablet core comprises ingredients with a molecular weight below about 300-1000 g/mol.
14. The pharmaceutical composition according to any of the preceding aspects, wherein said one or more tablet core comprises ingredients with a molecular weight below about 1000 g/mol.
15. The pharmaceutical composition according to any of the preceding aspects, wherein said one or more tablet core comprises ingredients with a molecular weight below about 800 g/mol.
16. The pharmaceutical composition according to any of the preceding aspects, wherein said one or more tablet core comprises ingredients with a molecular weight below about 700 g/mol.
17. The pharmaceutical composition according to any of the preceding aspects, wherein said one or more tablet core comprises ingredients with a molecular weight below about 600 g/mol.
18. The pharmaceutical composition according to any of the preceding aspects, wherein said one or more tablet core comprises ingredients with a molecular weight below about 500 g/mol.
19. The pharmaceutical composition according to any of the preceding aspects, wherein said one or more tablet core comprises ingredients with a molecular weight below about 400 g/mol.
   The pharmaceutical composition according to any of the preceding aspects, wherein said one or more tablet core comprises ingredients with a molecular weight below about 300 g/mol.
20. The pharmaceutical composition according to any of the preceding aspects, wherein said one or more tablet core comprises ingredients with a molecular weight above about 300-1000 g/mol.
21. The pharmaceutical composition according to any of the preceding aspects, wherein said one or more tablet core comprises ingredients with a molecular weight above about 1000 g/mol.
22. The pharmaceutical composition according to any of the preceding aspects, wherein said one or more tablet core comprises ingredients with a molecular weight above about 800 g/mol.
23. The pharmaceutical composition according to any of the preceding aspects, wherein said one or more tablet core comprises ingredients with a molecular weight above about 700 g/mol.
24. The pharmaceutical composition according to any of the preceding aspects, wherein said one or more tablet core comprises ingredients with a molecular weight above about 600 g/mol.
25. The pharmaceutical composition according to any of the preceding aspects, wherein said one or more tablet core comprises ingredients with a molecular weight above about 500 g/mol.
26. The pharmaceutical composition according to any of the preceding aspects, wherein said one or more tablet core comprises ingredients with a molecular weight above 400 g/mol.
27. The pharmaceutical composition according to any of the preceding aspects, wherein said one or more tablet core comprises ingredients with a molecular weight above about 300 g/mol.
28. The pharmaceutical composition according to any of the preceding aspects, wherein said one or more tablet core is not mucoadhesive.
29. The pharmaceutical composition according to any of the preceding aspects, wherein said one or more tablet core is not mucoadhesive and/or does not comprise mucoadhesive ingredients.
30. The pharmaceutical composition according to any of the preceding aspects, wherein said one or more tablet core is not mucoadhesive however comprise mucoadhesive ingredients.
The pharmaceutical composition according to any of the preceding aspects, wherein said one or more tablet core comprises ingredients and excipients with zero water uptake.
31. The pharmaceutical composition according to any of the preceding aspects, wherein said one or more tablet core comprises ingredients and excipients exerting a total water uptake of about 0-9%.
32. The pharmaceutical composition according to any of the preceding aspects, wherein said one or more tablet core comprises ingredients and excipients exerting a total water uptake of about 0-9%.
33. The pharmaceutical composition according to any of the preceding aspects, wherein said one or more tablet core comprises ingredients and excipients exerting a total water uptake of below about 10%.
34. The pharmaceutical composition according to any of the preceding aspects, wherein said one or more tablet core comprises ingredients and excipients exerting a total water uptake of about 9%.
35. The pharmaceutical composition according to any of the preceding aspects, wherein said one or more tablet core comprises ingredients and excipients exerting a total water uptake of below about 8%.
36. The pharmaceutical composition according to any of the preceding aspects, wherein said one or more tablet core comprises ingredients and excipients exerting a total water uptake of below about 10% or more.
37. The pharmaceutical composition according to any one of the preceding aspects wherein said one or more tablet core comprises about 50-85% (w/w) sodium caprate.
38. The pharmaceutical composition according to any one of the preceding aspects wherein said one or more tablet core comprises about 70-85% (w/w) sodium caprate.
39. The pharmaceutical composition according to any one of the preceding aspects wherein said one or more tablet core comprises about 70-80% (w/w) sodium caprate.
40. The pharmaceutical composition according to any one of the preceding aspects wherein said one or more tablet core comprises about 75% (w/w) sodium caprate.
41. The pharmaceutical composition according to any one of the preceding aspects wherein said one or more tablet core comprises about 75-80% (w/w) sodium caprate.
42. The pharmaceutical composition according to any one of the preceding aspects wherein said one or more tablet core comprises about 77% (w/w) sodium caprate.
43. The pharmaceutical composition according to any one of the preceding aspects wherein said one or more tablet core comprises about 80% (w/w) sodium caprate.
44. The pharmaceutical composition according to any one of the preceding aspects wherein said one or more tablet core comprises about 85% (w/w) sodium caprate.
45. The pharmaceutical composition according to any one of the preceding aspects wherein said one or more tablet core comprises about 77% (w/w) sodium caprate, about 22.5-X % (w/w) sorbitol, about X % (w/w) insulin and about 0.5% (w/w) stearic acid, wherein X is selected from the group consisting of: 0.1, 0.5, 1, 1.5, 2, 2.5, 3, 3.5, 4, 4.5 or 5.
46. The pharmaceutical composition according to any one of the preceding aspects wherein said one or more tablet core comprises about 77% (w/w) sodium caprate, about 22.5-X % (w/w) sorbitol, about X % (w/w) insulin and about 0.5% (w/w) stearic acid, wherein X is selected from the group consisting of: 5.5, 6, 6.5, 7, 7.5, 8, 8.5, 9, 9.5 or 10.
47. The pharmaceutical composition according to any one of the preceding aspects wherein said one or more tablet core comprises about 77% (w/w) sodium caprate, about 22.5-X % (w/w) sorbitol, about X % (w/w) insulin and about 0.5% (w/w) stearic acid, wherein X is selected from the group consisting of: 10.5, 11, 11.5, 12, 12.5, 13, 13.5, 14, 14.5 or 15.
48. The pharmaceutical composition according to any one of the preceding aspects wherein said one or more tablet core comprises about 77% (w/w) sodium caprate, about 22.5-X % (w/w) sorbitol, about X % (w/w) insulin and about 0.5% (w/w) stearic acid, wherein X is selected from the group consisting of: 15.5, 16, 16.5, 17, 17.5, 18, 18.5, 19, 19.5, 20, 20.5, 21 or 21.5.
49. The pharmaceutical composition according to any one of the preceding aspects wherein each of said one or more tablet core is below 50 mg and comprises about 75-% (w/w) sodium caprate, about 22.5-X % (w/w) sorbitol, about X % (w/w) insulin and about 0.5% (w/w) stearic acid, wherein X is selected from the group consisting of: 0.1, 0.5, 1, 1.5, 2, 2.5, 3, 3.5, 4, 4.5 or 5.
50. The pharmaceutical composition according to any one of the preceding aspects wherein each of said one or more tablet core is below 50 mg and comprises about 77% (w/w) sodium caprate, about 22.5-X % (w/w) sorbitol, about X % (w/w) insulin and about 0.5% (w/w) stearic acid, wherein X is selected from the group consisting of: 5.5, 6, 6.5, 7, 7.5, 8, 8.5, 9, 9.5 or 10.
51. The pharmaceutical composition according to any one of the preceding aspects wherein each of said one or more tablet core is below 50 mg and comprises about 77% (w/w) sodium caprate, about 22.5-X % (w/w) sorbitol, about X % (w/w) insulin and about 0.5% (w/w) stearic acid, wherein X is selected from the group consisting of: 10.5, 11, 11.5, 12, 12.5, 13, 13.5, 14, 14.5 or 15.
52. The pharmaceutical composition according to any one of the preceding aspects wherein said one or more tablet core comprises about 77% (w/w) sodium caprate, about 20.5-X % (w/w) sorbitol, about X % (w/w) insulin and about 0.5% (w/w) stearic acid, wherein X is selected from the group consisting of: 0.1, 0.5, 1, 1.5, 2, 2.5, 3, 3.5, 4, 4.5 or 5.
53. The pharmaceutical composition according to any one of the preceding aspects wherein said one or more tablet core comprises about 77% (w/w) sodium caprate, about 20.5-X % (w/w) sorbitol, about X % (w/w) insulin and about 0.5% (w/w) stearic acid, wherein X is selected from the group consisting of: 5.5, 6, 6.5, 7, 7.5, 8, 8.5, 9, 9.5 or 10.
54. The pharmaceutical composition according to any one of the preceding aspects wherein said one or more tablet core comprises about 77% (w/w) sodium caprate, about 22.0-X % (w/w) sorbitol, about X % (w/w) insulin and about 0.5% (w/w) stearic acid, wherein X is selected from the group consisting of: 10.5, 11, 11.5, 12, 12.5, 13, 13.5, 14, 14.5 or 15.
55. The pharmaceutical composition according to any one of the preceding aspects wherein said one or more tablet core comprises about 77% (w/w) sodium caprate, about 20.5-X % (w/w) sorbitol, about X % (w/w) insulin and about 0.5% (w/w) stearic acid, wherein X is selected from the group consisting of: 15.5, 16, 16.5, 17, 17.5, 18, 18.5, 19, 19.5, 20, 20.5, 21 or 21.5.
56. The pharmaceutical composition according to any one of the preceding aspects wherein each of said one or more tablet core is below 50 mg and comprises about 75-% (w/w) sodium caprate, about 20.5-X % (w/w) sorbitol, about X % (w/w) insulin and about 0.5% (w/w) stearic acid, wherein X is selected from the group consisting of: 0.1, 0.5, 1, 1.5, 2, 2.5, 3, 3.5, 4, 4.5 or 5.
57. The pharmaceutical composition according to any one of the preceding aspects wherein each of said one or more tablet core is below 50 mg and comprises about 77% (w/w) sodium caprate, about 20.5-X % (w/w) sorbitol, about X % (w/w) insulin and about 0.5% (w/w) stearic acid, wherein X is selected from the group consisting of: 5.5, 6, 6.5, 7, 7.5, 8, 8.5, 9, 9.5 or 10
58. The pharmaceutical composition according to any one of the preceding aspects wherein each of said one or more tablet core is below 50 mg and comprises about 77% (w/w) sodium caprate, about 22.5-X % (w/w) sorbitol, about X % (w/w) insulin and about 0.5% (w/w) stearic acid, wherein X is selected from the group consisting of: 10.5, 11, 11.5, 12, 12.5, 13, 13.5, 14, 14.5 or 15.
59. The pharmaceutical composition according to any one of the preceding aspects wherein each of said one or more tablet core is below 50 mg and comprises about 77% (w/w) sodium caprate, about 20.5-X % (w/w) sorbitol, about X % (w/w) insulin and about 0.5% (w/w) stearic acid, wherein X is selected from the group consisting of: 15.5, 16, 16.5, 17, 17.5, 18, 18.5, 19, 19.5, 20, 20.5, 21 or 21.5.
60. The pharmaceutical composition according to any one of the preceding aspects wherein said one or more tablet core are uncoated, i.e. do not comprise a coating.
61. The pharmaceutical composition according to any one of the preceding aspects wherein each of said one or more tablet core is uncoated and weighs about 1.5-800 mg or about 1.5-900 mg.
62. The pharmaceutical composition according to any one of the preceding aspects wherein each of said one or more tablet core is uncoated and weighs about 600-800 mg or about 600-900 mg
63. The pharmaceutical composition according to any one of the preceding aspects wherein each of said one or more tablet core is uncoated and weighs about 250-475 mg.
64. The pharmaceutical composition according to any one of the preceding aspects wherein each of said one or more tablet core is uncoated and weighs about 710 mg.
65. The pharmaceutical composition according to any one of the preceding aspects wherein each of said one or more tablet core is uncoated and weighs about 200-380 mg.
66. The pharmaceutical composition according to any one of the preceding aspects wherein each of said one or more tablet core is uncoated and weighs about 335 mg.
67. The pharmaceutical composition according to any one of the preceding aspects wherein each of said one or more tablet core is uncoated and weighs about 237 mg.
68. The pharmaceutical composition according to any one of the preceding aspects wherein each of said one or more tablet core is uncoated and weighs about 1.5-50 mg.
69. The pharmaceutical composition according to any one of the preceding aspects wherein each of said one or more tablet core is uncoated and weighs about 3.0-50 mg.
70. The pharmaceutical composition according to any one of the preceding aspects wherein each of said one or more tablet core is uncoated and weighs about 3.0-10 mg.
71. The pharmaceutical composition according to any one of the preceding aspects wherein each of said one or more tablet core is uncoated and weighs about 3.0-5.0 mg.
72. The pharmaceutical composition according to any one of the preceding aspects wherein each of said one or more tablet core is uncoated and weighs about 3.6 mg.
73. The pharmaceutical composition according to any one of the preceding aspects wherein each of said one or more tablet core is coated with said optional polyvinyl alcohol coating and weighs about 1.5-50 mg.
74. The pharmaceutical composition according to any one of the preceding aspects wherein each of said one or more tablet core is coated with said optional polyvinyl alcohol coating and weighs about 3.0-50 mg.
75. The pharmaceutical composition according to any one of the preceding aspects wherein each of said one or more tablet core is coated with said optional polyvinyl alcohol coating and weighs about 3.0-10 mg.
76. The pharmaceutical composition according to any one of the preceding aspects wherein each of said one or more tablet core is coated with said optional polyvinyl alcohol coating and weighs about 3.0-5.0 mg.
77. The pharmaceutical composition according to any one of the preceding aspects wherein each of said one or more tablet core is coated with said optional polyvinyl alcohol coating and weighs about 3.6 mg.
78. The pharmaceutical composition according to any one of the preceding aspects 61 and 68-77, wherein said one or more tablet core is formed by using a punch with a diameter of 1.0-5.0 mm, preferably 1.5-4.0 mm, more preferred 1.5 mm or 4.0 mm.
79. The pharmaceutical composition according to any one of the preceding aspects 61 and 68-77, wherein said one or more tablet core is formed by using a punch with a diameter of 1.0-5.0 mm.
80. The pharmaceutical composition according to any one of the preceding aspects 61 and 68-77, wherein said one or more tablet core is formed by using a punch with a diameter of 1.5-4.0 mm.
81. The pharmaceutical composition according to any one of the preceding aspects 61 and 68-77, wherein said one or more tablet core is formed by using a punch with a diameter of 1.5 mm or 4.0 mm.
82. The pharmaceutical composition according to any one of the preceding aspects, wherein each of said one or more tablet core comprises a polyvinyl alcohol coating and weighs about 280-500 mg.
83. The pharmaceutical composition according to any one of the preceding aspects, wherein each of said one or more tablet core comprises a polyvinyl alcohol coating and weighs about 600-900 mg.
84. The pharmaceutical composition according to any one of the preceding aspects, wherein each of said one or more tablet core comprises a polyvinyl alcohol coating and weighs about 745 mg.
85. The pharmaceutical composition according to any one of the preceding aspects, wherein each of said one or more tablet core comprises a polyvinyl alcohol coating and weighs about 742 mg.
86. The pharmaceutical composition according to any one of the preceding aspects, wherein each of said one or more tablet core comprises a polyvinyl alcohol coating and weighs about 373 mg.
87. The pharmaceutical composition according to any one of the preceding aspects, wherein each of said one or more tablet core comprises a polyvinyl alcohol coating and weighs about 258 mg.
88. The pharmaceutical composition according to any one of the preceding aspects, wherein each of said one or more tablet core comprises a polyvinyl alcohol coating and weighs about 240 mg.
89. The pharmaceutical composition according to any one of the preceding aspects about 20-300 tablet cores of this invention each weighing between about 1.5-50 mg are provided in one or more capsules.
90. The pharmaceutical composition according to any one of the preceding aspects comprising about 70-240 tablet cores of this invention each weighing between about 3.0-10 mg are provided in one or more capsules.
91. The pharmaceutical composition according to any one of the preceding aspects comprising about 150-250 tablet cores of this invention each weighing between about 1.5-50 mg are provided in one or more capsules.
92. The pharmaceutical composition according to any one of the preceding aspects comprising about 100-250 tablet cores of this invention each weighing between about 3.0-10 mg are provided in one or more capsules.
93. The pharmaceutical composition according to any one of the preceding aspects comprising about 20-300 tablet cores of this invention each weighing between about 3.0-10 mg are provided in one or more capsules.
94. The pharmaceutical composition according to any one of the preceding aspects comprising about 150-250 tablet cores of this invention each weighing between about 3.0-10 mg are provided in one or more capsules.
95. The pharmaceutical composition according to any one of the preceding aspects comprising about 20-100 tablet cores of this invention each weighing between about 3.0-10 mg are provided in one or more capsules.
96. The pharmaceutical composition according to any one of the preceding aspects comprising about 100-250 tablet cores of this invention each weighing between about 3.0-10 mg are provided in one or more capsules.
97. The pharmaceutical composition according to any one of the preceding aspects comprising about 150-250 tablet cores of this invention each weighing about 3.6 mg are provided in one or more capsules.
98. The pharmaceutical composition according to any one of the preceding aspects comprising about 150-250 tablet cores of this invention each weighing between about 3.0-5.0 mg are provided in one or more capsules.
99. The pharmaceutical composition according to any one of the preceding aspects comprising about 140-240 tablet cores of this invention each weighing between about 3.0-5.0 mg are provided in one or more capsules.
100. The pharmaceutical composition according to any one of the preceding aspects comprising about 150-250 tablet cores of this invention each weighing about 3.6 mg are provided in one or more capsules.
101. The pharmaceutical composition according to any one of the preceding aspects comprising about 200 tablet cores of this invention each weighing about 3.6 mg are provided in one or more capsules.
102. The pharmaceutical composition according to any one of the preceding aspects comprising about 600-1300 mg, preferably 600-900 mg tablet cores of this invention, wherein each tablet core weighs about 3.6 mg are provided in one or more capsules
103. The pharmaceutical composition according to any one of the preceding aspects comprising about 710 mg tablet cores of this invention, wherein each tablet core weighs between about 3.0-5.0 mg are provided in one or more capsules
104. The pharmaceutical composition according to any one of the preceding aspects comprising about 710 mg tablet cores of this invention, wherein each tablet core weighs about 3.6 mg are provided in one or more capsules
105. The pharmaceutical composition according to any one of the preceding aspects comprising about 588 mg tablet cores of this invention, wherein each tablet core weighs between about 3.0-5.0 mg are provided in one or more capsules
106. The pharmaceutical composition according to any one of the preceding aspects comprising about 710 mg tablet cores of this invention, wherein each tablet core weighs about 3.6 mg are provided in one or more capsules
107. The pharmaceutical composition according to any one of the preceding aspects comprising about 600 mg tablet cores of this invention, wherein each tablet core weighs between about 3.0-5.0 mg are provided in one or more capsules
108. The pharmaceutical composition according to any one of the preceding aspects comprising about 710 mg tablet cores of this invention, wherein each tablet core weighs about 3.6 mg are provided in one or more capsules.
109. The pharmaceutical composition according to any one of the preceding aspects wherein said tablet cores weighing 1.5-50 mg are formed by using punches with a diameter of about 1.0-5.0 mm, preferably about 1.5-4.0 mm, more preferred about 1.5 mm or about 4.0 mm.
110. The pharmaceutical composition according to any one of the preceding aspects comprising one or more tablet core, up to six tablet cores, up to three tablet cores or two tablet cores.
111. The pharmaceutical composition according to any one of the preceding aspects comprising one or more tablet core, up to six tablet cores, up to three tablet cores or two tablet cores.
112. The pharmaceutical composition according to any one of the preceding aspects comprising one or more tablet core weighing between about 200 mg and 900 mg comprising up to six tablet cores, up to three tablet cores or two tablet cores.
113. The pharmaceutical composition according to any one of the preceding aspects comprising one or more tablet core coated with a polyvinyl alcohol coating according to any one of the preceding aspects, comprising up to six tablet cores, up to three tablet cores or two tablet cores.
114. The pharmaceutical composition according to any one of the preceding aspects, wherein said polyvinyl alcohol coating which is in direct contact with an outer surface of said one or more tablet core is in direct contact with at about 100% of said outer surface of said one or more tablet core.
115. The pharmaceutical composition according to any one of the preceding aspects, wherein said polyvinyl alcohol coating which is in direct contact with an outer surface of said one or more tablet core is in direct contact with at about 99% of said outer surface of said one or more tablet core.
116. The pharmaceutical composition according to any one of the preceding aspects, wherein said polyvinyl alcohol coating which is in direct contact with an outer surface of said one or more tablet core is in direct contact with at about 90% of said outer surface of said one or more tablet core.
117. The pharmaceutical composition according to any one of the preceding aspects, wherein said polyvinyl alcohol coating which is in direct contact with an outer surface of said one or more tablet core is in direct contact with at about 85% of said outer surface of said one or more tablet core.
118. The pharmaceutical composition according to any one of the preceding aspects, wherein said polyvinyl alcohol coating which is in direct contact with an outer surface of said one or more tablet core is in direct contact with at about 80% of said outer surface of said one or more tablet core
119. The pharmaceutical composition according to any one of the preceding aspects, wherein said polyvinyl alcohol coating which is in direct contact with an outer surface of said one or more tablet core is in direct contact with at about 70% of said outer surface of said one or more tablet core.
120. The pharmaceutical composition according to any one of the preceding aspects, wherein said polyvinyl alcohol coating which is in direct contact with an outer surface of said one or more tablet core is in direct contact with at about 60% of said outer surface of said one or more tablet core.
121. The pharmaceutical composition according to any one of the preceding aspects, wherein said polyvinyl alcohol coating which is in direct contact with an outer surface of said one or more tablet core is in direct contact with at about 50% of said outer surface of said one or more tablet core.
122. The pharmaceutical composition according to any one of the preceding aspects, wherein said polyvinyl alcohol coating which is in direct contact with an outer surface of said one or more tablet core is in direct contact with at about 40% of said outer surface of said one or more tablet core.
123. The pharmaceutical composition according to any one of the preceding aspects, wherein said polyvinyl alcohol coating which is in direct contact with an outer surface of said one or more tablet core is in direct contact with at about 30% of said outer surface of said one or more tablet core.
124. The pharmaceutical composition according to any one of the preceding aspects, wherein said polyvinyl alcohol coating which is in direct contact with an outer surface of said one or more tablet core is in direct contact with at about 20% of said outer surface of said one or more tablet core.
125. The pharmaceutical composition according to any one of the preceding aspects, wherein said polyvinyl alcohol coating which is in direct contact with an outer surface of said one or more tablet core is in direct contact with at about 10% of said outer surface of said one or more tablet core.
126. The pharmaceutical composition according to any one of the preceding aspects, wherein said polyvinyl alcohol coating which is in direct contact with an outer surface of said one or more tablet core is in direct contact with at about 1% of said outer surface of said one or more tablet core.
127. The pharmaceutical composition according to any one of the preceding aspects, wherein said polyvinyl alcohol coating which is in direct contact with an outer surface of said one or more tablet core is in direct contact with at about 0% of said outer surface of said one or more tablet core.
128. The pharmaceutical composition according to any one of the preceding aspects, wherein said polyvinyl alcohol coating is present in at amount of about 0-10% (w/w) relative to said one or more tablet core.
129. The pharmaceutical composition according to any one of the preceding aspects, wherein said polyvinyl alcohol coating is present in at amount of about 0% (w/w) relative to said one or more tablet core.
130. The pharmaceutical composition according to any one of the preceding aspects, wherein said polyvinyl alcohol coating is present in at amount of about 2% (w/w) relative to said one or more tablet core.
131. The pharmaceutical composition according to any one of the preceding aspects, wherein said polyvinyl alcohol coating is present in at amount of about 4% (w/w) relative to said one or more tablet core.
132. The pharmaceutical composition according to any one of the preceding aspects, wherein said polyvinyl alcohol coating is present in at amount of about 4.5% (w/w) relative to said one or more tablet core.
133. The pharmaceutical composition according to any one of the preceding aspects, wherein said polyvinyl alcohol coating is present in at amount of about 5% (w/w) relative to said one or more tablet core.
134. The pharmaceutical composition according to any one of the preceding aspects, wherein said polyvinyl alcohol coating is present in at amount of about 6% (w/w) relative to said one or more tablet core.
135. The pharmaceutical composition according to any one of the preceding aspects, wherein said polyvinyl alcohol coating is present in at amount of about 8% (w/w) relative to said one or more tablet core.
136. The pharmaceutical composition according to any one of the preceding aspects, wherein said polyvinyl alcohol coating is present in at amount of about 10% (w/w) relative to said one or more tablet core.
137. pharmaceutical composition according to any one of the preceding aspects wherein said polyvinyl alcohol coating leads to a weight gain of about 20-30% (w/w), about 25%-26% (w/w) of the uncoated tablet core.
138. The pharmaceutical composition according to any one of the preceding aspects, wherein an additional non-functional coating is applied on top of said polyvinyl alcohol coating.
139. The pharmaceutical composition according to any one of the preceding aspects, wherein an additional continuous non-functional coating is applied on top of said polyvinyl alcohol coating.
140. The pharmaceutical composition according to any one of the preceding aspects, wherein an additional discontinuous non-functional coating is applied on top of said polyvinyl alcohol coating.
141. The pharmaceutical composition according to any one of the preceding aspects, wherein an additional non-functional coating is applied below said one or more tablet core and said polyvinyl alcohol coating.
142. The pharmaceutical composition according to any one of the preceding aspects, wherein an additional continuous non-functional coating is applied below said one or more tablet core and said polyvinyl alcohol coating.
143. The pharmaceutical composition according to any one of the preceding aspects, wherein an additional discontinuous non-functional coating is applied below said one or more tablet core and said polyvinyl alcohol coating.
144. The pharmaceutical composition according to any one of the preceding aspects, wherein no additional non-functional coating is applied below said one or more tablet core and said polyvinyl alcohol coating.
145. The pharmaceutical composition according to any one of the preceding aspects, wherein no additional continuous non-functional coating is applied between said one or more tablet core and said polyvinyl alcohol coating.
146. The pharmaceutical composition according to any one of the preceding aspects wherein no additional discontinuous non-functional coating is applied between said one or more tablet core and said polyvinyl alcohol coating.
147. The pharmaceutical composition according to any of the preceding aspects, wherein said composition is administered orally.
148. The pharmaceutical composition according to any one of the preceding aspects in the form of a tablet.
149. The pharmaceutical composition according to any one of the preceding aspects in the form of a multiparticulate system.
150. The pharmaceutical composition according to any one of the preceding aspects in the form of a multiparticulate system, wherein a multiparticulate system comprises one or more tablets, up to three tablets or two tablets.
151. The pharmaceutical composition according to any one of the preceding aspects in the form of a multiparticulate system compressed into a tablet, wherein said compressed tablet is fast disintegrating and has the size of a midi tablet or monolith tablet, i.e. weigh between about 50 mg to about 600 mg or about 600 mg to about 900 mg.
152. The pharmaceutical composition according to any one of the preceding aspects in the form of a multiparticulate system compressed into a tablet of wherein each tablet core weighs 1.5-50 mg and the compressed tablet between about 50 mg and about 600 mg.
153. The pharmaceutical composition according to any one of the preceding aspects in the form of a multiparticulate system compressed into a tablet of wherein each tablet core weighs 3.0-5.0 mg and the compressed tablet between about 600 mg and about 900 mg.
154. The pharmaceutical composition according to any one of the preceding aspects in the form of a multiparticulate system compressed into a tablet of wherein each tablet core weighs 3.6 mg and the compressed tablet between about 600 mg and about 900 mg or between about 600 mg and about 1300 mg.
155. The pharmaceutical composition according to any one of the aspects 148-154, wherein said pharmaceutical composition is coated with an polyvinyl alcohol coating as defined in this invention.
156. The pharmaceutical composition according to any one of the aspects 148-154, wherein said pharmaceutical composition is an uncoated tablet comprising one or more tablet core.
157. The pharmaceutical composition according to any one of the preceding aspects in the form of a multiparticulate system comprising one or more polyvinyl alcohol coated or uncoated tablets, wherein each coated or uncoated tablets each comprised one or more coated or uncoated tablet cores.
158. The pharmaceutical composition according to any one of the preceding aspects in the form of a multiparticulate system comprising one or more polyvinyl alcohol coated or uncoated tablets administered at the same time, wherein each coated or uncoated tablets each comprised one or more coated or uncoated tablet cores.
159. The pharmaceutical composition according to any one of the preceding aspects in the form of a multiparticulate system comprising one or more polyvinyl alcohol coated or uncoated tablets administered simultaneously, wherein each coated or uncoated tablets each comprised one or more coated or uncoated tablet cores.
160. The pharmaceutical composition according to any one of the preceding aspects in the form of a multiparticulate system comprising one or more polyvinyl alcohol coated or uncoated tablets administered within 5 minutes relative to each tablets administration, wherein each coated or uncoated tablets each comprised one or more coated or uncoated tablet cores.
161. The pharmaceutical composition according to any one of the preceding aspects in the form of a multiparticulate system comprising up to six polyvinyl alcohol coated or uncoated tablets, wherein each coated or uncoated tablets each comprised one or more coated or uncoated tablet cores.
162. The pharmaceutical composition according to any one of the preceding aspects in the form of a multiparticulate system comprising up to three polyvinyl alcohol coated or uncoated tablets, wherein each coated or uncoated tablets each comprised one or more coated or uncoated tablet cores.
163. The pharmaceutical composition according to any one of the preceding aspects in the form of a multiparticulate system comprising up to three polyvinyl alcohol coated or uncoated tablets administered at the same time, wherein each coated or uncoated tablets each comprised one or more coated or uncoated tablet cores.
164. The pharmaceutical composition according to any one of the preceding aspects in the form of a multiparticulate system comprising up to three polyvinyl alcohol coated or uncoated tablets administered simultaneously, wherein each coated or uncoated tablets each comprised one or more coated or uncoated tablet cores.
165. The pharmaceutical composition according to any one of the preceding aspects in the form of a multiparticulate system comprising up to three polyvinyl alcohol coated or uncoated tablets administered within 5 minutes relative to each tablets administration, wherein each coated or uncoated tablets each comprised one or more coated or uncoated tablet cores.
166. The pharmaceutical composition according to any one of the preceding aspects in the form of a multiparticulate system comprising two polyvinyl alcohol coated or uncoated tablets, wherein each coated or uncoated tablets each comprised one or more coated or uncoated tablet cores.
167. The pharmaceutical composition according to any one of the preceding aspects in the form of a multiparticulate system comprising two polyvinyl alcohol coated or uncoated tablets administered at the same time, wherein each coated or uncoated tablets each comprised one or more coated or uncoated tablet cores.
168. The pharmaceutical composition according to any one of the preceding aspects in the form of a multiparticulate system comprising two polyvinyl alcohol coated or uncoated tablets administered simultaneously, wherein each coated or uncoated tablets each comprised one or more coated or uncoated tablet cores.
169. The pharmaceutical composition according to any one of the preceding aspects in the form of a multiparticulate system comprising two polyvinyl alcohol coated or uncoated tablets administered within 5 minutes relative to each tablets administration, wherein each coated or uncoated tablets each comprised one or more coated or uncoated tablet cores.
170. The pharmaceutical composition according to any one of the preceding aspects in the form of a multiparticulate system, wherein a multiparticulate system comprises one or more coated or uncoated tablets, up to three coated or uncoated tablets or two coated or uncoated tablets, wherein the total weight of the multiparticulate system, i.e. the total weight of said one or more coated or uncoated tablets, the total weight of said up to coated or uncoated three tablets or the total weight of said two coated or uncoated tablets amounts to about 600-1300 mg, preferably 600-900 mg.
171. The pharmaceutical composition according to any one of the preceding aspects in the form of a multiparticulate system, wherein a multiparticulate system comprises one or more coated or uncoated tablets, up to three tablets or coated or uncoated two tablets, wherein the total weight of the multiparticulate system, i.e. the total weight of said one or more coated or uncoated tablets, the total weight of said up to three coated or uncoated tablets or the total weight of said two coated or uncoated tablets amounts to about 600-800 mg.
172. The pharmaceutical composition according to any one of the preceding aspects in the form of a multiparticulate system, wherein a multiparticulate system comprises one or more coated or uncoated tablets, up to three tablets or two coated or uncoated tablets, wherein the total weight of the multiparticulate system, i.e. the total weight of said one or more coated or uncoated tablets, the total weight of said up to three tablets or the total weight of said two coated or uncoated tablets amounts to about 250-475 mg.
173. The pharmaceutical composition according to any one of the preceding aspects in the form of a multiparticulate system, wherein a multiparticulate system comprises one or more coated or uncoated tablets, up to three tablets or two coated or uncoated tablets, wherein the total weight of the multiparticulate system, i.e. the total weight of said one or more coated or uncoated tablets, the total weight of said up to three coated or uncoated tablets or the total weight of said two tablets amounts to about 200-380 mg.
174. The pharmaceutical composition according to any one of the preceding aspects in the form of a multiparticulate system, wherein a multiparticulate system comprises one or more coated or uncoated tablets, up to three tablets or two coated or uncoated tablets, wherein the total weight of the multiparticulate system, i.e. the total weight of said one or more coated or uncoated tablets, the total weight of said up to three coated or uncoated tablets or the total weight of said two coated or uncoated tablets amounts to about 280-500 mg.
175. The pharmaceutical composition according to any one of the preceding aspects in the form of a multiparticulate system, wherein a multiparticulate system comprises up to 500 coated or uncoated tablet cores, wherein the total weight of the multiparticulate system, i.e. the total weight of said up to 500 coated or uncoated tablet cores amounts to about 600-900 mg.
176. The pharmaceutical composition according to any one of the preceding aspects in the form of a multiparticulate system, wherein a multiparticulate system comprises up to 500 coated or uncoated tablet cores, wherein the total weight of the multiparticulate system, i.e. the total weight of said up to 500 coated or uncoated tablet cores amounts to about 600-1300 mg.
177. The pharmaceutical composition according to any one of the preceding aspects in the form of a multiparticulate system, wherein a multiparticulate system comprises up to 500 coated or uncoated tablet cores, wherein the total weight of the multiparticulate system, i.e. the total weight of said up to 300 coated or uncoated tablet cores amounts to about 600-800 mg.
178. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 600-800 mg.
179. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 250-475 mg.
180. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 200-380 mg.
181. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 280-500 mg.
182. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 588 mg.
183. The pharmaceutical composition according to any one of the preceding aspects in the form of a multiparticulate system, wherein a multiparticulate system comprises up to about 300 coated or uncoated tablet cores, wherein the total weight of the multiparticulate system, i.e. the total weight of said up to about 300 coated or uncoated tablet cores amounts to about 600 mg.
184. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 710 mg.
185. The pharmaceutical composition according to any one of the preceding aspects in the form of a multiparticulate system, wherein a multiparticulate system comprises up to 300 coated or uncoated tablet cores, wherein the total weight of the multiparticulate system, i.e. the total weight of said up to 300 coated or uncoated tablet cores amounts to about 895 mg.
186. The pharmaceutical composition according to any one of the preceding aspects in the form a multiparticulate system comprising of one or more polyvinyl alcohol coated or uncoated tablets administered in a capsule.
187. The pharmaceutical composition according to any one of the preceding aspects in the form of a multiparticulate system comprising up to three polyvinyl alcohol coated or uncoated tablets administered in a capsule.
188. The pharmaceutical composition according to any one of the preceding aspects in the form of two polyvinyl alcohol coated or uncoated tablets administered in a capsule.
189. The pharmaceutical composition according to any one of the preceding aspects in the form of one or more polyvinyl alcohol coated or uncoated tablets administered in a capsule.
190. The pharmaceutical composition according to any one of the preceding aspects in the form of a multiparticulate system comprising one or more tablets administered in a capsule
191. The pharmaceutical composition according to any one of the preceding aspects in the form of a multi-particulate system, wherein said particles in said system are uncoated or individually or collectively coated with said polyvinyl alcohol coating.
192. The pharmaceutical composition according to any one of the preceding aspects in the form of a uniform tablet, a single or multi-layered tablet, a multiparticulate system, a capsule, a tablet contained in a capsule, a multiparticulate system comprising multiple tablets contained in a capsule, a multiparticulate system comprising multiple tablets compressed into a tablet, a multiparticulate system in the form of up to three tablets contained in a capsule, a multiparticulate system in the form of up to two tablets contained in a capsule said one or more tablet core.
193. The pharmaceutical composition according to any one of the preceding aspects, wherein said acylated insulin is a protease stabilised insulin comprising a linker and a fatty acid or fatty diacid chain having 14 carbon atoms.
194. The pharmaceutical composition according to any one of the preceding aspects, wherein said acylated insulin is a protease stabilised insulin comprising a linker and a fatty acid or fatty diacid chain having 16 carbon atoms.
195. The pharmaceutical composition according to any one of the preceding aspects, wherein said acylated insulin is a protease stabilised insulin comprising a linker and a fatty acid or fatty diacid chain having 18 carbon atoms.
196. The pharmaceutical composition according to any one of the preceding aspects, wherein said acylated insulin is a protease stabilised insulin comprising a linker and a fatty acid or fatty diacid chain having 20 carbon atoms.
197. The pharmaceutical composition according to any one of the preceding aspects, wherein said acylated insulin is a protease stabilised insulin comprising a linker and a fatty acid or fatty diacid chain having 22 carbon atoms.
198. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin.
199. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin, wherein said chain comprises a linker and a fatty acid or fatty diacid chain having 14-22 carbon atoms.
200. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin, wherein said chain comprises a linker and a fatty acid or fatty diacid chain having 14 carbon atoms.
201. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin, wherein said chain comprises a linker and a fatty acid or fatty diacid chain having 16 carbon atoms.
202. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin, wherein said chain comprises a linker and a fatty acid or fatty diacid chain having 18 carbon atoms.
203. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin, wherein said chain comprises a linker and a fatty acid or fatty diacid chain having 20 carbon atoms.
204. The pharmaceutical composition according to any one of the preceding aspects wherein said acylated insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded acylated insulin to allow for the formation of one or more additional disulfide bonds not present in human insulin, wherein said chain comprises a linker and a fatty acid or fatty diacid chain having 22 carbon atoms.
205. The pharmaceutical composition according to any one of the preceding aspects wherein the sites of cysteine substitutions are chosen in such a way that
(1) The introduced cysteine residues are placed in the three dimensional structure of the folded acylated insulin to allow for the formation of one or more additional disulfide bonds not present in human insulin, and
(2) The human acylated insulin retains the desired biological activities associated with human insulin.
206. The pharmaceutical composition according to any one of the preceding aspects wherein the sites of cysteine substitutions are chosen in such a way that
(1) The introduced cysteine residues are placed in the three dimensional structure of the folded acylated insulin to allow for the formation of one or more additional disulfide bonds not present in human insulin,
(2) The human acylated insulin retains the desired biological activities associated with human insulin, and
(3) The human acylated insulin has increased physical stability relative to human insulin and/or parent insulin
207. The pharmaceutical composition according to any one of the preceding aspects wherein the sites of cysteine substitutions are chosen in such a way that
(1) The introduced cysteine residues are placed in the three dimensional structure of the folded acylated insulin to allow for the formation of one or more additional disulfide bonds not present in human insulin,
(2) The human acylated insulin retains the desired biological activities associated with human insulin, and
(3) The human acylated insulin is stabilised against proteolytic degradation.
208. The pharmaceutical composition according to any one of the preceding aspects wherein the amino acid residue in position A10 of the A-chain is substituted with a cysteine, the amino acid residue in a position selected from the group consisting of B1, B2, B3 and B4 of the B-chain is substituted with a cysteine, and optionally the amino acid in position B30 is deleted.
209. The pharmaceutical composition according to any one of the preceding aspects wherein one or more additional disulfide bonds are obtained between the A-chain and the B-chain
210. The pharmaceutical composition according to any one of the preceding aspects wherein said acylated insulin comprises on or more additional disulfide bonds and has a more pro-tracted profile than an acylated insulin without one or more additional disulfide bonds.
211. The pharmaceutical composition according to any one of the preceding aspects wherein said side chain is attached to the N-terminal end of the insulin or the epsilon amino group of a lysine residue in the insulin.
212. The pharmaceutical composition according to any one of the preceding aspects, wherein said polyvinyl alcohol coating is an aqueous coating.
213. The pharmaceutical composition according to aspect 102, wherein said polyvinyl alcohol coating comprises at least 25-55% polyvinyl alcohol polymer.
214. The pharmaceutical composition according to aspect 102, wherein said polyvinyl alcohol coating comprises at least 38-46% polyvinyl alcohol polymer.
215. The pharmaceutical composition according to any one of the preceding aspects, wherein said polyvinyl alcohol coating is an OPADRY®II—Yellow film (such as e.g. from Colorcon® (as sold in 2013).
216. The pharmaceutical composition according to any one of the preceding aspects, wherein said polyvinyl alcohol coating is not an anionic copolymer coating.
217. The pharmaceutical composition according to any one of the preceding aspects, wherein said polyvinyl alcohol coating is not pH sensitive, i.e. does not have a dissolution profile depending on pH.
218. The pharmaceutical composition according to any one of the preceding aspects, wherein said polyvinyl alcohol coating is not bioadhesive.
219. The pharmaceutical composition according to any one of the preceding aspects, wherein said polyvinyl alcohol coating is not mucoadhesive.
220. The pharmaceutical composition according to any one of the preceding aspects, wherein said acylated insulin comprises a Glutamine in position A14, i.e. comprises the amino acid A14Glu.
221. The pharmaceutical composition according to any one of the preceding aspects, wherein said acylated insulin comprises a Histidine in position B25, i.e. comprises the amino acid B25His.
222. The pharmaceutical composition according to any one of the preceding aspects, wherein said acylated insulin comprises a Histidine in position B16, i.e. comprises the amino acid B16His.
223. The pharmaceutical composition according to any one of the preceding aspects, wherein the amino acid in position B27 of said acylated insulin is deleted, i.e. said acylated insulin comprises desB27.
224. The pharmaceutical composition according to any one of the preceding aspects, wherein the amino acid in position B30 of said acylated insulin is deleted, i.e. said acylated insulin comprises desB30.
225. The pharmaceutical composition according to any one of the preceding aspects, wherein said acylated insulin is selected from the group consisting of:
A14E,B25H,B29K(N^{ε}-Hexadecandioyl),desB30 human insulin,
A14E,B25H,B29K(N^{ε}Octadecandioyl-γGlu),desB30 human insulin,
A14E,B25H,B29K(N^{ε}Eicosanedioyl-γGlu),desB30 human insulin,
A14E,B25H,B29K(N^{ε}3-Carboxy-5-octadecanedioylaminobenzoyl),desB30 human insulin,
A14E,B25H,B29K(N^{ε}—N-octadecandioyl-N-(2-carboxyethyl)glycyl),desB30 human insulin
A14E,B25H,B29K(N^{ε}(N-Octadecandioyl-N-carboxymethyl)-beta-alanyl),desB30 human insulin,
A14E,B25H,B29K(N^{ε}4-([4-({19-Carboxynonadecanoylamino}methyl)trans-cyclohexanecarbonyl]-γGlu),desB30 human insulin,
A14E,B25H,B29K(N^{ε}Heptadecanedioyl-γGlu),desB30 human insulin,
A14E,B25H,B29K(N^{ε}Octadecanedioyl-γGlu-OEG-OEG),desB30 human insulin,
A14E,B25H,B29K(N^{ε}Myristyl),desB30 human insulin,
A14E,B25H,B29K(N^{ε}Eicosanedioyl-γGlu-γGlu),desB30 human insulin,
A14E,B25H,B29K(N^{ε}4-([4-({19-Carboxynonadecanoylamino}methyl)trans-cyclohexanecarbonyl]-γGlu-γGlu),desB30 human insulin,
A14E,B25H,B29K(N^{ε}Octadecanedioyl-γGlu-γGlu),desB30 human insulin,
A14E,B28D,B29K(N^{ε}octadecandioyl-γGlu),desB30 human insulin,
A14E,B25H,B29K(N^{ε}octadecandioyl-γGlu-PEG7),desB30 human insulin,
A14E,B25H,B29K(N^{ε}eicosanedioyl-γGlu-OEG-OEG), desB30 human insulin,
A14E,B25H,B29K(N^{ε}eicosanedioyl-γGlu-(3-(2-{2-[2-(2-aminoethoxy)ethoxy]ethoxy}ethoxy)propionyl-γGlu),desB30 human insulin,
A14E,B25H,B29K(N^{ε}Hexadecanedioyl-γGlu-OEG-OEG),desB30 human insulin,
A14E,B25H,B29K(N^{ε}Hexadecanedioyl-γGlu),desB30 human insulin,
A14E,B25H,B29K(N^{ε}heptadecanedioyl-γGlu-OEG-OEG),desB30 human insulin,
A14E,B25H,B29K(N^{ε}octadecanedioyl-γGlu-γGlu-γGlu-γGlu),desB30 human insulin,
A14E,B25H,B29K(N^{ε}Eicosanedioyl-γGlu-γGlu-γGlu),desB30 human insulin,
A14E,B25H,B27E,B29K(N^{ε}Octadecanedioyl-γGlu-OEG-OEG),desB30 human insulin,
A14E,B25H,B26G,B27G,B28G,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16E,B25H,B29K(N^{ε}Octadecanedioyl-γGlu-OEG-OEG),desB30 human insulin,
A14E,B16H,B25H,B29K(N^{ε}Hexadecanedioyl-γGlu),desB30 human insulin,
A14E,B25H,B29K(N^{ε}Eicosanedioyl-γGlu-OEG-γGlu),desB30 human insulin,
A14E,B16E,B25H,B29K(N^{ε}Hexadecandioyl-γGlu),desB30 human insulin,
A14E,B16H,B25H,B29K(N^{ε}Octadecanedioyl-γGlu-γGlu-γGlu),desB30 human insulin,
A14E,B25H,B26G,B27G,B28G,B29K(N^{ε}Hexadecandioyl-γGlu),desB30 human insulin,
A14E,B16H,B25H,B29K(N^{ε}Octadecanedioyl-γGlu-γGlu),desB30 human insulin,
A14E,B16H,B25H,B29K(N(eps)Eicosanedioyl-γGlu-OEG-OEG),desB30 human insulin,
A14E,B25H,B29K(N^{ε}Octadecanedioyl-OEG-γGlu-γGlu),desB30 human insulin,
A14E,A18L,B25H,B29K(N^{ε}Eicosanedioyl-γGlu-OEG-OEG),desB30 human insulin,
A14E,A18L,B25H,B29K(N^{ε}Octadecanedioyl-γGlu-OEG-OEG),desB30 human insulin,
A14E,B25H,B27E,B29K(N^{ε}Eicosanedioyl-γGlu-OEG-OEG),desB30 human insulin,
A1G(N^{α}Octadecandioyl-γGlu-OEG-OEG),A14E,B25H,B29R,desB30 human insulin,
A14E,B1F(N^{α}Octadecandioyl-γGlu-OEG-OEG),B25H,B29R,desB30 human insulin,
A1G(N^{α}Hexadecandioyl-γGlu),A14E,B25H,B29R,desB30 human insulin,
A14E,B25H,B29K(N^{ε}Octadecanedioyl-γGlu-Abu-Abu-Abu-Abu),desB30 human insulin,
A14E,B25H,B29K(N^{α}Eicosanedioyl),desB30 human insulin,
A14E,B25H,B29K(N^{α}4-[16-(1H-Tetrazol-5-yl)hexadecanoylsulfamoyl]butanoyl), desB30 human insulin,
A1G(N^{α}Octadecandioyl-γGlu-OEG-OEG),A14E,A21G,B25H,desB30 human insulin,
A14E,B25H,B29K(N^{ε}Eicosanedioyl-OEG),desB30 human insulin,
A14E,B25H,B27K(N^{ε}Octadecanedioyl-γGlu-OEG-OEG),desB28,desB29,desB30 human insulin,
A14E,B25H,B29K(N^{ε}(5-Eicosanedioylaminoisophthalic acid)),desB30 human insulin,
A14E,B25H,B29K(N^{ε}Octadecanedioyl),desB30 human insulin,
A14E,B29K(N^{ε}Octadecanedioyl-γGlu-OEG-OEG),desB30 human insulin,
A14E,B25H,B26G,B27G,B28G,B29K(N^{ε}Eicosanedioyl-γGlu-OEG-OEG),desB30 human insulin,
A14E,B25H,B29K(N^{ε}Octadecanedioyl-γGlu-OEG),desB30 human insulin,
A14E,B25H,B29K(N^{ε}Eicosanedioyl-OEG-OEG),desB30 human insulin,
A14E,B25H,B29K(N^{ε}Eicosanedioyl-Aoc),desB30 human insulin,
A14E,B25H,B26G,B27G,B28G,B29K(N^{ε}Eicosanedioyl-γGlu-γGlu),desB30 human insulin,
A14E,B25H,B26G,B27G,B28G,B29K(N^{ε}Eicosanedioyl-γGlu-γGlu),desB30 human insulin,
A14E,B25H,B29K(N^{ε}Octadecanedioyl-OEG),desB30 human insulin,
A14E,B25H,desB27,B29K(N^{ε}Octadecanedioyl-γGlu-OEG-OEG),desB30 human insulin,
A14E,B25H,B16H,B29K(N^{ε}Octadecanedioyl-γGlu),desB30 human insulin,
A1G(N^{α}Octadecanedioyl),A14E,B25H,B29R,desB30 human insulin,
A14E,B16H,B25H,B29K(N^{ε}Eicosanedioyl-γGlu),desB30 human insulin,
A14E,B25H,B27K(N^{ε}Eicosanedioyl-γGlu),desB28,desB29,desB30 human insulin,
A14E,B25H,B29K(N^{ε}Octadecanedioyl-γGlu-γGlu-γGlu),desB30 human insulin,
A14E,B25H,B26G,B27G,B28G,B29K(N^{ε}Octadecandioyl-γGlu),desB30 human insulin,
A14E,B25H,B26G,B27G,B28G,B29K(N^{ε}Eicosanedioyl-γGlu),desB30 human insulin,
A14E,B25H,B26G,B27G,B28G,B29K(N^{ε}Octadecandioyl),desB30 human insulin,
A14E,B25H,B26G,B27G,B28G,B29K(N^{ε}Eicosanedioyl),desB30 human insulin,
A14E,B25H,B29K(N^{ε}Docosanedioyl-γGlu),desB30 human insulin,
A14E,B25H,B29K(N^{ε}1 Docosanedioyl-γGlu-γGlu),desB30 human insulin,
A14E,B25H,B29K(N^{ε}Icosanedioyl-γGlu-OEG-OEG-γGlu),desB30 human insulin,
A14E,B25H,B29K(N^{ε}Octadecanedioyl-γGlu-OEG-OEG-γGlu),desB30 human insulin,
A14E,B25H,B29K(N^{ε}(N-Icosanedioyl-N-carboxymethyl)-βAla),desB30 human insulin,
A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
A14E,B25H,B29K(N^{ε}Octadecandioyl-γGlu-(3-(2-{2-[2-(2-aminoethoxy)ethoxy]ethoxy}ethoxy)propionyl),desB30 human insulin,
A14E,B25H,B29K(N^{ε}Octadecandioyl-γGlu-(3-(2-{2-[2-(2-aminoethoxy)ethoxy]ethoxy}ethoxy)propionyl-γGlu),desB30 human insulin,
A14E,B25H,B29K(N^{ε}Icosanedioyl-γGlu-(3-(2-{2-[2-(2-aminoethoxy)ethoxy]ethoxy}ethoxy)propionyl),desB30 human insulin, A14E,B25H,B29K(N^{ε}4-([4-({17-Carboxynonadecanoylamino}methyl)trans-cyclohexanecarbonyl]-γGlu),desB30 human insulin,
A14E,B25H,B29K(N^{ε}4-([4-({17-Carboxyheptadecanoylamino}methyl)trans-cyclohexanecarbonyl]-γGlu-γGlu),desB30 human insulin,
A14E,B28D,B29K(N^{ε}hexadecandioyl-γGlu),desB30 human insulin,
A14E,B28D,B29K(N^{ε}Eicosanedioyl-γGlu),desB30 human insulin,
A14E,B28D,B29K(N^{ε}Octadecandioyl-γGlu-OEG-OEG),desB30 human insulin,
A14E,B28D,B29K(N^{ε}Eicosanedioyl-γGlu-OEG-OEG),desB30 human insulin,
A14E,B28E,B29K(N^{ε}Hexadecandioyl-γGlu),desB30 human insulin,
A14E,B28E,B29K(N^{ε}Octadecandioyl-γGlu),desB30 human insulin,
A14E,B28E,B29K(N^{ε}Eicosanedioyl-γGlu),desB30 human insulin,
A14E,B28E,B29K(N^{ε}Octadecandioyl-γGlu-OEG-OEG),desB30 human insulin,
A14E,B28E,B29K(N^{ε}Eicosanedioyl-γGlu-OEG-OEG),desB30 human insulin,
A14E,B1E,B28E,B29K(N^{ε}Hexadecandioyl-γGlu),desB30 human insulin,
A14E,B1E,B28E,B29K(N^{ε}Octadecandioyl-γGlu),desB30 human insulin,
A14E,B1E,B28E,B29K(N^{ε}Eicosanedioyl-γGlu),desB30 human insulin,
A14E,B1E,B28E,B29K(N^{ε}Hexadecandioyl-γGlu-OEG-OEG),desB30 human insulin,
A14E,B1E,B28E,B29K(N^{ε}Octadecandioyl-γGlu-OEG-OEG),desB30 human insulin,
A14E,B1E,B28E,B29K(N^{ε}Eicosanedioyl-γGlu-OEG-OEG),desB30 human insulin,
A14E,B1E,B27E,B28E,B29K(N^{ε}Hexadecandioyl-γGlu),desB30 human insulin,
A14E,B1E,B27E,B28E,B29K(N^{ε}Octadecandioyl-γGlu),desB30 human insulin,
A14E,B1E,B27E,B28E,B29K(N^{ε}Eicosanedioyl-γGlu),desB30 human insulin,
A14E,B1E,B27E,B28E,B29K(N^{ε}Hexadecandioyl-γGlu-OEG-OEG),desB30 human insulin,
A14E,B1E,B27E,B28E,B29K(N^{ε}Octadecandioyl-γGlu-OEG-OEG),desB30 human insulin,
A14E,B1E,B27E,B28E,B29K(N^{ε}Eicosanedioyl-γGlu-OEG-OEG),desB30 human insulin,
A14E,B1E,B25H,B28E,B29K(N^{ε}Hexadecandioyl-γGlu),desB30 human insulin,
A14E,B1E,B25H,B28E,B29K(N^{ε}Octadecandioyl-γGlu),desB30 human insulin,
A14E,B1E,B25H,B28E,B29K(N^{ε}Eicosanedioyl-γGlu),desB30 human insulin,
A14E,B1E,B25H,B28E,B29K(N^{ε}Hexadecandioyl-γGlu-OEG-OEG),desB30 human insulin,
A14E,B1E,B25H,B28E,B29K(N^{ε}Octadecandioyl-γGlu-OEG-OEG),desB30 human insulin,
A14E,B1E,B25H,B28E,B29K(N^{ε}Eicosanedioyl-γGlu-OEG-OEG),desB30 human insulin,
A14E,B1E,B25H,B27E,B28E,B29K(N^{ε}Hexadecandioyl-γGlu),desB30 human
A14E,B1E,B25H,B27E,B28E,B29K(N^{ε}Octadecandioyl-γGlu),desB30 human insulin,
A14E,B1E,B25H,B27E,B28E,B29K(N^{ε}Eicosanedioyl-γGlu),desB30 human insulin,
A14E,B1E,B25H,B27E,B28E,B29K(N^{ε}Hexadecandioyl-γGlu-OEG-OEG),desB30 human insulin,
A14E, B1E, B25H, B27E, B28E, B29K(N^{ε}Octadecandioyl-γGlu-OEG-OEG),desB30 human insulin,
A14E,B1E,B25H,B27E,B28E,B29K(N^{ε}Eicosanedioyl-γGlu-OEG-OEG),desB30 human insulin,
A14E,B28D,B29K(N^{ε}Hexadecanedioyl-γGlu-OEG-OEG),desB30 human insulin,
A14E,B28E,B29K(N^{ε}Hexadecanedioyl-γGlu-OEG-OEG),desB30 human insulin,
B25N,B27E,B29K(N^{ε}Eicosanedioyl-γGlu-OEG-OEG),desB30 human insulin,
B25N,B27E,B29K(N^{ε}Octadecanedioyl-γGlu-OEG-OEG),desB30 human insulin,
B25N,B27E,B29K(N^{ε}Hexadecanedioyl-γGlu-OEG-OEG),desB30 human insulin,
B25N,B27E,B29K(N^{ε}Eicosanedioyl-γGlu),desB30 human insulin,
B25N,B27E,B29K(N^{ε}Octadecanedioyl-γGlu),desB30 human insulin,
B25N,B27E,B29K(N^{ε}Hexadecanedioyl-γGlu),desB30 human insulin,
A8H,B25N,B27E,B29K(N^{ε}Eicosanedioyl-γGlu-OEG-OEG),desB30 human insulin,
A8H,B25N,B27E,B29K(N^{ε}Octadecanedioyl-γGlu-OEG-OEG),desB30 human insulin,
A8H,B25N,B27E,B29K(N^{ε}Hexadecanedioyl-γGlu-OEG-OEG),desB30 human insulin,
A8H,B25N,B27E,B29K(N^{ε}Eicosanedioyl-γGlu),desB30 human insulin,
A8H,B25N,B27E,B29K(N^{ε}Octadecanedioyl-γGlu),desB30 human insulin,
A8H,B25N,B27E,B29K(N^{ε}Hexadecanedioyl-γGlu),desB30 human insulin,
14E,B25H,B29K(N^{ε}(N-Icosanedioyl-N-carboxymethyl)-βAla-OEG-OEG),desB30 human insulin,
A14E,B25H,B29K(N^{ε}(N-Octadecanedioyl-N-carboxymethyl)-βAla-OEG-OEG),desB30 human insulin,
A14E,B25H,B29K(N^{ε}(N-Hexadecanedioyl-N-carboxymethyl)-βAla-OEG-OEG),desB30 human insulin,
A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
A14E,B16H,B25H,B29K(N^{ε}Octadecanedioyl-γGlu-2-[(3-{2-[2-(3-aminopropoxy)-ethoxy]ethoxy}propylcarbamoyl)methoxy]acetyl),desB30 human insulin,
A14E, B16H, B25H, B29K(N^{ε}Eicosanedioyl-γGlu-2-[(3-{2-[2-(3-aminopropoxy)-ethoxy]ethoxy}propylcarbamoyl)methoxy]acetyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
B25H,B29K(N^{ε}Octadecanedioyl),desB30 human insulin,
B25H,B29K(N^{ε}Eicosanedioyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
21G,B25H,B29K(N^{ε}Octadecanedioyl),desB30 human insulin,
A21G,B25H,B29K(N^{ε}Eicosanedioyl),desB30 human insulin,
A21G,B25H,B29K(N^{ε}Octadecanedioyl-γGlu-OEG-OEG),desB30 human insulin,
A21G,B25H,B29K(N^{ε}Eicosanedioyl-γGlu-OEG-OEG),desB30 human insulin,
A21G,B25H,B29K(N^{ε}Octadecanedioyl-γGlu),desB30 human insulin,
A21G,B25H,B29K(N^{ε}Eicosanedioyl-γGlu),desB30 human insulin,
A14E,B25H,desB27,B29K(N^{ε}Octadecanedioyl),desB30 human insulin,
A14E,B25H,desB27,B29K(N^{ε}Eicosanedioyl),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,B25H,desB27,B29K(N^{ε}Eicosanedioyl-γGlu-OEG-OEG),desB30 human insulin,
A14E,A21G,B25H,desB27,B29K(N^{ε}Octadecanedioyl),desB30 human insulin,
A14E,A21G,B25H,desB27,B29K(N^{ε}Eicosanedioyl),desB30 human insulin,
A14E,A21G,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,A21G,B25H,desB27,B29K(N^{ε}Octadecanedioyl-γGlu-OEG-OEG),desB30 human insulin,
A14E,A21G,B25H,desB27,B29K(N^{ε}Eicosanedioyl-γGlu-OEG-OEG),desB30 human insulin,
A14E,A21G,B25H,B29K(N^{ε}Octadecanedioyl-γGlu-OEG-OEG),desB30 human insulin,
A14E,A21G,B25H,B29K(N^{ε}Eicosanedioyl-γGlu-OEG-OEG),desB30 human insulin,
A14E,A21G,B25H,B29K(N^{ε}Eicosanedioyl-γGlu),desB30 human insulin,
A14E,A21G,B25H,B29K(N^{ε}Eicosanedioyl),desB30 human insulin,
A14E,A21G,B25H,B29K(N^{ε}Octadecanedioyl-γGlu),desB30 human insulin,
A14E,A21G,B25H,B29K(N^{ε}Octadecanedioyl),desB30 human insulin,
A14E,B25H,B26G,B27G,B28G,B29K(N^{ε}Octadecanedioyl-γGlu),desB30 human insulin,
A14E,B25H,B26G,B27G,B28G,B29K(N^{ε}Octadecanedioyl),desB30 human insulin,
A14E,B25H,B26G,B27G,B28G,B29K(N^{ε}Eicosanedioyl-γGlu),desB30 human insulin,
A14E,B25H,B26G,B27G,B28G,B29K(N^{ε}Eicosanedioyl),desB30 human insulin,
A1G(N^{α}Octadecandioyl-γGlu),A14E,B25H,B26G,B27G,B28G,desB30 human insulin,
A1G(N^{α}Eicosanedioyl-γGlu),A14E,B25H,B26G,B27G,B28G,desB30 human insulin,
A1G(N^{α}Octadecandioyl-γGlu),A14E,B25H,B26G,B27G,B28G,B29R,desB30 human insulin,
A1G(N^{α}Eicosanedioyl-γGlu),A14E,B25H,B26G,B27G,B28G,B29R,desB30 human insulin,
A1G(N^{α}Octadecandioyl),A14E,B25H,B26G,B27G,B28G,desB30 human insulin,
A1G(N^{α}Eicosanedioyl),A14E,B25H,B26G,B27G,B28G,desB30 human insulin,
A1G(N^{α}Octadecandioyl),A14E,B25H,B26G,B27G,B28G,B29R,desB30 human insulin and
A1G(N^{α}Eicosanedioyl),A14E,B25H,B26G,B27G,B28G,B29R,desB30 human insulin.
226. The pharmaceutical composition according to any one of the preceding aspects, wherein said acylated insulin is selected from the group consisting of: A14E,B25H,B29K(N^{ε}-Hexadecandioyl),desB30 human insulin,
A14E,B25H,B29K(N^{ε}Octadecandioyl-γGlu),desB30 human insulin,
A14E,B25H,B29K(N^{ε}Eicosanedioyl-γGlu),desB30 human insulin,
A14E,B25H,B29K(N^{ε}3-Carboxy-5-octadecanedioylaminobenzoyl),desB30 human insulin,
A14E,B25H,B29K(N^{ε}—N-octadecandioyl-N-(2-carboxyethyl)glycyl),desB30 human insulin
A14E,B25H,B29K(N^{ε}(N-Octadecandioyl-N-carboxymethyl)-beta-alanyl),desB30 human insulin,
A14E,B25H,B29K(N^{ε}4-([4-({19-Carboxynonadecanoylamino}methyl)trans-cyclohexanecarbonyl]-γGlu),desB30 human insulin,
A14E,B25H,B29K(N^{ε}Heptadecanedioyl-γGlu),desB30 human insulin,
A14E,B25H,B29K(N^{ε}Octadecanedioyl-γGlu-OEG-OEG),desB30 human insulin,
A14E,B25H,B29K(N^{ε}Myristyl),desB30 human insulin,
A14E,B25H,B29K(N^{ε}Eicosanedioyl-γGlu-γGlu),desB30 human insulin,
A14E,B25H,B29K(N^{ε}4-([4-({19-Carboxynonadecanoylamino}methyl)trans-cyclohexanecarbonyl]-γGlu-γGlu),desB30 human insulin,
A14E,B25H,B29K(N^{ε}Octadecanedioyl-γGlu-γGlu),desB30 human insulin,
A14E,B28D,B29K(N^{ε}octadecandioyl-γGlu),desB30 human insulin,
A14E,B25H,B29K(N^{ε}octadecandioyl-γGlu-PEG7),desB30 human insulin,
A14E,B25H,B29K(N^{ε}eicosanedioyl-γGlu-OEG-OEG), desB30 human insulin,
A14E,B25H,B29K(N^{ε}eicosanedioyl-γGlu-(3-(2-{2-[2-(2-aminoethoxy)ethoxy]ethoxy}ethoxy)propionyl-γGlu),desB30 human insulin,
A14E,B25H,B29K(N^{ε}Hexadecanedioyl-γGlu-OEG-OEG),desB30 human insulin,
A14E,B25H,B29K(N^{ε}Hexadecanedioyl-γGlu),desB30 human insulin,
A14E,B25H,B29K(N^{ε}heptadecanedioyl-γGlu-OEG-OEG),desB30 human insulin,
A14E,B25H,B29K(N^{ε}octadecanedioyl-γGlu-γGlu-γGlu-γGlu),desB30 human insulin,
A14E,B25H,B29K(N^{ε}Eicosanedioyl-γGlu-γGlu-γGlu),desB30 human insulin,
A14E,B25H,B27E,B29K(N^{ε}Octadecanedioyl-γGlu-OEG-OEG),desB30 human insulin,
A14E,B25H,B26G,B27G,B28G,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human
A14E,B16E,B25H,B29K(N^{ε}Octadecanedioyl-γGlu-OEG-OEG),desB30 human insulin,
A14E,B16H,B25H,B29K(N^{ε}Hexadecanedioyl-γGlu),desB30 human insulin,
A14E,B25H,B29K(N^{ε}Eicosanedioyl-γGlu-OEG-γGlu),desB30 human insulin,
A14E,B16E,B25H,B29K(N^{ε}Hexadecandioyl-γGlu),desB30 human insulin,
A14E,B16H,B25H,B29K(N^{ε}Octadecanedioyl-γGlu-γGlu-γGlu),desB30 human insulin,
A14E,B25H,B26G,B27G,B28G,B29K(N^{ε}Hexadecandioyl-γGlu),desB30 human insulin,
A14E,B16H,B25H,B29K(N^{ε}Octadecanedioyl-γGlu-γGlu),desB30 human insulin,
A14E,B16H,B25H,B29K(N(eps)Eicosanedioyl-γGlu-OEG-OEG),desB30 human insulin,
A14E,B25H,B29K(N^{ε}Octadecanedioyl-OEG-γGlu-γGlu),desB30 human insulin,
A14E,A18L,B25H,B29K(N^{ε}Eicosanedioyl-γGlu-OEG-OEG),desB30 human insulin,
A14E,A18L,B25H,B29K(N^{ε}Octadecanedioyl-γGlu-OEG-OEG),desB30 human insulin,
A14E,B25H,B27E,B29K(N^{ε}Eicosanedioyl-γGlu-OEG-OEG),desB30 human insulin,
A1G(N^{α}Octadecandioyl-γGlu-OEG-OEG),A14E,B25H,B29R,desB30 human insulin,
A14E,B1F(N^{α}Octadecandioyl-γGlu-OEG-OEG),B25H,B29R,desB30 human insulin,
A1G(N^{α}Hexadecandioyl-γGlu),A14E,B25H,B29R,desB30 human insulin,
A14E,B25H,B29K(N^{ε}Octadecanedioyl-γGlu-Abu-Abu-Abu-Abu),desB30 human insulin,
A14E,B25H,B29K(N^{α}Eicosanedioyl),desB30 human insulin,
A14E,B25H,B29K(N^{ε}4-[16-(1H-Tetrazol-5-yl)hexadecanoylsulfamoyl]butanoyl), desB30 human insulin,
A1G(N^{α}Octadecandioyl-γGlu-OEG-OEG),A14E,A21G,B25H,desB30 human insulin,
A14E,B25H,B29K(N^{ε}Eicosanedioyl-OEG),desB30 human insulin,
A14E,B25H,B27K(N^{ε}Octadecanedioyl-γGlu-OEG-OEG),desB28,desB29,desB30 human insulin,
A14E,B25H,B29K(N^{ε}(5-Eicosanedioylaminoisophthalic acid)),desB30 human insulin,
A14E,B25H,B29K(N^{ε}Octadecanedioyl),desB30 human insulin,
A14E,B29K(N^{ε}Octadecanedioyl-γGlu-OEG-OEG),desB30 human insulin,
A14E,B25H,B26G,B27G,B28G,B29K(N^{ε}Eicosanedioyl-γGlu-OEG-OEG),desB30 human insulin,
A14E,B25H,B29K(N^{ε}Octadecanedioyl-γGlu-OEG),desB30 human insulin,
A14E,B25H,B29K(N^{ε}Eicosanedioyl-OEG-OEG),desB30 human insulin,
A14E,B25H,B29K(N^{ε}Eicosanedioyl-Aoc),desB30 human insulin,
A14E,B25H,B26G,B27G,B28G,B29K(N^{ε}Eicosanedioyl-γGlu-γGlu),desB30 human insulin,
A14E,B25H,B26G,B27G,B28G,B29K(N^{ε}Eicosanedioyl-γGlu-γGlu),desB30 human insulin,
A14E,B25H,B29K(N^{ε}Octadecanedioyl-OEG),desB30 human insulin,
A14E,B25H,desB27,B29K(N^{ε}Octadecanedioyl-γGlu-OEG-OEG),desB30 human insulin,
A14E,B25H,B16H,B29K(N^{ε}Octadecanedioyl-γGlu),desB30 human insulin,
A1G(N^{α}Octadecanedioyl),A14E,B25H,B29R,desB30 human insulin,
A14E,B16H,B25H,B29K(N^{ε}Eicosanedioyl-γGlu),desB30 human insulin,
A14E,B25H,B27K(N^{ε}Eicosanedioyl-γGlu),desB28,desB29,desB30 human insulin,
A14E,B25H,B29K(N^{ε}Octadecanedioyl-γGlu-γGlu-γGlu),desB30 human insulin,
A14E,B25H,B26G,B27G,B28G,B29K(N^{ε}Octadecandioyl-γGlu),desB30 human insulin,
A14E,B25H,B26G,B27G,B28G,B29K(N^{ε}Eicosanedioyl-γGlu),desB30 human insulin,
A14E,B25H,B26G,B27G,B28G,B29K(N^{ε}Octadecandioyl),desB30 human insulin,
A14E,B25H,B26G,B27G,B28G,B29K(N^{ε}Eicosanedioyl),desB30 human insulin,
A14E,B25H,B29K(N^{ε}1 Docosanedioyl-γGlu),desB30 human insulin,
A14E,B25H,B29K(N^{ε}1 Docosanedioyl-γGlu-γGlu),desB30 human insulin,
A14E,B25H,B29K(N^{ε}Icosanedioyl-γGlu-OEG-OEG-γGlu),desB30 human insulin,
A14E,B25H,B29K(N^{ε}Octadecanedioyl-γGlu-OEG-OEG-γGlu),desB30 human insulin,
A14E,B25H,B29K(N^{ε}(N-Icosanedioyl-N-carboxymethyl)-βAla),desB30 human insulin,
A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
A14E,B25H,B29K(N^{ε}Octadecandioyl-γGlu-(3-(2-{2-[2-(2-aminoethoxy)ethoxy]ethoxy}ethoxy)propionyl),desB30 human insulin,
A14E,B25H,B29K(N^{ε}Octadecandioyl-γGlu-(3-(2-{2-[2-(2-aminoethoxy)ethoxy]ethoxy}ethoxy)propionyl-γGlu),desB30 human insulin,
A14E,B25H,B29K(N^{ε}Icosanedioyl-γGlu-(3-(2-{2-[2-(2-aminoethoxy)ethoxy]ethoxy}ethoxy)propionyl),desB30 human insulin,
A14E,B25H,B29K(N^{ε}4-([4-({17-Carboxynonadecanoylamino}methyl)trans-cyclohexanecarbonyl]-γGlu),desB30 human insulin,
A14E,B25H,B29K(N^{ε}4-([4-({17-Carboxyheptadecanoylamino}methyl)trans-cyclohexanecarbonyl]-γGlu-γGlu),desB30 human insulin,
A14E,B28D,B29K(N^{ε}hexadecandioyl-γGlu),desB30 human insulin,
A14E,B28D,B29K(N^{ε}Eicosanedioyl-γGlu),desB30 human insulin,
A14E,B28D,B29K(N^{ε}Octadecandioyl-γGlu-OEG-OEG),desB30 human insulin,
A14E,B28D,B29K(N^{ε}Eicosanedioyl-γGlu-OEG-OEG),desB30 human insulin,
A14E,B28E,B29K(N^{ε}Hexadecandioyl-γGlu),desB30 human insulin,
A14E,B28E,B29K(N^{ε}Octadecandioyl-γGlu),desB30 human insulin,
A14E,B28E,B29K(N^{ε}Eicosanedioyl-γGlu),desB30 human insulin,
A14E,B28E,B29K(N^{ε}Octadecandioyl-γGlu-OEG-OEG),desB30 human insulin,
A14E,B28E,B29K(N^{ε}Eicosanedioyl-γGlu-OEG-OEG),desB30 human insulin,
A14E,B1E,B28E,B29K(N^{ε}Hexadecandioyl-γGlu),desB30 human insulin,
A14E,B1E,B28E,B29K(N^{ε}Octadecandioyl-γGlu),desB30 human insulin,
A14E,B1E,B28E,B29K(N^{ε}Eicosanedioyl-γGlu),desB30 human insulin,
A14E,B1E,B28E,B29K(N^{ε}Hexadecandioyl-γGlu-OEG-OEG),desB30 human insulin,
A14E,B1E,B28E,B29K(N^{ε}Octadecandioyl-γGlu-OEG-OEG),desB30 human insulin,
A14E,B1E,B28E,B29K(N^{ε}Eicosanedioyl-γGlu-OEG-OEG),desB30 human insulin,
A14E,B1E,B27E,B28E,B29K(N^{ε}Hexadecandioyl-γGlu),desB30 human insulin,
A14E,B1E,B27E,B28E,B29K(N^{ε}Octadecandioyl-γGlu),desB30 human insulin,
A14E,B1E,B27E,B28E,B29K(N^{ε}Eicosanedioyl-γGlu),desB30 human insulin,
A14E,B1E,B27E,B28E,B29K(N^{ε}Hexadecandioyl-γGlu-OEG-OEG),desB30 human
A14E,B1E,B27E,B28E,B29K(N^{ε}Octadecandioyl-γGlu-OEG-OEG),desB30 human insulin,
A14E,B1E,B27E,B28E,B29K(N^{ε}Eicosanedioyl-γGlu-OEG-OEG),desB30 human insulin,
A14E,B1E,B25H,B28E,B29K(N^{ε}Hexadecandioyl-γGlu),desB30 human insulin,
A14E,B1E,B25H,B28E,B29K(N^{ε}Octadecandioyl-γGlu),desB30 human insulin,
A14E,B1E,B25H,B28E,B29K(N^{ε}Eicosanedioyl-γGlu),desB30 human insulin,
A14E,B1E,B25H,B28E,B29K(N^{ε}Hexadecandioyl-γGlu-OEG-OEG),desB30 human insulin,
A14E,B1E,B25H,B28E,B29K(N^{ε}Octadecandioyl-γGlu-OEG-OEG),desB30 human insulin,
A14E,B1E,B25H,B28E,B29K(N^{ε}Eicosanedioyl-γGlu-OEG-OEG),desB30 human insulin,
A14E,B1E,B25H,B27E,B28E,B29K(N^{ε}Hexadecandioyl-γGlu),desB30 human insulin,
A14E,B1E,B25H,B27E,B28E,B29K(N^{ε}Octadecandioyl-γGlu),desB30 human insulin,
A14E,B1E,B25H,B27E,B28E,B29K(N^{ε}Eicosanedioyl-γGlu),desB30 human insulin,
A14E,B1E,B25H,B27E,B28E,B29K(N^{ε}Hexadecandioyl-γGlu-OEG-OEG),desB30 human insulin,
A14E, B1E, B25H, B27E, B28E, B29K(N^{ε}Octadecandioyl-γGlu-OEG-OEG),desB30 human insulin,
A14E,B1E,B25H,B27E,B28E,B29K(N^{ε}Eicosanedioyl-γGlu-OEG-OEG),desB30 human insulin,
A14E,B28D,B29K(N^{ε}Hexadecanedioyl-γGlu-OEG-OEG),desB30 human insulin,
A14E,B28E,B29K(N^{ε}Hexadecanedioyl-γGlu-OEG-OEG),desB30 human insulin,
B25N,B27E,B29K(N^{ε}Eicosanedioyl-γGlu-OEG-OEG),desB30 human insulin,
B25N,B27E,B29K(N^{ε}Octadecanedioyl-γGlu-OEG-OEG),desB30 human insulin,
B25N,B27E,B29K(N^{ε}Hexadecanedioyl-γGlu-OEG-OEG),desB30 human insulin,
B25N,B27E,B29K(N^{ε}Eicosanedioyl-γGlu),desB30 human insulin,
B25N,B27E,B29K(N^{ε}Octadecanedioyl-γGlu),desB30 human insulin,
B25N,B27E,B29K(N^{ε}Hexadecanedioyl-γGlu),desB30 human insulin,
A8H,B25N,B27E,B29K(N^{ε}Eicosanedioyl-γGlu-OEG-OEG),desB30 human insulin,
A8H,B25N,B27E,B29K(N^{ε}Octadecanedioyl-γGlu-OEG-OEG),desB30 human insulin,
A8H,B25N,B27E,B29K(N^{ε}Hexadecanedioyl-γGlu-OEG-OEG),desB30 human insulin,
A8H,B25N,B27E,B29K(N^{ε}Eicosanedioyl-γGlu),desB30 human insulin,
A8H,B25N,B27E,B29K(N^{ε}Octadecanedioyl-γGlu),desB30 human insulin,
A8H,B25N,B27E,B29K(N^{ε}Hexadecanedioyl-γGlu),desB30 human insulin,
14E,B25H,B29K(N^{ε}(N-Icosanedioyl-N-carboxymethyl)-βAla-OEG-OEG),desB30 human insulin,
A14E,B25H,B29K(N^{ε}(N-Octadecanedioyl-N-carboxymethyl)-βAla-OEG-OEG),desB30 human insulin,
A14E,B25H,B29K(N^{ε}(N-Hexadecanedioyl-N-carboxymethyl)-βAla-OEG-OEG),desB30 human insulin,
A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
A14E,B16H,B25H,B29K(N^{ε}Octadecanedioyl-γGlu-2-[(3-{2-[2-(3-aminopropoxy)-ethoxy]ethoxy}propylcarbamoyl)methoxy]acetyl),desB30 human insulin,
A14E, B16H, B25H, B29K(N^{ε}Eicosanedioyl-γGlu-2-[(3-{2-[2-(3-aminopropoxy)-ethoxy]ethoxy}propylcarbamoyl)methoxy]acetyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
B25H,B29K(N^{ε}Octadecanedioyl),desB30 human insulin,
B25H,B29K(N^{ε}Eicosanedioyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
21G,B25H,B29K(N^{ε}Octadecanedioyl),desB30 human insulin,
A21G,B25H,B29K(N^{ε}Eicosanedioyl),desB30 human insulin,
A21G,B25H,B29K(N^{ε}Octadecanedioyl-γGlu-OEG-OEG),desB30 human insulin,
A21G,B25H,B29K(N^{ε}Eicosanedioyl-γGlu-OEG-OEG),desB30 human insulin,
A21G,B25H,B29K(N^{ε}Octadecanedioyl-γGlu),desB30 human insulin,
A21G,B25H,B29K(N^{ε}Eicosanedioyl-γGlu),desB30 human insulin,
A14E,B25H,desB27,B29K(N^{ε}Octadecanedioyl),desB30 human insulin,
A14E,B25H,desB27,B29K(N^{ε}Eicosanedioyl),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,B25H,desB27,B29K(N^{ε}Eicosanedioyl-γGlu-OEG-OEG),desB30 human insulin,
A14E,A21G,B25H,desB27,B29K(N^{ε}Octadecanedioyl),desB30 human insulin,
A14E,A21G,B25H,desB27,B29K(N^{ε}Eicosanedioyl),desB30 human insulin,
A14E,A21G,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,A21G,B25H,desB27,B29K(N^{ε}Octadecanedioyl-γGlu-OEG-OEG),desB30 human insulin,
A14E,A21G,B25H,desB27,B29K(N^{ε}Eicosanedioyl-γGlu-OEG-OEG),desB30 human insulin,
A14E,A21G,B25H,B29K(N^{ε}Octadecanedioyl-γGlu-OEG-OEG),desB30 human insulin,
A14E,A21G,B25H,B29K(N^{ε}Eicosanedioyl-γGlu-OEG-OEG),desB30 human insulin,
A14E,A21G,B25H,B29K(N^{ε}Eicosanedioyl-γGlu),desB30 human insulin,
A14E,A21G,B25H,B29K(N^{ε}Eicosanedioyl),desB30 human insulin,
A14E,A21G,B25H,B29K(N^{ε}Octadecanedioyl-γGlu),desB30 human insulin,
A14E,A21G,B25H,B29K(N^{ε}Octadecanedioyl),desB30 human insulin,
A14E,B25H,B26G,B27G,B28G,B29K(N^{ε}Octadecanedioyl-γGlu),desB30 human insulin,
A14E,B25H,B26G,B27G,B28G,B29K(N^{ε}Octadecanedioyl),desB30 human insulin,
A14E,B25H,B26G,B27G,B28G,B29K(N^{ε}Eicosanedioyl-γGlu),desB30 human insulin,
A14E,B25H,B26G,B27G,B28G,B29K(N^{ε}Eicosanedioyl),desB30 human insulin,
A1G(N^{α}Octadecandioyl-γGlu),A14E,B25H,B26G,B27G,B28G,desB30 human insulin,
A1G(N^{α}Eicosanedioyl-γGlu),A14E,B25H,B26G,B27G,B28G,desB30 human insulin,
A1G(N^{α}Octadecandioyl-γGlu),A14E,B25H,B26G,B27G,B28G,B29R,desB30 human
A1G(N^{α}Eicosanedioyl-γGlu),A14E,B25H,B26G,B27G,B28G,B29R,desB30 human insulin,
A1G(N^{α}Octadecandioyl),A14E,B25H,B26G,B27G,B28G,desB30 human insulin,
A1G(N^{α}Eicosanedioyl),A14E,B25H,B26G,B27G,B28G,desB30 human insulin,
A1G(N^{α}Octadecandioyl),A14E,B25H,B26G,B27G,B28G,B29R,desB30 human insulin and
A1G(N^{α}Eicosanedioyl),A14E,B25H,B26G,B27G,B28G,B29R,desB30 human insulin.
227. In one embodiment a tablet core according to the present invention comprises an acylated insulin selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B3C,B25H,B29K(N^{ε}Octadecanedioyl),desB30 human insulin,
A10C,A14E,B3C,B25H,B29K(N^{ε}Octadecanedioyl-γGlu),desB30 human insulin,
A10C,A14E,desB1,B4C,B25H,B29K(N^{ε}Octadecanedioyl-γGlu-OEG-OEG),desB30 human insulin,
A10C,A14H,B4C,B25H,B29K(N^{ε}Octadecanedioyl-γGlu-OEG-OEG),desB30 human insulin,
A10C,A14E,B3C,B25H,B29K(N^{ε}Eicosanedioyl-γGlu-OEG-OEG),desB30 human insulin,
A10C,A14E,B1C, B25H,B29K(N^{ε}Eicosanedioyl-γGlu-OEG-OEG),desB30 human insulin,
A10C,A14E,B4C B25H,B29K(N^{ε}Octadecanedioyl-γGlu),desB30 human insulin,
A10C,A14E, B3C,B25H,B29K(N^{ε}Octadecanedioyl-γGlu-OEG-OEG),desB30 human insulin,
A10C,A14E,B3C,B25H,B29K(N^{ε}Octadecanedioyl-γGlu-γGlu),desB30 human insulin,
A10C,A14E,B4C,B25H,desB27,B29K(N^{ε}Octadecanedioyl-γGlu),desB30 human insulin,
A10C,A14E,B4C,B25H,B29K(N^{ε}Octadecanedioyl),desB30 human insulin,
A10C,A14E,B4C,B25H,B29K(N^{ε}Octadecanedioyl-γGlu-γGlu),desB30 human
A10C,A14E,B2C,B25H,B29K(N^{ε}Octadecanedioyl-γGlu-OEG-OEG),desB30 human insulin,
A10C,A14E,B1C,B25H,B29K(N^{ε}Octadecanedioyl-γGlu-OEG-OEG),desB30 human insulin,
A10C,A14E,B3C,B16H,B25H,B29K(N^{ε}Eicosanedioyl-γGlu-OEG-OEG),desB30 human insulin,
A10C,A14E, B4C,B25H,B29K(N^{ε}Myristyl),desB30 human insulin,
A10C,B4C, B29K(N^{ε}Myristyl),desB30 human insulin,
A10C,A14E,B3C,B25H,desB27,B29K(N(eps)octadecanedioyl-γGlu),desB30 human insulin,
A10C,A14E,B3C,B25H,desB27,B29K(N(eps)octadecanedioyl-γGlu-OEG-OEG),desB30 human insulin,
A10C,A14E,B3C,B25H,B29K(N^{ε}Eicosanedioyl-γGlu),desB30 human insulin,
A10C,A14E,B4C,B25H,B29K(N(eps)eicosanedioyl-γGlu-OEG-OEG),desB30 human insulin,
A10C,A14E,B3C,B25H,desB27,B29K(N(eps)eicosanedioyl-γGlu),desB30 human insulin,
A10C,A14E,B3C,B25H,desB27, B29K(N(eps)eicosanedioyl-γGlu-OEG-OEG),desB30 human insulin,
A10C,A14E, 4C,B25H,B29K(N^{ε}Hexadecanedioyl-γGlu),desB30 human insulin,
A10C,A14E,B4C,B25H,B29K(N^{ε}Hexadecanedioyl-γGlu-OEG-OEG),desB30 human insulin,
A10C,A14E,B4C,B25H,B29K(N^{ε}Hexadecanedioyl),desB30 human insulin,
A10C,A14E,B4C,B25H,B29K(N^{ε}Hexadecanedioyl-γGlu-γGlu),desB30 human insulin,
A10C,A14E,B4C,B25H,desB27,B29K(N^{ε}Octadecanedioyl-γGlu-OEG-OEG),desB30 human insulinA10C,A14E,B4C,B25H,desB27,B29K(N^{ε}Octadecanedioyl-γGlu-γGlu),desB30 human insulin,
A10C,A14E,B4C,B25H,desB27,B29K(N^{ε}Hexadecanedioyl-γGlu-OEG-OEG),desB30 human insulin,A10C,A14E,B4C,B25H,desB27,B29K(N^{ε}Hexadecanedioyl-γGlu),desB30 human insulin,
A10C,A14E,B3C,B25H,B29K(N^{ε}Hexadecanedioyl-γGlu),desB30 human insulin,
A10C,A14E,B3C,B25H,B29K(N^{ε}Hexadecanedioyl-γGlu-OEG-OEG),desB30 human insulin,
A10C,A14E,B2C,B25H,B29K(N^{ε}Hexadecanedioyl-γGlu-OEG-OEG),desB30 human insulin,
A10C,A14E,B2C,B25H,B29K(N^{ε}Hexadecanedioyl-γGlu),desB30 human insulin,
A10C,A14E,B2C,B25H,B29K(N^{ε}Octadecanedioyl-γGlu),desB30 human insulin,
A10C,A14E,B1C,B25H,B29K(N^{ε}Octadecanedioyl-γGlu),desB30 human insulin,
A10C,A14E,B1C,B25H,B29K(N^{ε}Hexadecanedioyl-γGlu-OEG-OEG),desB30 human insulin,
A10C,A14E,B1C,B25H,B29K(N^{ε}Hexadecanedioyl-γGlu),desB30 human insulin,
A10C,B1C,B29K(N^{ε}Hexadecanedioyl-γGlu-OEG-OEG),desB30 human insulin,
A10C,B1C,B29K(N^{ε}Hexadecanedioyl-γGlu),desB30 human insulin,
A10C,B1C,B29K(N^{ε}Octadecanedioyl-γGlu-OEG-OEG),desB30 human insulin,
A10C,B1C,B29K(N^{ε}Octadecanedioyl-γGlu),desB30 human insulin,
A10C,B2C,B29K(N^{ε}Octadecanedioyl-γGlu-OEG-OEG),desB30 human insulin,
A10C,B2C,B29K(N^{ε}Octadecanedioyl-γGlu),desB30 human insulin,
A10C,B2C,B29K(N^{ε}Hexadecanedioyl-γGlu-OEG-OEG),desB30 human insulin,
A10C,B2C,B29K(N^{ε}Hexadecanedioyl-γGlu),desB30 human insulin,
A10C,B3C,B29K(N^{ε}Hexadecanedioyl-γGlu),desB30 human insulin,
10C,B3C B29K(N^{ε}Hexadecanedioyl-γGlu-OEG-OEG),desB30 human insulin,
A10C,B3C,B29K(N^{ε}Octadecanedioyl-γGlu-OEG-OEG),desB30 human insulin,
A10C,B3C,B29K(N^{ε}Octadecanedioyl-γGlu),desB30 human insulin,
A10C,B4C,B29K(N^{ε}Octadecanedioyl-γGlu),desB30 human insulin,
A10C,B4C,B29K(N^{ε}Octadecanedioyl-γGlu-OEG-OEG),desB30 human insulin,
A10C,B4C B29K(N^{ε}Hexadecanedioyl-γGlu-OEG-OEG),desB30 human insulin,
A10C,B4C,B29K(N^{ε}Hexadecanedioyl-γGlu),desB30 human insulin,
A10C,A14E,B1C,B16H,B25H,B29K(N^{ε}eicosanedioyl-γGlu-OEG-OEG),desB30 human insulin,
A10C,A14E,B1C,B16H,B25H,B29K(N^{ε}eicosanedioyl-γGlu),desB30 human insulin,
A10C,A14E,B1C,B16H,B25H,B29K(N^{ε}Octadecanedioyl-γGlu-OEG-OEG),desB30 human insulin,
A10C,A14E,B1C,B16H,B25H,B29K(N^{ε}Octadecanedioyl-γGlu),desB30 human
A10C A14E,B1C,B16H,B25H, B29K(N^{ε}Hexadecanedioyl-γGlu),desB30 human insulin,
A10C,A14E,B1C,B16H, B25H,B29K(N^{ε}Hexadecanedioyl-γGlu-OEG-OEG),desB30 human insulin,
A10C,A14E,B2C,B16H,B25H,B29K(N^{ε}Hexadecanedioyl-γGlu-OEG-OEG),desB30 human insulin,
A10C,A14E,B2C,B16H,B25H,B29K(N^{ε}Hexadecanedioyl-γGlu),desB30 human insulin,
A10C,A14E,B2C,B16H,B25H,B29K(N^{ε}Octadecanedioyl-γGlu),desB30 human insulin,
A10C,A14E,B2C,B16H,B25H,B29K(N^{ε}Octadecanedioyl-γGlu-OEG-OEG),desB30 human insulin,
A10C,A14E,B2C,B16H,B25H,B29K(N^{ε}Eicosanedioyl-γGlu-OEG-OEG),desB30 human insulin,
A10C,A14E,B2C,B16H,B25H,B29K(N^{ε}Eicosanedioyl-γGlu),desB30 human insulin,
A10C,A14E,B3C,B16H,B25H,B29K(N^{ε}Eicosanedioyl-γGlu),desB30 human insulin,
A10C,A14E,B3C,B16H,B25H,B29K(N^{ε}Octadecanedioyl-γGlu-OEG-OEG),desB30 human insulin,
A10C,A14E,B3C,B16H, B25H,B29K(N^{ε}Octadecanedioyl-γGlu),desB30 human
insulinA10C,A14E,B3C,B16H,B25H,B29K(N^{ε}Hexadecanedioyl-γGlu),desB30 human insulin,
A10C,A14E,B3C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu),desB30 human insulin,
A10C,A14E,B4C,B16H,B25H,B29K(N^{ε}Octadecanedioyl-γGlu),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30
A10C,A14E,B4C,B16H,B25H,B29K(N^{ε}Eicosanedioyl-γGlu),desB30 human insulin,
A10C,A14E,B1C,B25H,B29K(N(eps)eicosanedioyl-γGlu),desB30 human insulin,
A10C,A14E,B2C,B25H,B29K(N(eps)eicosanedioyl-γGlu),desB30 human insulin,
A10C,A14E,B2C,B25H,B29K(N(eps)eicosanedioyl-γGlu-OEG-OEG),desB30 human insulin,
A10C,A14E,B4C,B25H,desB27,B29K(N(eps)eicosanedioyl-γGlu),desB30 human insulin,
A10C,A14E,B4C,B25H,desB27,B29K(N(eps)eicosanedioyl-γGlu-OEG-OEG),desB30 human insulin,
A10C,A14E,B4C,B25H,B29K(N(eps)eicosanedioyl-γGlu),desB30 human insulin,
A10C,A14E,B3C,B25H,desB27,B29K(N(eps)hexadecanedioyl-γGlu),desB30 human insulin,
A10C,A14E,B3C,B25H,desB27,B29K(N(eps)hexadecanedioyl-γGlu-OEG-OEG),desB30 human insulin,
A10C,A14E,B3C,desB27,B29K(N(eps)hexadecanedioyl-γGlu),desB30 human insulin,
A10C,A14E,B3C,desB27,B29K(N(eps)hexadecanedioyl-γGlu-OEG-OEG),desB30 human insulin,
A10C,A14E,B3C,desB27,B29K(N(eps)octadecanedioyl-γGlu),desB30 human insulin,
A10C,A14E,B3C,desB27,B29K(N(eps)octadecanedioyl-γGlu-OEG-OEG),desB30 human insulin,
A10C,A14E,B3C,desB27,B29K(N(eps)eicosanedioyl-γGlu),desB30 human insulin,
A10C,A14E,B3C,desB27,B29K(N(eps)eicosanedioyl-γGlu-OEG-OEG),desB30 human insulin,
A10C,A14E,B3C,B16H,B25H,B29K(N(eps)eicosanedioyl-γGlu-γGlu),desB30 human insulin,
A10C,A14E,B3C,B16E,B25H,B29K(N(eps)eicosanedioyl-γGlu-OEG-OEG),desB30 human insulin,
A10C,A14E,B4C,B16E,B25H,B29K(N(eps)eicosanedioyl-γGlu-OEG-OEG),desB30 human insulin,
A10C,A14E,B3C,B16H,B25H,B29K(N(eps)eicosanedioyl-γGlu-γGlu),desB30 human insulin and
A10C,A14E,B4C,B16E,B25H,B29K(N(eps)eicosanedioyl-γGlu-γGlu),desB30 human insulin.
228. The pharmaceutical composition according to any one of the preceding aspects, wherein said acylated insulin is selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B3C,B25H,B29K(N^{ε}Octadecanedioyl),desB30 human insulin,
A10C,A14E,B3C,B25H,B29K(N^{ε}Octadecanedioyl-γGlu),desB30 human insulin,
A10C,A14H,B4C,B25H,B29K(N^{ε}Octadecanedioyl-γGlu-OEG-OEG),desB30 human insulin,
A10C,A14E,B3C,B25H,B29K(N^{ε}Eicosanedioyl-γGlu-OEG-OEG),desB30 human insulin,
A10C,A14E,B4C B25H,B29K(N^{ε}Octadecanedioyl-γGlu),desB30 human insulin,
A10C,A14E, B3C,B25H,B29K(N^{ε}Octadecanedioyl-γGlu-OEG-OEG),desB30 human insulin,
A10C,A14E,B3C,B25H,B29K(N^{ε}Octadecanedioyl-γGlu-γGlu),desB30 human insulin,
A10C,A14E,B4C,B25H,desB27,B29K(N^{ε}Octadecanedioyl-γGlu),desB30 human insulin,
A10C,A14E,B4C,B25H,B29K(N^{ε}Octadecanedioyl),desB30 human insulin,
A10C,A14E,B4C,B25H,B29K(N^{ε}Octadecanedioyl-γGlu-γGlu),desB30 human insulin,
A10C,A14E,B3C,B16H,B25H,B29K(N^{ε}Eicosanedioyl-γGlu-OEG-OEG),desB30 human insulin,
A10C,A14E, B4C,B25H,B29K(N^{ε}Myristyl),desB30 human insulin,
A10C,B4C, B29K(N^{ε}Myristyl),desB30 human insulin,
A10C,A14E,B3C,B25H,desB27,B29K(N(eps)octadecanedioyl-γGlu),desB30 human insulin,
A10C,A14E,B3C,B25H,desB27,B29K(N(eps)octadecanedioyl-γGlu-OEG-OEG),desB30 human insulin,
A10C,A14E,B3C,B25H,B29K(N^{ε}Eicosanedioyl-γGlu),desB30 human insulin,
A10C,A14E,B4C,B25H,B29K(N(eps)eicosanedioyl-γGlu-OEG-OEG),desB30 human insulin,
A10C,A14E,B3C,B25H,desB27,B29K(N(eps)eicosanedioyl-γGlu),desB30 human insulin,
A10C,A14E,B3C,B25H,desB27, B29K(N(eps)eicosanedioyl-γGlu-OEG-OEG),desB30 human insulin,
A10C,A14E, 4C,B25H,B29K(N^{ε}Hexadecanedioyl-γGlu),desB30 human insulin,
A10C,A14E,B4C,B25H,B29K(N^{ε}Hexadecanedioyl-γGlu-OEG-OEG),desB30 human insulin,
A10C,A14E,B4C,B25H,B29K(N^{ε}Hexadecanedioyl),desB30 human insulin,
A10C,A14E,B4C,B25H,B29K(N^{ε}Hexadecanedioyl-γGlu-γGlu),desB30 human insulin,
A10C,A14E,B4C,B25H,desB27,B29K(N^{ε}Octadecanedioyl-γGlu-OEG-OEG),desB30 human insulin,A10C,A14E,B4C,B25H,desB27,B29K(N^{ε}Octadecanedioyl-γGlu-γGlu),desB30 human insulin,
A10C,A14E,B4C,B25H,desB27,B29K(N^{ε}Hexadecanedioyl-γGlu-OEG-OEG),desB30 human insulin,A10C,A14E,B4C,B25H,desB27,B29K(N^{ε}Hexadecanedioyl-γGlu),desB30 human insulin,
A10C,A14E,B3C,B25H,B29K(N^{ε}Hexadecanedioyl-γGlu),desB30 human insulin,
A10C,A14E,B3C,B25H,B29K(N^{ε}Hexadecanedioyl-γGlu-OEG-OEG),desB30 human insulin,
A10C,B3C,B29K(N^{ε}Hexadecanedioyl-γGlu),desB30 human insulin,
10C,B3C B29K(N^{ε}Hexadecanedioyl-γGlu-OEG-OEG),desB30 human insulin,
A10C,B3C,B29K(N^{ε}Octadecanedioyl-γGlu-OEG-OEG),desB30 human insulin,
A10C,B3C,B29K(N^{ε}Octadecanedioyl-γGlu),desB30 human insulin,
A10C,B4C,B29K(N^{ε}Octadecanedioyl-γGlu),desB30 human insulin,
A10C,B4C,B29K(N^{ε}Octadecanedioyl-γGlu-OEG-OEG),desB30 human insulin,
A10C,B4C B29K(N^{ε}Hexadecanedioyl-γGlu-OEG-OEG),desB30 human insulin,
A10C,B4C,B29K(N^{ε}Hexadecanedioyl-γGlu),desB30 human insulin,
A10C,A14E,B3C,B16H,B25H,B29K(N^{ε}Eicosanedioyl-γGlu),desB30 human insulin,
A10C,A14E,B3C,B16H,B25H,B29K(N^{ε}Octadecanedioyl-γGlu-OEG-OEG),desB30
A10C,A14E,B3C,B16H, B25H,B29K(N^{ε}Octadecanedioyl-γGlu),desB30 human insulinA10C,A14E,B3C,B16H,B25H,B29K(N^{ε}Hexadecanedioyl-γGlu),desB30 human insulin,
A10C,A14E,B3C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu),desB30 human insulin,
A10C,A14E,B4C,B16H,B25H,B29K(N^{ε}Octadecanedioyl-γGlu),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin,
A10C,A14E,B4C,B16H,B25H,B29K(N^{ε}Eicosanedioyl-γGlu),desB30 human insulin,
A10C,A14E,B4C,B25H,desB27,B29K(N(eps)eicosanedioyl-γGlu),desB30 human insulin,
A10C,A14E,B4C,B25H,desB27,B29K(N(eps)eicosanedioyl-γGlu-OEG-OEG),desB30 human insulin,
A10C,A14E,B4C,B25H,B29K(N(eps)eicosanedioyl-γGlu),desB30 human insulin,
A10C,A14E,B3C,B25H,desB27,B29K(N(eps)hexadecanedioyl-γGlu),desB30 human insulin,
A10C,A14E,B3C,B25H,desB27,B29K(N(eps)hexadecanedioyl-γGlu-OEG-OEG),desB30 human insulin,
A10C,A14E,B3C,desB27,B29K(N(eps)hexadecanedioyl-γGlu),desB30 human insulin,
A10C,A14E,B3C,desB27,B29K(N(eps)hexadecanedioyl-γGlu-OEG-OEG),desB30 human insulin,
A10C,A14E,B3C,desB27,B29K(N(eps)octadecanedioyl-γGlu),desB30 human insulin,
A10C,A14E,B3C,desB27,B29K(N(eps)octadecanedioyl-γGlu-OEG-OEG),desB30
A10C,A14E,B3C,desB27,B29K(N(eps)eicosanedioyl-γGlu),desB30 human insulin,
A10C,A14E,B3C,desB27,B29K(N(eps)eicosanedioyl-γGlu-OEG-OEG),desB30 human insulin,
A10C,A14E,B3C,B16H,B25H,B29K(N(eps)eicosanedioyl-γGlu-γGlu),desB30 human insulin,
A10C,A14E,B3C,B16E,B25H,B29K(N(eps)eicosanedioyl-γGlu-OEG-OEG),desB30 human insulin,
A10C,A14E,B4C,B16E,B25H,B29K(N(eps)eicosanedioyl-γGlu-OEG-OEG),desB30 human insulin,
A10C,A14E,B3C,B16H,B25H,B29K(N(eps)eicosanedioyl-γGlu-γGlu),desB30 human insulin and A10C,A14E,B4C,B16E,B25H,B29K(N(eps)eicosanedioyl-γGlu-γGlu),desB30 human insulin.
229. The pharmaceutical composition according to any one of the preceding aspects, wherein said acylated insulin is selected from the group consisting of:
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N(eps)Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin,
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B4C,B25H,desB27,B29K(N^{ε}Octadecanedioyl-γGlu),desB30 human
A10C,A14E,B3C,B16H,B25H,B29K(N^{ε}Eicosanedioyl-γGlu-OEG-OEG),desB30 human insulin,
A10C,A14E,B3C,B25H,desB27,B29K(N(eps)octadecanedioyl-γGlu-OEG-OEG),desB30 human insulin,
A10C,A14E,B3C,B25H,desB27, B29K(N(eps)eicosanedioyl-γGlu-OEG-OEG),desB30 human insulin,
A10C,A14E,B3C,B16H,B25H,B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N(eps)eicosanedioyl-γGlu-OEG-OEG),desB30 human insulin.
230. The pharmaceutical composition according to any one of the preceding aspects, wherein said acylated insulin is selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N(eps)octadecanedioyl-γGlu-OEG-OEG),desB30 human insulin,
A10C,A14E,B3C,B25H,desB27, B29K(N(eps)eicosanedioyl-γGlu-OEG-OEG),desB30 human insulin,
A10C,A14E,B3C,B16H,B25H,B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30
A10C,A14E,B4C,B16H B25H,B29K(N^{ε}Eicosanedioyl-γGlu-OEG-OEG),desB30 human insulin and
A10C,A14E,B4C,B25H,desB27,B29K(N(eps)eicosanedioyl-γGlu-OEG-OEG),desB30 human insulin.
231. The pharmaceutical composition according to any one of the preceding aspects, wherein said acylated insulin is selected from the group consisting of:
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N(eps)Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin.
232. The pharmaceutical composition according to any one of the preceding aspects for use as a medicament.
233. The pharmaceutical composition according to any one the preceding aspects for use in treating diabetes mellitus.
234. The pharmaceutical composition according to any one the preceding aspects for use in treating type 1 and/or type 2 diabetes mellitus.
235. The pharmaceutical composition according to any one of the aspects 1-234 provided in a kit comprising said pharmaceutical composition in a blister back and instructions for use.
236. The pharmaceutical composition according to any one of the aspects 1-234 provided in a kit comprising said pharmaceutical composition in a container and instructions for use.
237. The pharmaceutical composition according to any one of the aspects 1-234 provided in a kit comprising said pharmaceutical composition in the form of one or more tablets, capsules or multiparticulate system compressed into tablets provided in a container accompanied of instructions for use.
238. A method for producing a pharmaceutical composition according to any one the preceding aspects, comprising the steps of preparing a tablet core and coating said polyvinyl alcohol coating on said outer surface of said one or more tablet core.
239. The method according to aspect 238, wherein said one or more tablet core is in the form of a uniform tablet, a single or multi-layered tablet, a multiparticulate system, a capsule, a tablet contained in a capsule, a multiparticulate system comprising multiple tablets contained in a capsule or a multiparticulate system comprising multiple tablets contained in a tablet.

The invention may also solve further problems that will be apparent from the disclosure of the exemplary embodiments.

### Materials and Methods

### List of Abbreviations

βAla is beta-alanyl,
Aoc is 8-aminooctanoic acid,
tBu is tert-butyl,
CV is column volumes,
DCM is dichloromethane,
DIC is diisopropylcarbodiimide,

### DIPEA=DIEA is N,N-disopropylethylamine,

### DMF is N,N-dimethylformamide,

DMSO is dimethyl sulphoxide,
EtOAc is ethyl acetate,
Fmoc is 9-fluorenylmethyloxycarbonyl,
γGlu is gamma L-glutamyl,
HCl is hydrochloric acid,
HOBt is 1-hydroxybenzotriazole,

### NMP is N-methylpyrrolidone,

MeCN is acetonitrile,
OEG is [2-(2-aminoethoxy)ethoxy]ethylcarbonyl,
Su is succinimidyl-1-yl=2,5-dioxo-pyrrolidin-1-yl,
OSu is succinimidyl-1-yloxy=2,5-dioxo-pyrrolidin-1-yloxy,
RPC is reverse phase chromatography,
RT is room temperature,
TFA is trifluoroacetic acid,
THF is tetrahydrofuran,
TNBS is 2,4,6-trinitrobenzenesulfonic acid,
TRIS is tris(hydroxymethyl)aminomethane
TSTU is O—(N-succinimidyl)-1,1,3,3-tetramethyluronium tetrafluoroborate.

### Method 1: General Methods of Preparation of Insulin

The production of polypeptides and peptides such as insulin is well known in the art. Insulin may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. Insulin may also be produced by a method which comprises culturing a host cell containing a DNA sequence encoding the insulin and capable of expressing the insulin in a suitable nutrient medium under conditions permitting the expression of the peptide. For insulin comprising non-natural amino acid residues, the recombinant cell should be modified such that the non-natural amino acids are incorporated into the insulin, for instance by use of tRNA mutants.

To effect covalent attachment of side chain(s) to the insulin, the hydroxyl end group of the side chain is provided in activated form, i.e. with reactive functional groups. Suitable activated polymer molecules are commercially available, e.g. from Shearwater Corp., Huntsville, Ala., USA, or from PolyMASC Pharmaceuticals plc, UK. Alternatively, the side chains may be activated by conventional methods known in the art, e.g. as disclosed in WO 09/115469.

The conjugation of the insulin and the activated side chain is conducted by use of any conventional method, e.g. as described in the following references (which also describe suitable methods for activation of side chains): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the insulin (examples of which are given further above), as well as the functional groups of the side chain (e.g. being amine, hydroxyl, carboxyl, aldehyde, sulfydryl, succinimidyl, maleimide, vinysulfone or haloacetate).

The following examples are offered by way of illustration, not by limitation. The preparation of the acylated insulins used in the pharmaceutical compositions of the present invention is described by the chemical reactions described in their general applicability to the preparation. Occasionally, the reaction may not be applicable as described to each compound included within the disclosed scope of the invention. The acylated insulins for which this occurs will be readily recognised by those skilled in the art. In these cases the reactions may be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding acylated insulins for use in the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The acylated and non-acylated insulins used in the invention may be purified by employing one or more of the following procedures which are typical within the art. These procedures may—if needed—be modified with regard to gradients, pH, salts, concentrations, flow, columns and so forth. Depending on factors such as impurity profile, solubility of the insulins in question etcetera, these modifications may readily be recognised and made by a person skilled in the art. After acidic HPLC or desalting, the acylated insulin is isolated by lyophilisation of the pure fractions.

After neutral HPLC or anion exchange chromatography, the compounds are desalted, precipitated at isoelectric pH, or purified by acidic HPLC.

### Method 2: Typical Insulin Purification Procedures

The HPLC system is a Gilson system consisting of the following: Model 215 Liquid handler, Model 322-H2 Pump and a Model 155 UV Dector. Detection is typically at 210 nm and 280 nm.

The Äkta Purifier FPLC system (GE Health Care) consists of the following: Model P-900 Pump, Model UV-900 UV detector, Model pH/C-900 pH and conductivity detector, Model Frac-950 Fraction collector. UV detection is typically at 214 nm,
254 nm and 276 nm. The Akta Explorer Air FPLC system (Amersham BioGE Health Caresciences) consists of the following: Model P-900 Pump, Model UV-900 UV detector, Model pH/C-900 pH and conductivity detector, Model Frac-950 Fraction collector. UV detection is typically at 214 nm, 254 nm and 276 nm

### Acidic HPLC:

### Column: Phenomenex, Gemini, 5μ, C18, 110 Å, 250×30 cm

Flow: 20 ml/min′
Eluent: A: 0.1% TFA in water B: 0.1% TFA in CH₃CN

### Gradient: 0-7.5 min: 10% B

### 7.5-87.5 min: 10% B to 60% B

### 87.5-92.5 min: 60% B

### 92.5-97.5 min: 60% B to 100% B

### Neutral HPLC:

### Column: Phenomenex, Gemini, C18, 5 μm 250×30.00 mm, 110 Å

Flow: 20 ml/min
Eluent: A: 20% CH₃CN in aqueous 10 mM TRIS+15 mM (NH₄)SO₄
pH=7.3
B: 80% CH₃CN, 20% water

### Gradient: 0-7.5 min: 0% B

### 7.5-52.5 min: 0% B to 60% B

### 52.5-57.5 min: 60% B

### 57.5-58 min: 60% B to 100% B

### 58-60 min: 100% B

### 60-63 min: 10% B

### Anion Exchange Chromatography:

### Column: RessourceQ, 6 ml,

Flow: 6 ml/min
Buffer A: 0.09% NH₄HCO₃, 0.25% NH₄OAc, 42.5% ethanol pH 8.4
Buffer B: 0.09% NH₄HCO₃, 2.5% NH₄OAc, 42.5% ethanol pH 8.4
Gradient: 100% A to 100% B during 30 CV

### Column: Source 30Q, 30×250 mm

Flow: 80 ml/min

### Buffer A: 15 mM TRIS, 30 mM Ammoniumacetat i 50% Ethanol,

pH 7.5 (1.25 mS/cm)

### Buffer B: 15 mM TRIS, 300 mM Ammoniumacetat i 50% Ethanol

pH 7.5 (7.7 mS/cm)
Gradient: 15% B to 70% B over 40 CV

### Desalting:

### Column: Daiso 200 Å 15 um FeFgel 304, 30×250 mm

Buffer A: 20 v/v % Ethanol, 0.2% acetic acid
Buffer B: 80% v/v % Ethanol, 0.2% acetic acid
Gradient: 0-80% B over 1.5 CV
Flow: 80 ml/min

### Column: HiPrep 26/10

Flow: 10 ml/min,

### Gradient: 6 CV

Buffer: 10 mM NH₄HCO₃

### General Procedure for the Solid Phase Synthesis of Acylation Reagents of the General Formula CHEM 3:

Acy-AA1ₙ-AA2ₘ-AA3ₚ-Act, CHEM 3:

wherein AcyAA1, AA2, AA3, n, m, and p are as defined above and Act is the leaving group of an active ester, such as N-hydroxysuccinimide (OSu), or 1-hydroxybenzotriazole, and
wherein carboxylic acids within the Acy and AA2 moieties of the acyl moiety are protected as tert-butyl esters.

Insulin analogue or derivatives of general formula CHEM 3 used according to the invention may be synthesised on solid support using procedures well known to skilled persons in the art of solid phase peptide synthesis. This procedure comprises attachment of a Fmoc protected amino acid to a polystyrene 2-chloro-tritylchloride resin. The attachment can, e.g., be accomplished using the free N-protected amino acid in the presence of a tertiary amine, like triethyl amine or N,N-diisopropylethylamine (see references below). The C-terminal end (which is attached to the resin) of this amino acid is at the end of the synthetic sequence being coupled to the parent insulins of the invention. After attachment of the Fmoc amino acid to the resin, the Fmoc group is deprotected using, e.g., secondary amines, like piperidine or diethyl amine, followed by coupling of another (or the same) Fmoc protected amino acid and deprotection. The synthetic sequence is terminated by coupling of mono-tert-butyl protected fatty (α, ω) diacids, like hexadecanedioic, heptadecanedioic, octadecanedioic or eicosanedioic acid mono-tert-butyl esters. Cleavage of the compounds from the resin is accomplished using diluted acid like 0.5-5% TFA/DCM (trifluoroacetic acid in dichloromethane), acetic acid (e.g., 10% in DCM, or HOAc/triflouro-ethanol/DCM 1:1:8), or hecafluoroisopropanol in DCM (See, e.g., “Organic Synthesis on Solid Phase”, F. Z. Dorwald, Wiley-VCH, 2000. ISBN 3-527-29950-5, “Peptides: Chemistry and Biology”, N. Sewald & H.-D. Jakubke, Wiley-VCH, 2002, ISBN 3-527-30405-3 or “The Combinatorial Chemistry Catalog” 1999, Novabiochem A G, and references cited therein). This ensures that tert-butyl esters present in the compounds as carboxylic acid protecting groups are not deprotected. Finally, the C-terminal carboxy group (liberated from the resin) is activated, e.g., as the N-hydroxysuccinimide ester (OSu) and used either directly or after purification as coupling reagent in attachment to parent insulins of the invention. This procedure is described in example 9 in, WO09115469.

Alternatively, the acylation reagents of the general formula CHEM 3 above may be prepared by solution phase synthesis as described below.

Mono-tert-butyl protected fatty diacids, such as hexadecanedioic, heptadecanedioic, octadecanedioic or eicosanedioic acid mono-tert-butyl esters are activated, e.g., as OSu-esters as described below or as any other activated ester known to those skilled in the art, such as HOBt- or HOAt-esters. This active ester is coupled with one of the amino acids AA1, mono-tert-butyl protected AA2, or AA3 in a suitable solvent such as THF, DMF, NMP (or a solvent mixture) in the presence of a suitable base, such as DIPEA or triethylamine. The intermediate is isolated, e.g., by extractive procedures or by chromatographic procedures. The resulting intermediate is again subjected to activation (as described above) and to coupling with one of the amino acids AA1, mono-tert-butyl protected AA2, or AA3 as described above. This procedure is repeated until the desired protected intermediate Acy-AA1ₙ-AA2ₘ-AA3ₚ-OH is obtained. This is in turn activated to afford the acylation reagents of the general formula CHEM 3 Acy-AA1ₙ-AA2ₘ-AA3ₚ-Act. This procedure is described in example 11 in WO09115469. The acylation reagents prepared by any of the above methods may be (tert-butyl) de-protected after activation as OSu esters. This may be done by TFA treatment of the OSu-activated tert-butyl protected acylation reagent. After acylation of any insulin, the resulting unprotected acylated protease stabilized insulin of the invention is obtained. This procedure is described in example 16 in WO09115469.

If the reagents prepared by any of the above methods are not (tert-butyl) de-protected after activation as OSu esters, acylation of any insulin affords the corresponding tert-butyl protected acylated insulin of the invention. In order to obtain the unprotected acylated insulin of the invention, the protected insulin is to be de-protected. This may be done by TFA treatment to afford the unprotected acylated insulin of the invention. This procedure is described in example 1 in WO05012347.

Methods for preparation of acylated insulins may be found in WO09115469. In one embodiment of the invention, acylated insulin used in a composition according to the present invention, wherein the insulin is an acylated, protease stabilised insulin.

Method 3: Preparing a Tablet Core Weighing Between about 100 TO 900 mg (i.e. Midi- or Monolith Tablets Cores) According to this Invention

The tablets cores according to this invention weighing between about 100 to about 900 mg are prepared so that a person skilled in the art of pharmaceutical tablet production easily can make the tablets. The formulation of a tablet core material according to the present invention was performed as outlined here, this example concerns formulations of the present invention comprising:

| | |
|---|---|
| Acylated insulin | 1.17% (w/w) |
| Sodium decanoate (i.e. sodium salt of capric acid) | 77.00% (w/w) |
| Sorbitol | 21.33% (w/w) |
| Stearic acid | 0.50% (w/w) |

When 100 g of tablet core material comprising acylated insulin, sodium caprate (i.e. sodium salt of capric acid), sorbitol and stearic acid was manufactured according to the above listed ingredients and in the corresponding ratios, the following steps were used:

The procedure was performed as follows:

Insulin powder was put through a sieve with a mesh size of 0.25 mm. After sieving the correct amount of acylated insulin was weighed. Sorbitol powder was put through a sieve with a mesh size of 0.5 mm. After sieving the correct amount was weighed.

In a small container insulin and sorbitol was mixed. An amount of sorbitol equivalent to the amount of acylated insulin was added to said container and stirred by hand. Then the double amount of sorbitol relative to the previous addition was added and stirred by hand until insulin and all sorbitol were mixed well. This step was followed by a mechanical mixing in a Turbula-mixer to finalize the mixing to obtain a homogeneous powder.

Sodium salt of capric acid (in the form of granulate) was then added to the insulin-sorbitol powder according to equal volumes principle. This was done in two steps and finalized with a mechanical mixing step in a Turbula-mixer. Finally stearic acid was put through a sieve with a mesh size of 0.25 mm. Stearic acid was weighed and added to the powder and mixed mechanically.

The powder prepared was compressed into a tablet press to form tablets according to the insulin dose desired.

Method 4: Preparing a Tablet Core Weighing Between about 100 to about 900 mg (i.e. Midi- and/or Monolith Tablets) with a Polyvinyl Alcohol Coat, Such as Opadry®II Yellow from Colorcon® (as Sold in 2013)

To exemplify how midi-tablets and monolith-tablets are prepared, this method will describe the preparation of a monolith tablet. If smaller tablets, i.e. weighing less and thus having smaller dimensions are desired, the ingredients have to be adjusted accordingly to a lower total weight and compressed into the desired tablet dimensions. The powder prepared according to method 3 was compressed into a tablet press to form tablets for example weighing 710 mg. A tablet core prepared by this method was then coated with immediate release coating comprising polyvinyl alcohol. The coating solution was prepared by dispersing the 20 g immediate release coating material, comprising polyvinyl alcohol in 80 g pure water. The concentration of immediate release coating comprising polyvinyl alcohol in the coating solution was 20%-volume. Under intense mixing using a standard magnetic stirrer the polymer powder was added to the water. After addition of polymer the mixture was stirred at low intensity for 30 minutes. The resulting coating solution was sieved to remove lumps. The coating of tablet cores was performed in a pan coater or fluid bed coater. In a pan coater with the pan size of 8.5″, with a conventional patterned air Schlick spray nozzle with an orifice of 1.0 mm, an atomizing and pattern air pressure of 0.5 bar, inlet air temperature of 38° C. and air flow of 130 kg/hour, the coating was performed by pumping the polymer solution in through the nozzle. After addition of 4.5% (w/w) polymer distributed evenly on the tablet cores the spraying is stopped and the tablets are allowed to dry for up to 30 minutes inside the pan. The amount of coating polymer is adjusted to the surface area of the desired tablet weight and thus size.

Method 5: Preparing an Anionic Copolymer Coated Tablet Core Weighing Between about 100 to about 900 mg (i.e. Midi- and/or Monolith Tablets) According to this Invention or a Tablet Core with a Sub Coat

To exemplify how midi-tablets and monolith-tablets are prepared, this method will described the preparation of a monolith tablet. If smaller tablets, i.e. weighing less and thus having smaller dimensions are desired, the ingredients have to be adjusted in the same ratios between the ingredients to a lower total weight and compressed into the desired tablet dimensions.

If and when coating tablets with an anionic copolymer, the tablet core was prepared according to method 3 or method 4 and coated with an anionic copolymer as described below:

A tablet core according to method 3 or a tablet core with a sub coating according to method 4 was coated with an outer coating.

For this purpose polymers of the copolymer family denominated “methyl acrylate-co-methyl methacrylate-co-methacrylic acid” (Brand name Acryl-EZE®930 from Colorcon® (as sold in 2013)) were used.

200 g of an aqueous dispersion of methyl acrylate-co-methyl methacrylate-co-methacrylic acid (Brand name Acryl-EZE® 93O from Colorcon® (as sold in 2013)) was prepared in a beaker on a suitable stirring apparatus. 40 g of Acryl-EZE® 93O from Colorcon® (as sold in 2013) was mixed carefully into 160 ml purified water. The mixture was stirred for at least 30 minutes prior to a filtration through a 0.24 mm mesh filter to remove lumps. The coating of tablet cores with an inner coat as well as tablets without an inner coat was performed in a pan coater or fluid bed coater. In a pan coater with the pan size of 8.5″, with a conventional patterned air Schlick spray nozzle with an orifice of 1.0 mm, an atomizing and pattern air pressure of 0.5-0.6 bar, inlet air temperature of 36 C, air flow of 95 kg/hour, the coating was performed by pumping the polymer solution in through the nozzle. After addition of about 9% w/w polymer distributed evenly on the tablet cores including and excluding an inner coating as prepared in method 3 and 4, the spraying was stopped. The amount of coating material is adjusted to the surface area of the desired tablet weight and thus size.

### Method 6: Measuring Dissolution Rate In Vitro

In an appropriate dissolution apparatus e.g. USP dissolution apparatus 2 a standard dissolution test according to the pharmacopoeia may be performed to measure dissolution in-vitro. In this test the tablets were exposed to a dissolution medium with a pH of 6.8. Under stirring the tablet dissolution was followed by sampling at pre-defined time intervals and analysed by HPLC chromatography.

Method 7: Collecting Samples for Measuring Bioavailabilty, Tmax for a Composition from Beagle Dogs

The dogs were fasted overnight before the test, (no food—only tap water). The day before the experiment the dogs were weighed and dogs were taken out for a couple of hours.

On the day of the experiment the dogs were placed on test couch and a venflon 20 G was placed in v. cephalica. Blood samples were taken from the catheter. The venflon was removed 6 hours post dosing and the dogs were returned to their box, and offered exercise in the outside run. Hereafter the dogs were lead into a test room for blood sampling from v. jugularis (or v. cephalica).

Per Os Administration.

Blood samples for glucose and insulin were taken at: 0, 15, 30, 45, 60, 75, 90, 105, 120, 135, 150, 165, 180, 210, 240, 270, 300, 360, 480, 600, 720, 1440, 1800, 2880 and 4320 minutes.

The tablet was administrated right after the t=0 min sample was drawn. The tablet was placed in the back of the mouth so the dog would swallow the tablet without chewing it. After the dog had swallowed the tablet, 10 ml water was administrated into the mouth by a syringe.

Blood Sampling:

Before sampling the first drops of blood was collected on a tissue.

Approx. 800 μl blood was collected in 1.5 ml EDTA eppendorf tubes for plasma and a 10 μL capillary tube was filled with full blood for glucose analysis.

The EDTA blood samples were centrifuged at 4000×g (4° C.) for 4 min.

All samples were kept on wet ice until analysis or stored at −80° C. until analysis.

After each sampling were the Venflon flushed with 0.5 ml heparin (10 IU). Male Beagle dogs used weigh approximately from 12 to 18 kg approximately. Plasma samples were analysed by either sandwich immunoassay or Liquid chromatography-mass spectrometry. Plasma concentration-time profiles were analysed by non-compartmental pharmacokinetics analysis using WinNonlin Professional 5.2 (Pharsight Inc., Mountain View, Calif., USA).

### Method 8: Bioavailability and Pharmacokinetics Profile

Generally, the term bioavailability refers to the fraction of an administered dose of the active pharmaceutical ingredient (API), such as a derivative of the invention that reaches the systemic circulation unchanged. By definition, when an API is administered intravenously, its bioavailability is 100%. However, when it is administered via other routes (such as orally), its bioavailability decreases (due to degradation and/or incomplete absorption and first-pass metabolism). Knowledge about bioavailability is important when calculating dosages for non-intravenous routes of administration.

A plasma concentration versus time plot is made after both oral and intravenous administration. The absolute bioavailability (F) is the (AUC-oral divided by dose), divided by (AUC-intravenous divided by dose).

Increasing terminal half-life and/or decreasing of the clearance means that the compound in question is eliminated slower from the body. For the derivatives of the invention this entails an extended duration of pharmacological effect. Increased oral bioavailability means that a larger fraction of the dose administered orally reach the systemic circulation from where it may distribute to exhibit pharmacological effect.

The pharmacokinetic properties of the derivatives of the invention may suitably be determined in-vivo in pharmacokinetic (PK) studies. Such studies are conducted to evaluate how pharmaceutical compounds are absorbed, distributed, and eliminated in the body, and how these processes affect the concentration of the compound in the body, over the course of time.

In the discovery and preclinical phase of pharmaceutical drug development, animal models such as the mouse, rat, monkey, dog, or pig, may be used to perform this characterisation. Any of these models may be used to test the pharmacokinetic properties of the derivatives of the invention.

In such studies, animals are typically administered with a single dose of the drug, either intravenously, subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

For most compounds, the terminal part of the plasma-concentration profiles will be linear when drawn in a semi-logarithmic plot, reflecting that after the initial absorption and distribution, drug is removed from the body at a constant fractional rate. The rate (lambda Z or 2) is equal to minus the slope of the terminal part of the plot. From this rate, also a terminal half-life may be calculated, as t½=ln(2)/λ_{z} (see, e.g., Johan Gabrielsson and Daniel Weiner: Pharmacokinetics and Pharmacodynamic Data Analysis. Concepts & Applications, 3rd Ed., Swedish Pharmaceutical Press, Stockholm (2000)).

Clearance may be determined after i.v. administration and is defined as the dose (D) divided by area under the curve (AUC) on the plasma concentration versus time profile (Rowland, M and Tozer T N: Clinical Pharmacokinetics: Concepts and Applications, 3^{rd} edition, 1995 Williams Wilkins).

The estimate of terminal half-life and/or clearance is relevant for evaluation of dosing regimens and an important parameter in drug development, in the evaluation of new drug compounds.

### Method 9: Identifying “Absorbers” for Dog Studies

The oral exposure of acylated insulin, detectable in blood/plasma samples of Beagle dogs is known to vary from dog to dog. If a dog is not showing exposure, i.e. if no insulin is detectable in the blood/plasma samples after administration of oral insulin, then the dog is a “non-absorber”. When a dog however shows exposure, i.e. detectable values of acylated insulin in the blood/plasma samples are recognised, then this dog is an “absorber”.

For bioavailability studies “non-absorbers” are not excluded.

### Method 10: Testing Food Interaction

The testing of food interaction was investigated by sequential oral administration of pharmaceutical composition of this invention and food. The set-up was as this: A composition of this invention t was given orally according to the method described. After pre-defined intervals food was given to the dogs and bioavailability and pharmacokinetics were measured according to method 8 described above.

Method 11: Preparation of Uncoated Mini-Tablet Cores According to this Invention

The mini-tablet cores according to this invention were prepared by direct compression of one of the powder blends listed in Table 1a, 1b and 1c.

TABLE 1a
powder blend compositions of mini-tablet cores
Formulation 1A

| component | wt % |
|---|---|
| Acylated insulin | 1.68 |
| Sodium caprate | 77.46 |
| Sorbitol | 20.36 |
| Stearic acid | 0.5 |

TABLE 1b
powder blend compositions of mini-tablet cores
Formulation 1B

| component | wt % |
|---|---|
| Acylated insulin | 2.02 |
| Sodium caprate | 77.46 |
| Sorbitol | 20.01 |
| Stearic acid | 0.5 |

TABLE 1c
powder blend compositions of mini-tablet cores
Formulation 1C

| component | wt % |
|---|---|
| Acylated insulin A | 0.82 |
| Acylated insulin B | 0.83 |
| Sodium caprate | 77.46 |
| Sorbitol | 20.39 |
| Stearic acid | 0.5 |

The mini-tablet cores (i.e. tablet cores weighing 3.6 mg each) were prepared according to the following steps:

Powder blending: the acylated insulin analogue(s) and sorbitol were sieved through a 0.25 mm and 0.5 mm mesh sieves, respectively. After sieving, the total amount of acylated insulin analogue(s) and an equivalent amount of sorbitol were mixed by hand in an anti-static container. The remaining amount of diluent (sorbitol) was added to the previous powder blend by gradual additions. A final mechanical mixing was performed in Turbula at 32 rpm for 7 min.

Sodium caprate (in the form of granulate) was then added to the insulin-sorbitol mixture by gradual additions, and blended in a Turbula mixer.

Stearic acid, sieved through a 0.25 mm mesh size sieve, was accurately weighed and added to the previous powder mix.

Tableting: A rotary tablet press (Fette®) equipped with 1.5 mm (internal diameter) punches was used. Tableting was performed using a compression force of 3.2-3.9 KN, and at a rotation speed of 10 rpm. The mini-tablet cores produced had a diameter of 1.5 mm and a height between 2.0 and 2.5 mm. The average weight of each mini-table core was 3.6 mg.

For the preparation of bigger mini-tablets, the rotary tablet press was equipped with 4 mm punches. Tableting was performed using an average compression force of 3.7 KN and at a rotation speed of 10 rpm. The average height of the mini-tablet cores produced was 3.2 mm and the weight of 35.5 mg.

If different dimensions or weight/size of the mini-tablet cores is desired, the choice of ingredients should be adjusted with the same ratio between the ingredients and the punch size and form selected accordingly.

Method 12: Coating of Mini-Tablet Cores with a Polyvinyl Alcohol Coat, Such as OPADRY®II (as Sold in 2013)

Coating of mini-tablets was performed using a fluid bed apparatus equipped with a Wurster insert (mini-Glatt®, as sold in 2014) via the following steps:

Preparation of the coating solution: for the preparation of 100 g of coating solution, 20 g of Opadry® II (as sold by Colorcon® in 2014) were dispersed in 80 g RO water. The suspension was stirred using a standard magnetic stirrer for 30 minutes, and afterwards sieved to remove eventual lumps. The suspension was kept under stirring during the coating process.

Coating: Coating of mini-tablets was performed in a fluid bed apparatus equipped with a Wurster insert (mini-Glatt®, as sold in 2014). The fluid bed chamber was pre-heated until a temperature of 30-35° C. inside the chamber was reached. An accurately weighed amount of mini-tablets (20 g), prepared as described in method 1, was placed in the fluid bed chamber and warmed up for 2 min or until they reached 30° C. in temperature. Spray layering was performed by pumping the solution through a nozzle with an orifice of 0.8 mm, at an atomising pressure of 0.9 bar. The inlet air temperature, within the range 50-55° C., was adjusted throughout the process to keep the product temperature at 30-35° C. Coating was stopped when a coating level of 8 mg/cm2 (equivalent to a weight gain of 26%) was reached.

Drying of mini-tablets: mini-tablets were dried in the same equipment at 50° C. for 3 min.

If different dimensions or weight/size of the mini-tablet cores is coated, the amount of coating should be adjusted to the surface area.

### Method 13: Preparation of a Reference Monolith Tablet Cores for Comparison to Mini-Tablet Cores

A conventional monolith (19*8 mm) was prepared by direct compression of a powder blend described in table 1d.

TABLE 1d
composition of reference monolith core

| component | wt % |
|---|---|
| Acylated insulin | 1.68 |
| Sodium caprate | 77.46 |
| Sorbitol | 20.36 |
| Stearic acid | 0.5 |

The reference monoliths were prepared according to the following steps:

Powder blending: the powder blending step was the same as the one described for preparation of mini-tablet cores (see method 11).

Tableting: A rotary tablet press (Fette®) equipped with 19*8 mm punches was used. Tableting was performed using a compression force of 9-11 KN, and at a rotation speed of 10 rpm. The tablets produced had an average height of 6.3 mm and hardness of 120 kN.

To exemplify how midi-tablets and monolith-tablet cores were prepared. If smaller tablets, i.e weighing less and thus having smaller dimensions are desired, the ingredients have to be adjusted in the same ratios between the ingredients to a lower total weight and compressed into the desired tablet dimensions.

Method 14: Coating of Reference Monolith Tablet Cores for Comparison to Mini-Tablet Cores with a Polyvinyl Alcohol Coat, Such as OPADRY®II (as Sold in 2013)

Coating of monolith tablets was performed using a pan coater (O'Hara LabCoat M, as sold in 2013) via the following steps:

Preparation of the coating solution: the coating solution was prepared as described in method 12.

Coating: Coating of monolithic cores was performed in a pan coater, equipped with a pan size of 8.5″, and a conventional patterned air Schlick spray nozzle with an orifice of 1.0 mm. Coating was performed using an atomizing and pattern air pressure of 0.5 bar, inlet air temperature of 38° C. and air flow of 130 kg/hour. The pan coater chamber was pre-heated until a temperature of 30-35° C. inside the chamber was reached. An accurately weighed amount of tablets (230 g), prepared as described in method 3, was placed in pan coater chamber and warmed up until they reached 30° C. in temperature. Coating was stopped when a coating level of 8 mg/cm2 (equivalent to a weight gain of 4.5%) was reached.

Drying of mini-tablets: mini-tablets were dried in the same equipment at 50° C. for 10 min.

If different dimensions or weight/size of the tablet cores is coated, the amount of coating should be adjusted to the surface area.

Method 15: Preparation of a Capsule Dosage Form Containing Mini-Tablet or Monolith Cores According to this Invention

An accurately weighed amount of mini-tablet cores prepared as described in method 11, un-coated or coated as described in method 12, was manually filled into capsules (porcine gelatin, fish gelatin, HPMC or Pullulan). The amount of mini-tablets was chosen to have total insulin strength of 1600±100 nmol (equivalent to approx. 11.9 mg of acylated insulin) per capsule and an amount of sodium caprate of 550 mg or 450 mg, according to the experiment.

Monolith cores prepared as described in method 13, un-coated or coated as described in method 14, was filled manually into size 000 gelatin capsules.

Method 16: Compression of Mini-Tablets into a Fast-Disintegrating Monolith According to the Invention

Mini-table cores, prepared as described in method 11 (Formulation 1A) and coated with an OPADRY®II suspension up to 8 mg/cm2 according to method 12, were compressed into a fast disintegrating monolith. 894.6 mg of OPADRY®II coated mini-tablets (corresponding to 710 mg of un-coated cores) were admixed manually with 200 mg of microcrystalline cellulose (Avicel PH200, as sold in 2014) and 114 mg of Isolmalt 721 (as sold in 2013). The mini-tablet/powder mixture was compressed using a single punch tablet press (Diaf) equipped with a 9*18 mm punch. Each tablet was manufactured manually.

### Method 17: Determination of the Dissolution Rate In-Vitro

The dissolution set up was based on the USP Apparatus 1 (Basket Apparatus) using 100 ml of a 50 mM phosphate buffer, pH 6.8, as dissolution media. Quantification of samples was analysed using the Quantitative Method described below.

Quantitative Method: Quantitative determinations were performed using a C18 reversed phase liquid chromatography column and a TFA/CH3CN based eluent system. The content of the samples was calculated relative to a reference material of the compound to be tested.

Purity Method: For evaluation of the chemical stability, samples were analysed using a C18 reversed phase chromatographic column and a phosphate/CH3CN based eluent system. Purity was reported as the area.

### EXAMPLES

Unless otherwise stated coating or coating material described as OPADRY®II means OPADRY®II—Yellow.

### Example 1—Dissolution Rate of Compositions According to the Present Invention Comprising a Monolith Tablet Core with/without OPADRY®II—Yellow from Colorcon® (as Sold in 2013) and A14E, B25H, B29K(N^{ε}Octadecanedioyl-γGlu-OEG-OEG), desB30 Human Insulin

Monolith tablet cores were prepared by mixing ingredients of table 2 according to method 3, the dose of acylated insulin in dogs is in studies for the present patent application was set to 120 nmol/kg. Thus the absolute amount of acylated insulin in said tablet core was adjusted according to the weight of the dog which was to receive said tablet for oral administration. In the present example the dog weighed 18 kg and the insulin thus amounted to 14.8 mg (120 nmol/kg).

Dissolution was tested according to method 6.

One batch of monolith tablet cores was not coated. Another batch was coated according to method 4 with OPADRY®II—Yellow from Colorcon® (as sold in 2013).

Table 2 shows a composition according to the present invention comprising 14.8 mg A14E, B25H, B29K(N^{ε}Octadecanedioyl-γGlu-OEG-OEG), desB30 human insulin in a monolith tablet core comprising sodium caprate and is coated with OPADRY®II—Yellow coating from Colorcon® (as sold in 2013). The uncoated monolith tablet core weight was measured to 710.1 mg, the Opadry-coated tablet core, i.e. the finished monolith tablet weight was measured to 742.1 mg.

TABLE 2

| | | | Final |
|---|---|---|---|
| | | | coated |
| | | | tablet (% |
| | mg/ | Core | w/w) |
| Tablet Excipient | tablet | (% w/w) | Opadry ®II |
| A14E, B25H, | 14.8 | 2.1 | 2 |
| B29K(N^{ε}Octadecanedioyl-γGlu-OEG- | | | |
| OEG), desB30 human insulin | | | |
| Sodium caprate | 546.7 | 77 | 73.7 |
| Sorbitol | 145 | 20.4 | 19.5 |
| Stearic Acid | 3.6 | 0.5 | 0.5 |
| OPADRY ®II-Yellow | 32 | N/A | 4.3 |

The results are given in FIG. 1, which shows that the dissolution profiles of non-coated and Opadr®II coated tables are very similar, however the coated tablets had a slighty reduced dissolution rate relative to the non-coated ones.

### Example 2—Bioavailability and Tmax of A14E, B25H, B29K(N^{ε}Octadecanedioyl-γGlu-OEG-OEG), desB30 Human Insulin in Monolith Tablet Cores According to the Present Invention Coated with a OPADRY®II—Yellow from Colorcon® (as Sold in 2013) (Embodiment of the Present Invention) or Acryl-EZE® 93O from Colorcon® (as Sold in 2013) on Top of a Sub Coat of Opadry @II

The monolith tablet cores for this example were prepared by mixing ingredients according to table 1 (example 1) according to method 3. All tablet cores were coated with 4.5% (w/w) OPADRY®II—Yellow from Colorcon® (as sold in 2013) according to method 4 and the resulting composition will in this example be denominated “tablets”. One batch was left without further coating, whereas the other batch was further coated with 9% (w/w) Acryl-EZE® 93O coating from Colorcon® (as sold in 2013) according to method 5 on top of an OPADRY®II—Yellow coating from Colorcon® (as sold in 2013).

Samples for determining bioavailability where drawn according to method 6 in Beagle dogs.

FIG. 2A shows the bioavailability for A14E, B25H, B29K(N^{ε}Octadecanedioyl-γGlu-OEG-OEG), desB30 human insulin in tablets according to Table 2 with OPADRY®II—Yellow from Colorcon® (as sold in 2013); n=47 (checker) compared to the same insulin in tablet cores with Acryl-EZE® 93O coating from Colorcon® (as sold in 2013); n=24 (dotted).

FIG. 2B shows the Tmax for A14E, B25H, B29K(N^{ε}Octadecanedioyl-γGlu-OEG-OEG), desB30 human insulin in tablet cores according to Table 2 with OPADRY®II—Yellow from Colorcon® (as sold in 2013); n=47 (checker) compared to the same insulin in tablet cores with Acryl-EZE® 93O coating from Colorcon® (as sold in 2013); n=24 (dotted).

The results show that Bioavailability is increased and Tmax is decreased for A14E, B25H, B29K(N^{ε}Octadecanedioyl-γGlu-OEG-OEG), desB30 human insulin in tablet cores with OPADRY®II—Yellow from Colorcon® (as sold in 2013) relative to the ones with Acryl-EZE® 93O coating from Colorcon® (as sold in 2013). Statistical comparison was based on log (F) and log (Tmax).

### Example 3—Food Interaction and Bioavailability on A14E, B25H, B29K(N^{ε}Octadecanedioyl-γGlu-OEG-OEG), desB30 Human Insulin in Monolith Tablets According to the Present Invention

Monolith tablet cores according to the present invention were prepared according to table 3 and method 3 and coated according to method 4 and the resulting composition will in this example be denominated “tablets”. The tablets were administered to Beagle dogs and samples were collected as described in method 6. Food interaction was tested according to method 10.

The results are shown in table 3 below. The shorter the time between food intake and administration of said tablets, the more effect of food intake is seen on Bioavailability (F %) of A14E, B25H, B29K(N^{ε}Octadecanedioyl-γGlu-OEG-OEG), desB30 human insulin, but not as pronounced on Tmax for the formulation.

TABLE 3

| | Feeding post | Number of | | |
|---|---|---|---|---|
| | dosing | absorbers | Mean F | Median |
| Coating | (minuts) | (%) | (%) ± SD | Tₘₐₓ ± SD |
| OPADRY ®II- | 360 | 8 (100%) | 4.3 ± 3.0 | 53 ± 13 |
| Yellow | 60 | 8 (100%) | 2.9 ± 1.6 | 53 ± 17 |
| from | 30 | 8 (100%) | 1.6 ± 0.7 | 45 ± 8 |
| Colorcon ® | 15 | 8 (100%) | 1.2 ± 1.0 | 30 ± 8 |
| (as sold in | | | | |
| 2013) | | | | |

### Example 4—Real Time Stability Studies 0-12 Weeks Regarding the Bioavailability of A14E, B25H, B29K(N^{ε}Octadecanedioyl-γGlu-OEG-OEG), desB30 Human Insulin in a Monolith Tablet Core According to the Present Invention Coated with OPADRY®II—Yellow from Colorcon® (as Sold in 2013)

Monolith tablets (i.e. coated monolith tablet cores) with a composition of table 2 (example 1) were prepared according to method 3 and coated according to method 4 with OPADRY®II—Yellow from Colorcon® (as sold in 2013). The coated tablets were evaluated with respect to in-vivo performance stability. Thus tablets were produced and coated, packaged in duma-containers with a desiccant, stored at 5° C. and administered to Beagle dogs. Samples were collected as described in method 7.

Time points for this testing are specified in the table 3 to be 0, 3, 6, 9 and 12 weeks.

TABLE 4

| | | Bioavailability of A14E, B25H, | | | | | |
|---|---|---|---|---|---|---|---|
| | | B29K(N^{ε}Octadecanedioyl-γGlu-OEG-OEG), desB30 | | | | | |
| | | human insulin F(%) in dogs | | | | | |
| | | Week | | | | | |
| Coating | pH | 0 | 3 | 6 | 9 | 12 | mean |

| | | | | | | |
|---|---|---|---|---|---|---|
| OPADRY ®II -Yellow | 5.5 ± 3.2 | 3.0 ± 1.6 | 7.4 ± 5.4 | 3.4 ± 3.7 | 6.0 ± 3.4 | 5.1 ± 3.4 |
| from Colorcon ® | | | | | | |
| (as sold in 2013) | | | | | | |

FIG. 3 shows the PK profiles for the same insulin as tested above in tablet cores with OPADRY®II—Yellow from Colorcon® (as sold in 2013) as sub subcoat below an Eudragit®FS30D coating from Evonik Industries (as sold in 2013), squares show the PK profile for tablets tested at time 0 and circles show the PK profile for tablets tested after 14 weeks storage at 5° C. Mean±SEM; n=8. The PK profiles in this figure show that the bioavailability decreases upon storage of such compositions. Table 4 shows the bioavailability of the same insulin in OPADRY®II—Yellow from Colorcon® (as sold in 2013) coated monolith tablet cores. Comparing the results shown in FIG. 3 and table 4 it is evident that the bioavailability is surprisingly stable in compositions according to this invention relative to compositions also comprising an enteric coating.

### Example 5—Bioavailability of γGlu-OEG-OEG-γGlu Acylated Insulin in Monolith Tablet Cores According to the Present Invention

Monolith tablets (i.e. coated monolith tablet cores) according to the present invention were prepared according to table 2 (example 1) and method 3 and coated according to method 4. The tables were administered to 8 Beagle dogs and samples were collected as described in method 7. Results are shown in table 5.

| | Bioavailability |
|---|---|
| Insulin | F % |
| A10C, A14E, B3C, B25H, desB27, | 3.0 ± 3.9 |
| B29K(N(eps)octadecanedioyl-γGlu-OEG-OEG), | |
| desB30 human insulin | |
| A10C, A14E, B4C, B25H, B29K(N^{ε}Octadecanedioyl- | 2.4 ± 2.2 |
| γGlu-OEG-OEG), desB30 human insulin | |
| A14E, B25H, desB27, B29K(N(eps)octadecanedioyl- | 3.3 +/− 3.4 |
| γGlu-OEG-OEG), desB30 human insulin | |
| “B29K(N^{ε}Octadecanedioyl- | 0.3 +/− 0.2 |
| γGlu-OEG-OEG), desB30 human insulin | |

### Example 6—Bioavailability of Monolith Tablet Cores Comprising A14E, B25H, B29K(N^{ε}Octadecanedioyl-γGlu-OEG-OEG), desB30 Human Insulin with Enteric Coatings

Monolith tablet cores were prepared according to method 3 comprising A14E, B25H, B29K(N^{ε}Octadecanedioyl-γGlu-OEG-OEG), desB30 human insulin and coated either according to method 4 with OPADRY®II—Yellow (when sub coat was applied) and method 5 with EUDRAGIT®FS30D in combination or method 5 alone (when no sub coat was applied under said EUDRAGIT®FS30D coating). The bioavailability was tested at time 0 (i.e. shortly after the tablet preparation was completed) and after storage at 5° C. for 12 to 14 weeks after preparation was completed.

The results are given in table 6.

The bioavailability was assessed according to the method description of in-vivo experiments in method 8.

The dogs were fasted overnight before the test, (no food—only tap water). The day before the experiment the dogs were weighed and dogs were taken out for a couple of hours.

On the day of the experiment the dogs were placed on test couch and a Venflon 20 G was placed in v. cephalica. Blood samples were taken from the catheter. The venflon was removed 6 hours post dosing and the dogs were returned to their box, and offered exercise in the outside run. Hereafter the dogs were lead into a test room for blood sampling from v. jugularis (or v. cephalica).

Per os administration. Blood samples for glucose and insulin were taken at: 0, 15, 30, 45, 60, 75, 90, 105, 120, 135, 150, 165, 180, 210, 240, 270, 300, 360, 480, 600, 720, 1440, 1800, 2880 and 4320 minutes.

The tablet was administered right after the t=0 min sample was drawn. The tablet was placed in the back of the mouth so the dog would swallow the tablet without chewing it. After the dog had swallowed the tablet, 10 ml water was administered into the mouth by a syringe.

Blood sampling: Before sampling the first drops of blood was collected on a tissue. Approx. 800 μl blood was collected in 1.5 ml EDTA Eppendorf tubes for plasma and a 10 μL capillary tube was filled with full blood for glucose analysis. The EDTA blood samples were centrifuged at 4000×g (4° C.) for 4 min. All samples were kept on wet ice until analysis or stored at −80° C. until analysis. After each sampling, the Venflon was flushed with 0.5 ml heparin (10 IU). Male Beagle dogs weighed approximately from 12 to 18 kg. Plasma samples were analysed by either sandwich immunoassay or liquid chromatography-mass spectrometry (LC-MS). Plasma concentration-time profiles were analysed by non-compartmental pharmacokinetics analysis using WinNonlin Professional 5.2 (Pharsight Inc., Mountain View, Calif., USA).

TABLE 6

| | Bioavailability F (%) | |
|---|---|---|
| | | Time |
| Coating | Time 0 | 12-14 weeks |
| Opadry ® II (%) + Acryl-EZE ® | 2.3 ± 2.7% (n = 8) | N/A |
| 93O (%) | | |
| Opadry ® II (%) + Acryl-EZE ® | 1.5 ± 1.9% (n = 16) | N/A |
| 93A (%) | | |
| Opadry ® II + Eudragit ® | 7.4 ± 6.5% (n = 8) | 1.4 ± 1.5% |
| FS30D (%) | | (n = 8) |

### Example 7 In-Vitro Dissolution Rate of A14E, B25H, B29K(N^{ε}Octadecanedioyl-γGlu-OEG-OEG), desB30 Human Insulin from Capsules Containing Un-Coated Monolith Tablet Cores or Un-Coated Mini-Tablet Cores

Mini-tablet cores and monolith tablet cores were prepared as described in methods 11 and 13, respectively, and filled into size 000 porcine gelatin capsules according to method 15. A detailed composition of the multi-particulate and monolith capsule formulations is listed in Table 7.

TABLE 7
composition of size 000 porcine gelatin capsules containing
un-coated mini-tablet or monolith cores (*average weight
of each mini-tablet core was 3.6 mg)

| | mg/capsule | |
|---|---|---|
| | Monolith tablet | Mini-tablet |
| component | formulation | formulation |
| A14E, B25H, | 11.91 | 11.91 |
| B29K(N^{ε}Octadecanedioyl-γGlu-OEG- | | |
| OEG), desB30 human insulin | | |
| Sodium caprate | 550 | 550 |
| Sorbitol | 144.54 | 144.54 |
| Stearic Acid | 3.55 | 3.55 |
| Total | 710 | 710* |

The in-vitro dissolution rate of capsule formulations described in table 7 was determined according to method 16. Profiles are shown in FIG. 4, showing in-vitro dissolution rate of A14E, B25H, B29K(N^{ε}Octadecanedioyl-γGlu-OEG-OEG), desB30 human insulin (triangles) and sodium caprate (circles) from size 000 porcine gelatin capsules filled with mini-tablets (black lines) and monoliths (grey lines). Data are reported as mean (n=3)±SD.

In-vitro, the dissolution rate of un-coated mini-tablets was determined to be >3 folds higher than that of the equivalent monolith tablet cores in porcine gelatin capsules.

### Example 8 Bioavailability and Tₘₐₓ of A14E, B25H, B29K(N^{ε}Octadecanedioyl-γGlu-OEG-OEG), desB30 Human Insulin after Oral Administration of Porcine Gelatin Capsules Containing Un-Coated Mini-Tablet or Monolith Tablet Cores

Size 000 porcine gelatin capsules containing un-coated mini-tablet or monolith cores described in example 7, were dosed to male Beagle dogs. Oral bioavailability, Tmax and number of non-absorbers were determined according to methods 8 and 9. Results are shown in table 8.

TABLE 8
mean bioavailability (F), median Tₘₐₓ and number of
non-absorbers after oral administration of size 000 porcine
gelatin capsules containing un-coated mini-tablet cores or
monolith tablet cores.

| | | | | Non- |
|---|---|---|---|---|
| | Beagle | Mean F | Median Tₘₐₓ | absorbers |
| Formulation | dogs (n=) | (%) ± SD | (min) ± SD | (%) |
| Un-coated | 8 | 2.8 ± 1.8 | 45 ± 8 | 0 (0) |
| mini-tablets | | | | |
| in size 000 | | | | |
| porcine | | | | |
| gelatin | | | | |
| capsules | | | | |
| Un-coated | 8 | 2.0 ± 1.3 | 53 ± 11 | 0 (0) |
| monolith in | | | | |
| size 000 | | | | |
| porcine | | | | |
| gelatin | | | | |
| capsules | | | | |

The bioavailability and variation of A14E, B25H, B29K(N^{ε}Octadecanedioyl-γGlu-OEG-OEG), desB30 human insulin after oral administration of capsules containing monolith and mini-tablets was not significantly different. Tmax for mini-tablets was shorter than that of the equivalent monolithic formulation.

### Example 9 Food Interaction and Bioavailability on A14E, B25H, B29K(N^{ε}Octadecanedioyl-γGlu-OEG-OEG), desB30 Human Insulin after Oral Administration of Capsules Containing Un-Coated Mini-Tablets

Porcine gelatin capsules containing un-coated mini-tablet cores, as described in examples 7 and 8, were tested in a drug-food interaction study according to method 10. The results were compared to those of a similar food-interaction study performed on Opadry-II coated monolith (see example 3). Results are summarised in Table 9.

TABLE 9
Mean bioavailability (F), median Tₘₐₓ after oral administration
of Opadry-II coated monolith tablet cores or porcine gelatin
capsules containing un-coated mini-tablet cores. Dogs were
fed after 360 min, 60 min, 30 min or 15 min after dosing.

| | Mean F (%) ± SD | | Median Tₘₐₓ ± SD | |
|---|---|---|---|---|
| | | Un-coated | | Un-coated |
| | | mini- | | mini- |
| | Opadry-II | tablets in | | tablets in |
| | coated | porcine | Opadry-II | porcine |
| Feeding | monolith | gelatin | coated | gelatin |
| post dosing | talet | capsules | monolith | capsules |
| (min) | core (n = 8) | (n = 16) | (n = 8) | (n = 16) |
| 360 | 4.3 ± 3.0 | 2.2 ± 2.1 | 53 ± 13 | 47 ± 18 |
| 60 | 2.9 ± 1.6 | 2.8 ± 2.2 | 53 ± 17 | 43 ± 13 |
| 30 | 1.6 ± 0.7 | 2.4 ± 1.8 | 45 ± 8 | 48 ± 7 |
| 15 | 1.2 ± 1.0 | 1.0 ± 1.0 | 30 ± 8 | 38 ± 9 |

When the monolith tablet core was administered 60 min, 30 min and 15 min prior to feeding, a decrease in the acylated insulin bioavailability equal to 33%, 63% and 72% compared to dosing in the fasted state (360 min) was determined. Surprisingly, no decrease in bioavailability was observed when the mini-tablet formulation was dosed up to 30 min prior to feeding. A decrease in the acylated insulin bioavailability, equal to 55%, was only determined when dogs were fed 15 min after dosing. Un-coated mini-tablet formulation was proven to mitigate drug-food interaction compare to a monolithic dosage form with the same insulin and sodium caprate strength.

### Example 10 In-Vitro Dissolution Rate of A14E, B25H, B29K(N^{ε}Octadecanedioyl-γGlu-OEG-OEG), desB30 Human Insulin from Porcine Gelatin Capsules Containing Opadry-II-Coated Monolith or Mini-Tablet Cores

Mini-tablet cores were prepared as described in method 11 (formulation 1A) and coated with an Opadry-II suspension according to method 12, up to a coating level of 8 mg/cm2 (corresponding to a weight gain of 26%). Monolith tablets were prepared as described in method 13 and coated with Opadry-II yellow suspension as described in method 14, up to a coating level of 8 mg/cm2 (corresponding to a weight gain of 4.5%).

Opadry-II coated mini-tablet cores and Opadry-II coated monolith tablet cores were filled into size 000 porcine gelatin capsules as described in method 15. A detailed composition of the formulations tested in listed in Table 10.

TABLE 10
Composition of size 000 porcine gelatin capsules filled
with Opadry-II coated mini-tablet cores or Opadry-II
coated monolith tablet cores.

| | mg/capsule | |
|---|---|---|
| | | Opadry-II |
| | Opadry-II | coated mini- |
| | coated monolith | tablet |
| component | formulation | formulation |
| A14E, B25H, | 11.91 | 11.91 |
| B29K(N^{ε}Octadecanedioyl-γGlu-OEG- | | |
| OEG), desB30 human insulin | | |
| Sodium caprate | 550 | 550 |
| Sorbitol | 144.54 | 144.54 |
| Stearic Acid | 3.55 | 3.55 |
| Opadry-II Yellow (as sold in 2013) | 31.95 | 184.6 |
| tot | 741.95 | 894.6 |

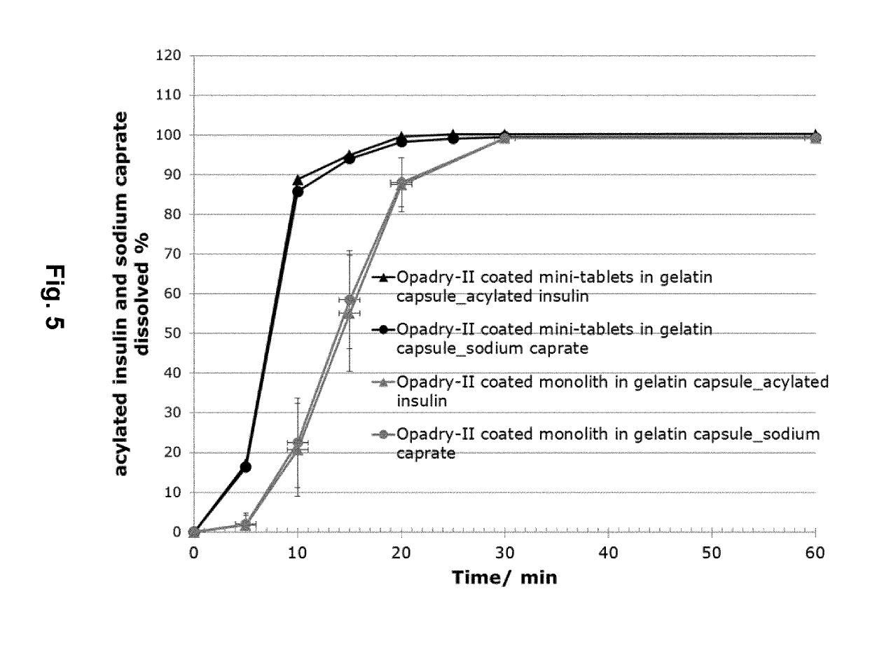

The in-vitro dissolution rate of Opadry-II coated monolith tablet cores and Opadry-II coated mini-tablet formulations was tested according to method 17. Profiles are shown in FIG. 5, showing in-vitro dissolution rate of A14E, B25H, B29K(N^{ε}Octadecanedioyl-γGlu-OEG-OEG), desB30 human insulin (triangles) and sodium caprate (circles) from size 000 porcine gelatin capsules containing Opadry-II coated mini-tablets (black lines) and monoliths (dark grey lines). Data are reported as mean (n=3)±SD.

The dissolution rate from Opadry-II coated mini-tablets was slightly slower than the equivalent un-coated formulation (example 7). However, the dissolution rate of Opadry-II coated mini-tablets in porcine gelatin capsules was >2 folds higher than that of the equivalent monolith table cores in porcine gelatin capsules.

### Example 11 Bioavailability and Tₘₐₓ of A14E, B25H, B29K(N^{ε}Octadecanedioyl-γGlu-OEG-OEG), desB30 Human Insulin after Oral Administration of Porcine Gelatin Capsules Containing Opadry-II-Coated Mini-Tablet and Opadry-II-Coated Monolith Tablet Cores

Size 000 porcine gelatin capsules containing Opadry-II-coated mini-tablet or monolith cores described in example 10, were dosed to male Beagle dogs. Oral bioavailability, Tmax and number of non-absorbers were determined according to methods 8 and 9. Results are shown in table 11.

TABLE 11
mean bioavailability (F), median Tₘₐₓ and number of
non-absorbers after oral administration of size 000 porcine
gelatin capsules containing Opadry-II coated mini-tablets
or Opadry-II coated monolith tablet cores.

| | Beagle | | | Non- |
|---|---|---|---|---|
| | dogs | Mean F | Median Tₘₐₓ | absorbers |
| Formulation | (n=) | (%) ± SD | (min) ± SD | (%) |
| Opadry-II | 8 | 3.0 ± 1.8 | 45 ± 5 | 0 (0%) |
| coated mini- | | | | |
| tablets in | | | | |
| porcine | | | | |
| gelatin | | | | |
| capsules | | | | |
| Opadry-II- | 8 | 0.96 ± 1.2 | 45 ± 18 | 1 (12%) |
| coated | | | | |
| monolith in | | | | |
| porcine | | | | |
| gelatin | | | | |
| capsules | | | | |

Bioavailability of A14E, B25H, B29K(N^{ε}Octadecanedioyl-γGlu-OEG-OEG), desB30 human insulin after oral administration of a capsule containing Opadry-II coated mini-tablets was determined to be significantly higher than that of the equivalent monolith formulation.

### Example 12 In-Vitro Dissolution Rate of A14E, B25H, B29K(N^{ε}Octadecanedioyl-γGlu-OEG-OEG), desB30 Human Insulin and Sodium Caprate from Opadry-II-Coated Mini-Tablet Cores Compressed in Fast-Disintegrating Monolith Tablets

Mini-tablet cores were prepared as described in method 11 (formulation 1A) and coated with Opadry-II Yellow up to a coating level of 8 mg/cm2, as described in method 12. Coated mini-tablet cores were compressed into a fast-disintegrating monolith as described in method 16. A detailed description of the formulation is listed in Table 12.

TABLE 12
composition of fast-disintegrating monoliths containing
Opadry-II coated mini-tablet cores.

| component | mg/tablet |
|---|---|
| A14E, B25H, B29K(N^{ε}Octadecanedioyl-γGlu-OEG- | 11.91 |
| OEG), desB30 human insulin | |
| Sodium caprate | 550 |
| Sorbitol | 144.54 |
| Stearic Acid | 3.55 |
| Opadry-II Yellow (as sold in 2013) | 184.6 |
| Microcrystalline cellulose (Avicel PH200, as sold | 200 |
| in 2013) | |
| Isomalt 721 (as sold in 2013) | 114 |
| Total | 1208.6 |

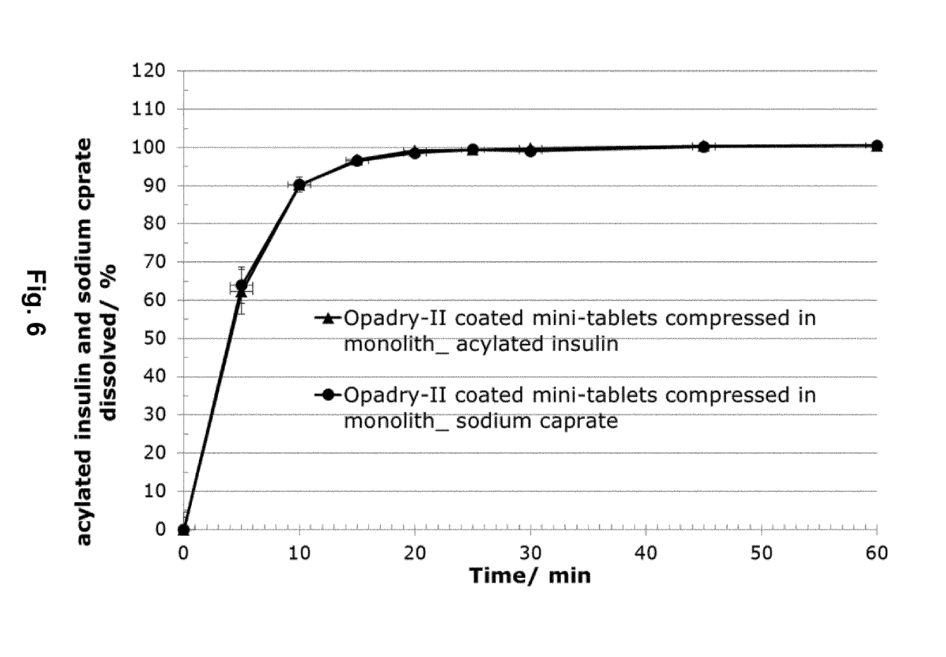

The in-vitro dissolution rate of compressed mini-tablets, tested as described in method 17, is shown in FIG. 6, showing in-vitro dissolution rate of A14E, B25H, B29K(N^{ε}Octadecanedioyl-γGlu-OEG-OEG), desB30 human insulin (triangles) and sodium caprate (circles) from Opadry-II coated mini-tablets compressed in a monolith. Data are reported as mean (n=3)±SD.

Faster dissolution onset of the acylated insulin and sodium caprate was determined for mini-tablet tablet cores compressed in the fast-disintegrating monolith compared to the equivalent capsule formulation. (See example 10, FIG. 2).

### Example 13 Bioavailability and Tₘₐₓ of A14E, B25H, B29K(N^{ε}Octadecanedioyl-γGlu-OEG-OEG), desB30 Human Insulin after Oral Administration of Opadry-II-Coated Mini-Tablet Cores Compressed in Fast-Disintegrating Monolith Tablets

Fast-disintegrating monolith tablets consisting of Opadry-II-coated mini-tablet cores described in example 12, were dosed to male Beagle dogs. Oral bioavailability, Tmax and number of non-absorbers were determined according to methods 7 and 8. Results are shown in table 13.

TABLE 13
mean bioavailability (F), median Tₘₐₓ and number of non-
absorbers after oral administration of fast-disintegrating
monoliths consisting of Opadry-II coated mini-tablet cores.

| | Beagle | | | Non- |
|---|---|---|---|---|
| | dogs | Mean F | Median Tₘₐₓ | absorbers |
| Formulation | (n=) | (%) ± SD | (min) ± SD | (%) |
| Opadry-II | 8 | 4.0 ± 2.4 | 60 ± 17 | 0 (0%) |
| coated mini- | | | | |
| tablets in fast- | | | | |
| disintegrating | | | | |
| monolith | | | | |

Surprisingly, bioavailability of A14E, B25H, B29K(N^{ε}Octadecanedioyl-γGlu-OEG-OEG), desB30 human insulin after oral administration of Opadry-II coated mini-tablets compressed in a fast-disintegrating monolith was determined to be approx. 33% higher (not statistically significant) than that of the equivalent cores delivered in a porcine gelatin capsule (see Table 11, example 11). Tmax of compressed mini-tablets was also higher than that of the encapsulated ones.

### Example 14 In-Vitro Dissolution Rate of A14E, B25H, B29K(N^{ε}Octadecanedioyl-γGlu-OEG-OEG), desB30 Human Insulin from Un-Coated Mini-Tablet Cores in Different Capsule Materials

Mini-tablet cores were prepared as described in method 11 (formulation 1B) and filled into size 00 capsules, as described in method 15. Different capsules materials were investigated, namely porcine gelatin (Licaps, as sold in 2014), fish gelatin (EMBO CAPS, as sold in 2014), HPMC (Vcaps plus, as sold in 2014) and pullulan (Plantcaps, as sold in 2014). A detailed description of the different capsule formulations is listed in Table 14.

TABLE 14
composition of size 00 capsules containing un-coated mini-
tablet cores.

| | mg/capsule | | | |
|---|---|---|---|---|
| | Porcine | Fish | | |
| Component | gelatin | gelatin | HPMC | Pullulan |
| A14E, B25H, | 11.91 | 11.91 | 11.91 | 11.91 |
| B29K(N^{ε}Octadecanedioyl- | | | | |
| γGlu-OEG-OEG), desB30 | | | | |
| human insulin | | | | |
| Sodium caprate | 455.49 | 455.49 | 455.49 | 455.49 |
| Sorbitol | 117.66 | 117.66 | 117.66 | 117.66 |
| Stearic Acid | 2.94 | 2.94 | 2.94 | 2.94 |
| Total | 588 | 588 | 588 | 588 |

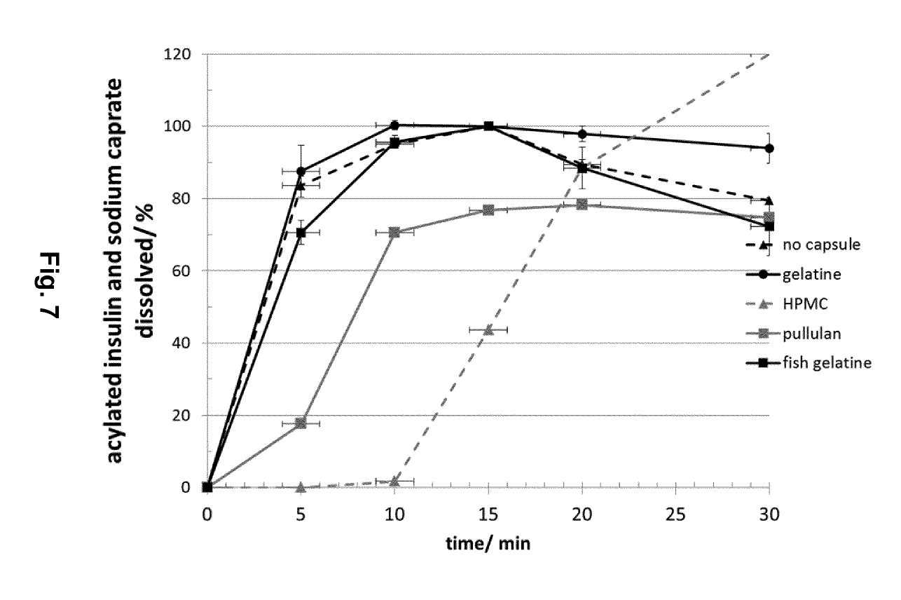

FIG. 7 shows in-vitro dissolution rate of A14E, B25H, B29K(N^{ε}Octadecanedioyl-γGlu-OEG-OEG), desB30 human insulin from un-coated mini-tablets without capsule (black dotted line, triangles) or filled into size 00 capsules: porcine gelatin (black line, circles), HPMC (grey dotted line, triangles), Pullulan (grey line, squares) and fish gelatin (black line, squares). Data are reported as mean (n=3)±SD. FIG. 7 shows that no significant differences in the dissolution rate of acylated insulin from mini-tablets filled in porcine gelatin capsules compared to that of mini-tablets tested without capsules were observed. The performance of fish gelatin capsules was similar to that of the porcine gelatin ones.

Longer lag-time and sustained-release profile were observed with HPMC capsules. A delayed-release of the insulin analogue was also observed with mini-tablets filled in pullulan capsules.

Overall the dissolution rate of acylated insulin from mini-tablets filled in different capsule materials was the following:

porcine gelatin>fish gelatin>Pullulan>HPMC

All the capsules tested were suitable for the development of a multi-particulate formulation based on mini-tablet cores.

### Example 15 Bioavailability and Tₘₐₓ of A14E, B25H, B29K(N^{ε}Octadecanedioyl-γGlu-OEG-OEG), desB30 Human Insulin after Oral Administration of Un-Coated Mini-Tablet Cores Filled in Different Capsule Materials

Different capsule formulations containing un-coated mini-tablets as described in example 14 were tested in male Beagle dogs. Oral bioavailability, Tmax and number of non-absorbers were determined according to methods 7 and 8. Results are shown in table 15.

TABLE 15
mean bioavailability (F), median Tₘₐₓ and number
of non-absorbers after oral administration of different
capsules filled with un-coated mini-tablet cores.

| | Beagle | | | Non- |
|---|---|---|---|---|
| | dogs | Mean F | Median Tₘₐₓ | absorbers |
| Formulation | (n=) | (%) ± SD | (min) ± SD | (%) |
| Un-coated mini- | 16 | 1.6 ± 2.1 | 45 ± 22 | 0 (0%) |
| tablets in | | | | |
| porcine gelatin | | | | |
| capsules | | | | |
| Un-coated mini- | 8 | 1.8 ± 2.1 | 53 ± 38 | 0 (0%) |
| tablets in fish | | | | |
| gelatin capsules | | | | |
| Un-coated mini- | 8 | 3.3 ± 3.8 | 75 ± 33 | 0 (0%) |
| tablets in HPMC | | | | |
| capsules | | | | |
| Un-coated mini- | 16 | 2.5 ± 2.1 | 45 ± 22 | 0 (0%) |
| tablets in | | | | |
| Pullulan | | | | |
| capsules | | | | |

Bioavailability of A14E, B25H, B29K(N^{ε}Octadecanedioyl-γGlu-OEG-OEG), desB30 human insulin after oral administration of size 00 porcine gelatin capsules filled with un-coated mini-tablets containing 450 mg of sodium caprate was determined to be approx. 43% lower (not significantly different) than that of un-coated cores containing 550 mg of the enhancer (table, example 2). Similar bioavailability and variation was determined for the fish gelatin capsules. On the other hand, higher bioavailability (not significant) was determined for pullulan and HPMC capsule formulations. A longer Tmax, in line with longer lag time observed in-vivo (example 14), was observed with HPMC capsules.

### Example 16 In-Vitro Dissolution Rate of A14E, B25H, B29K(N^{ε}Octadecanedioyl-γGlu-OEG-OEG), desB30 Human Insulin, A14E, B25H, desB27, B29K(N-(Eps)-(Octadecandioyl-gGlu-2×OEG), desB30 Human Insulin and Sodium Caprate from Porcine Gelatin Capsules Containing Un-Coated Mini-Tablet Cores

Mini-tablet cores were prepared as described in method 11 (formulation 1C) and filled into size 000 capsules, as described in method 15. A detailed description of the different capsule formulations is listed in Table 16.

TABLE 16
composition of size 000 porcine gelatin capsules containing
un-coated mini-tablet cores.

| component | mg/capsule |
|---|---|
| A14E, B25H, B29K(N^{ε}Octadecanedioyl-γGlu-OEG- | 5.82 |
| OEG), desB30 human insulin | |
| A14E, B25H, desB27, B29K(N-(eps)-(octadecandioyl- | 5.86 |
| gGlu-2xOEG), desB30 human insulin | |
| Sodium caprate | 550 |
| Sorbitol | 144.7 |
| Stearic Acid | 3.55 |
| Total | 710 |

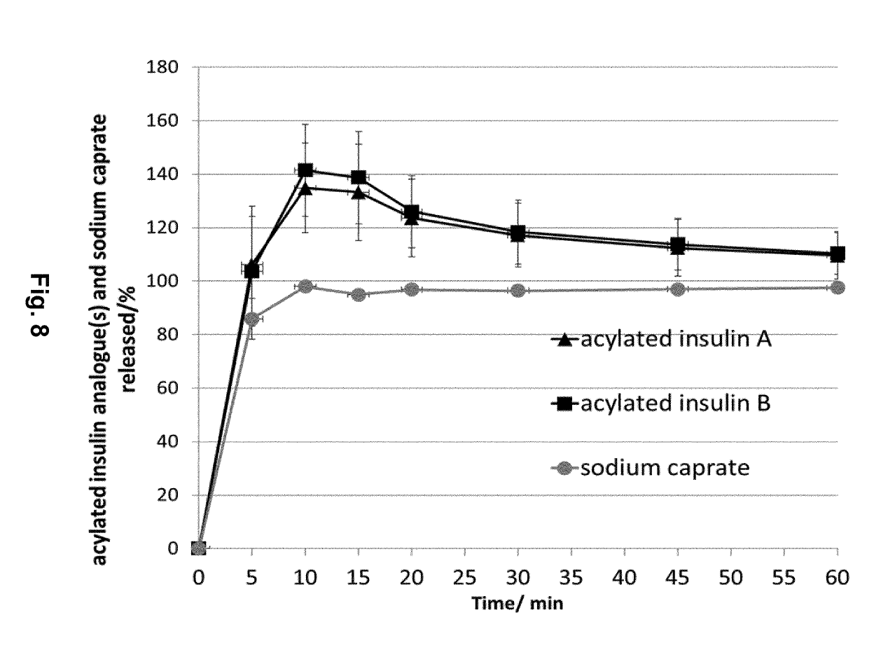

The in-vitro dissolution rate of capsules containing un-coated mini-tablet cores was tested according to method 17. Profiles are shown in FIG. 8, showing in-vitro dissolution rate from un-coated mini-tablets filled in size 000 porcine gelatin capsules of: 1) acylated insulin A (A14E, B25H, B29K(N^{ε}Octadecanedioyl-γGlu-OEG-OEG), desB30 human insulin) (black line, triangles); 2) acylated insulin B (A14E, B25H, desB27, B29K(N-(eps)-(octadecandioyl-gGlu-2×OEG), desB30 human insulin) (black line, squares) and 3) sodium caprate (grey line, circles). Data are reported as mean (n=3)±SD.

### Example 17 Bioavailability and Tₘₐₓ of A14E, B25H, B29K(N^{ε}Octade-Canedioyl-γGlu-OEG-OEG), desB30 Human Insulin and A14E, B25H, desB27, B29K(N-(Eps)-(Octadecandioyl-gGlu-2×OEG), desB30 Human Insulin from Porcine Gelatin Capsules Containing Un-Coated Mini-Tablet Cores

Mini-tablet cores described in example 16 were tested in male Beagle dogs. Oral bioavailability, Tmax and number of non-absorbers were determined according to methods 7 and 8. Results are shown in table 17.

TABLE 17
mean bioavailability (F), median Tₘₐₓ and number of non-absorbers after
oral administration of size 000 capsules containing un-coated mini-tablet cores.

| | Beagle | | | Median | Non- |
|---|---|---|---|---|---|
| | dogs | | Mean F | Tₘₐₓ | absorbers |
| Formulation | (n=) | acylated insulin | (%) ± SD | (min) ± SD | (%) |
| Un- | 8 | A14E, B25H, | 3.0 ± 3.0 | 46 ± 21 | 0 |
| coated | | B29K(N^{ε}Octadecanedioyl- | | | (0%) |
| mini- | | γGlu-OEG-OEG), desB30 | | | |
| tablets in | | human insulin | | | |
| porcine | | A14E, B25H, desB27, | 5.4 ± 5.1 | 46 ± 22 | 0 |
| gelatin | | B29K(N-(eps)- | | | (0%) |
| capsules | | (octadecandioyl-gGlu- | | | |
| | | 2xOEG), desB30 human | | | |
| | | insulin | | | |

### Example 18 In-Vitro Dissolution Rate of A14E, B16H, B25H, B29K(N-(Eps)-(Eicosanedioyl-gGlu-2×OEG), desB30 Human Insulin and Sodium Caprate from Porcine Gelatin Capsules Containing Un-Coated Mini-Tablet Cores

Mini-tablet cores were prepared as described in method 11 (formulation 1A) and filled into size 000 capsules, as described in method 15. A detailed description of the capsule formulation is listed in Table 18.

TABLE 18
composition of size 000 capsules containing un-coated mini-
tablet cores.

| component | mg/capsule |
|---|---|
| A14E, B16H, B25H, B29K(N-(eps)-(eicosanedioyl-gGlu- | 11.91 |
| 2xOEG), desB30 human insulin | |
| Sodium caprate | 550 |
| Sorbitol | 144.54 |
| Stearic Acid | 3.55 |
| Total | 710 |

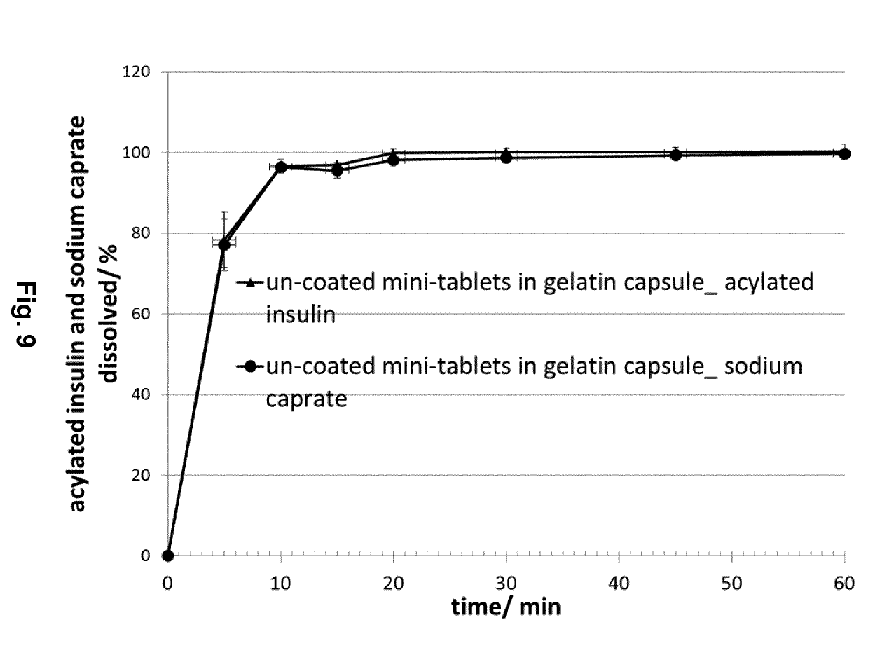

The in-vitro dissolution rate of capsules containing un-coated mini-tablet cores was tested according to method 17. Profiles are shown in FIG. 9, showing in-vitro dissolution rate of A14E, B16H, B25H, B29K(N-(eps)-(eicosanedioyl-gGlu-2×OEG), desB30 human insulin (triangles) and sodium caprate (circles) from size 000 porcine gelatin capsules containing un-coated mini-tablet cores. Data are reported as mean (n=3)±SD.

### Example 19 In-Vitro Dissolution Rate of A14E, B25H, desB27, B29K(N-(Eps)-(Octadecandioyl-gGlu), desB30 Human Insulin and Sodium Caprate from Porcine Gelatin Capsules Containing Un-Coated Mini-Tablet Cores

Mini-tablet cores were prepared as described in method 11 (formulation 1A) and filled into size 000 capsules, as described in method 15. A detailed description of the capsule formulation is listed in Table 19.

TABLE 19
composition of size 000 capsules containing un-coated mini-
tablet cores.

| component | mg/capsule |
|---|---|
| A14E, B25H, desB27, B29K(N-(eps)-(octadecandioyl- | 11.91 |
| gGlu), desB30 human insulin | |
| Sodium caprate | 550 |
| Sorbitol | 144.54 |
| Stearic Acid | 3.55 |
| Total | 710 |

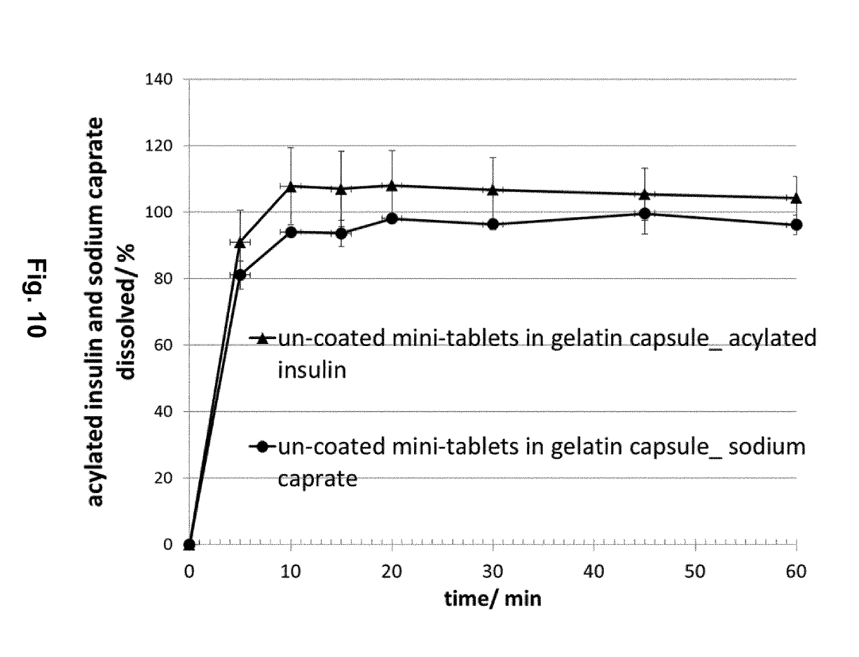

The in-vitro dissolution rate of capsules containing un-coated mini-tablet cores was tested according to method 17. Profiles are shown in FIG. 10, showing in-vitro dissolution rate of A14E, B25H, desB27, B29K(N-(eps)-(octadecandioyl-gGlu), desB30 human insulin (triangles) and sodium caprate (circles) from size 000 porcine gelatin capsules containing un-coated mini-tablets. Data are reported as mean (n=3)±SD.

### Example 20 In-Vitro Dissolution Rate of A14E, B25H, desB27, B29K(N-(Eps)-(Octadecandioyl-gGlu-2×OEG), desB30 Human Insulin and Sodium Caprate from Porcine Gelatin Capsules Containing 4.0 mm Un-Coated Mini-Tablet Cores

4.0 mm mini-tablet cores were prepared as described in method 11 (formulation 1A) and filled into size 000 capsules, as described in method 15. A detailed description of the capsule formulation is listed in Table 20.

TABLE 20
composition of size 000 capsules containing un-coated 4.0
mini-tablet cores.

| component | mg/capsule |
|---|---|
| A14E, B25H, desB27, B29K(N-(eps)-(octadecandioyl- | 11.91 |
| gGlu), desB30 human insulin | |
| Sodium caprate | 550 |
| Sorbitol | 144.54 |
| Stearic Acid | 3.55 |
| Total | 710 |

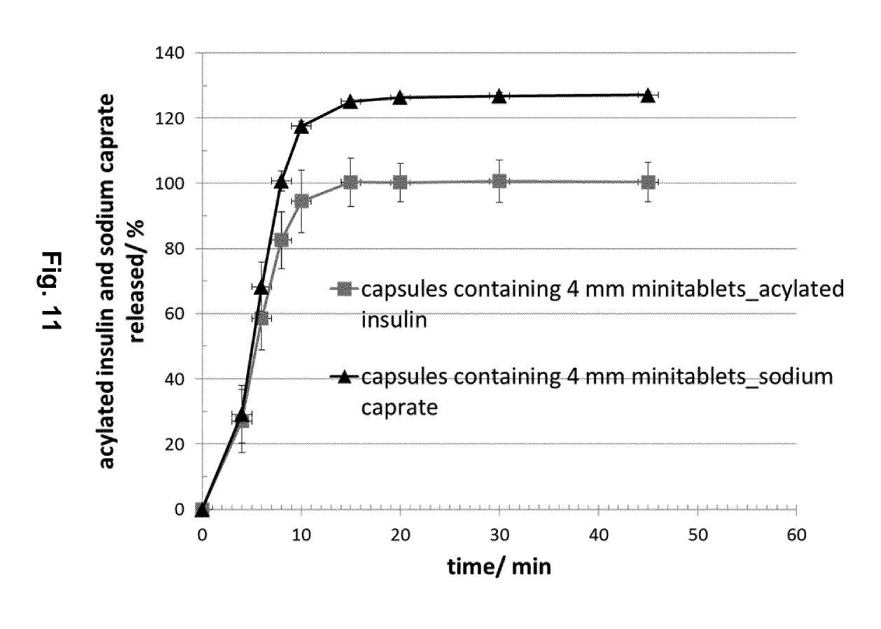

The in-vitro dissolution rate of capsules containing un-coated mini-tablets was tested according to method 17. Profiles are shown in FIG. 11, showing in-vitro dissolution rate of A14E, B25H, desB27, B29K(N-(eps)-(octadecandioyl-gGlu-2×OEG), desB30 human insulin (triangles, black line) and sodium caprate (squares, grey line) from size 000 porcine gelatin capsules containing un-4.0 mm coated mini-tablets. Data are reported as mean (n=3)±SD.

### Example 21—Bioavailability and Tmax of A14E, B25H, B29K(N^{ε}Octadecanedioyl-γGlu-OEG-OEG), desB30 Human Insulin in the Form of Six Tablet Cores According to the Present Invention in a Gelatin Capsule (Embodiment of the Present Invention)

Tablet cores for this example were prepared by mixing ingredients according to table 1 (example 1) according to method 3. Individual tablets were then compressed into midi-tablets to a weight of 118 mg each. Six tablets the uncoated version were placed in hard gelatin capsules.

The tablets in capsules were administered to 16 Beagle dogs and samples were collected as described in method 6. Results are shown in table 21.

TABLE 21

| | Bioavailability |
|---|---|
| Formulation | F % ± SD, CV % |
| A10C, A14E, B4C, B25H, B29K(N^{ε}Octadecanedioyl- | 1.9 ± 1.1, 60% |
| γGlu-OEG-OEG), desB30 human insulin | |

1. A pharmaceutical composition comprising one or more tablet core and optionally a polyvinyl alcohol coating, wherein said one or more tablet core comprises a salt of a medium-chain fatty acid and an acylated insulin,
wherein said acylated insulin comprises an additional disulfide bridge, or, wherein said acylated insulin is a protease stabilised insulin comprising a linker and a fatty acid or fatty diacid side chain having 14-22 carbon atoms and optionally comprises an additional disulfide bond.

2. The pharmaceutical composition according to claim 1, wherein said polyvinyl alcohol coating dissolves in aqueous medium at any pH.

3. The pharmaceutical composition according to claim 1, wherein said polyvinyl alcohol coating is OPADRY®II—Yellow from Colorcon® comprising polyvinyl alcohol (as sold in 2013).

4. The pharmaceutical composition according to claim 1, wherein said salt of a medium-chain fatty acid is a salt of capric acid.

5. The pharmaceutical composition according to claim 1, wherein said tablet core further comprises sorbitol, stearic acid and insulin and optionally further pharmaceutically acceptable excipients.

6. The pharmaceutical composition according to claim 1, wherein said tablet core comprises about 50-85% (w/w) sodium caprate.

7. The pharmaceutical composition according to claim 1, wherein said polyvinyl alcohol coating is present in at amount of about 0-10% (w/w) relative to said tablet core.

8. The pharmaceutical composition according to claim 4, wherein said salt of capric acid is sodium caprate.

9. The pharmaceutical composition according to claim 1, wherein said acylated insulin comprising a fatty acid or fatty diacid side chain having 18 or 20 carbon atoms.

10. The pharmaceutical composition according to claim 1, wherein said acylated insulin is selected from the group consisting of:
A14E,B25H,B29K(N^{ε}Octadecanedioyl-7Glu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N(eps)Eicosanedioyl-γGlu-OEG-OEG),desB30 human insulin,
A14E,B25H,desB27,B29K(N^{ε}Octadecanedioyl-7Glu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-7Glu),desB30 human insulin,
A14E,B25H,desB27,B29K(N^{ε}Eicosanedioyl-7Glu),desB30 human insulin and
A14E,B25H,desB27,B29K(N^{ε}Eicosanedioyl-7Glu-OEG-OEG),desB30 human insulin,
A10C,A14E,B4C,B25H,B29K(N^{ε}Octadecanedioyl-γGlu-OEG-OEG),desB30 human insulin,
A10C,A14E,B3C,B25H,B29K(N(eps)Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N(eps)octadecanedioyl-γGlu-OEG-OEG),desB30 human insulin,
A10C,A14E,B3C,B25H,desB27, B29K(N(eps)eicosanedioyl-γGlu-OEG-OEG),desB30 human insulin,
A10C,A14E,B3C,B16H,B25H,B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N(eps)eicosanedioyl-γGlu-OEG-OEG),desB30 human insulin.

11. The pharmaceutical composition according to claim 1 in the form of a tablet or a capsule comprising one or more tablet cores.

12. The pharmaceutical composition according to claim 1, wherein said tablet core weighs between about 1.5-50 mg, about 100-600 mg, about 600-900 mg or about 600-1300 mg.

13. (canceled)

14. (canceled)

15. A method for producing a pharmaceutical composition according to claim 1, comprising the steps of preparing a tablet core and coating of said polyvinyl alcohol coating on said outer surface of said tablet core.

16. A method of treating diabetes mellitus comprising administering the pharmaceutical composition of claim 1 to a patient in need thereof.
